SEQUENCE LISTING (102 sequences)

SEQ ID NO: 1 - DNA, 357 bp, Artificial Sequence
  Description of Artificial Sequence Synthetic polynucleotide
gaggtgcagctggtggagtctggggggggcttggtaaagcctggggggtcccttagactctcctgtgcagcctctggattcactttcagtaacacctggatgagctgggtccgccaggctccagggaaggggctggagtgggttggccgtattaaaagcaaatctgatggtgggacaacagactacgctgcacccgtgaaaggcagattcaccatctcaagagatgattcaaaaaacacgctgtatctgcaaatgaacagcctgaaaaccgaggacacagccgtgtattactgtacccagtattattggaactcctttgactactggggccagggaaccctggtcaccgtctcctca

SEQ ID NO: 2 - protein, 119 aa, Artificial Sequence
  Description of Artificial Sequence Synthetic polypeptide
EVQLVESGGGLVKPGGSLRLSCAASGFTFSNTWMSWVRQAPGKGLEWVGRIKSKSDGGTTDYAAPVKGRFTISRDDSKNTLYLQMNSLKTEDTAVYYCTQYYWNSFDYWGQGTLVTVSS

SEQ ID NO: 3 - protein, 8 aa, Artificial Sequence
  Description of Artificial Sequence Synthetic peptide
GFTFSNTW

SEQ ID NO: 4 - protein, 10 aa, Artificial Sequence
  Description of Artificial Sequence Synthetic peptide
IKSKSDGGTT

SEQ ID NO: 5 - protein, 10 aa, Artificial Sequence
  Description of Artificial Sequence Synthetic peptide
TQYYWNSFDY

SEQ ID NO: 6 - DNA, 321 bp, Artificial Sequence
  Description of Artificial Sequence Synthetic polynucleotide
gacatccagatgacccagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatattgcaacatattactgtcaacagtatgataatctcccgctcactttcggcggagggaccaaggtggagatcaaa

SEQ ID NO: 7 - protein, 107 aa, Artificial Sequence
  Description of Artificial Sequence Synthetic polypeptide
DIQMTQSPSSLSASVGDRVTITCQASQDISNYLNWYQQKPGKAPKLLIYDASNLETGVPSRFSGSGSGTDFTFTISSLQPEDIATYYCQQYDNLPLTFGGGTKVEIK

SEQ ID NO: 8 - protein, 11 aa, Artificial Sequence
  Description of Artificial Sequence Synthetic peptide
QASQDISNYLN

SEQ ID NO: 9 - protein, 7 aa, Artificial Sequence
  Description of Artificial Sequence Synthetic peptide
DASNLET

SEQ ID NO: 10 - protein, 9 aa, Artificial Sequence
  Description of Artificial Sequence Synthetic peptide
QQYDNLPLT

SEQ ID NO: 11 - DNA, 354 bp, Artificial Sequence
  Description of Artificial Sequence Synthetic polynucleotide
gaggtgcagctggtggagtctgggggaggcttggtccagcctggggggtcccagagactctcctgtgcagcctctggattcacctttagtagctattggatgaactgggtccgccaggctccagggaaggggctggagtgggtggccaacataaaggaagatggaagtgagaaatactatgtggactctgtgaagggccgattcaccatctccagagacaacgccaagaactcactgtatctgcaaatgaacagcctgagagccgaggacacggctgtgtattactgtgcgagagatccgggctgggctccctttgactactggggccagggaaccctggtcaccgtctcctca

SEQ ID NO: 12 - protein, 118 aa, Artificial Sequence
  Description of Artificial Sequence Synthetic polypeptide
EVQLVESGGGLVQPGGSQRLSCAASGFTFSSYWMNWVRQAPGKGLEWVANIKEDGSEKYYVDSVKGRFTISRDNAKNSLYLQMNSLRAEDTAVYYCARDPGWAPFDYWGQGTLVTVSS

SEQ ID NO: 13 - protein, 7 aa, Artificial Sequence
  Description of Artificial Sequence Synthetic peptide
GFTFSSY

SEQ ID NO: 14 - protein, 6 aa, Artificial Sequence
  Description of Artificial Sequence Synthetic peptide
KEDGSE

SEQ ID NO: 15 - protein, 9 aa, Artificial Sequence
  Description of Artificial Sequence Synthetic peptide
DPGWAPFDY

SEQ ID NO: 16 - DNA, 321 bp, Artificial Sequence
  Description of Artificial Sequence Synthetic polynucleotide
gacatccagatgtcccagtctccatcctcactgtctgcatctgtaggagacagagtcaccatcacttgtcgggcgagtcagggcattagcaattatttagcctggtttcagcagaaaccagggaaagcccctaagtccctgatctatgctgcatccagtttgcaaagtggggtcccatcaaagttcagcggcagtggatctgggacagatttcactctcgccatcagcagcctgcagcctgaagattttgcaacttattactgccaacagtataatagtttcccgtacacttttggccaggggaccacgctggagatcaaa

SEQ ID NO: 17 - protein, 107 aa, Artificial Sequence
  Description of Artificial Sequence Synthetic polypeptide
DIQMSQSPSSLSASVGDRVTITCRASQGISNYLAWFQQKPGKAPKSLIYAASSLQSGVPSKFSGSGSGTDFTLAISSLQPEDFATYYCQQYNSFPYTFGQGTTLEIK

SEQ ID NO: 18 - protein, 11 aa, Artificial Sequence
  Description of Artificial Sequence Synthetic peptide
RASQGISNYLA

SEQ ID NO: 19 - protein, 7 aa, Artificial Sequence
  Description of Artificial Sequence Synthetic peptide
AASSLQS

SEQ ID NO: 20 - protein, 9 aa, Artificial Sequence
  Description of Artificial Sequence Synthetic peptide
QQYNSFPYT

SEQ ID NO: 21 - DNA, 354 bp, Artificial Sequence
  Description of Artificial Sequence Synthetic polynucleotide
caggtgcagctgcaggagtcgggcccaggactggtgaagccttcggagaccctgtccctcacctgcactgtctctggtggctccatcaatagttactactggagctggatccggcagcccccagggaagggactggagtggattgggtatatcttttacagtgggatcaccaactacaacccctccctcaagagtcgagtcaccatatcattagacacgtccaagaaccagttctccctgaagctgagctctgtgaccgctgcggacacggccgtgtattactgtgcgagaatcggcgtggctggtttttactttgactactggggccagggaaccctggtcaccgtctcctca

SEQ ID NO: 22 - protein, 118 aa, Artificial Sequence
  Description of Artificial Sequence Synthetic polypeptide
QVQLQESGPGLVKPSETLSLTCTVSGGSINSYYWSWIRQPPGKGLEWIGYIFYSGITNYNPSLKSRVTISLDTSKNQFSLKLSSVTAADTAVYYCARIGVAGFYFDYWGQGTLVTVSS

SEQ ID NO: 23 - protein, 7 aa, Artificial Sequence
  Description of Artificial Sequence Synthetic peptide
GGSINSY

SEQ ID NO: 24 - protein, 5 aa, Artificial Sequence
  Description of Artificial Sequence Synthetic peptide
FYSGI

SEQ ID NO: 25 - protein, 10 aa, Artificial Sequence
  Description of Artificial Sequence Synthetic peptide
IGVAGFYFDY

SEQ ID NO: 26 - DNA, 324 bp, Artificial Sequence
  Description of Artificial Sequence Synthetic polynucleotide
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtacctcaccgctcactttcggcggagggaccaaggtggagatcaaa

SEQ ID NO: 27 - protein, 108 aa, Artificial Sequence
  Description of Artificial Sequence Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGTSPLTFGGGTKVEIK

SEQ ID NO: 28 - protein, 12 aa, Artificial Sequence
  Description of Artificial Sequence Synthetic peptide
RASQSVSSSYLA

SEQ ID NO: 29 - protein, 7 aa, Artificial Sequence
  Description of Artificial Sequence Synthetic peptide
GASSRAT

SEQ ID NO: 30 - protein, 9 aa, Artificial Sequence
  Description of Artificial Sequence Synthetic peptide
QQYGTSPLT

SEQ ID NO: 31 - DNA, 360 bp, Artificial Sequence
  Description of Artificial Sequence Synthetic polynucleotide
caggtgcagctggtgcagtctggggctgaggtgaagaagcctggggcctcagtgaaggtttcctgcaaggcatctggatacaccttcaccagctactatatacactgggtgcgacaggcccctggacaagggcttgagtggatgggaataatcgacccaagtgatggtagcacaaactacgcacagaagttccagggcagagtcaccatgaccagggacacgtccacgagcacagtctacatggagctgagcagcctgagatctgaggacacggccgtgtattactgtgcgagagatcgggaatataactactacggtttggacgtctggggccaagggaccacggtcaccgtctcctca

SEQ ID NO: 32 - protein, 120 aa, Artificial Sequence
  Description of Artificial Sequence Synthetic polypeptide
QVQLVQSGAEVKKPGASVKVSCKASGYTFTSYYIHWVRQAPGQGLEWMGIIDPSDGSTNYAQKFQGRVTMTRDTSTSTVYMELSSLRSEDTAVYYCARDREYNYYGLDVWGQGTTVTVSS

SEQ ID NO: 33 - protein, 7 aa, Artificial Sequence
  Description of Artificial Sequence Synthetic peptide
GYTFTSY

SEQ ID NO: 34 - protein, 6 aa, Artificial Sequence
  Description of Artificial Sequence Synthetic peptide
DPSDGS

SEQ ID NO: 35 - protein, 11 aa, Artificial Sequence
  Description of Artificial Sequence Synthetic peptide
DREYNYYGLDV

SEQ ID NO: 36 - DNA, 336 bp, Artificial Sequence
  Description of Artificial Sequence Synthetic polynucleotide
gatgttgtgatgactcagtctccactctccctgcccgtcacccttggacagccggcctccatctcctgcaggtctagtcaaagcctcgtataccgtgatggaaacacctacttgaattggtttcagcagaggccaggccaatctccaaggcgcctaatttataaggtttctaaccgggactctggggtcccagacagattccgcggcagtgggtcaggcactgatttcacactgaaaatcagccgggtggaggctgaggatgttggggtttattactgcatgcaaggtacacactggcctccgacgttcggccaagggaccaaggtggaaatcaaa

SEQ ID NO: 37 - protein, 112 aa, Artificial Sequence
  Description of Artificial Sequence Synthetic polypeptide
DVVMTQSPLSLPVTLGQPASISCRSSQSLVYRDGNTYLNWFQQRPGQSPRRLIYKVSNRDSGVPDRFRGSGSGTDFTLKISRVEAEDVGVYYCMQGTHWPPTFGQGTKVEIK

SEQ ID NO: 38 - protein, 16 aa, Artificial Sequence
  Description of Artificial Sequence Synthetic peptide
RSSQSLVYRDGNTYLN

SEQ ID NO: 39 - protein, 7 aa, Artificial Sequence
  Description of Artificial Sequence Synthetic peptide
KVSNRDS

SEQ ID NO: 40 - protein, 9 aa, Artificial Sequence
  Description of Artificial Sequence Synthetic peptide
MQGTHWPPT

SEQ ID NO: 41 - protein, 384 aa, Homo sapiens
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDHGPMK

SEQ ID NO: 42 - protein, 330 aa, Homo sapiens [sequence omitted]

SEQ ID NO: 43 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 44 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 45 - protein, 452 aa, Homo sapiens
GSASAPTLFPLVSCENSPSDTSSVAVGCLAQDFLPDSITLSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVSLVMSDTAGTCY

SEQ ID NO: 46 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 47 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 48 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRMAGKPTHVNVSVVMAEVDGTCY

SEQ ID NO: 49 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 618 aa, Homo sapiens
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREVATPLFPPLHTGRAGQRQHLLFPYPSSILSVK

SEQ ID NO: 51 - protein, 587 aa, Homo sapiens
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREA

SEQ ID NO: 52 - protein, 587 aa, Pan troglodytes
MVSPRMSRLLSQTVILALIFLPQTRPAGVFELQIHSFGPGPGPGAPRSPCSARVPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGTWARDIQRAGAWELRFSYRARCEPPAVGTACTRLCRPRSAPSRCGPGLRPCAPLEDECEAPPVCRAGCSPEHGFCEQPGECRCLEGWTGPLCTVPVSTSSCLSPRGPSSATTGCLVPGPGPCDGNPCANGGSCSETPGSFECACPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHAQDAGARLLAGTPEPSVHALPDALNNLRTQEGAGDGPSSSVDWNRPEDVDPRGIYVISAPSIYAREA

SEQ ID NO: 53 - protein, 585 aa, Mus musculus
MVSLQVSPLSQTLILAFLLPQALPAGVFELQIHSFGPGPGLGTPRSPCNARGPCRLFFRVCLKPGVSQEATESLCALGAALSTSVPVYTEHPGESAAALPLPDGLVRVPFRDAWPGTFSLVIETWREQLGEHAGGPAWNLLARVVGRRRLAAGGPWARDVQRTGTWELHFSYRARCEPPAVGAACARLCRSRSAPSRCGPGLRPCTPFPDECEAPSVCRPGCSPEHGYCEEPDECRCLEGWTGPLCTVPVSTSSCLNSRVPGPASTGCLLPGPGPCDGNPCANGGSCSETSGSFECACPRGFYGLRCEVSGVTCADGPCFNGGLCVGGEDPDSAYVCHCPPGFQGSNCEKRVDRCSLQPCQNGGLCLDLGHALRCRCRAGFAGPRCEHDLDDCAGRACANGGTCVEGGGSRRCSCALGFGGRDCRERADPCASRPCAHGGRCYAHFSGLVCACAPGYMGVRCEFAVRPDGADAVPAAPRGLRQADPQRFLLPPALGLLVAAGLAGAALLVIHVRRRGPGQDTGTRLLSGTREPSVHTLPDALNNLRLQDGAGDGPSSSADWNHPEDGDSRSIYVIPAPSIYAREA

SEQ ID NO: 54 - protein, 589 aa, Rattus norvegicus
MVSLQVSSLPQTLILAFLLPQALPAGVFELQIHSFGPGPGPGTPRSPCNARGPCRLFFRVCLKPGVSQEAAESLCALGAALSTSGPVYTEQPGVPAAALSLPDGLVRVPFLDAWPGTFSLIIETWREQLGERAAGPAWNLLARVAGRRRLAAGAPWARDVQRTGAWELHFSYRARCEPPAVGAACARLCRSRSAPSRCGPGLRPCTPFPDECEAPRESLTVCRAGCSPEHGYCEEPDECHCLEGWTGPLCTVPVSTSSCLNSRVSGPAGTGCLLPGPGPCDGNPCANGGSCSETPGSFECACPRGFYGPRCEVSGVTCADGPCFNGGLCVGGEDPDSAYVCHCPPAFQGSNCERRVDRCSLQPCQNGGLCLDLGHALRCRCRAGFAGPRCEHDLDDCAGRACANGGTCVEGGGARRCSCALGFGGRDCRERADPCASRPCAHGGRCYAHFSGLVCACAPGYMGVRCEFAVRPDGADAVPAAPRGLRQADSQRFLLPPALGLLAAAALAGAALLLIHVRRRGPGRDTGTRLLSGTREPSVHTLPDALNNLRLQDGAGDGPTSSADWNHPEDGDSRSIYVIPAPSIYAREA

SEQ ID NO: 55 - DNA, 2341 bp, Homo sapiens
actcccgagacccccccaccagaaggccatggtctccccacggatgtccgggctcctctcccagactgtgatcctagcgctcattttcctcccccagacacggcccgctggcgtcttcgagctgcagatccactctttcgggccgggtccaggccctggggccccgcggtccccctgcagcgcccggctcccctgccgcctcttcttcagagtctgcctgaagcctgggctctcagaggaggccgccgagtccccgtgcgccctgggcgcggcgctgagtgcgcgcggaccggtctacaccgagcagcccggagcgcccgcgcctgatctcccactgcccgacggcctcttgcaggtgcccttccgggacgcctggcctggcaccttctctttcatcatcgaaacctggagagaggagttaggagaccagattggagggcccgcctggagcctgctggcgcgcgtggctggcaggcggcgcttggcagccggaggcccgtgggcccgggacattcagcgcgcaggcgcctgggagctgcgcttctcgtaccgcgcgcgctgcgagccgcctgccgtcgggaccgcgtgcacgcgcctctgccgtccgcgcagcgccccctcgcggtgcggtccgggactgcgcccctgcgcaccgctcgaggacgaatgtgaggcgccgctggtgtgccgagcaggctgcagccctgagcatggcttctgtgaacagcccggtgaatgccgatgcctagagggctggactggacccctctgcacggtccctgtctccaccagcagctgcctcagccccaggggcccgtcctctgctaccaccggatgccttgtccctgggcctgggccctgtgacgggaacccgtgtgccaatggaggcagctgtagtgagacacccaggtcctttgaatgcacctgcccgcgtgggttctacgggctgcggtgtgaggtgagcggggtgacatgtgcagatggaccctgcttcaacggcggcttgtgtgtcgggggtgcagaccctgactctgcctacatctgccactgcccacccggtttccaaggctccaactgtgagaagagggtggaccggtgcagcctgcagccatgccgcaatggcggactctgcctggacctgggccacgccctgcgctgccgctgccgcgccggcttcgcgggtcctcgctgcgagcacgacctggacgactgcgcgggccgcgcctgcgctaacggcggcacgtgtgtggagggcggcggcgcgcaccgctgctcctgcgcgctgggcttcggcggccgcgactgccgcgagcgcgcggacccgtgcgccgcgcgcccctgtgctcacggcggccgctgctacgcccacttctccggcctcgtctgcgcttgcgctcccggctacatgggagcgcggtgtgagttcccagtgcaccccgacggcgcaagcgccttgcccgcggccccgccgggcctcaggcccggggaccctcagcgctaccttttgcctccggctctgggactgctcgtggccgcgggcgtggccggcgctgcgctcttgctggtccacgtgcgccgccgtggccactcccaggatgctgggtctcgcttgctggctgggaccccggagccgtcagtccacgcactcccggatgcactcaacaacctaaggacgcaggagggttccggggatggtccgagctcgtccgtagattggaatcgccctgaagatgtagaccctcaagggatttatgtcatatctgctccttccatctacgctcgggaggtagcgacgccccttttccccccgctacacactgggcgcgctgggcagaggcagcacctgctttttccctacccttcctcgattctgtccgtgaaatgaattgggtagagtctctggaaggttttaagcccattttcagttctaacttactttcatcctattttgcatccctcttatcgttttgagctacctgccatcttctctttgaaaaacctatgggcttgaggaggtcacgatgccgactccgccagagcttttccactgattgtactcagcggggaggcaggggaggcagaggggcagcctctctaatgcttcctactcattttgtttctaggcctgacgcgtctcctccatccgcacctggagtcagagcgtggatttttgtatttgctcggtggtgcccagtctctgccccagaggctttggagttcaatcttgaaggggtgtctgggggaactttactgttgcaagttgtaaataatggttatttatatcctattttttctcaccccatctctctagaaacacctataaaggctattattgtgatca

SEQ ID NO: 56 - DNA, 2004 bp, Homo sapiens
actcccgagacccccccaccagaaggccatggtctccccacggatgtccgggctcctctcccagactgtgatcctagcgctcattttcctcccccagacacggcccgctggcgtcttcgagctgcagatccactctttcgggccgggtccaggccctggggccccgcggtccccctgcagcgcccggctcccctgccgcctcttcttcagagtctgcctgaagcctgggctctcagaggaggccgccgagtccccgtgcgccctgggcgcggcgctgagtgcgcgcggaccggtctacaccgagcagcccggagcgcccgcgcctgatctcccactgcccgacggcctcttgcaggtgcccttccgggacgcctggcctggcaccttctctttcatcatcgaaacctggagagaggagttaggagaccagattggagggcccgcctggagcctgctggcgcgcgtggctggcaggcggcgcttggcagccggaggcccgtgggcccgggacattcagcgcgcaggcgcctgggagctgcgcttctcgtaccgcgcgcgctgcgagccgcctgccgtcgggaccgcgtgcacgcgcctctgccgtccgcgcagcgccccctcgcggtgcggtccgggactgcgcccctgcgcaccgctcgaggacgaatgtgaggcgccgctggtgtgccgagcaggctgcagccctgagcatggcttctgtgaacagcccggtgaatgccgatgcctagagggctggactggacccctctgcacggtccctgtctccaccagcagctgcctcagccccaggggcccgtcctctgctaccaccggatgccttgtccctgggcctgggccctgtgacgggaacccgtgtgccaatggaggcagctgtagtgagacacccaggtcctttgaatgcacctgcccgcgtgggttctacgggctgcggtgtgaggtgagcggggtgacatgtgcagatggaccctgcttcaacggcggcttgtgtgtcgggggtgcagaccctgactctgcctacatctgccactgcccacccggtttccaaggctccaactgtgagaagagggtggaccggtgcagcctgcagccatgccgcaatggcggactctgcctggacctgggccacgccctgcgctgccgctgccgcgccggcttcgcgggtcctcgctgcgagcacgacctggacgactgcgcgggccgcgcctgcgctaacggcggcacgtgtgtggagggcggcggcgcgcaccgctgctcctgcgcgctgggcttcggcggccgcgactgccgcgagcgcgcggacccgtgcgccgcgcgcccctgtgctcacggcggccgctgctacgcccacttctccggcctcgtctgcgcttgcgctcccggctacatgggagcgcggtgtgagttcccagtgcaccccgacggcgcaagcgccttgcccgcggccccgccgggcctcaggcccggggaccctcagcgctaccttttgcctccggctctgggactgctcgtggccgcgggcgtggccggcgctgcgctcttgctggtccacgtgcgccgccgtggccactcccaggatgctgggtctcgcttgctggctgggaccccggagccgtcagtccacgcactcccggatgcactcaacaacctaaggacgcaggagggttccggggatggtccgagctcgtccgtagattggaatcgccctgaagatgtagaccctcaagggatttatgtcatatctgctccttccatctacgctcgggaggcctgacgcgtctcctccatccgcacctggagtcagagcgtggatttttgtatttgctcggtggtgcccagtctctgccccagaggctttggagttcaatcttgaaggggtgtctgggggaactttactgttgcaagttgtaaataatggttatttatatcctattttttctcaccccatctctctagaaacacctataaaggctattattgtgatca

SEQ ID NO: 57 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 448 aa, Artificial Sequence
  Description of Artificial Sequence Synthetic polypeptide
EVQLVESGGGLVQPGGSQRLSCAASGFTFSSYWMNWVRQAPGKGLEWVANIKEDGSEKYYVDSVKGRFTISRDNAKNSLYLQMNSLRAEDTAVYYCARDPGWAPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 448 aa, Artificial Sequence
  Description of Artificial Sequence Synthetic polypeptide
EVQLVESGGGLVQPGGSQRLSCAASGFTFSSYWMNWVRQAPGKGLEWVANIKEDGSEKYYVDSVKGRFTISRDNAKNSLYLQMNSLRAEDTAVYYCARDPGWAPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 448 aa, Artificial Sequence
  Description of Artificial Sequence Synthetic polypeptide
EVQLVESGGGLVQPGGSQRLSCAASGFTFSSYWMNWVRQAPGKGLEWVANIKEDGSEKYYVDSVKGRFTISRDNAKNSLYLQMNSLRAEDTAVYYCARDPGWAPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 214 aa, Artificial Sequence
  Description of Artificial Sequence Synthetic polypeptide
DIQMSQSPSSLSASVGDRVTITCRASQGISNYLAWFQQKPGKAPKSLIYAASSLQSGVPSKFSGSGSGTDFTLAISSLQPEDFATYYCQQYNSFPYTFGQGTTLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 450 aa, Artificial Sequence
  Description of Artificial Sequence Synthetic polypeptide
QVQLVQSGAEVKKPGASVKVSCKASGYTFTSYYIHWVRQAPGQGLEWMGIIDPSDGSTNYAQKFQGRVTMTRDTSTSTVYMELSSLRSEDTAVYYCARDREYNYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 450 aa, Artificial Sequence
  Description of Artificial Sequence Synthetic polypeptide
QVQLVQSGAEVKKPGASVKVSCKASGYTFTSYYIHWVRQAPGQGLEWMGIIDPSDGSTNYAQKFQGRVTMTRDTSTSTVYMELSSLRSEDTAVYYCARDREYNYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 450 aa, Artificial Sequence
  Description of Artificial Sequence Synthetic polypeptide
QVQLVQSGAEVKKPGASVKVSCKASGYTFTSYYIHWVRQAPGQGLEWMGIIDPSDGSTNYAQKFQGRVTMTRDTSTSTVYMELSSLRSEDTAVYYCARDREYNYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 219 aa, Artificial Sequence
  Description of Artificial Sequence Synthetic polypeptide
DVVMTQSPLSLPVTLGQPASISCRSSQSLVYRDGNTYLNWFQQRPGQSPRRLIYKVSNRDSGVPDRFRGSGSGTDFTLKISRVEAEDVGVYYCMQGTHWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 448 aa, Artificial Sequence
  Description of Artificial Sequence Synthetic polypeptide
QVQLQESGPGLVKPSETLSLTCTVSGGSINSYYWSWIRQPPGKGLEWIGYIFYSGITNYNPSLKSRVTISLDTSKNQFSLKLSSVTAADTAVYYCARIGVAGFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 448 aa, Artificial Sequence
  Description of Artificial Sequence Synthetic polypeptide
QVQLQESGPGLVKPSETLSLTCTVSGGSINSYYWSWIRQPPGKGLEWIGYIFYSGITNYNPSLKSRVTISLDTSKNQFSLKLSSVTAADTAVYYCARIGVAGFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 448 aa, Artificial Sequence
  Description of Artificial Sequence Synthetic polypeptide
QVQLQESGPGLVKPSETLSLTCTVSGGSINSYYWSWIRQPPGKGLEWIGYIFYSGITNYNPSLKSRVTISLDTSKNQFSLKLSSVTAADTAVYYCARIGVAGFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 215 aa, Artificial Sequence
  Description of Artificial Sequence Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGTSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 448 aa, Artificial Sequence
  Description of Artificial Sequence Synthetic polypeptide
QVQLVQSGAEVKKPGASVKVSCKASGYTFTNYGMNWVRQAPGQGLEWMGWINTYTGEPTYADDFKGRVTMTTDTSTSTAYMELRSLRSDDTAVYYCARIGDSSPSDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 214 aa, Artificial Sequence
  Description of Artificial Sequence Synthetic polypeptide
EIVMTQSPATLSVSPGERATLSCKASQSVSNDVVWYQQKPGQAPRLLIYYASNRYTGIPARFSGSGSGTEFTLTISSLQSEDFAVYYCQQDYTSPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 455 aa, Artificial Sequence
  Description of Artificial Sequence Synthetic polypeptide
QVQLVQSGAEVKKPGASVKVSCKASGYTFTSYWINWVRQAPGQGLEWMGNIYPGSSSINYNEKFKSRVTITADTSTSTAYMELSSLRSEDTAVYYCARTIYNYGSSGYNYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 214 aa, Artificial Sequence
  Description of Artificial Sequence Synthetic polypeptide
DIVMTQSPDSLAVSLGERATINCKASENVGNSVSWYQQKPGQPPKLLIYGASNRYTGVPDRFSGSGSGTDFTLTISSLQAEDVAVYYCGQSYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - DNA, 1117 bp, Artificial Sequence
  Description of Artificial Sequence Synthetic polynucleotide
gggtctagagccaccatgaaacacctgtggttcttcctcctgctggtggcagctcccagatgggtgctgagccaggtgcaattgtgcaggcggttagctcagcctccaccaagggcccaagcgtcttccccctggcaccctcctccaagagcacctctggcggcacagccgccctgggctgcctggtcaaggactacttccccgaacccgtgaccgtgagctggaactcaggcgccctgaccagcggcgtgcacaccttccccgctgtcctgcagtcctcaggactctactccctcagcagcgtggtgaccgtgccctccagcagcttgggcacccagacctacatctgcaacgtgaatcacaagcccagcaacaccaaggtggacaagaaggttgagcccaaatcttgtgacaaaactcacacatgcccaccctgcccagcacctgaactcctggggggaccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccccgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggccagccccgggaaccacaggtgtacaccctgcccccatcccgggaggagatgaccaagaaccaggtcagcctgacctgcctggtcaaaggcttctatcccagcgacatcgccgtggagtgggagagcaatggccagcccgagaacaactacaagaccacccctcccgtgctggactccgacggctccttcttcctctacagcaagctcaccgtggacaagagcaggtggcagcagggcaacgtcttctcatgctccgtgatgcatgaggctctgcacaaccactacacccagaagagcctctccctgtctcccggcaaatgagatatcgggcccgtttaaacggg

SEQ ID NO: 76 - DNA, 1401 bp, Artificial Sequence
  Description of Artificial Sequence Synthetic polynucleotide
atgaaacacctgtggttcttcctcctgctggtggcagctcccagatgggtgctgagcgaggtgcagctggttgaatctggcggaggactggttcagcctggcggatctcagagactgtcttgtgccgccagcggcttcaccttcagcagctactggatgaactgggtccgacaggcccctggcaaaggccttgaatgggtcgccaacatcaaagaggacggcagcgagaagtactacgtggacagcgtgaagggcagattcaccatctccagagacaacgccaagaacagcctgtacctgcagatgaactccctgagagccgaggacaccgccgtgtactactgtgccagagatcctggctgggcccctttcgattattggggccagggcacactggtcaccgttagctcagcctccaccaagggcccaagcgtcttccccctggcaccctcctccaagagcacctctggcggcacagccgccctgggctgcctggtcaaggactacttccccgaacccgtgaccgtgagctggaactcaggcgccctgaccagcggcgtgcacaccttccccgctgtcctgcagtcctcaggactctactccctcagcagcgtggtgaccgtgccctccagcagcttgggcacccagacctacatctgcaacgtgaatcacaagcccagcaacaccaaggtggacaagaaggttgagcccaaatcttgtgacaaaactcacacatgcccaccctgcccagcacctgaactcctggggggaccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccccgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggccagccccgggaaccacaggtgtacaccctgcccccatcccgggaggagatgaccaagaaccaggtcagcctgacctgcctggtcaaaggcttctatcccagcgacatcgccgtggagtgggagagcaatggccagcccgagaacaactacaagaccacccctcccgtgctggactccgacggctccttcttcctctacagcaagctcaccgtggacaagagcaggtggcagcagggcaacgtcttctcatgctccgtgatgcatgaggctctgcacaaccactacacccagaagagcctctccctgtctcccggcaaa

SEQ ID NO: 77 - DNA, 1401 bp, Artificial Sequence
  Description of Artificial Sequence Synthetic polynucleotide
atgaagcacctgtggttctttctgctgctggtggccgctcctagatgggtgctgtctgaagtgcagctggtggaatctggcggaggactggttcaacctggcggctctcagagactgtcttgtgccgccagcggcttcaccttcagcagctactggatgaactgggtccgacaggcccctggcaaaggccttgaatgggtcgccaacatcaaagaggacggcagcgagaagtactacgtggacagcgtgaagggcagattcaccatctccagagacaacgccaagaacagcctgtacctgcagatgaactccctgcgcgccgaagataccgccgtgtactactgtgccagagatcctggctgggcccctttcgattattggggccagggaaccctggtcaccgtgtcatctgcctccaccaagggcccaagcgtcttccccctggcaccctcctccaagagcacctctggcggcacagccgccctgggctgcctggtcaaggactacttccccgaacccgtgaccgtgagctggaactcaggcgccctgaccagcggcgtgcacaccttccccgctgtcctgcagtcctcaggactctactccctcagcagcgtggtgaccgtgccctccagcagcttgggcacccagacctacatctgcaacgtgaatcacaagcccagcaacaccaaggtggacaagaaggttgagcccaaatcttgtgacaaaactcacacatgcccaccctgcccagcacctgaagccgcggggggaccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccccgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggccagccccgggaaccacaggtgtacaccctgcccccatcccgggaggagatgaccaagaaccaggtcagcctgacctgcctggtcaaaggcttctatcccagcgacatcgccgtggagtgggagagcaatggccagcccgagaacaactacaagaccacccctcccgtgctggactccgacggctccttcttcctctacagcaagctcaccgtggacaagagcaggtggcagcagggcaacgtcttctcatgctccgtgatgcatgaggctctgcacaaccactacacccagaagagcctctccctgtctcccggcaaa

SEQ ID NO: 78 - DNA, 1401 bp, Artificial Sequence
  Description of Artificial Sequence Synthetic polynucleotide
atgaaacacctgtggttcttcctcctgctggtggcagctcccagatgggtgctgagccaggttcagctgcaagagtctggccctggcctggtcaagcctagcgaaacactgagcctgacctgtaccgtgtctggcggcagcatcaacagctactactggtcctggatccggcagcctcctggcaaaggactggaatggatcggctacatcttctacagcggcatcaccaactacaaccccagcctgaagtccagagtgaccatcagcctggacaccagcaagaaccagttctccctgaagctgagcagcgtgacagccgccgatacagccgtgtactactgtgccagaatcggcgtggccggcttctacttcgattattggggccagggcaccctggtcacagttagctcagcctccaccaagggcccaagcgtcttccccctggcaccctcctccaagagcacctctggcggcacagccgccctgggctgcctggtcaaggactacttccccgaacccgtgaccgtgagctggaactcaggcgccctgaccagcggcgtgcacaccttccccgctgtcctgcagtcctcaggactctactccctcagcagcgtggtgaccgtgccctccagcagcttgggcacccagacctacatctgcaacgtgaatcacaagcccagcaacaccaaggtggacaagaaggttgagcccaaatcttgtgacaaaactcacacatgcccaccctgcccagcacctgaactcctggggggaccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccccgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggccagccccgggaaccacaggtgtacaccctgcccccatcccgggaggagatgaccaagaaccaggtcagcctgacctgcctggtcaaaggcttctatcccagcgacatcgccgtggagtgggagagcaatggccagcccgagaacaactacaagaccacccctcccgtgctggactccgacggctccttcttcctctacagcaagctcaccgtggacaagagcaggtggcagcagggcaacgtcttctcatgctccgtgatgcatgaggctctgcacaaccactacacccagaagagcctctccctgtctcccggcaaa

SEQ ID NO: 79 - DNA, 1401 bp, Artificial Sequence
  Description of Artificial Sequence Synthetic polynucleotide
atgaagcacctgtggttctttctgctgctggtggccgctcctagatgggtgctgtctcaggttcagctgcaagagtctggccctggcctggtcaagcctagcgaaacactgagcctgacctgtaccgtgtctggcggcagcatcaacagctactactggtcctggatccggcagcctcctggcaaaggactggaatggatcggctacatcttctacagcggcatcaccaactacaaccccagcctgaagtccagagtgaccatcagcctggacaccagcaagaaccagttctccctgaagctgagcagcgtgacagccgccgatacagccgtgtactactgtgccagaatcggcgtggccggcttctacttcgattattggggccagggcaccctggtcaccgtttcttctgcctccaccaagggcccaagcgtcttccccctggcaccctcctccaagagcacctctggcggcacagccgccctgggctgcctggtcaaggactacttccccgaacccgtgaccgtgagctggaactcaggcgccctgaccagcggcgtgcacaccttccccgctgtcctgcagtcctcaggactctactccctcagcagcgtggtgaccgtgccctccagcagcttgggcacccagacctacatctgcaacgtgaatcacaagcccagcaacaccaaggtggacaagaaggttgagcccaaatcttgtgacaaaactcacacatgcccaccctgcccagcacctgaagccgcggggggaccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccccgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggccagccccgggaaccacaggtgtacaccctgcccccatcccgggaggagatgaccaagaaccaggtcagcctgacctgcctggtcaaaggcttctatcccagcgacatcgccgtggagtgggagagcaatggccagcccgagaacaactacaagaccacccctcccgtgctggactccgacggctccttcttcctctacagcaagctcaccgtggacaagagcaggtggcagcagggcaacgtcttctcatgctccgtgatgcatgaggctctgcacaaccactacacccagaagagcctctccctgtctcccggcaaa

SEQ ID NO: 80 - DNA, 702 bp, Artificial Sequence
  Description of Artificial Sequence Synthetic polynucleotide
atggtgctgcagacccaggtgttcatctccctgctgctgtggatctccggcgcgtacggcgacatccagatgtctcagagccctagcagcctgtctgccagcgtgggagacagagtgaccatcacctgtagagccagccagggcatcagcaactacctggcctggttccagcagaagcctggcaaggcccctaagagcctgatctatgccgctagctctctgcagtctggcgtgccctctaagtttagcggctctggcagcggcaccgatttcacactggccatatctagcctgcagcctgaggacttcgccacctactactgccagcagtacaacagcttcccctacaccttcggccagggcaccacactggaaatcaagcgtacggtggccgccccctccgtgttcatcttccccccctccgacgagcagctgaagtccggcaccgcctccgtggtgtgcctgctgaataacttctaccccagagaggccaaggtgcagtggaaggtggacaacgccctgcagtccgggaactcccaggagagcgtgaccgagcaggacagcaaggacagcacctacagcctgagcagcaccctgaccctgagcaaagccgactacgagaagcacaaggtgtacgcctgcgaggtgacccaccagggcctgagctcccccgtcaccaagagcttcaacaggggggagtgt

SEQ ID NO: 81 - DNA, 705 bp, Artificial Sequence
  Description of Artificial Sequence Synthetic polynucleotide
atggtgctgcagacccaggtgttcatctccctgctgctgtggatctccggcgcgtacggcgagatcgtgctgacacagagccctggcacactgtcactgtctccaggcgaaagagccacactgagctgtagagccagccagagcgtgtccagctcttacctggcttggtatcagcagaagcccggacaggctcccagactgctgatctatggcgcctcttctagagccacaggcatccccgatagattcagcggctctggcagcggcaccgatttcaccctgacaatcagcagactggaacccgaggacttcgccgtgtactactgtcagcagtacggcacaagccctctgacctttggcggcggaacaaaggtggaaatcaagcgtacggtggccgccccctccgtgttcatcttccccccctccgacgagcagctgaagtccggcaccgcctccgtggtgtgcctgctgaataacttctaccccagagaggccaaggtgcagtggaaggtggacaacgccctgcagtccgggaactcccaggagagcgtgaccgagcaggacagcaaggacagcacctacagcctgagcagcaccctgaccctgagcaaagccgactacgagaagcacaaggtgtacgcctgcgaggtgacccaccagggcctgagctcccccgtcaccaagagcttcaacaggggggagtgt

SEQ ID NO: 82 - protein, 5 aa, Artificial Sequence
  Description of Artificial Sequence Synthetic peptide
GGGGS

SEQ ID NO: 83 - protein, 75 aa, Artificial Sequence
  Description of Artificial Sequence Synthetic polypeptide
  SITE (1)..(75): This sequence may encompass 1-15 “Gly Gly Gly Gly Ser” repeating units
  See specification as filed for detailed description of substitutions and preferred embodiments
GGGGSGGGGSGGGGSGGGGSGGGGSGGGGSGGGGSGGGGSGGGGSGGGGSGGGGSGGGGSGGGGSGGGGSGGGGS

SEQ ID NO: 84 - protein, 6 aa, Artificial Sequence
  Description of Artificial Sequence Synthetic 6xHis tag
HHHHHH

SEQ ID NO: 85 - protein, 4 aa, Artificial Sequence
  Description of Artificial Sequence Synthetic peptide
GGVA

SEQ ID NO: 86 - protein, 4 aa, Artificial Sequence
  Description of Artificial Sequence Synthetic peptide
GGFG

SEQ ID NO: 87 - protein, 4 aa, Artificial Sequence
  Description of Artificial Sequence Synthetic peptide
GGPI

SEQ ID NO: 88 - protein, 4 aa, Artificial Sequence
  Description of Artificial Sequence Synthetic peptide
  MOD_RES (4)..(4): Citrulline
GGVX

SEQ ID NO: 89 - protein, 4 aa, Artificial Sequence
  Description of Artificial Sequence Synthetic peptide
GGVK

SEQ ID NO: 90 - protein, 4 aa, Artificial Sequence
  Description of Artificial Sequence Synthetic peptide
GGPL

SEQ ID NO: 91 - protein, 5 aa, Artificial Sequence
  Description of Artificial Sequence Synthetic peptide
EGGVA

SEQ ID NO: 92 - protein, 4 aa, Artificial Sequence
  Description of Artificial Sequence Synthetic peptide
DGGF

SEQ ID NO: 93 - protein, 4 aa, Artificial Sequence
  Description of Artificial Sequence Synthetic peptide
EGGF

SEQ ID NO: 94 - protein, 4 aa, Artificial Sequence
  Description of Artificial Sequence Synthetic peptide
SGGF

SEQ ID NO: 95 - protein, 4 aa, Artificial Sequence
  Description of Artificial Sequence Synthetic peptide
KGGF

SEQ ID NO: 96 - protein, 5 aa, Artificial Sequence
  Description of Artificial Sequence Synthetic peptide
DGGFG

SEQ ID NO: 97 - protein, 5 aa, Artificial Sequence
  Description of Artificial Sequence Synthetic peptide
GGFGG

SEQ ID NO: 98 - protein, 6 aa, Artificial Sequence
  Description of Artificial Sequence Synthetic peptide
DDGGFG

SEQ ID NO: 99 - protein, 6 aa, Artificial Sequence
  Description of Artificial Sequence Synthetic peptide
KDGGFG

SEQ ID NO: 100 - protein, 7 aa, Artificial Sequence
  Description of Artificial Sequence Synthetic peptide
GGFGGGF

SEQ ID NO: 101 - DNA, 25 bp, Artificial Sequence
  Description of Artificial Sequence Synthetic oligonucleotide
ccagcctccggactctagagccacc

SEQ ID NO: 102 - DNA, 25 bp, Artificial Sequence
  Description of Artificial Sequence Synthetic oligonucleotide
tgagtttaaacgggggaggctaact

(19) United States
(12) Patent Application Publication
(10) Pub. No.: US 2024/0115720 A1
(43) Pub. Date: Apr. 11, 2024

(54) ANTIBODY-PYRROLOBENZODIAZEPINE DERIVATIVE CONJUGATE

(71) Applicants: MEMORIAL SLOAN KETTERING CANCER CENTER, New York, NY (US); TRI-INSTITUTIONAL THERAPEUTICS DISCOVERY INSTITUTE, New York, NY (US); DAIICHI SANKYO COMPANY, LIMITED, Tokyo (JP)

(72) Inventors: John T. POIRIER, New York, NY (US); Charles RUDIN, New York, NY (US); Jason LEWIS, New York, NY (US); Abdul KHAN, New York, NY (US); David ANDREW, New York, NY (US); Xinlei CHEN, New York, NY (US); Ivo LORENZ, New York, NY (US); Hironori MATSUNAGA, Tokyo (JP)

(73) Assignees: MEMORIAL SLOAN KETTERING CANCER CENTER, New York, NY (US); TRI-INSTITUTIONAL THERAPEUTICS DISCOVERY INSTITUTE, New York, NY (US); DAIICHI SANKYO COMPANY, LIMITED, Tokyo (JP)

(21) Appl. No.: 18/272,049

(22) PCT Filed: Jan. 13, 2022
(86) PCT No.: PCT/IB2022/050219

§ 371 Date: Jul. 12, 2023

Related U.S. Application Data
(60) Provisional application No. 63/136,928, filed on Jan. 13, 2021.

(51) Int. Cl. A61K 47/68; A61K 31/5517; A61P 35/00; C07K 16/28

(52) U.S. Cl. CPC A61K 47/68035; A61K 31/5517; A61K 47/6849; A61P 35/00; C07K 16/28; A61K 2039/505

(57) ABSTRACT

The present invention provides a novel anti-DLL3 antibody-pyrrolodiazepine derivative and a novel anti-DLL3 anti-body-pyrrolodiazepine derivative conjugate using the same.

### CROSS REFERENCE TO RELATED APPLICATION

This application claims priority to U.S. Provisional Application No. 63/136,928, filed Jan. 13, 2021, the disclosures of which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Jan. 12, 2022, is named 098065-0303_SL.txt and is 153,716 bytes in size.

### TECHNICAL FIELD

The present invention relates to an antibody-drug conjugate useful as an antitumor drug, the antibody-drug conjugate having an antibody capable of targeting tumor cells and a pyrrolobenzodiazepine derivative that are conjugated to each other via a linker structure moiety.

### BACKGROUND

The following description of the background of the present technology is provided simply as an aid in understanding the present technology and is not admitted to describe or constitute prior art to the present technology.

Antibody-drug conjugates (ADCs) have a drug with cytotoxic activity conjugated to an antibody that binds to an antigen expressed on the surface of cancer cells and is capable of cellular internalization of the antigen through the binding. ADCs can effectively deliver the drug to cancer cells, and are thus expected to cause accumulation of the drug within the cancer cells and to kill the cancer cells.

For example, the ADC Adcetris™ (brentuximab vedotin), which has monomethyl auristatin E conjugated to an anti-CD30 monoclonal antibody, has been approved as a therapeutic drug for Hodgkin's lymphoma and anaplastic large-cell lymphoma. Kadcyla™ (trastuzumab emtansine), which has emtansine conjugated to an anti-HER2 monoclonal antibody, is used for treatment of HER2-positive advanced and recurrent breast cancers.

A useful example of drugs to be conjugated for ADCs is pyrrolobenzodiazepine (PBD). PBD exhibits cytotoxicity, for example, by binding to the PuGPu sequence in the DNA minor groove. Anthramycin, a naturally-occurring PBD, was first discovered in 1965, and since this discovery various naturally-occurring PBDs and analog PBDs thereof have been discovered (see Mantaj, et al., Angewandte Chemie, Internationl Edition 2016, 55, 2-29; Antonow. et al., Chemical Reviews, 2010, 111, 2815-2864; In Antibiotics III. Springer Verlag, New York, pp. 3-11; and Accounts of Chemical Research, 1986, 19, 230).

The general structural formula of PBDs is represented by the following formula:

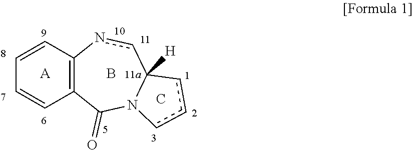

Known are PBDs different in the number of, types of, and sites of substituents in the A and C ring parts, and those different in degree of unsaturation in the B and C ring parts.

PBDs are known to come to have dramatically enhanced cytotoxicity through formation of a dimer structure (see Journal of the American Chemical Society, 1992, 114, 4939; Journal of Organic Chemistry, 1996, 61, 8141), and various ADCs with a dimer PBD have been reported (see WO 2013/173496, WO 2014/130879, WO 2017/004330, WO 2017/004025, WO 2017/020972, WO 2016/036804, WO 2015/095124, WO 2015/052322, WO 2015/052534, WO 2016/115191, WO 2015/052321, WO 2015/031693, WO 2011/130613 and WO2019/065964).

A dimer structure PBD is used in some known ADCs. For example, ADCs, in which dimer structure PBDs are conjugated to antibodies that target DLL3, are reported (see WO2013/126746 and Saunders et al., Sci Translational Medicine 7(302): 302ra136 (2015)).

DLL3 (i.e., delta-like ligand 3 or delta-like protein 3) is a single-pass type I transmembrane protein, and is one of Notch ligands (see Owen et al. J Hematol Oncol 12, 61 (2019)). DLL3 is selectively expressed in high grade pulmonary neuroendocrine tumors including SCLC and LCNEC. Increased expression of DLL3 was observed in SCLC and LCNEC patient-derived xenograft tumors and was also confirmed in primary tumors. See Saunders et al., Sci Translational Medicine 7(302):302ra136 (2015). Increased expression of DLL3 has also been observed in extrapulmonary neuroendocrine cancers including prostate neuroendocrine carcinoma (Puca et al., Sci Transl Med 11(484): pii: eaav0891 (2019). While DLL3 is expressed on the surface of such tumor cells, its expression in normal tissues in adults is limited.

Various pharmaceutical compositions containing anti-DLL3 antibodies as active ingredients are known. See Giffin et al., Clin Cancer Res 2021; 277:1526-37, WO2011/093097, and WO2013/126746. But thus far, no drugs targeting DLL3 are approved for use as a pharmaceutical agent

There is a need in the art for efficient and effective targeted therapeutics, such as ADC, for treating various types of cancer. The present application fulfills that need and include ADCs that utilize PBDs.

### SUMMARY

Problems to be resolved by the Invention: The present invention provides a novel anti-DLL-3 antibody-pyrrolobenzodiazepine (PBD) derivative conjugate. In addition, the present invention provides a pharmaceutical composition containing the anti-DLL-3 antibody-PBD derivative conjugate. Further, the present invention provides a method for treating cancer by using the anti-DLL3 antibody-PBD derivative conjugate.

Means of solving the Problems: The present inventors diligently examined to find that a novel anti-DLL-3 antibody-pyrrolobenzodiazepine (PBD) derivative conjugate has unexpectedly strong antitumor activity, thereby completing the present invention. The present invention includes the following aspects and embodiments of the invention:

[1] In one aspect, the present disclosure provides an antibody-drug conjugate represented by the following formula:

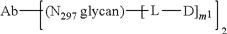

wherein
m¹ represents an integer of 1 to 2;
D represents a drug represented by one of the following formulas:

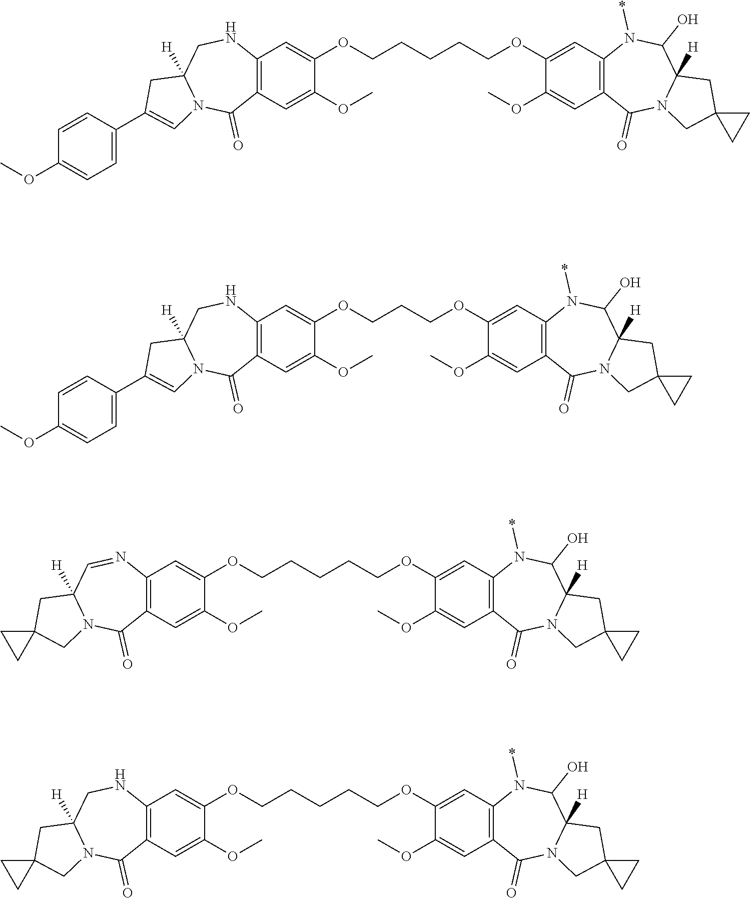 [SMILES: *N1c2cc(OCCCCCOc3cc4c(cc3OC)C(=O)N3C=C(c5ccc(OC)cc5)C[C@H]3CN4)c(OC)cc2C(=O)N2CC3(CC3)C[C@H]2C1O ; *N1c2cc(OCCCOc3cc4c(cc3OC)C(=O)N3C=C(c5ccc(OC)cc5)C[C@H]3CN4)c(OC)cc2C(=O)N2CC3(CC3)C[C@H]2C1O ; *N1c2cc(OCCCCCOc3cc4c(cc3OC)C(=O)N3CC5(CC5)C[C@H]3C=N4)c(OC)cc2C(=O)N2CC3(CC3)C[C@H]2C1O ; *N1c2cc(OCCCCCOc3cc4c(cc3OC)C(=O)N3CC5(CC5)C[C@H]3CN4)c(OC)cc2C(=O)N2CC3(CC3)C[C@H]2C1O]

wherein the asterisk represents bonding to L;
L is a linker linking the N297 glycan of Ab and D;
N297 glycan optionally is remodeled;
Ab represents an anti-DLL3 antibody that specifically binds to DLL-3.

[2] In some embodiments according to [1], the anti-DLL3 antibody comprises a heavy chain immunoglobulin variable domain (VH) and a light chain immunoglobulin variable domain (VL), wherein (a) the VH comprises a VH-CDR1 sequence, a VH-CDR2 sequence, and a VH-CDR3 sequence selected from the group consisting of
(i) SEQ ID NO: 3, SEQ ID NO: 4, and SEQ ID NO: 5, respectively;
(ii) SEQ ID NO: 13, SEQ ID NO: 14, and SEQ ID NO: 15, respectively;
(iii) SEQ ID NO: 23, SEQ ID NO: 24, and SEQ ID NO: 25, respectively; and
(iv) SEQ ID NO: 33, SEQ ID NO: 34, and SEQ ID NO: 35, respectively; and/or
(b) the V_{L} comprises a V_{L}-CDR1 sequence, a V_{L}-CDR2 sequence, and a V_{L}-CDR3 sequence selected from the group consisting of:
   (i) SEQ ID NO: 8, SEQ ID NO: 9, and SEQ ID NO: 10, respectively;
   (ii) SEQ ID NO: 18, SEQ ID NO: 19, and SEQ ID NO: 20, respectively;
   (iii) SEQ ID NO: 28, SEQ ID NO: 29, and SEQ ID NO: 30, respectively; and
   (iv) SEQ ID NO: 38, SEQ ID NO: 39, and SEQ ID NO: 40, respectively.

[3] In some embodiments according to [1] or [2]. the anti-DLL3 antibody comprises a heavy chain immunoglobulin variable domain (V_{H}) and a light chain immunoglobulin variable domain (V_{L}), wherein the combination of (a) the V_{H} comprising a V_{H}-CDR1 sequence, a V_{H}-CDR2 sequence, and a V_{H}-CDR3 sequence and (b) the V_{L} comprising a V_{L}-CDR1 sequence, a V_{L}-CDR2 sequence, and a V_{L}-CDR3 sequence is selected from the group consisting of:
(i) (a) SEQ ID NO: 3, SEQ ID NO: 4, and SEQ ID NO: 5 and (b) SEQ ID NO: 8, SEQ ID NO: 9, and SEQ ID NO: 10, respectively;
(ii) (a) SEQ ID NO: 13, SEQ ID NO: 14, and SEQ ID NO: 15 and (b) SEQ ID NO: 18, SEQ ID NO: 19, and SEQ ID NO: 20, respectively;
(iii) (a) SEQ ID NO: 23, SEQ ID NO: 24, and SEQ ID NO: 25 and (b) SEQ ID NO: 28, SEQ ID NO: 29, and SEQ ID NO: 30, respectively; and
(iv) (a) SEQ ID NO: 33, SEQ ID NO: 34, and SEQ ID NO: 35 and (b) SEQ ID NO: 38, SEQ ID NO: 39, and SEQ ID NO: 40, respectively.

[4] In some embodiments according to [1] or [2], the anti-DLL3 antibody comprises one or more of the following characteristics:
(a) a light chain immunoglobulin variable domain sequence that is at least 80%, at least 85%, at least 90%, at least 95%, or at least 99% identical to the light chain immunoglobulin variable domain sequence present in any one of SEQ ID NOs: 7, 17, 27, 37, 62, 66, or 70; and/or
(b) a heavy chain immunoglobulin variable domain sequence that is at least 80%, at least 85%, at least 90%, at least 95%, or at least 99% identical to the heavy chain immunoglobulin variable domain sequence present in any one of SEQ ID NOs: 2, 12, 22, 32, 59, 60, 61, 63, 64, 65, 67, 68, or 69.

[5] In some embodiments according to any one of [1]-[4], the anti-DLL3 antibody comprises one or more of the following characteristics:
(a) a light chain immunoglobulin variable domain sequence that is at least 80%, at least 85%, at least 90%, at least 95%, or at least 99% identical to the light chain immunoglobulin variable domain sequence present in any one of SEQ ID NOs: 17, 27 or 37; and
(b) a heavy chain immunoglobulin variable domain sequence that is at least 80%, at least 85%, at least 90%, at least 95%, or at least 99% identical to the heavy chain immunoglobulin variable domain sequence present in any one of SEQ ID NOs: 12, 22 or 32.

[6] In some embodiments according to any one of [1]-[5], the anti-DLL3 antibody comprises a heavy chain immunoglobulin variable domain (V_{H}) and a light chain immunoglobulin variable domain (V_{L}), wherein: (a) the V_{H} comprises an amino acid sequence selected from the group consisting of SEQ ID NO: 2, SEQ ID NO: 12, SEQ ID NO: 22, and SEQ ID NO: 32; and/or (b) the V_{L} comprises an amino acid sequence selected from the group consisting of SEQ ID NO: 7, SEQ ID NO: 17, SEQ ID NO: 27, and SEQ ID NO: 37.

[7] In some embodiments according to any one of [1]-[6], the anti-DLL3 antibody comprises a heavy chain immunoglobulin variable domain (V_{H}) and a light chain immunoglobulin variable domain (V_{L}), wherein: (a) the V_{H} comprises an amino acid sequence selected from SEQ ID NO: 12, SEQ ID NO: 22 or SEQ ID NO: 32, and (b) the V_{L} comprises an amino acid sequence selected from SEQ ID NO: 17, SEQ ID NO: 27 or SEQ ID NO: 37.

[8] In some embodiments according to [6], the V_{H} amino acid sequence and the V_{L} amino acid sequence is selected from the group consisting of: SEQ ID NO: 2 and SEQ ID NO: 7 (7-I1-B), respectively; SEQ ID NO: 12 and SEQ ID NO: 17 (2-C8-A), respectively; SEQ ID NO: 22 and SEQ ID NO: 27 (10-O18-A), respectively; and SEQ ID NO: 32 and SEQ ID NO: 37 (6-G23-F), respectively.

[9] In some embodiments according to any one of [1]-[8], the antibody-drug conjugate undergoes intracellular internalization when the anti-DLL3 antibody binds to a DLL3 polypeptide expressed on a cell surface (e.g., a tumor cell surface).

[10] In some embodiments according to any one of [1]-[9], the anti-DLL3 comprises a Fc domain of an isotype selected from the group consisting of IgG1 or the variant thereof, IgG2, IgG3, IgG4, IgA1, IgA2, IgM, IgD, and IgE.

[11] In some embodiments according to any one of [1]-[10], the anti-DLL3 comprises a heavy chain constant region of SEQ ID NO: 42, 57 or 58.

[12] In some embodiments according to any one of [1]-[11], the anti-DLL3 antibody is a monoclonal antibody, a chimeric antibody, a humanized antibody, a human antibody or a bispecific antibody.

[13] In some embodiments according to any one of [1]-[12], the antibody comprises:
(a) a heavy chain comprises amino acid sequence of any one of SEQ ID NOs: 59 to 61 and a light chain comprises amino acid sequence of any one of SEQ ID NOs: 62;
(b) a heavy chain comprises amino acid sequence of any one of SEQ ID NOs: 63 to 65 and a light chain comprises amino acid sequence of any one of SEQ ID NOs: 66; and/or
(c) a heavy chain comprises amino acid sequence of any one of SEQ ID NOs: 67 to 69 and a light chain comprises amino acid sequence of any one of SEQ ID NOs: 70.

[14] In some embodiments according to any one of [1]-[13], the antibody comprises:
(a) a heavy chain comprises amino acid sequence of SEQ ID NO: 61 and a light chain comprises amino acid sequence of SEQ ID NO: 62;
(b) a heavy chain comprises amino acid sequence of SEQ ID NO: 65 and a light chain comprises amino acid sequence of SEQ ID NO: 66; or
(c) a heavy chain comprises amino acid sequence of SEQ ID NO: 69 and a light chain comprises amino acid sequence of SEQ ID NO: 70.

[15] In some embodiments according to claim 1, the anti-DLL-3 antibody competes with the antibody according to claim 9 or Claim V for binding to DLL-3.

[16] In some embodiments according to any one of [1]-[15], the anti-DLL3 antibody binds to an epitope on DLL3 that is a conformational epitope or a non-conformational epitope.

[17] In some embodiments according to any one of [1]-[16], the anti-DLL3 antibody binds to a mammalian DLL3 polypeptide that has an amino acid sequence comprising amino acid residues 27-492 of SEQ ID NO: 50 or SEQ ID NO: 51.

[18] In some embodiments according to any one of [1]-[17], the heavy chain or the light chain of the antibody has one or two or more modifications or sets of amino acid residues selected from the group consisting of N-linked glycosylation, O-linked glycosylation, N-terminal processing, C-terminal processing, deamidation, isomerization of aspartic acid, oxidation of methionine, addition of a methionine residue to the N-terminus, amidation of a proline residue, the substitutions of two leucine (L) residues to alanine (A) at position 234 and 235 (EU Numbering) of the heavy chain (LALA), a set of amino acid residues Glu (E) at positions 356 and Met (M) at position 358 (EU Numbering) of the heavy chain, a set of Asp (D) at positions 356 and Leucine (L) at position 358 (EU Numbering) of the heavy chain or any combination thereof, conversion of N-terminal glutamine or N-terminal glutamic acid to pyroglutamic acid, and a deletion of one or two amino acids from the carboxyl terminus.

[19] In some embodiments according to [18], one or two amino acids are deleted from the carboxyl terminus of a heavy chain thereof.

[20] In some embodiments according to [19], one amino acid is deleted from each of the carboxyl termini of both of the heavy chains thereof.

[21] In some embodiments according to any one of [18]-[20], a proline residue at the carboxyl terminus of a heavy chain thereof is further amidated.

[22] In some embodiments according to any one of [1]-[21], the anti-DLL3 antibody comprises a sugar chain modification that is regulated in order to enhance antibody-dependent cellular cytotoxic activity.

[23] In some embodiments according to any one of [1]-[22], D is:

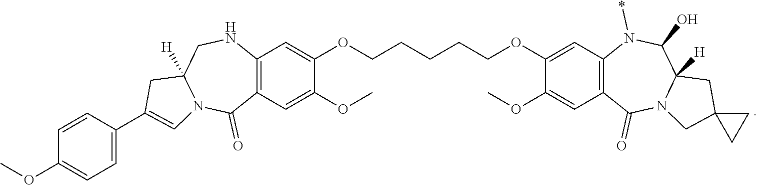 [SMILES: *N1c2cc(OCCCCCOc3cc4c(cc3OC)C(=O)N3C=C(c5ccc(OC)cc5)C[C@H]3CN4)c(OC)cc2C(=O)N2CC3(CC3)C[C@H]2[C@@H]1O]

[24] In some embodiments according to any one of [1]-[22], D is:

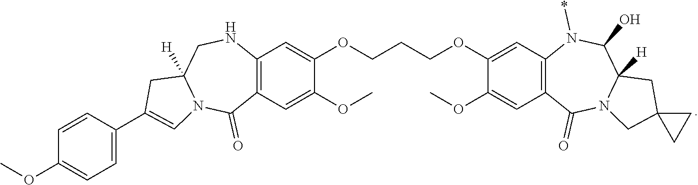 [SMILES: *N1c2cc(OCCCOc3cc4c(cc3OC)C(=O)N3C=C(c5ccc(OC)cc5)C[C@H]3CN4)c(OC)cc2C(=O)N2CC3(CC3)C[C@H]2[C@@H]1O]

[25] In some embodiments according to any one of [1]-[22], D is:

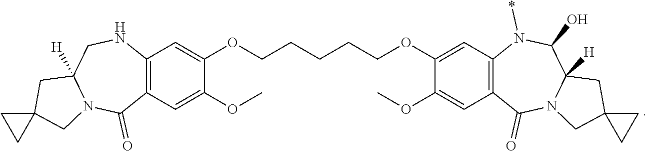 [SMILES: *N1c2cc(OCCCCCOc3cc4c(cc3OC)C(=O)N3CC5(CC5)C[C@H]3CN4)c(OC)cc2C(=O)N2CC3(CC3)C[C@H]2[C@@H]1O]

[26] In some embodiments according to any one of [1]-[22], D is:

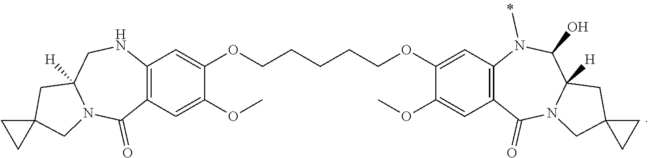 [SMILES: COc1cc2c(cc1OCCCCCOc1cc3c(cc1OC)C(=O)N1CC4(CC4)C[C@H]1[C@H](O)N3C)NC[C@@H]1CC3(CC3)CN1C2=O]

[27] In some embodiments according to any one of [23]-[26], the —OH is at the position 11′.

[28] In some embodiments according to any one of [1]-[27],
L is represented by -Lb-La-Lp-NH—B—CH₂—O(C═O)—*, the asterisk representing bonding to D; B represents a phenyl group or a heteroaryl group;
Lp represents a linker consisting of an amino acid sequence cleavable in a target cell;
La represents any one selected from the group:
—C(═O)—(CH₂CH₂)n²-C(═O)—,
—C(═O)—(CH₂CH₂)n²-C(═O)—NH—(CH₂CH₂)n³-C(═O)—,
—C(═O)—(CH₂CH₂)n²-C(═O)—NH—(CH₂CH₂O)n³-CH₂—C(═O)—,
—C(═O)—(CH₂CH₂)n²-NH—C(═O)—(CH₂CH₂O)n³-CH₂CH₂—C(═O)—, and
(CH₂)n⁴-O—C(═O)—;
n² represents an integer of 1 to 3, n³ represents an integer of 1 to 5, and n⁴ represents an integer of 0 to 2; and
Lb represents a spacer bonding La and a glycan or remodeled glycan of Ab.

[29] In some embodiments according to [28], B is any one selected from a 1,4-phenyl group, a 2,5-pyridyl group, a 3,6-pyridyl group, a 2,5-pyrimidyl group, and a 2,5-thienyl group.

[30] In some embodiments according to [29], B is a 1,4-phenyl group.

[31] In some embodiments according to any one of [28]-[30], Lp is amino acid residues composed of two to seven amino acids.

[32] In some embodiments according to any one of [28]-[31], Lp is amino acid residues consisting of amino acids selected from glycine, valine, alanine, phenylalanine, glutamic acid, isoleucine, proline, citrulline, leucine, serine, lysine, and aspartic acid.

[33] In some embodiments according to any one of [28]-[32], Lp is selected from the following group: -GGVA- (SEQ ID NO: 85), -GG-(D-)VA-, -VA-, -GGFG- (SEQ ID NO: 86), -GGPI- (SEQ ID NO: 87), -GGVCit- (SEQ ID NO: 88), -GGVK- (SEQ ID NO: 89), -GG(D-)PI-, and -GGPL- (SEQ ID NO: 90).

[34] In some embodiments according to any one of [28]-[33], La is selected from the following group:
—C(═O)—CH₂CH₂—C(═O)—, —C(═O)—(CH₂CH₂)₂—C(═O)—,
—C(═O)—CH₂CH₂—C(═O)—NH—(CH₂CH₂)₂—C(═O)—,
—C(═O)—CH₂CH₂—C(═O)—NH—(CH₂CH₂O)₂—CH₂—C(═O)—,
—C(═O)—CH₂CH₂—NH—C(═O)—(CH₂CH₂O)₄—CH₂CH₂—C(═O)—,
—CH₂—OC(═O)—, and —OC(═O)—.

[35] In some embodiments according to any one of [28]-[34], Lb is represented by the following formula:

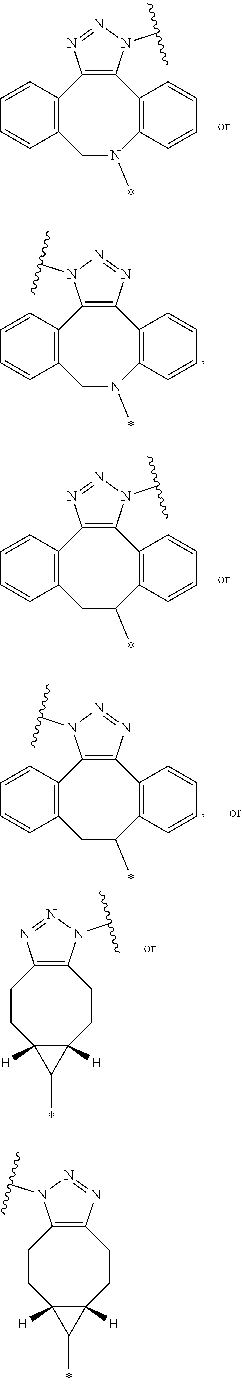

wherein, in each structural formula for Lb shown above,
each asterisk represents bonding to La, and each wavy line represents bonding to a glycan or remodeled glycan of Ab.

[36] In some embodiments according to any one of [28]-[35],
L is represented by -Lb-La-Lp-NH—B—CH₂—O(C═O)—*, wherein
B is a 1,4-phenyl group;
Lp represents any one selected from the following group: -GGVA- (SEQ ID NO: 85), -GG-(D-)VA-, -VA-, -GGFG- (SEQ ID NO: 86), -GGPI- (SEQ ID NO: 87), -GGVCit- (SEQ ID NO: 88), -GGVK- (SEQ ID NO: 89), and -GGPL- (SEQ ID NO: 90);
La represents any one selected from the following group:
—C(═O)—CH₂CH₂—C(═O)—, —C(═O)—(CH₂CH₂)₂—C(═O)—,
—C(═O)—CH₂CH₂—C(═O)—NH—(CH₂CH₂)₂—C(═O)—,
—C(═O)—CH₂CH₂—C(═O)—NH—(CH₂CH₂O)₂—CH₂—C(═O)—,
—C(═O)—CH₂CH₂—NH—C(═O)—(CH₂CH₂O)₄—CH₂CH₂—C(═O)—,
—CH₂—OC(═O)—, and —OC(═O)—; and
Lb is represented by the following formula:

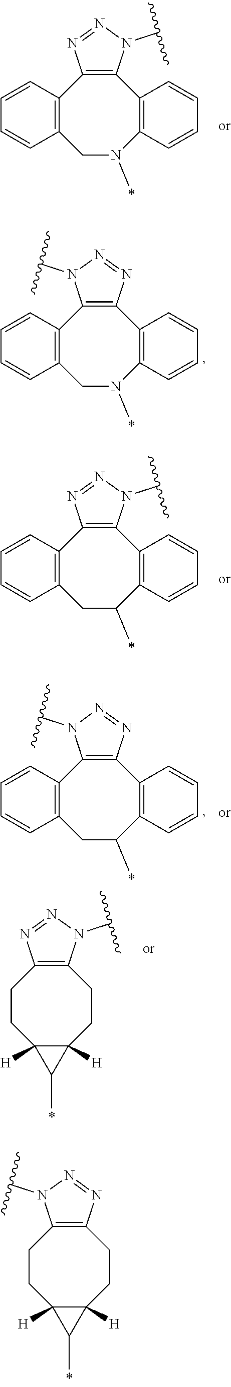

wherein, in each structural formula for Lb shown above,
each asterisk represents bonding to La, and each wavy line represents bonding to a glycan or remodeled glycan of Ab.

[37] In some embodiments according to any one of [28]-[36],
L is selected from the following group:
—Z¹—C(═O)—CH₂CH₂—C(═O)-GGVA-NH—B—CH₂—OC(═O)— (“GGVA” disclosed as SEQ ID NO: 85),
—Z¹—C(═O)—CH₂CH₂—C(═O)-GG-(D-)VA-NH—B—CH₂—OC(═O)—,
—Z¹—C(═O)—CH₂CH₂—C(═O)—VA-NH—B—CH₂—OC(═O)—,
—Z¹—C(═O)—(CH₂CH₂)₂—C(═O)—VA-NH—B—CH₂—OC(═O)—,
—Z¹—C(═O)—CH₂CH₂—C(═O)-GGPI-NH—B—CH₂—OC(═O)— (“GGPI” disclosed as SEQ ID NO: 87),
—Z¹—C(═O)—CH₂CH₂—C(═O)-GGFG-NH—B—CH₂—OC(═O)— (“GGFG” disclosed as SEQ ID NO: 86),
—Z¹—C(═O)—CH₂CH₂—C(═O)-GGVCit-NH—B—CH₂—OC(═O)— (“GGVCit” disclosed as SEQ ID NO: 88),
—Z¹—C(═O)—CH₂CH₂—C(═O)-GGVK-NH—B—CH₂—OC(═O)— (“GGVK” disclosed as SEQ ID NO: 89),
—Z¹—C(═O)—CH₂CH₂—C(═O)-GGPL-NH—B—CH₂—OC(═O)— (“GGPL” disclosed as SEQ ID NO: 90),
—Z¹—C(═O)—CH₂CH₂—C(═O)—NH—(CH₂CH₂)₂—C(═O)—VA-NH—B—CH₂—OC(═O)—,
—Z¹—C(═O)—CH₂CH₂—C(═O)—NH—(CH₂CH₂O)₂—CH₂—C(═O)—VA-NH—B—CH₂—OC(═O)—,
—Z¹—C(═O)—CH₂CH₂—NH—C(═O)—(CH₂CH₂O)₄—CH₂CH₂—C(═O)—VA-NH—B—CH₂—OC(═O)—,
—Z²—OC(═O)-GGVA-NH—B—CH₂—OC(═O)— (“GGVA” disclosed as SEQ ID NO: 85), and
—Z³—CH₂—OC(═O)-GGVA-NH—B—CH₂—OC(═O)— (“GGVA” disclosed as SEQ ID NO: 85), wherein
Z¹ represents the following structural formula:

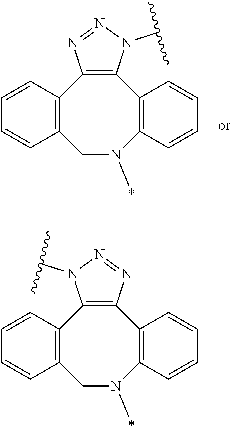 [SMILES: *n1nnc2c1-c1ccccc1N(C)Cc1ccccc1-2 ; *n1nnc2c1-c1ccccc1CN(C)c1ccccc1-2]

Z² represents the following structural formula:

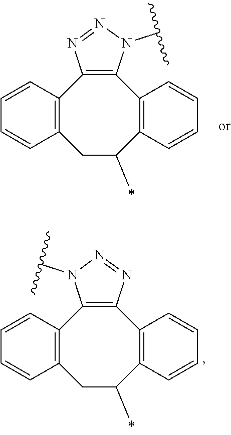 [SMILES: *n1nnc2c1-c1ccccc1C(C)Cc1ccccc1-2 ; *n1nnc2c1-c1ccccc1CC(C)c1ccccc1-2]

and
Z³ represents the following structural formula:

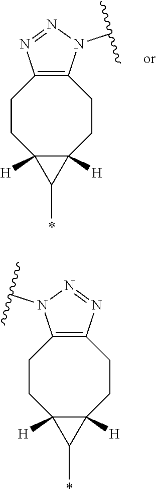

wherein, in each structural formula for Z¹, Z², and Z³,
each asterisk represents bonding to La, each wavy line represents bonding to a glycan or remodeled glycan of Ab; and
B represents a 1,4-phenyl group.

[38] In some embodiments according to [37],
L is selected from the following group:
—Z¹—C(═O)—CH₂CH₂—C(═O)-GGVA-NH—B—CH₂—OC(═O)— (“GGVA” disclosed as SEQ ID NO: 85),
—Z¹—C(═O)—CH₂CH₂—C(═O)—VA-NH—B—CH₂—OC(═O)—,
—Z¹—C(═O)—(CH₂CH₂)₂—C(═O)—VA-NH—B—CH₂—OC(═O)—,
—Z¹—C(═O)—CH₂CH₂—C(═O)-GGVCit-NH—B—CH₂—OC(═O)—, —Z¹—C(═O)—CH₂CH₂—C(═O)—NH—(CH₂CH₂)₂—C(═O)—VA-NH—B—CH₂—OC(═O)— (“GGVCit” disclosed as SEQ ID NO: 88),
—Z¹—C(═O)—CH₂CH₂—C(═O)—NH—(CH₂CH₂O)₂—CH₂—C(═O)—VA-NH—B—CH₂—OC(═O)—, and
—Z¹—C(═O)—CH₂CH₂—NH—C(═O)—(CH₂CH₂O)₄—CH₂CH₂—C(═O)—VA-NH—B—CH₂—OC(═O)—, wherein
B is a 1,4-phenyl group, and Z¹ represents the following structural formula:

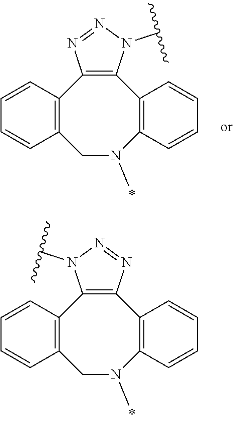

wherein, in the structural formula for Z¹,
each asterisk represents bonding to C(═O) neighboring to Z¹, and each wavy line represents bonding to a glycan or remodeled glycan of Ab.

[39] In some embodiments according to any one of [1]-[38],
L is represented by -Lb-La-Lp-NH—B—CH₂—O(C═O)—*, wherein
the asterisk represents bonding to D;
B represents a 1,4-phenyl group;
Lp represents -GGVA- (SEQ ID NO: 85) or -VA;
La represents —(CH₂)n⁹-C(═O)— or —(CH₂CH₂)n¹⁰-C(═O)—NH—(CH₂CH₂O)n¹¹-CH₂CH₂—C(═O)—, wherein n⁹ represents an integer of 2 to 7, n¹⁰ represents an integer of 1 to 3, and n¹¹ represents an integer of 6 to 10; and
Lb is -(succinimid-3-yl-N)—.

[40] In some embodiments according to [39],
L represents any one selected from the following group:
-(succinimid-3-yl-N)—(CH₂)₅—C(═O)—VA-NH—B—CH₂—OC(═O)—,
-(succinimid-3-yl-N)—(CH₂)₅—C(═O)-GGVA-NH—B—CH₂—OC(═O)—)— (“GGVA” disclosed as SEQ ID NO: 85), and
-(succinimid-3-yl-N)—CH₂CH₂—C(═O)—NH—(CH₂CH₂O)₈—CH₂CH₂—C(═O)—VA-NH—B—CH₂—OC(═O)—, wherein B is a 1,4-phenyl group.

[41] In some embodiments according to any one of [1]-[40], the antibody is IgG.

[42] In some embodiments according to [41], the antibody is IgG1 or the variant thereof, IgG2, or IgG4.

[43] In some embodiments according to any one of [1]-[42], the antibody binds to a tumor cell, and is incorporated and internalizes in the tumor cell.

[44] In some embodiments according to [43], the antibody further has antitumor effect.

[45] In some embodiments according to any one of [1]-[44], the N297 glycan is a remodeled glycan.

[46] In some embodiments according to [45], the N297 glycan is N297-(Fuc)MSG1, N297-(Fuc)MSG2, or a mixture thereof, or N297-(Fuc)SG, with N297-(Fuc)MSG1, N297-(Fuc)MSG2, and N297-(Fuc)SG having structures represented by the following formulas:

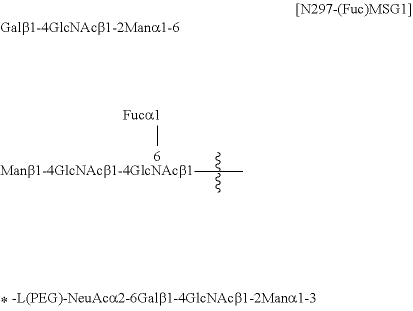

wherein
the wavy line represents bonding to Asn297 of the antibody;
L(PEG) represents —(CH₂CH₂—O)n⁵-CH₂CH₂—NH—, wherein the amino group at the right end is bound via an amide bond to carboxylic acid at the 2-position of a sialic acid at the non-reducing terminal in the 1-3 branched chain of β-Man in the N297 glycan;
the asterisk represents bonding to linker L; and
n⁵ represents an integer of 2 to 10,

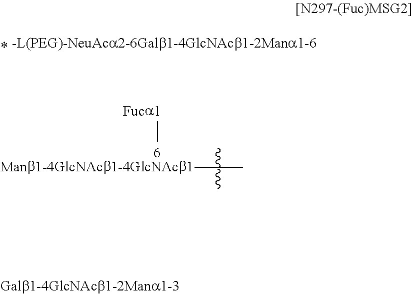

wherein
the wavy line represents bonding to Asn297 of the antibody;
L(PEG) represents —(CH₂CH₂—O)n⁵-CH₂CH₂—NH—, wherein the amino group at the right end is bound via an amide bond to carboxylic acid at the 2-position of a sialic acid at the non-reducing terminal in the 1-6 branched chain of β-Man in the N297 glycan;
the asterisk represents bonding to linker L; and
n⁵ represents an integer of 2 to 10, and

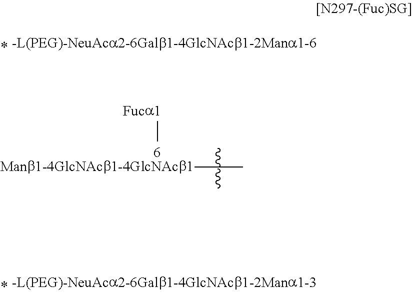

wherein
the wavy line represents bonding to Asn297 of the antibody;
L(PEG) represents —(CH₂CH₂—O)n⁵-CH₂CH₂—NH—, wherein the amino group at the right end is bound via an amide bond to carboxylic acid at the 2-position of a sialic acid at the non-reducing terminal in each of the 1-3 and 1-6 branched chains of β-Man in the N297 glycan;
the asterisk represents bonding to linker L; and
n⁵ represents an integer of 2 to 10.

[47] In some embodiments according to [46], n⁵ is an integer of 2 to 5.

[48] In some embodiments according to any one of [1]-[47], m² represents an integer of 1 or 2;
L is selected from the following group:
—Z¹—C(═O)—CH₂CH₂—C(═O)-GGVA-NH—B—CH₂—OC(═O)— (“GGVA” disclosed as SEQ ID NO: 85),
—Z¹—C(═O)—CH₂CH₂—C(═O)—VA-NH—B—CH₂—OC(═O)—,
—Z¹—C(═O)—(CH₂CH₂)₂—C(═O)—VA-NH—B—CH₂—OC(═O)—,
—Z¹—C(═O)—CH₂CH₂—C(═O)-GGVCit-NH—B—CH₂—OC(═O)— (“GGVCit” disclosed as SEQ ID NO: 88),
—Z¹—C(═O)—CH₂CH₂—C(═O)—NH—(CH₂CH₂)₂—C(═O)—VA-NH—B—CH₂—OC(═O)—,
—Z¹—C(═O)—CH₂CH₂—C(═O)—NH—(CH₂CH₂O)₂—CH₂—C(═O)—VA-NH—B—CH₂—OC(═O)—, and —Z¹—C(═O)—CH₂CH₂—NH—C(═O)—(CH₂CH₂O)₄—CH₂CH₂—C(═O)—VA-NH—B—CH₂—OC(═O)—, wherein
B is a 1,4-phenyl group, and Z¹ represents the following structural formula:

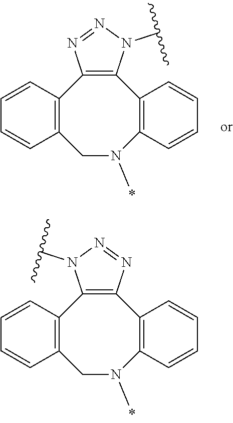

wherein, in the structural formulas for Z¹,
each asterisk represents bonding to C(═O) neighboring to Z¹, and each wavy line represents bonding to the N297 glycan of Ab;
Ab represents an IgG antibody;
the N297 glycan of Ab represents any one of N297-(Fuc)MSG1, N297-(Fuc)MSG2, and a mixture thereof, and N297-(Fuc)SG, with N297-(Fuc)MSG1, N297-(Fuc)MSG2, and N297-(Fuc)SG having structures represented by the following formulas:

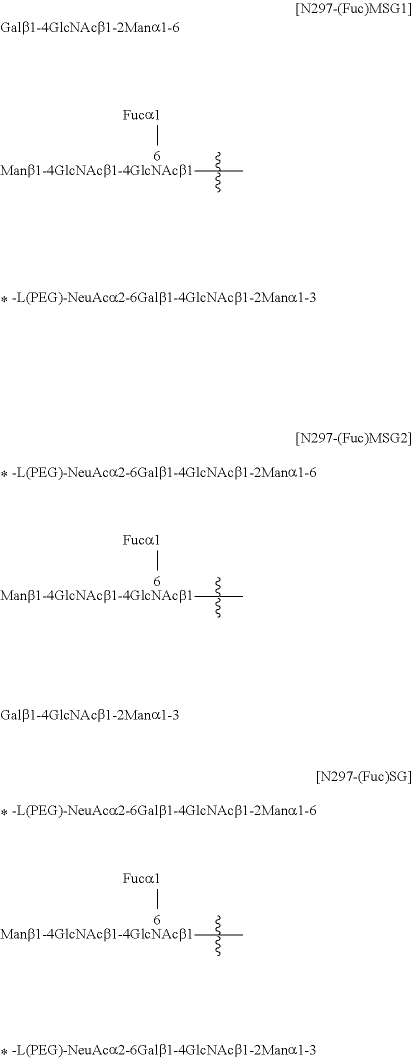

wherein
each wavy line represents bonding to Asn297 of the antibody,
L(PEG) in the N297 glycan represents —NH—CH₂CH₂—(O—CH₂CH₂)n⁵-*, wherein
n⁵ represents an integer of 2 to 5, the amino group at the left end is bound via an amide bond to carboxylic acid at the 2-position of a sialic acid at the non-reducing terminal in each or either one of the 1-3 and 1-6 branched chains of β-Man in the N297 glycan, and each asterisk represents bonding to a nitrogen atom at the 1- or 3-position of the triazole ring of Z¹ in linker L.

[49] In another aspect, the present disclosure provides an antibody-drug conjugate selected from the following group:

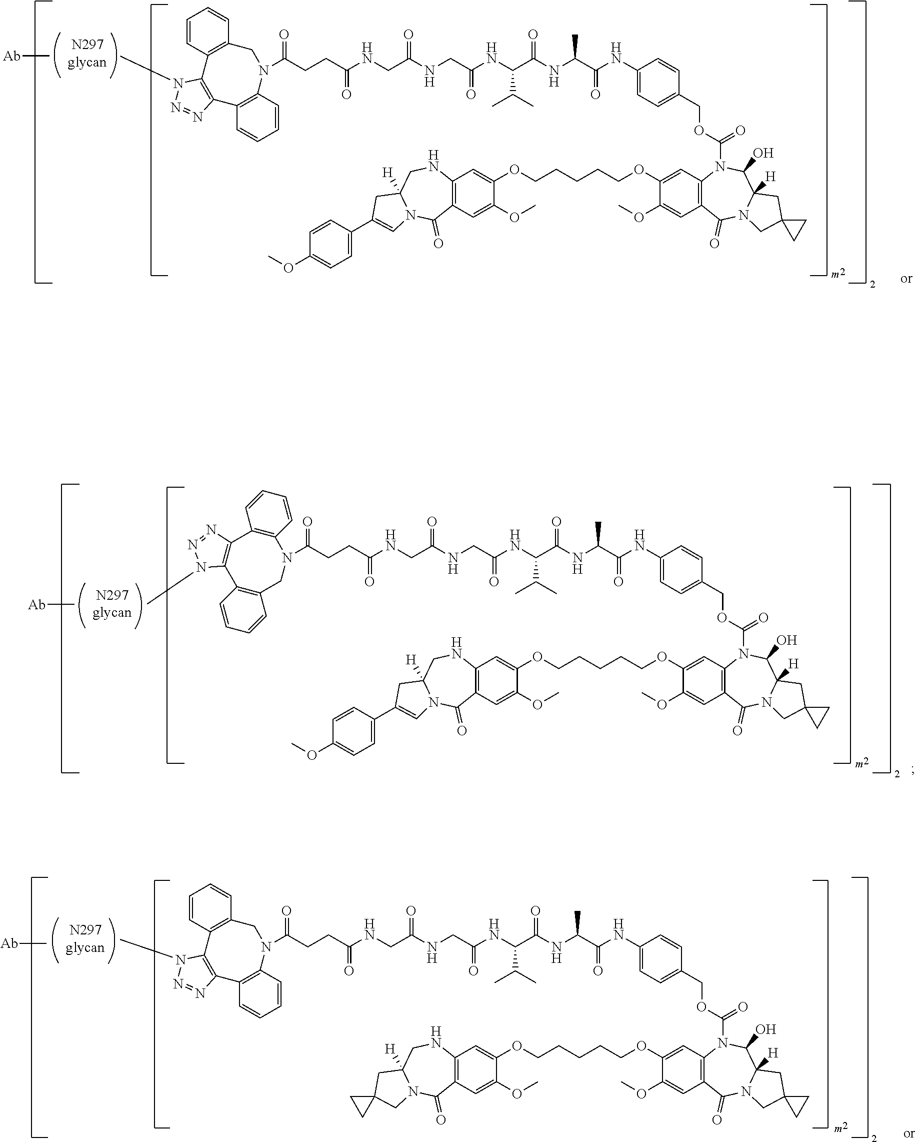

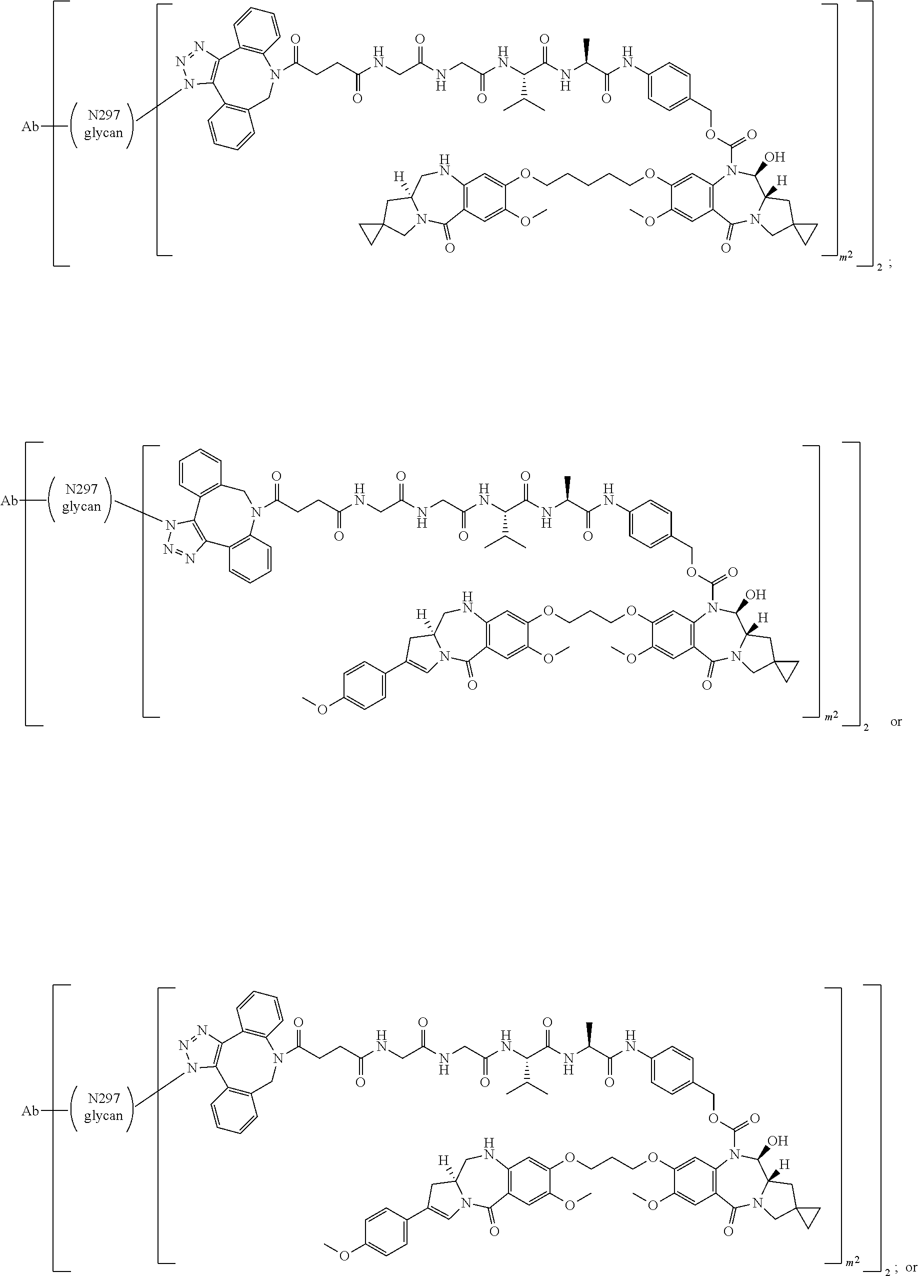

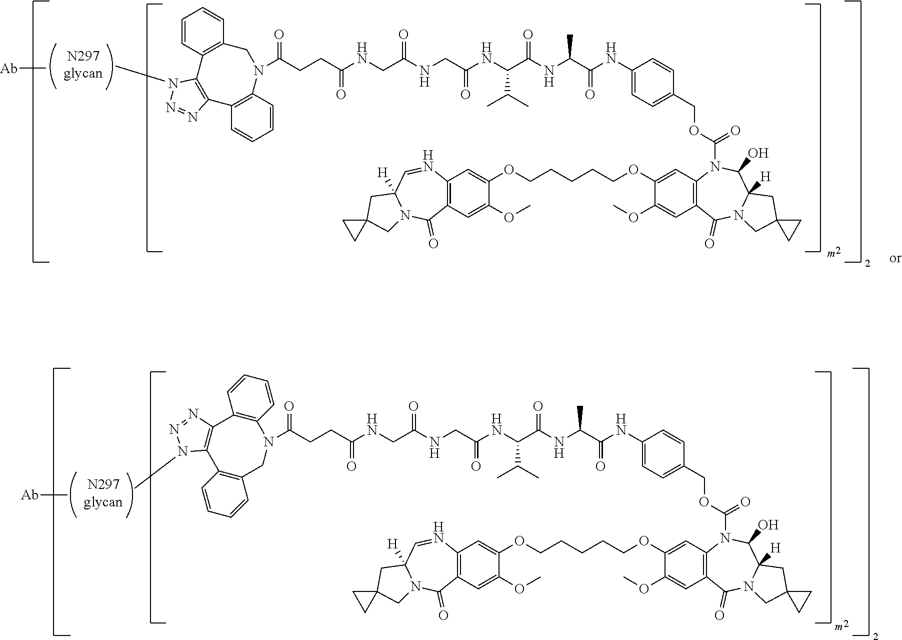

wherein, in each structural formula shown above,
m² represents an integer of 1 or 2;
Ab represents an anti-DLL3 IgG antibody or a functional fragment of the antibody;
the N297 glycan of Ab represents any one of N297-(Fuc)MSG1, N297-(Fuc)MSG2, and a mixture thereof, and N297-(Fuc)SG, with N297-(Fuc)MSG1, N297-(Fuc)MSG2, and N297-(Fuc)SG having structures represented by the following formulas:

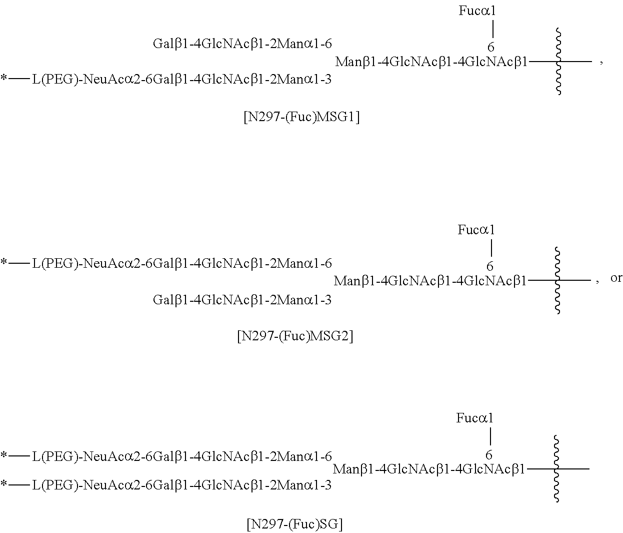

wherein
each wavy line represents bonding to Asn297 of the antibody,
L(PEG) in the N297 glycan represents —NH—CH₂CH₂—(O—CH₂CH₂)₃—*, wherein
the amino group at the left end is bound via an amide bond to carboxylic acid at the 2-position of a sialic acid at the non-reducing terminal in each or either one of the 1-3 and 1-6 branched chains of β-Man in the N297 glycan, and each asterisk represents bonding to a nitrogen atom at the 1- or 3-position of the triazole ring in the corresponding structural formula.

[50] In some embodiments according to [49], the antibody comprising a heavy chain constant region of human IgG1 or the variant thereof, human IgG2, or human IgG4.

[51] In some embodiments according to [49], the antibody comprises a heavy chain constant region of SEQ ID NO: 42, 57 or 58.

[52] In some embodiments according to any one of [48]-[51], the anti-DLL3 antibody comprises a heavy chain immunoglobulin variable domain (V_{H}) and a light chain immunoglobulin variable domain (V_{L}), wherein (a) the V_{H} comprises a V_{H}-CDR1 sequence, a V_{H}-CDR2 sequence, and a V_{H}-CDR3 sequence selected from the group consisting of
(i) SEQ ID NO: 3, SEQ ID NO: 4, and SEQ ID NO: 5, respectively;
(ii) SEQ ID NO: 13, SEQ ID NO: 14, and SEQ ID NO: 15, respectively;
(iii) SEQ ID NO: 23, SEQ ID NO: 24, and SEQ ID NO: 25, respectively; and
(iv) SEQ ID NO: 33, SEQ ID NO: 34, and SEQ ID NO: 35, respectively; and/or
(b) the V_{L} comprises a V_{L}-CDR1 sequence, a V_{L}-CDR2 sequence, and a V_{L}-CDR3 sequence selected from the group consisting of:
   (i) SEQ ID NO: 8, SEQ ID NO: 9, and SEQ ID NO: 10, respectively;
   (ii) SEQ ID NO: 18, SEQ ID NO: 19, and SEQ ID NO: 20, respectively;
   (iii) SEQ ID NO: 28, SEQ ID NO: 29, and SEQ ID NO: 30, respectively; and
   (iv) SEQ ID NO: 38, SEQ ID NO: 39, and SEQ ID NO: 40, respectively.

[53] In some embodiments according to [52], the anti-DLL3 antibody comprises a heavy chain immunoglobulin variable domain (V_{H}) and a light chain immunoglobulin variable domain (V_{L}), wherein the combination of (a) the V_{H} comprising a V_{H}-CDR1 sequence, a V_{H}-CDR2 sequence, and a V_{H}-CDR3 sequence and (b) the V_{L} comprising a V_{L}-CDR1 sequence, a V_{L}-CDR2 sequence, and a V_{L}-CDR3 sequence is selected from the group consisting of:
(i) (a) SEQ ID NO: 3, SEQ ID NO: 4, and SEQ ID NO: 5 and (b) SEQ ID NO: 8, SEQ ID NO: 9, and SEQ ID NO: 10, respectively;
(ii) (a) SEQ ID NO: 13, SEQ ID NO: 14, and SEQ ID NO: 15 and (b) SEQ ID NO: 18, SEQ ID NO: 19, and SEQ ID NO: 20, respectively;
(iii) (a) SEQ ID NO: 23, SEQ ID NO: 24, and SEQ ID NO: 25 and (b) SEQ ID NO: 28, SEQ ID NO: 29, and SEQ ID NO: 30, respectively; and
(iv) (a) SEQ ID NO: 33, SEQ ID NO: 34, and SEQ ID NO: 35 and (b) SEQ ID NO: 38, SEQ ID NO: 39, and SEQ ID NO: 40, respectively.

[54] In some embodiments according to any one of [49]-[53], the antibody is an antibody comprising a light chain and a heavy chain in any one combination selected from the group consisting of the following combinations (1) to (4), or a functional fragment of the antibody:
(1) a light chain comprising a variable domain sequence of SEQ ID NO: 7 and a heavy chain comprising a variable domain sequence of SEQ ID NO: 2,
(2) a light chain comprising a variable domain sequence of SEQ ID NO: 17 and a heavy chain comprising a variable domain sequence of SEQ ID NO: 12,
(3) a light chain comprising a variable domain sequence of SEQ ID NO: 27 and a heavy chain comprising a variable domain sequence of SEQ ID NO: 22, and
(4) a light chain comprising a variable domain sequence of SEQ ID NO: 37 and a heavy chain comprising a variable domain sequence of SEQ ID NO: 32.

[55] In some embodiments according to [54], the antibody is an antibody comprising a light chain comprising a variable domain sequence of SEQ ID NO: 17 and a heavy chain comprising a variable domain sequence of SEQ ID NO: 12.

[56] In some embodiments according to [54], the antibody is an antibody comprising a light chain comprising a variable domain sequence of SEQ ID NO: 27 and a heavy chain comprising a variable domain sequence of SEQ ID NO: 22.

[57] In some embodiments according to [54], the antibody is an antibody comprising a light chain comprising a variable domain sequence of SEQ ID NO: 37 and a heavy chain comprising a variable domain sequence of SEQ ID NO: 32.

[58] In some embodiments according to [54], wherein the antibody is an antibody comprising a light chain comprising a variable domain sequence of SEQ ID NO: 7 and a heavy chain comprising a variable domain sequence of SEQ ID NO: 2.

[59] In some embodiments according to any one of [49]-[58], the antibody comprises:
(a) a heavy chain comprises amino acid sequence of any one of SEQ ID NOs: 59 to 61 and a light chain comprises amino acid sequence of SEQ ID NO: 62;
(b) a heavy chain comprises amino acid sequence of any one of SEQ ID NOs: 63 to 65 and a light chain comprises amino acid sequence of SEQ ID NO: 66; or
(c) a heavy chain comprises amino acid sequence of any one of SEQ ID NOs: 67 to 69 and a light chain comprises amino acid sequence of SEQ ID NO: 70.

[60] In some embodiments according to any one of [49]-[59], the antibody comprises:
(a) a heavy chain comprises amino acid sequence of SEQ ID NO: 61 and a light chain comprises amino acid sequence of SEQ ID NO: 62;
(b) a heavy chain comprises amino acid sequence of SEQ ID NO: 65 and a light chain comprises amino acid sequence of SEQ ID NO: 66; or
(c) a heavy chain comprises amino acid sequence of SEQ ID NO: 69 and a light chain comprises amino acid sequence of SEQ ID NO: 70.

[61] In some embodiments according to any one of [49]-[60], the heavy chain or the light chain has one or two or more modifications or sets of amino acid residues selected from the group consisting of N-linked glycosylation, O-linked glycosylation, N-terminal processing, C-terminal processing, deamidation, isomerization of aspartic acid, oxidation of methionine, addition of a methionine residue to the N-terminus, amidation of a proline residue, the substitutions of two leucine (L) residues to alanine (A) at position 234 and 235 (according to EU index) of the heavy chain (LALA), a set of amino acid residues Glu (E) at positions 356 and Met (M) at position 358 (according to EU index) of the heavy chain, a set of Asp (D) at positions 356 and Leucine (L) at position 358 (according to EU index) of the heavy chain or any combination thereof, conversion of N-terminal glutamine or N-terminal glutamic acid to pyroglutamic acid, and a deletion of one or two amino acids from the carboxyl terminus.

[62] In another aspect, the present disclosure provides an antibody-drug conjugate represented by the following formula:

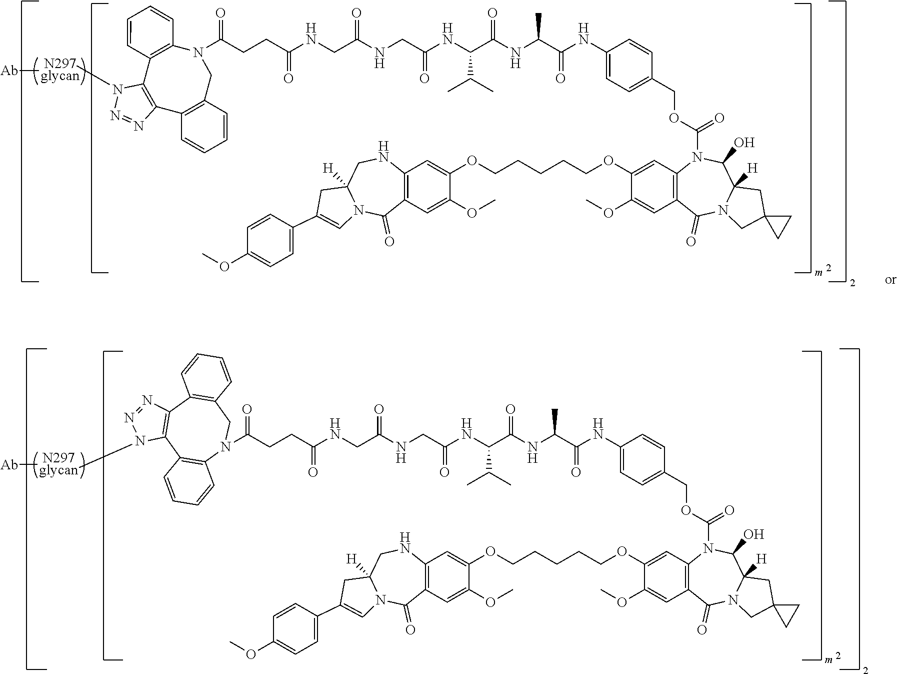

wherein,
m² represents an integer of 1 or 2;
Ab represents an anti-DLL3 IgG antibody which comprises a heavy chain of an amino acid sequence of SEQ ID NO: 61 and a light chain of an amino acid sequence of SEQ ID NO: 62;
the N297 glycan of Ab represents any one of N297-(Fuc)MSG1, N297-(Fuc)MSG2, and a mixture thereof, and N297-(Fuc)SG, with N297-(Fuc)MSG1, N297-(Fuc)MSG2, and N297-(Fuc)SG having structures represented by the following formulas:

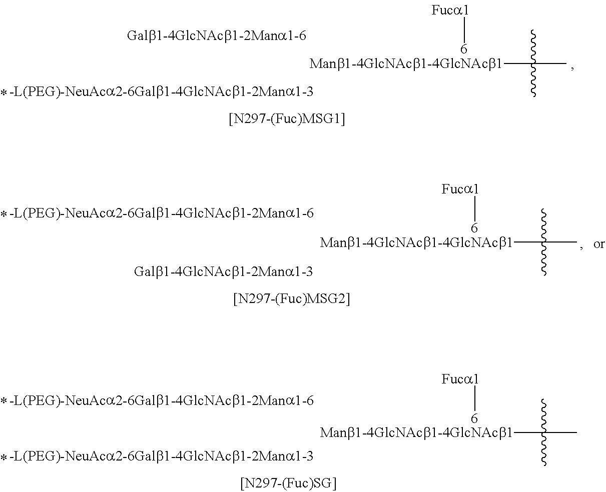

wherein
each wavy line represents bonding to Asn297 of the antibody,
L(PEG) in the N297 glycan represents —NH—CH₂CH₂—(O—CH₂CH₂)₃—*, wherein
the amino group at the left end is bound via an amide bond to carboxylic acid at the 2-position of a sialic acid at the non-reducing terminal in each or either one of the 1-3 and 1-6 branched chains of β-Man in the N297 glycan, and each asterisk represents bonding to a nitrogen atom at the 1- or 3-position of the triazole ring in the corresponding structural formula.

[63] In another aspect, the present disclosure provides an antibody-drug conjugate represented by the following formula:

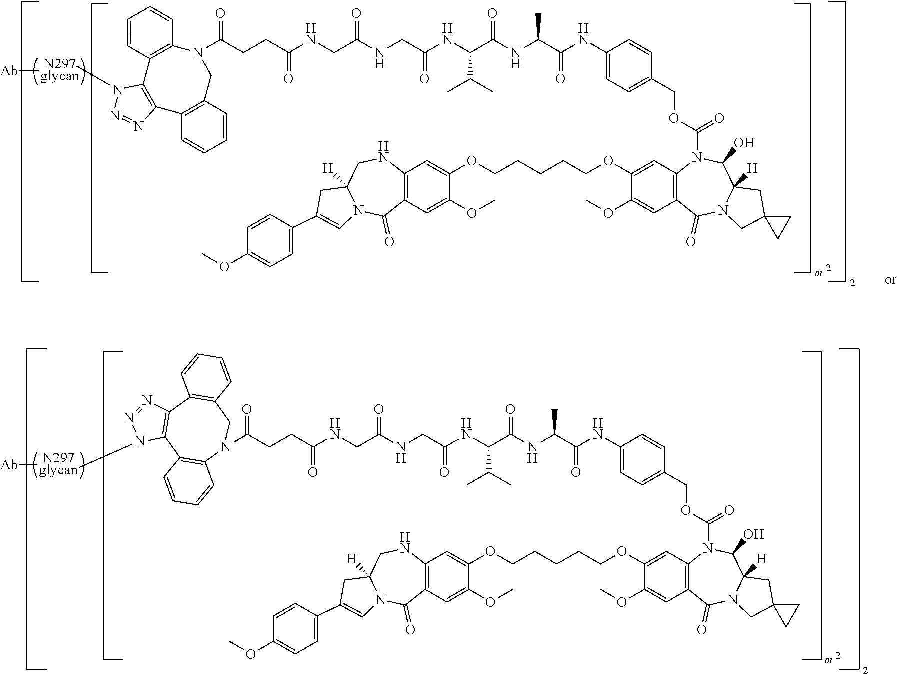

wherein,
m² represents an integer of 1 or 2;
Ab represents an anti-DLL3 IgG antibody which comprises a heavy chain of an amino acid sequence of SEQ ID NO: 65 and a light chain of an amino acid sequence of SEQ ID NO: 66;
the N297 glycan of Ab represents any one of N297-(Fuc)MSG1, N297-(Fuc)MSG2, and a mixture thereof, and N297-(Fuc)SG, with N297-(Fuc)MSG1, N297-(Fuc)MSG2, and N297-(Fuc)SG having structures represented by the following formulas:

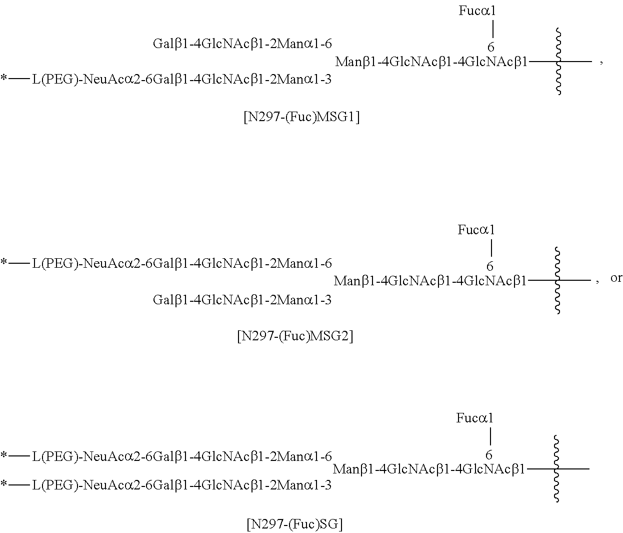

wherein
each wavy line represents bonding to Asn297 of the antibody,
L(PEG) in the N297 glycan represents —NH—CH₂CH₂—(O—CH₂CH₂)₃—*, wherein
the amino group at the left end is bound via an amide bond to carboxylic acid at the 2-position of a sialic acid at the non-reducing terminal in each or either one of the 1-3 and 1-6 branched chains of β-Man in the N297 glycan, and each asterisk represents bonding to a nitrogen atom at the 1- or 3-position of the triazole ring in the corresponding structural formula.

[64] In another aspect, the present disclosure provides an antibody-drug conjugate represented by the following formula:

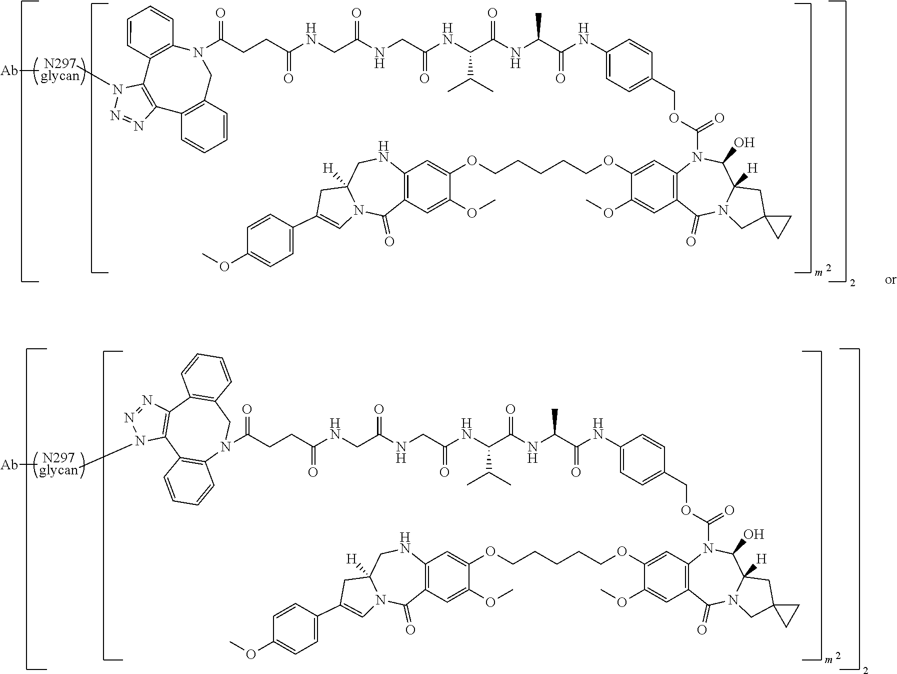

wherein,
m² represents an integer of 1 or 2;
Ab represents an anti-DLL3 IgG antibody which comprises a heavy chain of an amino acid sequence of SEQ ID NO: 69 and a light chain of an amino acid sequence of SEQ ID NO: 70;
the N297 glycan of Ab represents any one of N297-(Fuc)MSG1, N297-(Fuc)MSG2, and a mixture thereof, and N297-(Fuc)SG, with N297-(Fuc)MSG1, N297-(Fuc)MSG2, and N297-(Fuc)SG having structures represented by the following formulas:

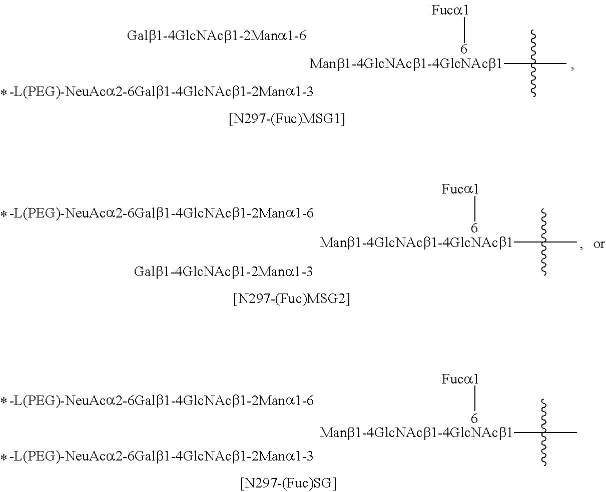

wherein
each wavy line represents bonding to Asn297 of the antibody,
L(PEG) in the N297 glycan represents —NH—CH₂CH₂—(O—CH₂CH₂)₃—*, wherein
the amino group at the left end is bound via an amide bond to carboxylic acid at the 2-position of a sialic acid at the non-reducing terminal in each or either one of the 1-3 and 1-6 branched chains of β-Man in the N297 glycan, and each asterisk represents bonding to a nitrogen atom at the 1- or 3-position of the triazole ring in the corresponding structural formula.

[65] In another aspect, the present disclosure provides a pharmaceutical composition comprising the antibody-drug conjugate according to any one of [1]-[64], a salt thereof, or a hydrate of the conjugate or the salt.

[66] In some embodiments according to [65], the pharmaceutical composition is an antitumor drug.

[67] In some embodiments according to [65] or [66], the pharmaceutical composition is for use in treating a tumor, wherein the tumor is a tumor expressing DLL3.

[68] In some embodiments according to [66] or [67], the tumor is small cell lung cancer (SCLC), large cell neuroendocrine carcinoma (LCNEC), neuroendocrine tumors of various tissues including kidney, genitourinary tract (bladder, prostate, ovary, cervix, and endometrium), gastrointestinal tract (stomach, colon), thyroid (medullary thyroid cancer), pancreas and lung, gliomas or pseudo neuroendocrine tumors (pNETs).

[69] In another aspect the present disclosure provides a method for producing a glycan-remodeled antibody, the method comprising the steps of:
i) culturing the host cell comprising a nucleic acid encoding an anti-DLL3 antibody and collecting a targeted antibody from the culture obtained;
ii) treating the antibody obtained in step i) with hydrolase to produce a (Fucα1,6)GlcNAc-antibody; and
iii)-1 reacting a glycan donner molecule and the (Fucα1,6)GlcNAc-antibody in the presence of transglycosidase, the glycan donner molecule obtained by introducing a PEG linker having an azide group to the carbonyl group of carboxylic acid at the 2-position of a sialic acid in MSG (9) or SG (10) and oxazolinating the reducing terminal, or
iii)-2 reacting the (Fucα1,6)GlcNAc-antibody and a glycan donner molecule in the presence of transglycosidase, the glycan donner molecule obtained by introducing a PEG linker having an azide group to the carbonyl group of carboxylic acid at the 2-position of a sialic acid in (MSG-)Asn or (SG-)Asn with an α-amino group optionally protected and to the carbonyl group of carboxylic acid in the Asn, causing action of hydrolase, and then oxazolinating the reducing terminal.

[70] In some embodiments according to [69], the methods further comprises the step of purifying the (Fucα1,6)GlcNAc-antibody through purification of a reaction solution in step ii) with a hydroxyapatite column.

[71] In another aspect the present disclosure provides a method for producing the antibody-drug conjugate form according to any one of [1]-[64], the method comprising the steps of:
i) producing a glycan-remodeled antibody by using the method according to [69] or [70]; and
ii) reacting a drug-linker having DBCO and an azide group in a glycan of the glycan-remodeled antibody made in step i).

[72] In another aspect the present disclosure provides a glycan-remodeled antibody obtained by using the method according to [69] or [70].

[73] In another aspect the present disclosure provides an antibody-drug conjugate obtained by using the method according to [71].

[74] In another aspect the present disclosure provides a method for treating a tumor, comprising administering to an individual with a tumor an antibody-drug conjugate according to any one of [1]-[64], a salt of the antibody-drug conjugate, or a hydrate of the antibody-drug conjugate or the salt.

[75] In another aspect the present disclosure provides a method for treating a tumor, which comprises administering a pharmaceutical composition comprising at least one component selected from the antibody-drug conjugate according to any one of [1]-[64], a salt thereof, and a hydrate of the conjugate or the salt, and at least one antitumor drug to an individual, simultaneously, separately, or continuously.

[76] In some embodiments according to [74] or [75], the tumor is a tumor expressing DLL3.

[77] In some embodiments according to any one of [74]-[76], the tumor is small cell lung cancer (SCLC), large cell neuroendocrine carcinoma (LCNEC), neuroendocrine tumors of various tissues including kidney, genitourinary tract (bladder, prostate, ovary, cervix, and endometrium), gastrointestinal tract (stomach, colon), thyroid (medullary thyroid cancer), pancreas and lung, gliomas or pseudo neuroendocrine tumors (pNETs).

Advantageous Effects of Invention: the novel anti-DLL-3 antibody-pyrrolobenzodiazepine (PBD) derivative conjugate provided by the present invention is superior in antitumor activity and safety, and hence useful as an antitumor agent.

### BRIEF DESCRIPTION OF DRAWINGS

FIG. 1 (FIG. 1) is a schematic diagram of the drug-conjugate form of the present invention (the molecule of (I)). (a) indicates drug D, (b) indicates linker L, (c) indicates N₃-L(PEG)-, and (d) indicates N297 glycan (open ellipse: NeuAc(Sia), open hexagon: Man, filled hexagon: GlcNAc, open diamond: Gal, open inverted triangle: Fuc). (b) and (c) are combined together to form a triazole ring by reaction between the azide group (filled teardrop shape) of (c) and the spacer (open semicircle) of (b). The Y-shaped diagram represents antibody Ab. For convenience, in this schematic diagram, N297 glycan is indicated as N297-(Fuc)MSG and the diagram shows an embodiment wherein any one of two branches in each of N297 glycans has a sialic acid to which a PEG linker having an azide group (N₃-L(PEG)-) bonds while other branch has no sialic acid at the non-reducing terminal (i.e. N297-(Fuc)MSG); however, another embodiment wherein each of two branches of N297 glycan has a sialic acid to which a PEG linker having an azide group bonds at the non-reducing terminal (i.e. N297-(Fuc)SG) is also acceptable. Unless otherwise stated, such a manner of illustration is applied throughout the present specification.

FIG. 2 (FIG. 2) is schematic diagrams illustrating the structures of a (Fucα1,6)GlcNAc-antibody (the molecule of A in (II) of FIG. 2), which is a production intermediate of the drug-conjugate form of the present invention, and an MSG-type glycan-remodeled antibody (the molecule of (III) in B of FIG. 2). In each of the diagrams, the Y-shaped diagram represents antibody Ab as in FIG. 1. In A in FIG. 2, (e) indicates N297 glycan consisting only of GlcNAc at the 6-positon connected to 1-positions of Fuc via an α glycosidic bond. In B in FIG. 2, (d) indicates the same N297 glycan as in FIG. 1, and (f) indicates a structure of a PEG linker portion having an azide group, specifically, an azide group to be bonded to liker L at the end. The bonding mode of the PEG linker having an azide group is as described for FIG. 1.

FIG. 3 (FIG. 3) is a schematic diagram for the step of producing an MSG-type glycan-remodeled antibody from an antibody produced in an animal cell. As in FIG. 2, molecules (II) and (III) in this Figure represent an (Fucα1,6)GlcNAc-antibody and an MSG-type glycan-remodeled antibody, respectively. Molecule (IV) is an antibody produced in an animal cell, and is a mixture of molecules with heterogeneous N297 glycan moieties. FIG. 3A illustrates the step of producing homogeneous (Fucα1,6)GlcNAc-antibody (II) by treating heterogeneous N297 glycan moieties of (IV) with hydrolase such as EndoS. FIG. 3B illustrates the step of producing the MSG-type glycan-remodeled antibody of (III) by subjecting GlcNAc of N297 glycan in antibody (II) to transglycosidase such as an EndoS D233Q/Q303L variant to transglycosylate the glycan of an MSG-type glycan donor molecule. The MSG-type glycan donor molecule used here has a sialic acid at the non-reducing terminal of MSG modified with a PEG linker having an azide group. Thus, resulting MSG-type N297 glycan-remodeled antibody also has a sialic acid at the non-reducing terminal modified in the same manner as described for FIG. 2B. For convenience, FIG. 3B shows MSG as a donor molecule. However, a glycan-remodeled antibody in which a linker molecule having an azide group bonds to each non-reducing terminal of N297 glycan also can be synthesized as the remodeled antibody of (III) by using SG (10) as a glycan donor.

FIG. 4 (FIG. 4): FIG. 4A shows the nucleotide sequence and the amino acid sequence of Homo sapiens delta like canonical Notch ligand 3 (DLL3) isoform 1, represented as SEQ ID NO: 55 and SEQ ID NO: 50, respectively. FIG. 4B shows the nucleotide sequence and the amino acid sequence of Homo sapiens delta like canonical Notch ligand 3 (DLL3) isoform 2, represented as SEQ ID NO: 56 and SEQ ID NO: 51, respectively.

FIG. 5 (FIG. 5): FIG. 5A shows the nucleotide sequence and the amino acid sequence of the V_{H} domain of the antibody 7-I1-B, represented as SEQ ID NO: 1 and SEQ ID NO: 2, respectively. The V_{H} CDR1 (SEQ ID NO: 3) is shown in a boldface font, V_{H} CDR2 (SEQ ID NO: 4) is underlined, and V_{H} CDR3 (SEQ ID NO: 5) is indicated in italicized, underlined font.

FIG. 5B shows the nucleotide sequence and the amino acid sequence of the V_{L} domain of the antibody 7-I1-B, represented as SEQ ID NO: 6 and SEQ ID NO: 7, respectively. The V_{L} CDR1 (SEQ ID NO: 8) is shown in a boldface font, V_{L} CDR2 (SEQ ID NO: 9) is underlined, and V_{L} CDR3 (SEQ ID NO: 10) is indicated in italicized, underlined font.

FIG. 6 (FIG. 6): FIG. 6A shows the nucleotide sequence and the amino acid sequence of the V_{H} domain of the antibody 2-C8-A, represented as SEQ ID NO: 11 and SEQ ID NO: 12, respectively. The V_{H} CDR1 (SEQ ID NO: 13) is shown in a boldface font, V_{H} CDR2 (SEQ ID NO: 14) is underlined, and V_{H} CDR3 (SEQ ID NO: 15) is indicated in italicized, underlined font. FIG. 6B shows the nucleotide sequence and the amino acid sequence of the V_{L} domain of the antibody 2-C8-A, represented as SEQ ID NO: 16 and SEQ ID NO: 17, respectively. The V_{L} CDR1 (SEQ ID NO: 18) is shown in a boldface font, V_{L} CDR2 (SEQ ID NO: 19) is underlined, and V_{L} CDR3 (SEQ ID NO: 20) is indicated in italicized, underlined font.

FIG. 7 (FIG. 7): FIG. 7A shows the nucleotide sequence and the amino acid sequence of the V_{H} domain of the antibody 10-O18-A, represented as SEQ ID NO: 21 and SEQ ID NO: 22, respectively. The V_{H} CDR1 (SEQ ID NO: 23) is shown in a boldface font, V_{H} CDR2 (SEQ ID NO: 24) is underlined, and V_{H} CDR3 (SEQ ID NO: 25) is indicated in italicized, underlined font. FIG. 7B shows the nucleotide sequence and the amino acid sequence of the V_{L} domain of the antibody 10-O18-A, represented as SEQ ID NO: 26 and SEQ ID NO: 27, respectively. The V_{L} CDR1 (SEQ ID NO: 28) is shown in a boldface font, V_{L} CDR2 (SEQ ID NO: 29) is underlined, and V_{L} CDR3 (SEQ ID NO: 30) is indicated in italicized, underlined font.

FIG. 8 (FIG. 8): FIG. 8A shows the nucleotide sequence and the amino acid sequence of the V_{H} domain of the antibody 6-G23-F, represented as SEQ ID NO: 31 and SEQ ID NO: 32, respectively. The V_{H} CDR1 (SEQ ID NO: 33) is shown in a boldface font, V_{H} CDR2 (SEQ ID NO: 34) is underlined, and V_{H} CDR3 (SEQ ID NO: 35) is indicated in italicized, underlined font. FIG. 8B shows the nucleotide sequence and the amino acid sequence of the V_{L} domain of the antibody 6-G23-F, represented as SEQ ID NO: 36 and SEQ ID NO: 37, respectively. The V_{L} CDR1 (SEQ ID NO: 38) is shown in a boldface font, V_{L} CDR2 (SEQ ID NO: 39) is underlined, and V_{L} CDR3 (SEQ ID NO: 40) is indicated in italicized, underlined font.

FIG. 9 (FIG. 9) shows the results of the Fab ZAP assay, a cytotoxicity-based internalization assay, conducted to assay internalization of DLL3 by the indicated antibodies. A reference DLL3 monoclonal antibody SC16 was used as a positive control. See WO2015127407. All tested anti-DLL3 antibodies exhibited killing activity that was comparable to the reference monoclonal antibody. A hook effect was observed at higher concentrations of the anti-DLL3 antibodies as free anti-DLL3 competed with cell bound anti-DLL3 for the Fab ZAP.

FIG. 10 (FIG. 10): FIGS. 10A-10D show binding curves for the antibodies 7-I1-B (FIG. 10A), 6-G23-F (FIG. 10B), 10-O18-A (FIG. 10C), and 2-C8-A (FIG. 10D), as measured via the Octet HTX at 25° C. using PBS 0.1% BSA 0.02% Tween 20 as the binding buffer and 10 mM Glycine pH 1.7 as the regeneration buffer. The monoclonal antibodies (5 μg/mL each) were loaded onto anti-mouse Fc sensors, and the loaded sensors were dipped into the indicated dilutions of Recombinant Human DLL3 Protein, (amino acids Ala27-Ala479, Cat #9749-DL, R&D Systems) at a 200 nM starting concentration, with 7 serial 1:3 dilutions. For each DLL3 dilution, the actual measurement and curve fits are shown. FIG. 10E shows the values for dissociation constants (K_{D}) for the four monoclonal antibodies described herein (6-G23-F, 2-C8-A, 7-I1-B and 10-O18-A), which were calculated using the binding curves shown in FIGS. 2A-2D and applying a monovalent (1:1) binding model.

FIG. 11 (FIG. 11) shows that the 6-G23-F, 10-O18-A, and 2-C8-A monoclonal antibodies (mAbs) selectively bind DLL3, but not DLL1 or DLL4. The 7-I1-B mAb binds both DLL3 and DLL4, but not DLL1.

FIG. 12 (FIG. 12) shows the reaction scheme for “Scheme R: Preparation of Antibody”, which is described in Section VI(E) below.

FIG. 13 (FIG. 13) shows a glycan remodeling scheme for H2-C8-A.

FIG. 14 (FIG. 14) shows a glycan remodeling scheme for H6-G23-F.

FIG. 15 (FIG. 15) shows a glycan remodeling scheme for H10-O18-A.

FIG. 16 (FIG. 16) shows synthesis steps for preparing a H2-C8-AADC.

FIG. 17 (FIG. 17) shows synthesis steps for preparing a H6-G23-F ADC.

FIG. 18 (FIG. 18) shows synthesis steps for preparing a H10-O18-A ADC.

FIG. 19 (FIG. 19) shows the in vivo antitumor effects of three human anti-DLL3-drug antibody-drug conjugates (H2-C8-A-Conjugate, H6-G23-F-Conjugate, H10-O18-A-Conjugate), or anti-LPS antibody-Conjugate. The evaluation was conducted using animal models in which DLL3-positive human small cell lung cancer cell line NCI-H209 was inoculated in immunodeficient mice. The abscissa depicts the days after inoculation, and the ordinate depicts estimated tumor volume. The error range depicts a standard error (SE) value. An arrow indicates the date of administration.

FIG. 20 (FIG. 20) shows the in vivo antitumor effects of three human anti-DLL3 antibody-drug conjugates (H2-C8-A-Conjugate, H6-G23-F-Conjugate, H10-O18-A-Conjugate), or anti-LPS antibody-Conjugate. The evaluation was conducted using animal models in which DLL3-positive human small cell lung cancer cell line NCI-H524 was inoculated in immunodeficient mice. The abscissa depicts the days after inoculation, and the ordinate depicts estimated tumor volume. The error range depicts a standard error (SE) value. An arrows indicate the date of administration.

FIG. 21 (FIG. 21) shows the in vivo antitumor effects of three human anti-DLL3 antibody-drug conjugates (H2-C8-A-Conjugate, H6-G23-F-Conjugate, H10-O18-A-Conjugate), or anti-DLL3 antibody-drug conjugate (SC16LD6.5). The evaluation was conducted using animal models in which DLL3-positive human small cell lung cancer cell line NCI-H510A was inoculated in immunodeficient mice. The abscissa depicts the days after inoculation, and the ordinate depicts estimated tumor volume. The error range depicts a standard error (SE) value. An arrow indicates the date of administration.

FIG. 22 (FIG. 22) shows a glycan remodeling scheme for H2-C8-A-3.

FIG. 23 (FIG. 23) shows a glycan remodeling scheme for H10-O18-A-3.

FIG. 24 (FIG. 24) shows synthesis steps for preparing a H2-C8-A-3 ADC.

FIG. 25 (FIG. 25) shows synthesis steps for preparing a H10-O18-A-3 ADC.

FIG. 26 (FIG. 26) shows the in vivo antitumor effects of two human anti-DLL3 antibody-drug Conjugates (H2-C8-A-3-Conjugate, H10-O18-A-3-Conjugate), anti-DLL3 antibody-drug conjugate (SC16LD6.5), or anti-LPS antibody-Conjugate. The evaluation was conducted using animal models in which DLL3-positive human small cell lung cancer cell line NCI-H510A was inoculated in immunodeficient mice. The abscissa depicts the days after administration, and the ordinate depicts estimated tumor volume. The error range depicts a standard error (SE) value.

FIG. 27 (FIG. 27) shows the in vivo antitumor effects of two human anti-DLL3 antibody-drug Conjugates (H2-C8-A-3-Conjugate, H10-O18-A-3-Conjugate). The evaluation was conducted using animal models in which DLL3-positive human small cell lung cancer cell line NCI-H209 was inoculated in immunodeficient mice. The abscissa depicts the days after administration, and the ordinate depicts estimated tumor volume. The error range depicts a standard error (SE) value.

FIG. 28 (FIG. 28) shows the in vivo antitumor effects of two human anti-DLL3 antibody-drug Conjugates (H2-C8-A-3-Conjugate, H10-O18-A-3-Conjugate), anti-DLL3 antibody-drug conjugate (SC16LD6.5), or anti-LPS antibody-Conjugate. The evaluation was conducted using animal models in which DLL3-positive human small cell lung cancer cell line NCI-H82 was inoculated in immunodeficient mice. The abscissa depicts the days after administration, and the ordinate depicts estimated tumor volume. The error range depicts a standard error (SE) value.

### DETAILED DESCRIPTION

The antibody-drug conjugate of the present invention is an antitumor drug having an antitumor compound conjugated via a linker structure moiety to an antibody capable of recognizing or binding to tumor cells.

It is to be appreciated that certain aspects, modes, embodiments, variations and features of the present technology are described below in various levels of detail in order to provide a substantial understanding of the present technology.

In practicing the present technology, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

Hereinafter, the preferred embodiments for carrying out the present invention will be described with reference to the drawings. It is to be noted that the embodiments described below merely illustrate the representative embodiments of the present invention, and the scope of the present invention shall not be narrowly interpreted due to these examples.

### I. Definitions

In the present description, the term “cancer” is used to have the same meaning as that of the term “tumor”.

In the present description, the term “gene” is used to include not only DNA but also its mRNA and cDNA, and cRNA thereof.

In the present description, the term “polynucleotide” or “nucleotide” is used to have the same meaning as that of a nucleic acid, and also includes DNA, RNA, a probe, an oligonucleotide, and a primer. In the present description, the terms “polynucleotide” and “nucleotide” can be used interchangeably with each other unless otherwise specified.

In the present description, the terms “polypeptide” and “protein” can be used interchangeably with each other.

In the present description, the term “cell” includes cells in an individual animal, and cultured cells.

In the present description, the term “DLL3” can be used to have the same meaning as that of the DLL3 protein. In the present description, human DLL3 is also referred to as “hDLL3”.

In the present description, the term “cytotoxic activity” is used to mean that a pathologic change is caused to cells in any given way. The term not only means a direct trauma, but also means all types of structural or functional damage caused to cells, such as DNA cleavage, formation of a base dimer, chromosomal cleavage, damage to cell mitotic apparatus, and a reduction in the activities of various types of enzymes.

In the present description, the phrase “exerting toxicity in cells” is used to mean that toxicity is exhibited in cells in any given way. The term not only means a direct trauma, but also means all types of structural, functional, or metabolic influences caused to cells, such as DNA cleavage, formation of a base dimer, chromosomal cleavage, damage to cell mitotic apparatus, a reduction in the activities of various types of enzymes, and suppression of effects of cell growth factors.

In the present description, the term “functional fragment of an antibody”, also called “antigen-binding fragment of an antibody”, is used to mean a partial fragment of the antibody having binding activity against an antigen, and includes Fab, F(ab′)2, scFv, a diabody, a linear antibody and a multispecific antibody formed from antibody fragments, and the like. Fab′, which is a monovalent fragment of antibody variable regions obtained by treating F(ab′)2 under reducing conditions, is also included in the antigen-binding fragment of an antibody. However, the antigen-binding fragment of an antibody is not limited to these molecules, as long as the antigen-binding fragment has antigen-binding ability. These antigen-binding fragments include not only those obtained by treating a full-length molecule of an antibody protein with an appropriate enzyme, but proteins produced in appropriate host cells using a genetically engineered antibody gene.

In the present description, the term “epitope” is used to mean the partial peptide or partial three-dimensional structure of DLL3, to which a specific anti-DLL3 antibody binds. Such an epitope, which is the above-described partial peptide of DLL3, can be determined by a method well known to a person skilled in the art, such as an immunoassay. First, various partial structures of an antigen are produced. As regards production of such partial structures, a known oligopeptide synthesis technique can be applied. For example, a series of polypeptides, in which DLL3 has been successively truncated at an appropriate length from the C-terminus or N-terminus thereof, are produced by a genetic recombination technique well known to a person skilled in the art. Thereafter, the reactivity of an antibody to such polypeptides is studied, and recognition sites are roughly determined. Thereafter, further shorter peptides are synthesized, and the reactivity thereof to these peptides can then be studied, so as to determine an epitope. When an antibody binding to a membrane protein having a plurality of extracellular domains is directed to a three-dimensional structure composed of a plurality of domains as an epitope, the domain to which the antibody binds can be determined by modifying the amino acid sequence of a specific extracellular domain, and thereby modifying the three-dimensional structure. The epitope, which is a partial three-dimensional structure of an antigen that binds to a specific antibody, can also be determined by specifying the amino acid residues of an antigen adjacent to the antibody by X-ray structural analysis.

In the present description, “humanized antibodies” refer to antibodies which comprise at least one chain comprising variable region framework residues from a human antibody chain and at least one complementarity determining region (CDR) from a non-human-antibody (e.g., mouse).

The term “human antibody,” as used herein, is intended to include antibodies having variable and constant regions derived from human immunoglobulin sequences. However, the term “human antibody,” as used herein, is not intended to include antibodies in which CDR sequences derived from another mammalian species, such as a mouse, have been grafted onto human framework sequences.

In the present description, the phrase “antibodies binding to the same epitope” is used to mean antibodies that bind to a common epitope. If a second antibody binds to a partial peptide or a partial three-dimensional structure to which a first antibody binds, it can be determined that the first antibody and the second antibody bind to the same epitope. Alternatively, by confirming that a second antibody competes with a first antibody for the binding of the first antibody to an antigen (i.e., a second antibody interferes with the binding of a first antibody to an antigen), it can be determined that the first antibody and the second antibody bind to the same epitope, even if the specific sequence or structure of the epitope has not been determined. In the present description, the phrase “binding to the same epitope” refers to the case where it is determined that the first antibody and the second antibody bind to a common epitope by any one or both of these determination methods. When a first antibody and a second antibody bind to the same epitope and further, the first antibody has special effects such as antitumor activity or internalization activity, the second antibody can be expected to have the same activity as that of the first antibody.

In the present description, the term “CDR” is used to mean a complementarity determining region. It is known that the heavy chain and light chain of an antibody molecule each have three CDRs. Such a CDR is also referred to as a hypervariable region, and is located in the variable regions of the heavy chain and light chain of an antibody. These regions have a particularly highly variable primary structure and are separated into three sites on the primary structure of the polypeptide chain in each of the heavy chain and light chain. In the present description, with regard to the CDR of an antibody, the CDRs of a heavy chain are referred to as CDRH1, CDRH2 and CDRH3, respectively, from the amino-terminal side of the amino acid sequence of the heavy chain, whereas the CDRs of a light chain are referred to as CDRL1, CDRL2 and CDRL3, respectively, from the amino-terminal side of the amino acid sequence of the light chain. These sites are located close to one another on the three-dimensional structure, and determine the specificity of the antibody to an antigen to which the antibody binds.

As used herein, the term “CDR-grafted antibody” means an antibody in which at least one CDR of an “acceptor” antibody is replaced by a CDR “graft” from a “donor” antibody possessing a desirable antigen specificity.

As used herein, the terms “individual”, “patient”, or “subject” can be an individual organism, a vertebrate, a mammal, or a human. In some embodiments, the individual, patient or subject is a human.

As used herein, the term “pharmaceutically-acceptable carrier” is intended to include any and all solvents, dispersion media, coatings, antibacterial and antifungal compounds, isotonic and absorption delaying compounds, and the like, compatible with pharmaceutical administration. Pharmaceutically-acceptable carriers and their formulations are known to one skilled in the art and are described, for example, in Remington's Pharmaceutical Sciences (20^{th} edition, ed. A. Gennaro, 2000, Lippincott, Williams & Wilkins, Philadelphia, PA.).

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

As used herein, “specifically binds” refers to a molecule (e.g., an antibody or antigen binding fragment thereof) which recognizes and binds another molecule (e.g., an antigen), but that does not substantially recognize and bind other molecules. The terms “specific binding,” “specifically binds to,” or is “specific for” a particular molecule (e.g., a polypeptide, or an epitope on a polypeptide), as used herein, can be exhibited, for example, by a molecule having a K_{D} for the molecule to which it binds to of about 10⁻⁴ M, 10⁻⁵ M, 10⁻⁶ M, 10⁻⁷ M, 10⁻¹ M, 10⁻⁹ M, 10⁻⁰ M, 10⁻¹¹ M, or 10⁻¹²M. The term “specifically binds” may also refer to binding where a molecule (e.g., an antibody or antigen binding fragment thereof) binds to a particular polypeptide (e.g., a DLL3 polypeptide), or an epitope on a particular polypeptide, without substantially binding to any other polypeptide, or polypeptide epitope.

In the present description, the term “one to several” is used to mean 1 to 10, 1 to 9, 1 to 8, 1 to 7, 1 to 6, 1 to 5, 1 to 4, 1 to 3, or 1 or 2.

In the present invention, examples of “halogen atom” may include, but are not limited to, a fluorine atom, a chlorine atom, a bromine atom, and an iodine atom.

In the present invention, “C1 to C6 alkyl group” refers to a linear or branched alkyl group having one to six carbon atoms. Examples of “C1 to C6 alkyl group” may include, but are not limited to, a methyl group, an ethyl group, a n-propyl group, an i-propyl group, a n-butyl group, an i-butyl group, a s-butyl group, a t-butyl, a n-pentyl group, and a n-hexyl.

In the present invention, “C1 to C6 alkoxy group” refers to an alkoxy group having a linear or branched alkyl group having one to six carbon atoms. Examples of “C1 to C6 alkoxy group” may include, but are not limited to, a methoxy group, an ethoxy group, a n-propoxy group, an i-propoxy group, a n-butoxy group, an i-butoxy, a s-butoxy group, a n-pentyloxy group, and a n-hexyloxy.

In the present invention, “C1 to C6 alkylthio group” refers to an alkylthio group having a linear or branched alkyl group having one to six carbon atoms. Examples of “C1 to C6 alkylthio group” may include, but are not limited to, a methylthio group, an ethylthio group, a n-propylthio group, an i-propylthio group, a n-butylthio group, an i-butylthio group, a s-butylthio group, a t-butylthio group, a n-pentylthio group, and a n-hexylthio group.

In the present invention, “three- to five-membered saturated hydrocarbon ring” refers to a saturated cyclic hydrocarbon group having three to five carbon atoms. Examples of “three- to five-membered saturated hydrocarbon ring” may include, but are not limited to, a cyclopropyl group, a cyclobutyl group, and a cyclopentyl group.

In the present invention, “C3 to C5 cycloalkoxy group” refers to a cycloalkoxy group having a saturated cyclic hydrocarbon group having three to five carbon atoms. Examples of “C3 to C5 cycloalkoxy group” may include, but are not limited to, a cyclopropoxy group, a cyclobutoxy group, and a cyclopentyloxy group.

In the present invention, examples of “three- to five-membered saturated heterocycle” may include, but are not limited to, 1,3-propylene oxide, azacyclobutane, trimethylene sulfide, tetrahydrofuran, and pyrrolidine.

In the present invention, examples of “aryl group” may include, but are not limited to, a phenyl group, a benzyl group, an indenyl group, a naphthyl group, a fluorenyl group, an anthranyl group, and a phenanthrenyl group.

In the present invention, examples of “heteroaryl group” may include, but are not limited to, a thienyl group, a pyrrolyl group, a pyrazolyl group, a triazolyl group, an oxazolyl group, an oxadiazolyl group, a thiazolyl group, a pyridyl group, a pyrimidyl group, a pyridazyl group, a pyrazinyl group, a quinolyl group, a quinoxalyl group, a benzothiophenyl group, a benzimidazolyl group, a benzotriazolyl group, and a benzofuranyl group.

In the present invention, examples of “six-membered heterocycle” may include, but are not limited to, a pyridine ring, a pyrimidine ring, and a pyridazine ring.

In the present invention, “spiro-bonded” refers to the situation in which, as exemplified in Examples, A and a pyrrolidine ring to which A bonds, or E and a pyrrolidine ring to which E bonds form a spiro ring.

### II. Delta-Like Ligand 3 (DLL3)

DLL3 (i.e., delta-like ligand 3 or delta-like protein 3) is selectively expressed in high grade pulmonary neuroendocrine tumors including SCLC and LCNEC. Increased expression of DLL3 was observed in SCLC and LCNEC patient-derived xenograft tumors and was also confirmed in primary tumors. See Saunders et al., Sci Translational Medicine 7(302): 302ra136 (2015). Increased expression of DLL3 has also been observed in extrapulmonary neuroendocrine cancers including prostate neuroendocrine carcinoma (Puca et al., Sci Transl Med 11(484): pii: eaav0891 (2019). While DLL3 is expressed on the surface of such tumor cells, it is not expressed in normal tissues. The present disclosure provides immunoglobulin-related compositions (e.g., antibodies or antigen binding fragments thereof), which internalize on binding to DLL3 on tumor cells, and are thus useful for delivering a toxic payload to these tumor cells. The immunoglobulin-related compositions of the present technology are useful in methods for detecting or treating DLL3-associated cancers in a subject in need thereof. Accordingly, the various aspects of the present methods relate to the preparation, characterization, and manipulation of anti-DLL3 antibodies. The immunoglobulin-related compositions of the present technology are useful alone or in combination with additional therapeutic agents for treating cancer. In some embodiments, the immunoglobulin-related composition is a humanized antibody, a chimeric antibody, or a bispecific antibody.

In Drosophila, Notch signaling is mediated primarily by the Notch receptor. Delta is one of the Drosophila ligands of Notch that activate signaling in adjacent cells. Humans have four known Notch receptors (NOTCH1 to NOTCH4), and three homologs of Delta, termed delta-like ligands: DLL1, DLL3 and DLL4. It has been reported that unlike DLL1 and DLL4, DLL3 inhibits Notch signaling rather than activating it.

DLL3 (also known as Delta-like 3 or SCDO1) is a member of the Delta-like family of Notch DSL ligands. Representative DLL3 protein orthologs include, but are not limited to: human

(Accession Nos. NP_058637:
MVSPRMSGLLSQTVILALIFLPQTRPAGVFELQIHSFGPGPGPGAPRSPC
SARLPCRLFFRVCLKPGLSEEAAESPCALGAALSARGPVYTEQPGAPAPD
LPLPDGLLQVPFRDAWPGTFSFIIETWREELGDQIGGPAWSLLARVAGRR
RLAAGGPWARDIQRAGAWELRFSYRARCEPPAVGTACTRLCRPRSAPSRC
GPGLRPCAPLEDECEAPLVCRAGCSPEHGFCEQPGECRCLEGWTGPLCTV
PVSTSSCLSPRGPSSATTGCLVPGPGPCDGNPCANGGSCSETPRSFECTC
PRGFYGLRCEVSGVTCADGPCFNGGLCVGGADPDSAYICHCPPGFQGSNC
EKRVDRCSLQPCRNGGLCLDLGHALRCRCRAGFAGPRCEHDLDDCAGRAC
ANGGTCVEGGGAHRCSCALGFGGRDCRERADPCAARPCAHGGRCYAHFSG
LVCACAPGYMGARCEFPVHPDGASALPAAPPGLRPGDPQRYLLPPALGLL
VAAGVAGAALLLVHVRRRGHSQDAGSRLLAGTPEPSVHALPDALNNLRTQ
EGSGDGPSSSVDWNRPEDVDPQGIYVISAPSIYAREVATPLFPPLHTGRA
GQRQHLLFPYPSSILSVK (SEQ ID NO: 50)
and
NP_982353:
MVSPRMSGLLSQTVILALIFLPQTRPAGVFELQIHSFGPGPGPGAPRSPC
SARLPCRLFFRVCLKPGLSEEAAESPCALGAALSARGPVYTEQPGAPAPD
LPLPDGLLQVPFRDAWPGTFSFIIETWREELGDQIGGPAWSLLARVAGRR
RLAAGGPWARDIQRAGAWELRFSYRARCEPPAVGTACTRLCRPRSAPSRC
GPGLRPCAPLEDECEAPLVCRAGCSPEHGFCEQPGECRCLEGWTGPLCTV
PVSTSSCLSPRGPSSATTGCLVPGPGPCDGNPCANGGSCSETPRSFECTC
PRGFYGLRCEVSGVTCADGPCFNGGLCVGGADPDSAYICHCPPGFQGSNC
EKRVDRCSLQPCRNGGLCLDLGHALRCRCRAGFAGPRCEHDLDDCAGRAC
ANGGTCVEGGGAHRCSCALGFGGRDCRERADPCAARPCAHGGRCYAHFSG
LVCACAPGYMGARCEFPVHPDGASALPAAPPGLRPGDPQRYLLPPALGLL
VAAGVAGAALLLVHVRRRGHSQDAGSRLLAGTPEPSVHALPDALNNLRTQ
EGSGDGPSSSVDWNRPEDVDPQGIYVISAPSIYAREA (SEQ ID NO:
51));
chimpanzee (Accession No. XP_003316395:
MVSPRMSRLLSQTVILALIFLPQTRPAGVFELQIHSFGPGPGPGAPRSPC
SARVPCRLFFRVCLKPGLSEEAAESPCALGAALSARGPVYTEQPGAPAPD
LPLPDGLLQVPFRDAWPGTFSFIIETWREELGDQIGGPAWSLLARVAGRR
RLAAGGTWARDIQRAGAWELRFSYRARCEPPAVGTACTRLCRPRSAPSRC
GPGLRPCAPLEDECEAPPVCRAGCSPEHGFCEQPGECRCLEGWTGPLCTV
PVSTSSCLSPRGPSSATTGCLVPGPGPCDGNPCANGGSCSETPGSFECAC
PRGFYGLRCEVSGVTCADGPCFNGGLCVGGADPDSAYICHCPPGFQGSNC
EKRVDRCSLQPCRNGGLCLDLGHALRCRCRAGFAGPRCEHDLDDCAGRAC
ANGGTCVEGGGAHRCSCALGFGGRDCRERADPCAARPCAHGGRCYAHFSG
LVCACAPGYMGARCEFPVHPDGASALPAAPPGLRPGDPQRYLLPPALGLL
VAAGVAGAALLLVHVRRRGHAQDAGARLLAGTPEPSVHALPDALNNLRTQ
EGAGDGPSSSVDWNRPEDVDPRGIYVISAPSIYAREA (SEQ ID NO:
52));
mouse (Accession No. NP_031892:
MVSLQVSPLSQTLILAFLLPQALPAGVFELQIHSFGPGPGLGTPRSPCNA
RGPCRLFFRVCLKPGVSQEATESLCALGAALSTSVPVYTEHPGESAAALP
LPDGLVRVPFRDAWPGTFSLVIETWREQLGEHAGGPAWNLLARVVGRRRL
AAGGPWARDVQRTGTWELHFSYRARCEPPAVGAACARLCRSRSAPSRCGP
GLRPCTPFPDECEAPSVCRPGCSPEHGYCEEPDECRCLEGWTGPLCTVPV
STSSCLNSRVPGPASTGCLLPGPGPCDGNPCANGGSCSETSGSFECACPR
GFYGLRCEVSGVTCADGPCFNGGLCVGGEDPDSAYVCHCPPGFQGSNCEK
RVDRCSLQPCQNGGLCLDLGHALRCRCRAGFAGPRCEHDLDDCAGRACAN
GGTCVEGGGSRRCSCALGFGGRDCRERADPCASRPCAHGGRCYAHFSGLV
CACAPGYMGVRCEFAVRPDGADAVPAAPRGLRQADPQRFLLPPALGLLVA
AGLAGAALLVIHVRRRGPGQDTGTRLLSGTREPSVHTLPDALNNLRLQDG
AGDGPSSSADWNHPEDGDSRSIYVIPAPSIYAREA (SEQ ID NO:
53)),
and
rat (Accession No. NP_446118:
MVSLQVSSLPQTLILAFLLPQALPAGVFELQIHSFGPGPGPGTPRSPCNA
RGPCRLFFRVCLKPGVSQEAAESLCALGAALSTSGPVYTEQPGVPAAALS
LPDGLVRVPFLDAWPGTFSLIIETWREQLGERAAGPAWNLLARVAGRRRL
AAGAPWARDVQRTGAWELHFSYRARCEPPAVGAACARLCRSRSAPSRCGP
GLRPCTPFPDECEAPRESLTVCRAGCSPEHGYCEEPDECHCLEGWTGPLC
TVPVSTSSCLNSRVSGPAGTGCLLPGPGPCDGNPCANGGSCSETPGSFEC
ACPRGFYGPRCEVSGVTCADGPCFNGGLCVGGEDPDSAYVCHCPPAFQGS
NCERRVDRCSLQPCQNGGLCLDLGHALRCRCRAGFAGPRCEHDLDDCAGR
ACANGGTCVEGGGARRCSCALGFGGRDCRERADPCASRPCAHGGRCYAHF
SGLVCACAPGYMGVRCEFAVRPDGADAVPAAPRGLRQADSQRFLLPPALG
LLAAAALAGAALLLIHVRRRGPGRDTGTRLLSGTREPSVHTLPDALNNLR
LQDGAGDGPTSSADWNHPEDGDSRSIYVIPAPSIYAREA (SEQ ID
NO: 54)).

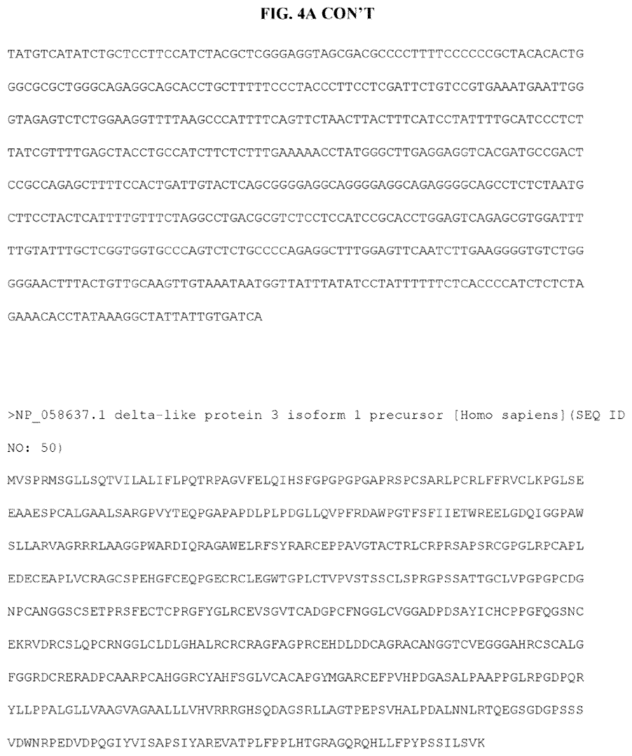

In humans, the DLL3 gene consists of 8 exons spanning 9.5 kBp located on chromosome 19q13. Alternate splicing within the last exon gives rise to a 2389 bp transcript (Accession No. NM_016941 (SEQ ID NO: 55)) and a 2052 bp transcript (Accession No. NM_203486 (SEQ ID NO: 56)). The former transcript encodes a protein that is 618 amino acids in length (Accession No. NP_058637 (SEQ ID NO: 50)), whereas the latter encodes a protein that is 587 amino acids in length (Accession No. NP_982353 (SEQ ID NO: 51)). See FIGS. 4A-4B. These two protein isoforms of DLL3 share overall 100% identity across their extracellular domains and their transmembrane domains, differing only in that the longer isoform contains an extended cytoplasmic tail containing 32 additional residues at the carboxy terminus of the protein.

Both isoforms can be detected in tumor cells. In fact, aberrant DLL3 expression (genotypic and/or phenotypic) is associated with various tumorigenic cell subpopulations such as cancer stem cells and tumor initiating cells. Accordingly, the present disclosure provides DLL3 antibodies that may be particularly useful for targeting such cells (e.g., cancer stem cells, tumor initiating cells, and cancers, e.g., small cell lung cancer, large cell neuroendocrine carcinoma, pulmonary neuroendocrine cancers, extrapulmonary neuroendocrine cancers, and melanoma), thereby facilitating the treatment, management or prevention of neoplastic disorders.

The DLL3 protein used in the present invention can be directly purified from a DLL3-expressing cells of a human or a non-human mammal (e.g., a rat, a mouse or a monkey) and can then be used, or a cell membrane fraction of the aforementioned cells can be prepared and can be used as the DLL3 protein. Alternatively, DLL3 can also be obtained by synthesizing it in vitro, or by allowing host cells to produce DLL3 by genetic manipulation. According to such genetic manipulation, the DLL3 protein can be obtained, specifically, by incorporating DLL3 cDNA into a vector capable of expressing the DLL3 cDNA, and then synthesizing DLL3 in a solution containing enzymes, substrate and energetic materials necessary for transcription and translation, or by transforming the host cells of other prokaryotes or eukaryotes, so as to allow them to express DLL3. Also, DLL3-expressing cells based on the above-described genetic manipulation, or a cell line expressing DLL3 may be used to present the DLL3 protein. Alternatively, the expression vector into which DLL3 cDNA has been incorporated can be directly administered to an animal to be immunized, and DLL3 can be expressed in the body of the animal thus immunized.

Moreover, a protein which consists of an amino acid sequence comprising a substitution, deletion and/or addition of one or several amino acids in the above-described amino acid sequence of DLL3, and has a biological activity equivalent to that of the DLL3 protein, is also included within the term “DLL3”.

### III. Anti-DLL3 Antibodies

The present technology describes methods and compositions for the generation and use of anti-DLL3 immunoglobulin-related compositions (e.g., anti-DLL3 antibodies or antigen binding fragments thereof). The anti-DLL3 immunoglobulin-related compositions of the present disclosure may be useful in the diagnosis, or treatment of the DLL3 associated cancers (e.g., small-cell lung cancer, large cell neuroendocrine carcinoma, pulmonary neuroendocrine cancers, extrapulmonary neuroendocrine cancers, and melanoma). Anti-DLL3 immunoglobulin-related compositions within the scope of the present technology include, e.g., but are not limited to, monoclonal, chimeric, humanized, human, bispecific antibodies and diabodies that specifically bind the target polypeptide, a homolog, derivative or a fragment thereof. The present disclosure also provides antigen binding fragments of any of the anti-DLL3 antibodies disclosed herein, wherein the antigen binding fragment is selected from the group consisting of Fab, F(ab)′2, Fab′, scFv, and Fv.

The present technology discloses anti-DLL3 antibodies that can promote internalization of DLL3-antibody complex and are thus useful for delivering toxic payloads to tumor cells.

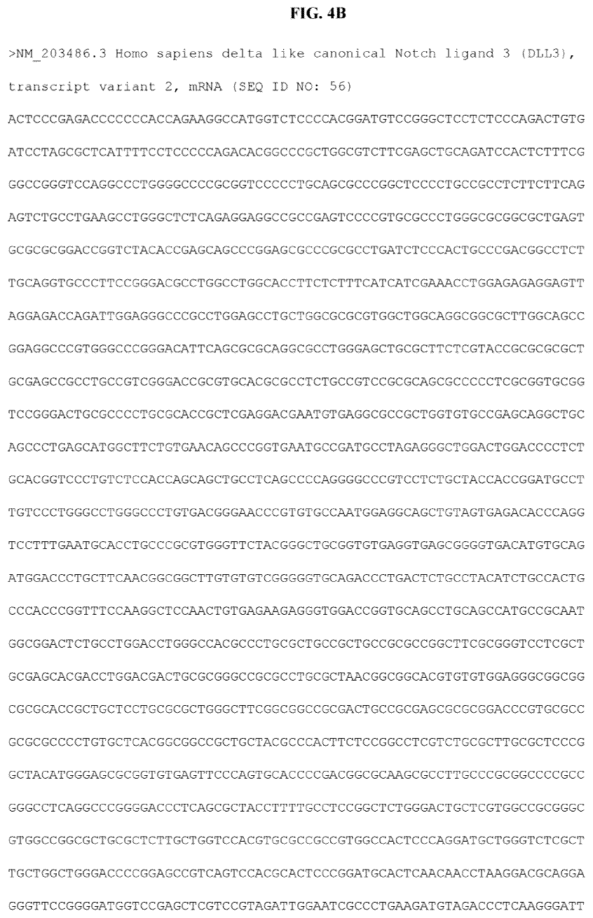

FIGS. 5-8 provides the nucleotide and amino acid sequences for VH and VL as well as the CDR sequences for the antibodies discloses herein (SEQ ID NOs: 1-40). The Table below also provides amino acid sequences for V_{H} and V_{L} as well as the CDR sequences for the antibodies discloses herein.

| SEQ ID NO: | Description | Sequence |
|---|---|---|
| SEQ ID NO: 2 | Amino acid Sequence of | EVQLVESGGGLVKPGGSLRLSCAASGFTFSNT |
| | V_{H} of 7-I1-B | WMSWVRQAPGKGLEWVGRIKSKSDGGTTDY |
| | | AAPVKGRFTISRDDSKNTLYLQMNSLKTEDTA |
| | | VYYCTQYYWNSFDYWGQGTLVTVSS |
| SEQ ID NO: 3 | Amino acid Sequence of | GFTFSNTW |
| | V_{H} CDR1 of 7-I1-B | |
| SEQ ID NO: 4 | Amino acid Sequence of | IKSKSDGGTT |
| | V_{H} CDR2 of 7-I1-B | |
| SEQ ID NO: 5 | Amino acid Sequence of | TQYYWNSFDY |
| | V_{H} CDR3 of 7-I1-B | |
| SEQ ID NO: 7 | Amino acid Sequence of | DIQMTQSPSSLSASVGDRVTITCQASQDISNYL |
| | V_{L} of 7-I1-B | NWYQQKPGKAPKLLIYDASNLETGVPSRFSGS |
| | | GSGTDFTFTISSLQPEDIATYYCQQYDNLPLTF |
| | | GGGTKVEIK |
| SEQ ID NO: 8 | Amino acid Sequence of | QASQDISNYLN |
| | V_{L} CDR1 of 7-I1-B | |
| SEQ ID NO: 9 | Amino acid Sequence of | DASNLET |
| | V_{L} CDR2 of 7-I1-B | |
| SEQ ID NO: 10 | Amino acid Sequence of | QQYDNLPLT |
| | V_{L} CDR3 of 7-I1-B | |
| SEQ ID NO: 12 | Amino acid Sequence of | EVQLVESGGGLVQPGGSQRLSCAASGFTFSSY |
| | V_{H} of 2-C8-A | WMNWVRQAPGKGLEWVANIKEDGSEKYYV |
| | | DSVKGRFTISRDNAKNSLYLQMNSLRAEDTA |
| | | VYYCARDPGWAPFDYWGQGTLVTVSS |
| SEQ ID NO: 13 | Amino acid Sequence of | GFTFSSY |
| | V_{H} CDR1 of 2-C8-A | |
| SEQ ID NO: 14 | Amino acid Sequence of | KEDGSE |
| | V_{H} CDR2 of 2-C8-A | |
| SEQ ID NO: 15 | Amino acid Sequence of | DPGWAPFDY |
| | V_{H} CDR3 of 2-C8-A | |
| SEQ ID NO: 17 | Amino acid Sequence of | DIQMSQSPSSLSASVGDRVTITCRASQGISNYL |
| | V_{L} of 2-C8-A | AWFQQKPGKAPKSLIYAASSLQSGVPSKFSGS |
| | | GSGTDFTLAISSLOPEDFATYYCQQYNSFPYTF |
| | | GQGTTLEIK |
| SEQ ID NO: 18 | Amino acid Sequence of | RASQGISNYLA |
| | V_{L} CDR1 of 2-C8-A | |
| SEQ ID NO: 19 | Amino acid Sequence of | AASSLQS |
| | V_{L} CDR2 of 2-C8-A | |
| SEQ ID NO: 20 | Amino acid Sequence of | QQYNSFPYT |
| | V_{L} CDR3 of 2-C8-A | |
| SEQ ID NO: 59 | Amino acid Sequence of | EVQLVESGGGLVQPGGSQRLSCAASGFTFSSY |
| | V_{H} of H2-C8-A | WMNWVRQAPGKGLEWVANIKEDGSEKYYV |
| | | DSVKGRFTISRDNAKNSLYLQMNSLRAEDTA |
| | | VYYCARDPGWAPFDYWGQGTLVTVSSASTK |
| | | GPSVFPLAPSSKSTSGGTAALGCLVKDYFPEP |
| | | VTVSWNSGALTSGVHTFPAVLQSSGLYSLSSV |
| | | VTVPSSSLGTQTYICNVNHKPSNTKVDKKVEP |
| | | KSCDKTHTCPPCPAPELLGGPSVFLFPPKPKDT |
| | | LMISRTPEVTCVVVDVSHEDPEVKFNWYVDG |
| | | VEVHNAKTKPREEQYNSTYRVVSVLTVLHQD |
| | | WLNGKEYKCKVSNKALPAPIEKTISKAKGQPR |
| | | EPQVYTLPPSRDELTKNQVSLTCLVKGFYPSDI |
| | | AVEWESNGQPENNYKTTPPVLDSDGSFFLYSK |
| | | LTVDKSRWQQGNVFSCSVMHEALHNHYTQK |
| | | SLSLSPGK |
| SEQ ID NO: 60 | Amino acid Sequence of | EVQLVESGGGLVQPGGSQRLSCAASGFTFSSY |
| | V_{H} of H2-C8-A-2 | WMNWVRQAPGKGLEWVANIKEDGSEKYYV |
| | | DSVKGRFTISRDNAKNSLYLQMNSLRAEDTA |
| | | VYYCARDPGWAPFDYWGQGTLVTVSSASTK |
| | | GPSVFPLAPSSKSTSGGTAALGCLVKDYFPEP |
| | | VTVSWNSGALTSGVHTFPAVLQSSGLYSLSSV |
| | | VTVPSSSLGTQTYICNVNHKPSNTKVDKKVEP |
| | | KSCDKTHTCPPCPAPELLGGPSVFLFPPKPKDT |
| | | LMISRTPEVTCVVVDVSHEDPEVKFNWYVDG |
| | | VEVHNAKTKPREEQYNSTYRVVSVLTVLHQD |
| | | WLNGKEYKCKVSNKALPAPIEKTISKAKGQPR |
| | | EPQVYTLPPSREEMTKNQVSLTCLVKGFYPSD |
| | | IAVEWESNGQPENNYKTTPPVLDSDGSFFLYS |
| | | KLTVDKSRWQQGNVFSCSVMHEALHNHYTQ |
| | | KSLSLSPGK |
| SEQ ID NO: 61 | Amino acid Sequence of | EVQLVESGGGLVQPGGSQRLSCAASGFTFSSY |
| | V_{H} of H2-C8-A-3 | WMNWVRQAPGKGLEWVANIKEDGSEKYYV |
| | | DSVKGRFTISRDNAKNSLYLQMNSLRAEDTA |
| | | VYYCARDPGWAPFDYWGQGTLVTVSSASTK |
| | | GPSVFPLAPSSKSTSGGTAALGCLVKDYFPEP |
| | | VTVSWNSGALTSGVHTFPAVLQSSGLYSLSSV |
| | | VTVPSSSLGTQTYICNVNHKPSNTKVDKKVEP |
| | | KSCDKTHTCPPCPAPEAAGGPSVFLFPPKPKD |
| | | TLMISRTPEVTCVVVDVSHEDPEVKFNWYVD |
| | | GVEVHNAKTKPREEQYNSTYRVVSVLTVLHQ |
| | | DWLNGKEYKCKVSNKALPAPIEKTISKAKGQP |
| | | REPQVYTLPPSREEMTKNQVSLTCLVKGFYPS |
| | | DIAVEWESNGQPENNYKTTPPVLDSDGSFFLY |
| | | SKLTVDKSRWQQGNVFSCSVMHEALHNHYT |
| | | QKSLSLSPGK |
| SEQ ID NO: 62 | Amino acid Sequence of | DIQMSQSPSSLSASVGDRVTITCRASQGISNYL |
| | V_{L} of H2-C8-A, | AWFQQKPGKAPKSLIYAASSLQSGVPSKFSGS |
| | H2-C8-A-2, and | GSGTDFTLAISSLQPEDFATYYCQQYNSFPYTF |
| | H2-C8-A-3 | GQGTTLEIKRTVAAPSVFIFPPSDEQLKSGTAS |
| | | VVCLLNNFYPREAKVQWKVDNALQSGNSQES |
| | | VTEQDSKDSTYSLSSTLTLSKADYEKHKVYAC |
| | | EVTHQGLSSPVTKSFNRGEC |
| SEQ ID NO: 22 | Amino acid Sequence of | QVQLQESGPGLVKPSETLSLTCTVSGGSINSYY |
| | V_{H} of 10-O18-A | WSWIRQPPGKGLEWIGYIFYSGITNYNPSLKSR |
| | | VTISLDTSKNQFSLKLSSVTAADTAVYYCARI |
| | | GVAGFYFDYWGQGTLVTVSS |
| SEQ ID NO: 23 | Amino acid Sequence of | GGSINSY |
| | V_{H} CDR1 of 10-O18-A | |
| SEQ ID NO: 24 | Amino acid Sequence of | FYSGI |
| | V_{H} CDR2 of 10-O18-A | |
| SEQ ID NO: 25 | Amino acid Sequence of | IGVAGFYFDY |
| | V_{H} CDR3 of 10-O18-A | |
| SEQ ID NO: 27 | Amino acid Sequence of | EIVLTQSPGTLSLSPGERATLSCRASQSVSSSYL |
| | V_{L} of 10-O18-A | AWYQQKPGQAPRLLIYGASSRATGIPDRFSGS |
| | | GSGTDFTLTISRLEPEDFAVYYCQQYGTSPLTF |
| | | GGGTKVEIK |
| SEQ ID NO: 28 | Amino acid Sequence of | RASQSVSSSYLA |
| | V_{L} CDR1 of 10-O18-A | |
| SEQ ID NO: 29 | Amino acid Sequence of | GASSRAT |
| | V_{L} CDR2 of 10-O18-A | |
| SEQ ID NO: 30 | Amino acid Sequence of | QQYGTSPLT |
| | V_{L} CDR3 of 10-O18-A | |
| SEQ ID NO: 67 | Amino acid Sequence of | QVQLQESGPGLVKPSETLSLTCTVSGGSINSYY |
| | V_{H} of H10-O18-A | WSWIRQPPGKGLEWIGYIFYSGITNYNPSLKSR |
| | | VTISLDTSKNQFSLKLSSVTAADTAVYYCARI |
| | | GVAGFYFDYWGQGTLVTVSSASTKGPSVFPL |
| | | APSSKSTSGGTAALGCLVKDYFPEPVTVSWNS |
| | | GALTSGVHTFPAVLQSSGLYSLSSVVTVPSSSL |
| | | GTQTYICNVNHKPSNTKVDKKVEPKSCDKTH |
| | | TCPPCPAPELLGGPSVFLFPPKPKDTLMISRTPE |
| | | VTCVVVDVSHEDPEVKFNWYVDGVEVHNAK |
| | | TKPREEQYNSTYRVVSVLTVLHQDWLNGKEY |
| | | KCKVSNKALPAPIEKTISKAKGQPREPQVYTL |
| | | PPSRDELTKNQVSLTCLVKGFYPSDIAVEWES |
| | | NGQPENNYKTTPPVLDSDGSFFLYSKLTVDKS |
| | | RWQQGNVFSCSVMHEALHNHYTQKSLSLSPG |
| | | K |
| SEQ ID NO: 68 | Amino acid Sequence of | QVQLQESGPGLVKPSETLSLTCTVSGGSINSYY |
| | V_{H} of H10-O18-A-2 | WSWIRQPPGKGLEWIGYIFYSGITNYNPSLKSR |
| | | VTISLDTSKNQFSLKLSSVTAADTAVYYCARI |
| | | GVAGFYFDYWGQGTLVTVSSASTKGPSVFPL |
| | | APSSKSTSGGTAALGCLVKDYFPEPVTVSWNS |
| | | GALTSGVHTFPAVLQSSGLYSLSSVVTVPSSSL |
| | | GTQTYICNVNHKPSNTKVDKKVEPKSCDKTH |
| | | TCPPCPAPELLGGPSVFLFPPKPKDTLMISRTPE |
| | | VTCVVVDVSHEDPEVKFNWYVDGVEVHNAK |
| | | TKPREEQYNSTYRVVSVLTVLHQDWLNGKEY |
| | | KCKVSNKALPAPIEKTISKAKGQPREPQVYTL |
| | | PPSREEMTKNQVSLTCLVKGFYPSDIAVEWES |
| | | NGQPENNYKTTPPVLDSDGSFFLYSKLTVDKS |
| | | RWQQGNVFSCSVMHEALHNHYTQKSLSLSPG |
| | | K |
| SEQ ID NO: 69 | Amino acid Sequence of | QVQLQESGPGLVKPSETLSLTCTVSGGSINSYY |
| | V_{H} of H10-O18-A-3 | WSWIRQPPGKGLEWIGYIFYSGITNYNPSLKSR |
| | | VTISLDTSKNQFSLKLSSVTAADTAVYYCARI |
| | | GVAGFYFDYWGQGTLVTVSSASTKGPSVFPL |
| | | APSSKSTSGGTAALGCLVKDYFPEPVTVSWNS |
| | | GALTSGVHTFPAVLQSSGLYSLSSVVTVPSSSL |
| | | GTQTYICNVNHKPSNTKVDKKVEPKSCDKTH |
| | | TCPPCPAPEAAGGPSVFLFPPKPKDTLMISRTP |
| | | EVTCVVVDVSHEDPEVKFNWYVDGVEVHNA |
| | | KTKPREEQYNSTYRVVSVLTVLHQDWLNGKE |
| | | YKCKVSNKALPAPIEKTISKAKGQPREPQVYT |
| | | LPPSREEMTKNQVSLTCLVKGFYPSDIAVEWE |
| | | SNGQPENNYKTTPPVLDSDGSFFLYSKLTVDK |
| | | SRWQQGNVFSCSVMHEALHNHYTQKSLSLSP |
| | | GK |
| SEQ ID NO: 70 | Amino acid Sequence of | EIVLTQSPGTLSLSPGERATLSCRASQSVSSSYL |
| | V_{L} of | AWYQQKPGQAPRLLIYGASSRATGIPDRFSGS |
| | H10-O18-A, | GSGTDFTLTISRLEPEDFAVYYCQQYGTSPLTF |
| | H10-O18-A-2, and | GGGTKVEIKRTVAAPSVFIFPPSDEQLKSGTAS |
| | H10-O18-A-3 | VVCLLNNFYPREAKVQWKVDNALQSGNSQES |
| | | VTEQDSKDSTYSLSSTLTLSKADYEKHKVYAC |
| | | EVTHQGLSSPVTKSFNRGEC |
| SEQ ID NO: 32 | Amino acid Sequence of | QVQLVQSGAEVKKPGASVKVSCKASGYTFTS |
| | V_{H} of 6-G23-F | YYIHWVRQAPGQGLEWMGIIDPSDGSTNYAQ |
| | | KFQGRVTMTRDTSTSTVYMELSSLRSEDTAV |
| | | YYCARDREYNYYGLDVWGQGTTVTVSS |
| SEQ ID NO: 33 | Amino acid Sequence of | GYTFTSY |
| | V_{H} CDR1 of 6-G23-F | |
| SEQ ID NO: 34 | Amino acid Sequence of | DPSDGS |
| | V_{H} CDR2 of 6-G23-F | |
| SEQ ID NO: 35 | Amino acid Sequence of | DREYNYYGLDV |
| | V_{H} CDR3 of 6-G23-F | |
| SEQ ID NO: 37 | Amino acid Sequence of | DVVMTQSPLSLPVTLGQPASISCRSSQSLVYR |
| | V_{L} of 6-G23-F | DGNTYLNWFQQRPGQSPRRLIYKVSNRDSGV |
| | | PDRFRGSGSGTDFTLKISRVEAEDVGVYYCM |
| | | QGTHWPPTFGQGTKVEIK |
| SEQ ID NO: 38 | Amino acid Sequence of | RSSQSLVYRDGNTYLN |
| | V_{L} CDR1 of 6-G23-F | |
| SEQ ID NO: 39 | Amino acid Sequence of | KVSNRDS |
| | V_{L} CDR2 of 6-G23-F | |
| SEQ ID NO: 40 | Amino acid Sequence of | MQGTHWPPT |
| | V_{L} CDR3 of 6-G23-F | |
| SEQ ID NO: 63 | Amino acid Sequence of | QVQLVQSGAEVKKPGASVKVSCKASGYTFTS |
| | V_{H} of H6-G23-A | YYIHWVRQAPGQGLEWMGIIDPSDGSTNYAQ |
| | | KFQGRVTMTRDTSTSTVYMELSSLRSEDTAV |
| | | YYCARDREYNYYGLDVWGQGTTVTVSSAST |
| | | KGPSVFPLAPSSKSTSGGTAALGCLVKDYFPE |
| | | PVTVSWNSGALTSGVHTFPAVLQSSGLYSLSS |
| | | VVTVPSSSLGTQTYICNVNHKPSNTKVDKKVE |
| | | PKSCDKTHTCPPCPAPELLGGPSVFLFPPKPKD |
| | | TLMISRTPEVTCVVVDVSHEDPEVKFNWYVD |
| | | GVEVHNAKTKPREEQYNSTYRVVSVLTVLHQ |
| | | DWLNGKEYKCKVSNKALPAPIEKTISKAKGQP |
| | | REPQVYTLPPSRDELTKNQVSLTCLVKGFYPS |
| | | DIAVEWESNGQPENNYKTTPPVLDSDGSFFLY |
| | | SKLTVDKSRWQQGNVFSCSVMHEALHNHYT |
| | | QKSLSLSPGK |
| SEQ ID NO: 64 | Amino acid Sequence of | QVQLVQSGAEVKKPGASVKVSCKASGYTFTS |
| | V_{H} of H6-G23-A-2 | YYIHWVRQAPGQGLEWMGIIDPSDGSTNYAQ |
| | | KFQGRVTMTRDTSTSTVYMELSSLRSEDTAV |
| | | YYCARDREYNYYGLDVWGQGTTVTVSSAST |
| | | KGPSVFPLAPSSKSTSGGTAALGCLVKDYFPE |
| | | PVTVSWNSGALTSGVHTFPAVLQSSGLYSLSS |
| | | VVTVPSSSLGTQTYICNVNHKPSNTKVDKKVE |
| | | PKSCDKTHTCPPCPAPELLGGPSVFLFPPKPKD |
| | | TLMISRTPEVTCVVVDVSHEDPEVKFNWYVD |
| | | GVEVHNAKTKPREEQYNSTYRVVSVLTVLHQ |
| | | DWLNGKEYKCKVSNKALPAPIEKTISKAKGQP |
| | | REPQVYTLPPSREEMTKNQVSLTCLVKGFYPS |
| | | DIAVEWESNGQPENNYKTTPPVLDSDGSFFLY |
| | | SKLTVDKSRWQQGNVFSCSVMHEALHNHYT |
| | | QKSLSLSPGK |
| SEQ ID NO: 65 | Amino acid Sequence of | QVQLVQSGAEVKKPGASVKVSCKASGYTFTS |
| | V_{H} of H6-G23-A-3 | YYIHWVRQAPGQGLEWMGIIDPSDGSTNYAQ |
| | | KFQGRVTMTRDTSTSTVYMELSSLRSEDTAV |
| | | YYCARDREYNYYGLDVWGQGTTVTVSSAST |
| | | KGPSVFPLAPSSKSTSGGTAALGCLVKDYFPE |
| | | PVTVSWNSGALTSGVHTFPAVLQSSGLYSLSS |
| | | VVTVPSSSLGTQTYICNVNHKPSNTKVDKKVE |
| | | PKSCDKTHTCPPCPAPEAAGGPSVFLFPPKPKD |
| | | TLMISRTPEVTCVVVDVSHEDPEVKFNWYVD |
| | | GVEVHNAKTKPREEQYNSTYRVVSVLTVLHQ |
| | | DWLNGKEYKCKVSNKALPAPIEKTISKAKGQP |
| | | REPQVYTLPPSREEMTKNQVSLTCLVKGFYPS |
| | | DIAVEWESNGQPENNYKTTPPVLDSDGSFFLY |
| | | SKLTVDKSRWQQGNVFSCSVMHEALHNHYT |
| | | QKSLSLSPGK |
| SEQ ID NO: 66 | Amino acid Sequence of | DVVMTQSPLSLPVTLGQPASISCRSSQSLVYR |
| | V_{L} of H6-G23-A, | DGNTYLNWFQQRPGQSPRRLIYKVSNRDSGV |
| | H6-G23-A-2, and | PDRFRGSGSGTDFTLKISRVEAEDVGVYYCM |
| | H6-G23-A-3 | QGTHWPPTFGQGTKVEIKRTVAAPSVFIFPPSD |
| | | EQLKSGTASVVCLLNNFYPREAKVQWKVDN |
| | | ALQSGNSQESVTEQDSKDSTYSLSSTLTLSKA |
| | | DYEKHKVYACEVTHQGLSSPVTKSFNRGEC |

In one aspect, the present disclosure provides an antibody or antigen binding fragment thereof comprising a heavy chain immunoglobulin variable domain (VH) and a light chain immunoglobulin variable domain (VL), wherein (a) the VH comprises a VH-CDR1 sequence, a VH-CDR2 sequence, and a VH-CDR3 sequence selected from the group consisting of (i) SEQ ID NO: 3, SEQ ID NO: 4, and SEQ ID NO: 5, respectively; (ii) SEQ ID NO: 13, SEQ ID NO: 14, and SEQ ID NO: 15, respectively; (iii) SEQ ID NO: 23, SEQ ID NO: 24, and SEQ ID NO: 25, respectively; and (iv) SEQ ID NO: 33, SEQ ID NO: 34, and SEQ ID NO: 35, respectively; and/or (b) the VL comprises a VL-CDR1 sequence, a VL-CDR2 sequence, and a VL-CDR3 sequence selected from the group consisting of (i) SEQ ID NO: 8, SEQ ID NO: 9, and SEQ ID NO: 10, respectively; (ii) SEQ ID NO: 18, SEQ ID NO: 19, and SEQ ID NO: 20, respectively; (iii) SEQ ID NO: 28, SEQ ID NO: 29, and SEQ ID NO: 30, respectively; and (iv) SEQ ID NO: 38, SEQ ID NO: 39, and SEQ ID NO: 40, respectively. In some embodiments, the present disclosure provides an antibody or antigen binding fragment thereof comprising a heavy chain immunoglobulin variable domain (VH) and a light chain immunoglobulin variable domain (VL), wherein the combination of (a) the V_{H} comprising a V_{H}-CDR1 sequence, a V_{H}-CDR2 sequence, and a V_{H}-CDR3 sequence and (b) the V_{L} comprising a V_{L}-CDR1 sequence, a V_{L}—CDR2 sequence, and a V_{L}-CDR3 sequence is selected from the group consisting of: (i) (a) SEQ ID NO: 3, SEQ ID NO: 4, and SEQ ID NO: 5 and (b) SEQ ID NO: 8, SEQ ID NO: 9, and SEQ ID NO: 10, respectively; (ii) (a) SEQ ID NO: 13, SEQ ID NO: 14, and SEQ ID NO: 15 and (b) SEQ ID NO: 18, SEQ ID NO: 19, and SEQ ID NO: 20, respectively; (iii) (a) SEQ ID NO: 23, SEQ ID NO: 24, and SEQ ID NO: 25 and (b) SEQ ID NO: 28, SEQ ID NO: 29, and SEQ ID NO: 30, respectively; and (iv) (a) SEQ ID NO: 33, SEQ ID NO: 34, and SEQ ID NO: 35 and (b) SEQ ID NO: 38, SEQ ID NO: 39, and SEQ ID NO: 40, respectively. In some embodiments, the antibody further comprises a Fc domain of any isotype, e.g., but are not limited to, IgG (including IgG1 and the variant (SEQ ID NO: 42, 57, and 58), IgG2, IgG3, and IgG4), IgA (including IgA1 and IgA2), IgD, IgE, or IgM, and IgY In some embodiments, the antibody comprises a heavy chain constant region of SEQ ID NO: 42, 57, or 58, preferably SEQ ID NO: 57, or 58, more preferably SEQ ID NO: 58. Non-limiting examples of constant region sequences include: Human IgD constant region, Uniprot: P01880 (SEQ ID NO: 41) APTKAPDVFPIISGCRHPKDNSPVVLACLITGYHPTSVTVTWYMGTQ SQPQRTFPEIQ RRDSYYMTSSQL STPLQQWRQGEYKCVVQHTASKSKKEIFRWPESPKAQASSVPTA QPQAEGSLAKATTAPATTRNTGRGGEEKKKEKEKEEQEERETKTPECPSHTQPLGVY LLTPAVQDLWLRDKATFTCFVVGSDLKDAHLTWEVAGKVPTGGVEEGLLERHSNG SQSQHSRLTLPRSLWNAGTSVTCTLNHPSLPPQRLMALREPAAQAPVKLSLNLLASS DPPEAASWLLCEVSGFSPPNILLMWLEDQREVNTSGFAPARPPPQPGSTTFWAWSVL RVPAPPSPQPATYTCVVSHEDSRTLLNASRSLEVSYVTDHGPMK

Human IgG1 constant region, Uniprot: P01857
(SEQ ID NO: 42)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK
Human IgG1 variant constant region
(SEQ ID NO: 57)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHNHYTQKSLSLSPGK
Human IgG1 variant constant region
(SEQ ID NO: 58)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPEAAGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHNHYTQKSLSLSPGK
Human IgG2 constant region, Uniprot: P01859
(SEQ ID NO: 43)
ASTKGPSVFPLAPCSRSTSESTAALGCLVKDYFPEPVTVSWNSGALTSG
VHTFPAVLQSSGLYSLSSVVTVPSSNFGTQTYTCNVDHKPSNTKVDKTV
ERKCCVECPPCPAPPVAGPSVFLFPPKPKDTLMISRTPEVTCVVVDVSH
EDPEVQFNWYVDGVEVHNAKTKPREEQFNSTFRVVSVLTVVHQDWLNGK
EYKCKVSNKGLPAPIEKTISKTKGQPREPQVYTLPPSREEMTKNQVSLT
CLVKGFYPSDISVEWESNGQPENNYKTTPPMLDSDGSFFLYSKLTVDKS
RWQQGNVFSCSVMHEALHNHYTQKSLSLSPGK
Human IgG3 constant region, Uniprot: P01860
(SEQ ID NO: 44)
ASTKGPSVFPLAPCSRSTSGGTAALGCLVKDYFPEPVTVSWNSGALTSG
VHTFPAVLQSSGLYSLSSVVTVPSSSLGTQTYTCNVNHKPSNTKVDKRV
ELKTPLGDTTHTCPRCPEPKSCDTPPPCPRCPEPKSCDTPPPCPRCPEP
KSCDTPPPCPRCPAPELLGGPSVFLFPPKPKDTLMISRTPEVTCVVVDV
SHEDPEVQFKWYVDGVEVHNAKTKPREEQYNSTFRVVSVLTVLHQDWLN
GKEYKCKVSNKALPAPIEKTISKTKGQPREPQVYTLPPSREEMTKNQVS
LTCLVKGFYPSDIAVEWESSGQPENNYNTTPPMLDSDGSFFLYSKLTVD
KSRWQQGNIFSCSVMHEALHNRFTQKSLSLSPGK
Human IgM constant region, Uniprot: P01871
(SEQ ID NO: 45)
GSASAPTLFPLVSCENSPSDTSSVAVGCLAQDFLPDSITLSWKYKNNSD
ISSTRGFPSVLRGGKYAATSQVLLPSKDVMQGTDEHVVCKVQHPNGNKE
KNVPLPVIAELPPKVSVFVPPRDGFFGNPRKSKLICQATGFSPRQIQVS
WLREGKQVGSGVTTDQVQAEAKESGPTTYKVTSTLTIKESDWLGQSMFT
CRVDHRGLTFQQNASSMCVPDQDTAIRVFAIPPSFASIFLTKSTKLTCL
VTDLTTYDSVTISWTRQNGEAVKTHTNISESHPNATFSAVGEASICEDD
WNSGERFTCTVTHTDLPSPLKQTISRPKGVALHRPDVYLLPPAREQLNL
RESATITCLVTGFSPADVFVQWMQRGQPLSPEKYVTSAPMPEPQAPGRY
FAHSILTVSEEEWNTGETYTCVAHEALPNRVTERTVDKSTGKPTLYNVS
LVMSDTAGTCY
Human IgG4 constant region, Uniprot: P01861
(SEQ ID NO: 46)
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPSCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK
Human IgA1 constant region, Uniprot: P01876
(SEQ ID NO: 47)
ASPTSPKVFPLSLCSTQPDGNVVIACLVQGFFPQEPLSVTWSESGQGVT
ARNFPPSQDASGDLYTTSSQLTLPATQCLAGKSVTCHVKHYTNPSQDVT
VPCPVPSTPPTPSPSTPPTPSPSCCHPRLSLHRPALEDLLLGSEANLTC
TLTGLRDASGVTFTWTPSSGKSAVQGPPERDLCGCYSVSSVLPGCAEPW
NHGKTFTCTAAYPESKTPLTATLSKSGNTFRPEVHLLPPPSEELALNEL
VTLTCLARGFSPKDVLVRWLQGSQELPREKYLTWASRQEPSQGTTTFAV
TSILRVAAEDWKKGDTFSCMVGHEALPLAFTQKTIDRLAGKPTHVNVSV
VMAEVDGTCY
Human IgA2 constant region, Uniprot: P01877
(SEQ ID NO: 48)
ASPTSPKVFPLSLDSTPQDGNVVVACLVQGFFPQEPLSVTWSESGQNVT
ARNFPPSQDASGDLYTTSSQLTLPATQCPDGKSVTCHVKHYTNPSQDVT
VPCPVPPPPPCCHPRLSLHRPALEDLLLGSEANLTCTLTGLRDASGATF
TWTPSSGKSAVQGPPERDLCGCYSVSSVLPGCAQPWNHGETFTCTAAHP
ELKTPLTANITKSGNTFRPEVHLLPPPSEELALNELVTLTCLARGFSPK
DVLVRWLQGSQELPREKYLTWASRQEPSQGTTTFAVTSILRVAAEDWKK
GDTFSCMVGHEALPLAFTQKTIDRMAGKPTHVNVSVVMAEVDGTCY
Human Ig kappa constant region, Uniprot: P01834
(SEQ ID NO: 49)
TVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVT
KSFNRGEC

In some embodiments, the immunoglobulin-related compositions of the present technology comprise a heavy chain constant region that is at least 80%, at least 85%, at least 90%, at least 95%, at least 99%, or is 100% identical to SEQ ID NOS: 41-48, 57, 58. Additionally or alternatively, in some embodiments, the immunoglobulin-related compositions of the present technology comprise a light chain constant region that is at least 80%, at least 85%, at least 90%, at least 95%, at least 99%, or is 100% identical to SEQ ID NO: 49. In some embodiments, the immunoglobulin-related compositions of the present technology bind to the extracellular domain of DLL3. In some embodiments, the epitope is a conformational epitope.

In another aspect, the present disclosure provides an isolated immunoglobulin-related composition (e.g., an antibody or antigen binding fragment thereof) comprising a heavy chain immunoglobulin variable domain (VH) amino acid sequence comprising SEQ ID NO: 2, SEQ ID NO: 12, SEQ ID NO: 22, or SEQ ID NO: 32, or a variant thereof having one or more conservative amino acid substitutions or a heavy chain amino acid sequence comprising SEQ ID NO: 59, SEQ ID NO: 60, SEQ ID NO: 61, SEQ ID NO: 63, SEQ ID NO: 64, SEQ ID NO: 65, SEQ ID NO: 67, SEQ ID NO: 68, or SEQ ID NO: 69 or a variant thereof having one or more conservative amino acid substitutions.

Additionally or alternatively, in some embodiments, the immunoglobulin-related compositions of the present technology comprise a light chain immunoglobulin variable domain (VL) amino acid sequence comprising SEQ ID NO: 7, SEQ ID NO: 17, SEQ ID NO: 27, or SEQ ID NO: 37, or a variant thereof having one or more conservative amino acid substitutions or a light chain amino acid sequence comprising SEQ ID NO: 62, SEQ ID NO: 66, or SEQ ID NO: 70 or a variant thereof having one or more conservative amino acid substitutions.

In some embodiments, the immunoglobulin-related compositions of the present technology comprise a heavy chain immunoglobulin variable domain (VH) or heavy chain amino acid sequence and a light chain immunoglobulin variable domain (VL) or light chain amino acid sequence selected from the group consisting of: SEQ ID NO: 2 and SEQ ID NO: 7 (7-I1-B), respectively; SEQ ID NO: 12 and SEQ ID NO: 17 (2-C8-A), respectively; SEQ ID NO: 59 and SEQ ID NO: 62 (H2-C8-A), respectively; SEQ ID NO: 60 and SEQ ID NO: 62 (H2-C8-A-2), respectively; SEQ ID NO: 61 and SEQ ID NO: 62 (H2-C8-A-3), respectively; SEQ ID NO: 22 and SEQ ID NO: 27 (10-O18-A), respectively; SEQ ID NO: 67 and SEQ ID NO: 70 (H10-O18-A), respectively; SEQ ID NO: 68 and SEQ ID NO: 70 (H10-O18-A-2), respectively; SEQ ID NO: 69 and SEQ ID NO: 70 (H10-O18-A-3); SEQ ID NO: 32 and SEQ ID NO: 37 (6-G23-F), respectively.

In any of the above embodiments of the immunoglobulin-related compositions, the HC and LC immunoglobulin variable domain sequences form an antigen binding site that binds to the extracellular domain of DLL3. In any of the above embodiments of the immunoglobulin-related compositions, the HC and LC immunoglobulin variable domain sequences form an antigen binding site that binds to DLL3 and promote internalization of the immunoglobulin-related composition. In some embodiments, the epitope is a conformational epitope.

In some embodiments, the HC and LC immunoglobulin variable domain sequences are components of the same polypeptide chain. In other embodiments, the HC and LC immunoglobulin variable domain sequences are components of different polypeptide chains. In certain embodiments, the antibody is a full-length antibody.

In some embodiments, the immunoglobulin-related compositions of the present technology bind specifically to at least one DLL3 polypeptide. In some embodiments, the immunoglobulin-related compositions of the present technology bind at least one DLL3 polypeptide with a dissociation constant (KD) of about 10-3 M, 10-4 M, 10-5 M, 10-6 M, 10-7 M, 10-8 M, 10-9 M, 10-10 M, 10-11 M, or 10-12 M. In certain embodiments, the immunoglobulin-related compositions are monoclonal antibodies, chimeric antibodies, humanized antibodies, human antibodies, or bispecific antibodies. In some embodiments, the antibodies comprise a human antibody framework region.

In certain embodiments, the immunoglobulin-related composition includes one or more of the following characteristics: (a) a light chain immunoglobulin variable domain sequence that is at least 80%, at least 85%, at least 90%, at least 95%, or at least 99% identical to the light chain immunoglobulin variable domain sequence present in any one of SEQ ID NOs: 7, 17, 27, 37, 62, 66, or 70; and/or (b) a heavy chain immunoglobulin variable domain sequence that is at least 80%, at least 85%, at least 90%, at least 95%, or at least 99% identical to the heavy chain immunoglobulin variable domain sequence present in any one of SEQ ID NOs: 2, 12, 22, 32, 59, 60, 61, 63, 64, 65, 67, 68, or 69. In another aspect, one or more amino acid residues in the immunoglobulin-related compositions provided herein are substituted with another amino acid. The substitution may be a “conservative substitution” as defined herein.

In certain embodiments, the immunoglobulin-related compositions contain an IgG1 constant region comprising one or more amino acid substitutions or sets of amino acid residues selected from the group consisting of N297A and K322A, two amino acid substitutions of two leucine (L) residues to alanine (A) at position 234 and 235 (according to EU index) of the heavy chain (LALA), a set of amino acid residues Glu (E) at positions 356 and Met (M) at position 358 (according to EU index) of the heavy chain, or a set of Asp (D) at positions 356 and Leucine (L) at position 358 (according to EU index) of the heavy chain or any combination thereof. Additionally or alternatively, in some embodiments, the immunoglobulin-related compositions contain an IgG4 constant region comprising a S228P mutation. An engineered antibody including the above LALA substitution shows anti-tumor effect without any undesirable effects of toxicity, PK profile and impaired stability caused by the Fc-mediated effector immune functions (Pharmacol Ther. 2019; 200: 110-125).

In some aspects, the anti-DLL3 immunoglobulin-related compositions described herein contain structural modifications to facilitate rapid binding and cell uptake and/or slow release. In some aspects, the anti-DLL3 immunoglobulin-related composition of the present technology (e.g., an antibody) may contain a deletion in the CH2 constant heavy chain region to facilitate rapid binding and cell uptake and/or slow release. In some aspects, a Fab fragment is used to facilitate rapid binding and cell uptake and/or slow release. In some aspects, a F(ab)′2 fragment is used to facilitate rapid binding and cell uptake and/or slow release.

Amino acid sequence modification(s) of the anti-DLL3 antibodies described herein are contemplated. For example, it may be desirable to improve the binding affinity and/or other biological properties of the antibody. Amino acid sequence variants of an anti-DLL3 antibody are prepared by introducing appropriate nucleotide changes into the antibody nucleic acid, or by peptide synthesis. Such modifications include, for example, deletions from, and/or insertions into and/or substitutions of, residues within the amino acid sequences of the antibody. Any combination of deletion, insertion, and substitution is made to obtain the antibody of interest, as long as the obtained antibody possesses the desired properties. The modification also includes the change of the pattern of glycosylation of the protein. The sites of greatest interest for substitutional mutagenesis include the hypervariable regions, but FR alterations are also contemplated. “Conservative substitutions” are shown in the Table below.

Amino Acid Substitutions

| Original | | Conservative |
|---|---|---|
| Residue | Exemplary Substitutions | Substitutions |
| Ala (A) | val; leu; ile | val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; phe; norleucine | leu |
| Leu (L) | norleucine; ile; val; met; ala; phe | ile |
| Lys (K) | arg; gln; asn | arg |
| Met (M) | leu; phe; ile | leu |
| Phe (F) | leu; val; ile; ala; tyr | tyr |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; norleucine | leu |

One type of substitutional variant involves substituting one or more hypervariable region residues of a parent antibody. A convenient way for generating such substitutional variants involves affinity maturation using phage display. Specifically, several hypervariable region sites (e.g., 6-7 sites) are mutated to generate all possible amino acid substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and the antigen. Such contact residues and neighboring residues are candidates for substitution according to the techniques elaborated herein. Once such variants are generated, the panel of variants is subjected to screening as described herein and antibodies with similar or superior properties in one or more relevant assays may be selected for further development.

In one aspect, the present technology provides a nucleic acid sequence encoding any of the immunoglobulin-related compositions described herein. Also disclosed herein are recombinant nucleic acid sequences encoding any of the antibodies described herein. In some embodiments, the nucleic acid sequence is selected from the group consisting of SEQ ID NOs: 1,6, 11, 16, 21, 26, 31, and 36.

In another aspect, the present technology provides a host cell or expression vector expressing any nucleic acid sequence encoding any of the immunoglobulin-related compositions described herein.

The immunoglobulin-related compositions of the present technology (e.g., an anti-DLL3 antibody) can be monospecific, bispecific, trispecific or of greater multispecificity. Multispecific antibodies can be specific for different epitopes of one or more DLL3 polypeptides or can be specific for both the DLL3 polypeptide(s) as well as for heterologous compositions, such as a heterologous polypeptide or solid support material. See, e.g., WO 93/17715; WO 92/08802; WO 91/00360; WO 92/05793; Tutt et al., J. Immunol. 147: 60-69 (1991); U.S. Pat. Nos. 5,573,920, 4,474,893, 5,601,819, 4,714,681, 4,925,648; 6,106,835; Kostelny et al., J. Immunol. 148: 1547-1553 (1992). In some embodiments, the immunoglobulin-related compositions are chimeric. In certain embodiments, the immunoglobulin-related compositions are humanized.

The immunoglobulin-related compositions of the present technology can further be recombinantly fused to a heterologous polypeptide at the N- or C-terminus or chemically conjugated (including covalently and non-covalently conjugations) to polypeptides or other compositions. For example, the immunoglobulin-related compositions of the present technology can be recombinantly fused or conjugated to molecules useful as labels in detection assays and effector molecules such as heterologous polypeptides, drugs, or toxins. See, e.g., WO 92/08495; WO 91/14438; WO 89/12624; U.S. Pat. No. 5,314,995; and EP 0 396 387.

In any of the above embodiments of the immunoglobulin-related compositions of the present technology, the antibody or antigen binding fragment may be optionally conjugated to an agent selected from the group consisting of isotopes, dyes, chromagens, contrast agents, drugs, toxins, cytokines, enzymes, enzyme inhibitors, hormones, hormone antagonists, growth factors, radionuclides, metals, liposomes, nanoparticles, RNA, DNA or any combination thereof. In some embodiments, the antibody or antigen binding fragment of the present technology may be combined with a pharmaceutically-acceptable carrier. For a chemical bond or physical bond, a functional group on the immunoglobulin-related composition typically associates with a functional group on the agent. Alternatively, a functional group on the agent associates with a functional group on the immunoglobulin-related composition.

The functional groups on the agent and immunoglobulin-related composition can associate directly. For example, a functional group (e.g., a sulfhydryl group) on an agent can associate with a functional group (e.g., sulfhydryl group) on an immunoglobulin-related composition to form a disulfide. Alternatively, the functional groups can associate through a cross-linking agent (i.e., linker). Some examples of cross-linking agents are described below. The cross-linker can be attached to either the agent or the immunoglobulin-related composition. The number of agents or immunoglobulin-related compositions in a conjugate is also limited by the number of functional groups present on the other. For example, the maximum number of agents associated with a conjugate depends on the number of functional groups present on the immunoglobulin-related composition. Alternatively, the maximum number of immunoglobulin-related compositions associated with an agent depends on the number of functional groups present on the agent.

In yet another embodiment, the conjugate comprises one immunoglobulin-related composition associated to one agent. In one embodiment, a conjugate comprises at least one agent chemically bonded (e.g., conjugated) to at least one immunoglobulin-related composition. The agent can be chemically bonded to an immunoglobulin-related composition by any method known to those in the art. For example, a functional group on the agent may be directly attached to a functional group on the immunoglobulin-related composition. Some examples of suitable functional groups include, for example, amino, carboxyl, sulfhydryl, maleimide, isocyanate, isothiocyanate and hydroxyl.

The agent may also be chemically bonded to the immunoglobulin-related composition by means of cross-linking agents, such as dialdehydes, carbodiimides, dimaleimides, and the like. Cross-linking agents can, for example, be obtained from Pierce Biotechnology, Inc., Rockford, Ill. The Pierce Biotechnology, Inc. web-site can provide assistance. Additional cross-linking agents include the platinum cross-linking agents described in U.S. Pat. Nos. 5,580,990; 5,985,566; and 6,133,038 of Kreatech Biotechnology, B.V, Amsterdam, The Netherlands.

Alternatively, the functional group on the agent and immunoglobulin-related composition can be the same. Homobifunctional cross-linkers are typically used to cross-link identical functional groups. Examples of homobifunctional cross-linkers include EGS (i.e., ethylene glycol bis[succinimidylsuccinate]), DSS (i.e., disuccinimidyl suberate), DMA (i.e., dimethyl adipimidate·2HCl), DTSSP (i.e., 3,3′-dithiobis[sulfosuccinimidylpropionate])), DPDPB (i.e., 1,4-di-[3′-(2′-pyridyldithio)-propionamido]butane), and BMH (i.e., bis-maleimidohexane). Such homobifunctional cross-linkers are also available from Pierce Biotechnology, Inc.

In other instances, it may be beneficial to cleave the agent from the immunoglobulin-related composition. The web-site of Pierce Biotechnology, Inc. described above can also provide assistance to one skilled in the art in choosing suitable cross-linkers which can be cleaved by, for example, enzymes in the cell. Thus the agent can be separated from the immunoglobulin-related composition. Examples of cleavable linkers include SMPT (i.e., 4-succinimidyloxycarbonyl-methyl-a-[2-pyridyldithio]toluene), Sulfo-LC-SPDP (i.e., sulfosuccinimidyl 6-(3-[2-pyridyldithio]-propionamido)hexanoate), LC-SPDP (i.e., succinimidyl 6-(3-[2-pyridyldithio]-propionamido)hexanoate), Sulfo-LC-SPDP (i.e., sulfosuccinimidyl 6-(3-[2-pyridyldithio]-propionamido)hexanoate), SPDP (i.e., N-succinimidyl 3-[2-pyridyldithio]-propionamidohexanoate), and AEDP (i.e., 3-[(2-aminoethyl)dithio]propionic acid HCl).

In another embodiment, a conjugate comprises at least one agent physically bonded with at least one immunoglobulin-related composition. Any method known to those in the art can be employed to physically bond the agents with the immunoglobulin-related compositions. For example, the immunoglobulin-related compositions and agents can be mixed together by any method known to those in the art. The order of mixing is not important. For instance, agents can be physically mixed with immunoglobulin-related compositions by any method known to those in the art. For example, the immunoglobulin-related compositions and agents can be placed in a container and agitated, by for example, shaking the container, to mix the immunoglobulin-related compositions and agents.

The immunoglobulin-related compositions can be modified by any method known to those in the art. For instance, the immunoglobulin-related composition may be modified by means of cross-linking agents or functional groups, as described above.

The antibody of the present invention also includes a modification of an antibody. The modification is used to mean the antibody of the present invention, which is chemically or biologically modified. Examples of such a chemical modification include the binding of a chemical moiety to an amino acid skeleton, and the chemical modification of an N-linked or O-linked carbohydrate chain. Examples of such a biological modification include antibodies which have undergone a posttranslational modification (e.g., N-linked or O-linked glycosylation, N-terminal or C-terminal processing, deamidation, isomerization of aspartic acid, oxidation of methionine, and conversion of N-terminal glutamine or N-terminal glutamic acid to pyroglutamic acid), and antibodies, to the N-terminus of which a methionine residue is added as a result of having been allowed to be expressed using prokaryote host cells. In addition, such a modification is also meant to include labeled antibodies for enabling detection or isolation of the antibody of the present invention or an antigen, for example, an enzymatically labeled antibody, a fluorescently labeled antibody, and an affinity-labeled antibody. Such a modification of the antibody of the present invention is useful for the improvement of the stability and retention in blood of an antibody; a reduction in antigenicity; detection or isolation of an antibody or an antigen; etc.

Moreover, by regulating a sugar chain modification (glycosylation, de-fucosylation, etc.) that binds to the antibody of the present invention, antibody-dependent cellular cytotoxic activity can be enhanced. As techniques of regulating the sugar chain modification of an antibody, those described in International Publication Nos. WO1999/54342, WO2000/61739, and WO2002/31140, WO2007/133855 etc. are known, though the techniques are not limited thereto. The antibody of the present invention also includes antibodies in respect of which the aforementioned sugar chain modification has been regulated.

Once an antibody gene is isolated, the gene can be introduced into an appropriate host to produce an antibody, using an appropriate combination of a host and an expression vector. A specific example of the antibody gene can be a combination of a gene encoding the heavy chain sequence of the antibody described in the present description and a gene encoding the light chain sequence of the antibody described therein. Upon transformation of host cells, such a heavy chain sequence gene and a light chain sequence gene may be inserted into a single expression vector, or these genes may instead each be inserted into different expression vectors.

When eukaryotic cells are used as hosts, animal cells, plant cells or eukaryotic microorganisms can be used. In particular, examples of the animal cells can include mammalian cells such as COS cells which are monkey cells (Gluzman, Y, Cell (1981) 23, p. 175-182, ATCC CRL-1650), mouse fibroblasts NIH3T3 (ATCC No. CRL-1658), a dihydrofolate reductase-deficient cell line of Chinese hamster ovary cells (CHO cells, ATCC CCL-61) (Urlaub, G. and Chasin, L. A. Proc. Natl. Acad. Sci. U.S.A. (1980) 77, p. 4126-4220), and FreeStyle 293F cells (Invitrogen Corp.).

When prokaryotic cells are used as hosts, Escherichia coli or Bacillus subtilis can be used, for example.

An antibody gene of interest is introduced into these cells for transformation, and the transformed cells are then cultured in vitro to obtain an antibody. In the aforementioned culture, there are cases where yield is different depending on the sequence of the antibody, and thus, it is possible to select an antibody, which is easily produced as a medicament, from antibodies having equivalent binding activity, using the yield as an indicator. Accordingly, the antibody of the present invention also includes an antibody obtained by the above-described method for producing an antibody, which comprises a step of culturing the transformed host cells and a step of collecting an antibody of interest or a functional fragment of the antibody from the culture obtained in the aforementioned step.

It is known that the lysine residue at the carboxyl terminus of the heavy chain of an antibody produced in cultured mammalian cells is deleted (Journal of Chromatography A, 705: 129-134 (1995)), and also, it is known that the two amino acid residues at the heavy chain carboxyl terminus, glycine and lysine, are deleted, and that the proline residue newly positioned at the carboxyl terminus is amidated (Analytical Biochemistry, 360: 75-83 (2007)). However, such deletion and modification of these heavy chain sequences does not have an influence on the antigen-binding activity and effector function (activation of complement, antibody-dependent cellular cytotoxicity, etc.) of an antibody. Accordingly, the antibody according to the present invention also includes an antibody that has undergone the aforementioned modification, and a functional fragment of the antibody, and specific examples of such an antibody include a deletion mutant comprising a deletion of 1 or 2 amino acids at the heavy chain carboxyl terminus, and a deletion mutant formed by amidating the aforementioned deletion mutant (e.g., a heavy chain in which the proline residue at the carboxyl-terminal site is amidated). However, deletion mutants involving a deletion at the carboxyl terminus of the heavy chain of the antibody according to the present invention are not limited to the above-described deletion mutants, as long as they retain antigen-binding activity and effector function. Two heavy chains constituting the antibody according to the present invention may be any one type of heavy chain selected from the group consisting of a full-length antibody and the above-described deletion mutants, or may be a combination of any two types selected from the aforementioned group. The ratio of individual deletion mutants can be influenced by the types of cultured mammalian cells that produce the antibody according to the present invention, and the culture conditions. Examples of the main ingredient of the antibody according to the present invention can include antibodies where one amino acid residue is deleted at each of the carboxyl termini of the two heavy chains.

Examples of the biological activity of an antibody can generally include antigen-binding activity, activity of being internalized into cells expressing an antigen by binding to the antigen, activity of neutralizing the activity of an antigen, activity of enhancing the activity of an antigen, antibody-dependent cellular cytotoxic (ADCC) activity, complement-dependent cytotoxic (CDC) activity, and antibody-dependent cellular phagocytosis (ADCP). The function of the antibody according to the present invention is binding activity against DLL3 and is preferably the activity of being internalized into DLL3-expressing cells by binding to DLL3. Moreover, the antibody of the present invention may have ADCC activity, CDC activity and/or ADCP activity, as well as cellular internalization activity.

### IV. Production of Anti-DLL3 Antibody

The anti-DLL3 antibody of the present invention may be derived from any species. Preferred examples of the species can include humans, monkeys, rats, mice and rabbits. When the anti-DLL3 antibody of the present invention is derived from a species other than humans, it is preferred to chimerize or humanize the anti-DLL3 antibody by a well-known technique. The antibody of the present invention may be a polyclonal antibody or may be a monoclonal antibody, and a monoclonal antibody is preferred.

The anti-DLL3 antibody of the present invention is an antibody that can target tumor cells. Specifically, the anti-DLL3 antibody of the present invention possesses the property of being able to recognize tumor cells, the property of being able to bind to tumor cells, and/or the property of being internalized into tumor cells by cellular uptake, and the like. Accordingly, the anti-DLL3 antibody of the present invention can be conjugated to a compound having antitumor activity via a linker to prepare an antibody-drug conjugate.

The binding activity of an antibody against tumor cells can be confirmed by flow cytometry. The uptake of an antibody into tumor cells can be confirmed by (1) an assay of visualizing a cellularly taken-up antibody under a fluorescent microscope using a secondary antibody (fluorescently labeled) binding to the antibody (Cell Death and Differentiation, 2008, 15, 751-761), (2) an assay of measuring the amount of cellularly taken-up fluorescence using a secondary antibody (fluorescently labeled) binding to the antibody (Molecular Biology of the Cell Vol. 15, 5268-5282, December 2004) or (3) a Mab-ZAP assay using an immunotoxin binding to the antibody, wherein the toxin is released upon cellular uptake, so as to suppress cell growth (Bio Techniques 28: 162-165, January 2000). A recombinant conjugated protein of a catalytic region of diphtheria toxin and protein G may be used as the immunotoxin.

In the present description, the term “high internalization ability” is used to mean that the survival rate (which is indicated by a ratio relative to a cell survival rate without antibody addition defined as 100%) of DLL3-expressing cells to which the aforementioned antibody and a saporin-labeled anti-rat IgG antibody have been administered is preferably 70% or less, and more preferably 60% or less.

The antitumor antibody-drug conjugate of the present invention comprises a conjugated compound exerting an antitumor effect. Therefore, it is preferred, but not essential, that the antibody itself should have an antitumor effect. For the purpose of specifically and/or selectively exerting the cytotoxicity of the antitumor compound in tumor cells, it is important and preferred that the antibody should have a property of being internalized and transferred into tumor cells.

The anti-DLL3 antibody can be obtained by immunizing an animal with a polypeptide serving as an antigen by a method usually performed in this field, and then collecting and purifying an antibody produced in a living body thereof. It is preferred to use DLL3 retaining a three-dimensional structure as an antigen. Examples of such a method can include a DNA immunization method.

The origin of the antigen is not limited to a human, and thus, an animal can also be immunized with an antigen derived from a non-human animal such as a mouse or a rat. In this case, an antibody applicable to the disease of a human can be selected by examining the cross-reactivity of the obtained antibody binding to the heterologous antigen with the human antigen.

Furthermore, antibody-producing cells that produce an antibody against the antigen can be fused with myeloma cells according to a known method (e.g., Kohler and Milstein, Nature (1975) 256, 495-497; and Kennet, R. ed., Monoclonal Antibodies, 365-367, Plenum Press, N. Y (1980)) to establish hybridomas, so as to obtain a monoclonal antibody.

Hereinafter, the method for obtaining an antibody against DLL3 will be specifically described.

General Overview. Initially, a target polypeptide is chosen to which an antibody of the present technology can be raised. For example, an antibody may be raised against the full-length DLL3 protein, or to a portion of the extracellular domain of the DLL3 protein. Techniques for generating antibodies directed to such target polypeptides are well known to those skilled in the art. Examples of such techniques include, for example, but are not limited to, those involving display libraries, xeno or human mice, hybridomas, and the like. Target polypeptides within the scope of the present technology include any polypeptide derived from DLL3 protein containing the extracellular domain which is capable of eliciting an immune response.

It should be understood that recombinantly engineered antibodies and antibody fragments, e.g., antibody-related polypeptides, which are directed to DLL3 protein and fragments thereof are suitable for use in accordance with the present disclosure.

Anti-DLL3 antibodies that can be subjected to the techniques set forth herein include monoclonal and polyclonal antibodies, and antibody fragments such as Fab, Fab′, F(ab′)₂, Fd, scFv, diabodies, antibody light chains, antibody heavy chains and/or antibody fragments. Methods useful for the high yield production of antibody Fv-containing polypeptides, e.g., Fab′ and F(ab′)₂ antibody fragments have been described. See U.S. Pat. No. 5,648,237.

Generally, an antibody is obtained from an originating species. More particularly, the nucleic acid or amino acid sequence of the variable portion of the light chain, heavy chain or both, of an originating species antibody having specificity for a target polypeptide antigen is obtained. An originating species is any species which was useful to generate the antibody of the present technology or library of antibodies, e.g., rat, mouse, rabbit, chicken, monkey, human, and the like.

Phage or phagemid display technologies are useful techniques to derive the antibodies of the present technology. Techniques for generating and cloning monoclonal antibodies are well known to those skilled in the art. Expression of sequences encoding antibodies of the present technology, can be carried out in E. coli.

Due to the degeneracy of nucleic acid coding sequences, other sequences which encode substantially the same amino acid sequences as those of the naturally occurring proteins may be used in the practice of the present technology These include, but are not limited to, nucleic acid sequences including all or portions of the nucleic acid sequences encoding the above polypeptides, which are altered by the substitution of different codons that encode a functionally equivalent amino acid residue within the sequence, thus producing a silent change. It is appreciated that the nucleotide sequence of an immunoglobulin according to the present technology tolerates sequence homology variations of up to 25% as calculated by standard methods (“Current Methods in Sequence Comparison and Analysis,” Macromolecule Sequencing and Synthesis, Selected Methods and Applications, pp. 127-149, 1998, Alan R. Liss, Inc.) so long as such a variant forms an operative antibody which recognizes DLL3 proteins. For example, one or more amino acid residues within a polypeptide sequence can be substituted by another amino acid of a similar polarity which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Also included within the scope of the present technology are proteins or fragments or derivatives thereof which are differentially modified during or after translation, e.g., by glycosylation, proteolytic cleavage, linkage to an antibody molecule or other cellular ligands, etc. Additionally, an immunoglobulin encoding nucleic acid sequence can be mutated in vitro or in vivo to create and/or destroy translation, initiation, and/or termination sequences or to create variations in coding regions and/or form new restriction endonuclease sites or destroy pre-existing ones, to facilitate further in vitro modification. Any technique for mutagenesis known in the art can be used, including but not limited to in vitro site directed mutagenesis, J. Biol. Chem. 253:6551, use of Tab linkers (Pharmacia), and the like.

Preparation of Polyclonal Antisera and Immunogens. Methods of generating antibodies or antibody fragments of the present technology typically include immunizing a subject (generally a non-human subject such as a mouse or rabbit) with a purified DLL3 protein or fragment thereof or with a cell expressing the DLL3 protein or fragment thereof. An appropriate immunogenic preparation can contain, e.g., a recombinantly-expressed DLL3 protein or a chemically-synthesized DLL3 peptide. The extracellular domain of the DLL3 protein, or a portion or fragment thereof, can be used as an immunogen to generate an anti-DLL3 antibody that binds to the DLL3 protein, or a portion or fragment thereof using standard techniques for polyclonal and monoclonal antibody preparation.

The full-length DLL3 protein or fragments thereof, are useful as fragments as immunogens. In some embodiments, a DLL3 fragment comprises the extracellular domain of DLL3 such that an antibody raised against the peptide forms a specific immune complex with DLL3 protein.

The extracellular domain of DLL3 is 466 amino acids in length, spanning amino acids 27-492 of the full length DLL3 protein. In some embodiments, the antigenic DLL3 peptide comprises at least 5, 8, 10, 15, 20, 30, 40, 50, 60, 70, 80, 90, 100, 150, 200, 250, 300, 350, 400, or 450 amino acid residues. Longer antigenic peptides are sometimes desirable over shorter antigenic peptides, depending on use and according to methods well known to those skilled in the art. Multimers of a given epitope are sometimes more effective than a monomer.

If needed, the immunogenicity of the DLL3 protein (or fragment thereof) can be increased by fusion or conjugation to a carrier protein such as keyhole limpet hemocyanin (KLH) or ovalbumin (OVA). Many such carrier proteins are known in the art. One can also combine the DLL3 protein with a conventional adjuvant such as Freund's complete or incomplete adjuvant to increase the subject's immune reaction to the polypeptide. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory compounds. These techniques are standard in the art.

In describing the present technology, immune responses may be described as either “primary” or “secondary” immune responses. A primary immune response, which is also described as a “protective” immune response, refers to an immune response produced in an individual as a result of some initial exposure (e.g., the initial “immunization”) to a particular antigen, e.g., DLL3 protein. In some embodiments, the immunization can occur as a result of vaccinating the individual with a vaccine containing the antigen. For example, the vaccine can be a DLL3 vaccine comprising one or more DLL3 protein-derived antigens. Aprimary immune response can become weakened or attenuated over time and can even disappear or at least become so attenuated that it cannot be detected. Accordingly, the present technology also relates to a “secondary” immune response, which is also described here as a “memory immune response.” The term secondary immune response refers to an immune response elicited in an individual after a primary immune response has already been produced.

Thus, a secondary immune response can be elicited, e.g., to enhance an existing immune response that has become weakened or attenuated, or to recreate a previous immune response that has either disappeared or can no longer be detected. The secondary or memory immune response can be either a humoral (antibody) response or a cellular response. A secondary or memory humoral response occurs upon stimulation of memory B cells that were generated at the first presentation of the antigen. Delayed type hypersensitivity (DTH) reactions are a type of cellular secondary or memory immune response that are mediated by CD4⁺ T cells. A first exposure to an antigen primes the immune system and additional exposure(s) results in a DTH.

Following appropriate immunization, the anti-DLL3 antibody can be prepared from the subject's serum. If desired, the antibody molecules directed against the DLL3 protein can be isolated from the mammal (e.g., from the blood) and further purified by well-known techniques, such as polypeptide A chromatography to obtain the IgG fraction.

Monoclonal Antibody. In one embodiment of the present technology, the antibody is an anti-DLL3 monoclonal antibody. For example, in some embodiments, the anti-DLL3 monoclonal antibody may be a human or a mouse anti-DLL3 monoclonal antibody. For preparation of monoclonal antibodies directed towards the DLL3 protein, or derivatives, fragments, analogs or homologs thereof, any technique that provides for the production of antibody molecules by continuous cell line culture can be utilized. Such techniques include, but are not limited to, the hybridoma technique (See, e.g., Kohler & Milstein, 1975. Nature 256: 495-497); the trioma technique; the human B-cell hybridoma technique (See, e.g., Kozbor, et al., 1983. Immunol. Today 4: 72) and the EBV hybridoma technique to produce human monoclonal antibodies (See, e.g., Cole, et al., 1985. In: MONOCLONAL ANTIBODIES AND CANCER THERAPY, Alan R. Liss, Inc., pp. 77-96). Human monoclonal antibodies can be utilized in the practice of the present technology and can be produced by using human hybridomas (See, e.g., Cote, et al., 1983. Proc. Natl. Acad. Sci. USA 80: 2026-2030) or by transforming human B-cells with Epstein Barr Virus in vitro (See, e.g., Cole, et al., 1985. In: MONOCLONAL ANTIBODIES AND CANCER THERAPY, Alan R. Liss, Inc., pp. 77-96). For example, a population of nucleic acids that encode regions of antibodies can be isolated. PCR utilizing primers derived from sequences encoding conserved regions of antibodies is used to amplify sequences encoding portions of antibodies from the population and then DNAs encoding antibodies or fragments thereof, such as variable domains, are reconstructed from the amplified sequences. Such amplified sequences also can be fused to DNAs encoding other proteins—e.g., a bacteriophage coat, or a bacterial cell surface protein—for expression and display of the fusion polypeptides on phage or bacteria. Amplified sequences can then be expressed and further selected or isolated based, e.g., on the affinity of the expressed antibody or fragment thereof for an antigen or epitope present on the DLL3 protein. Alternatively, hybridomas expressing anti-DLL3 monoclonal antibodies can be prepared by immunizing a subject and then isolating hybridomas from the subject's spleen using routine methods. See, e.g., Milstein et al., (Galfre and Milstein, Methods Enzymol (1981) 73: 3-46). Screening the hybridomas using standard methods will produce monoclonal antibodies of varying specificity (i.e., for different epitopes) and affinity. A selected monoclonal antibody with the desired properties, e.g., DLL3 binding, can be used as expressed by the hybridoma, it can be bound to a molecule such as polyethylene glycol (PEG) to alter its properties, or a cDNA encoding it can be isolated, sequenced and manipulated in various ways. Synthetic dendromeric trees can be added to reactive amino acid side chains, e.g., lysine, to enhance the immunogenic properties of DLL3 protein. Also, CPG-dinucleotide techniques can be used to enhance the immunogenic properties of the DLL3 protein. Other manipulations include substituting or deleting particular amino acyl residues that contribute to instability of the antibody during storage or after administration to a subject, and affinity maturation techniques to improve affinity of the antibody of the DLL3 protein.

Hybridoma Technique. In some embodiments, the antibody of the present technology is an anti-DLL3 monoclonal antibody produced by a hybridoma which includes a B cell obtained from a transgenic non-human animal, e.g., a transgenic mouse, having a genome comprising a human heavy chain transgene and a light chain transgene fused to an immortalized cell. Hybridoma techniques include those known in the art and taught in Harlow et al., Antibodies: A Laboratory Manual Cold Spring Harbor Laboratory, Cold Spring Harbor, NY, 349 (1988); Hammerling et al., Monoclonal Antibodies And T-Cell Hybridomas, 563-681 (1981). Other methods for producing hybridomas and monoclonal antibodies are well known to those of skill in the art.

Phage Display Technique. As noted above, the antibodies of the present technology can be produced through the application of recombinant DNA and phage display technology. For example, anti-DLL3 antibodies, can be prepared using various phage display methods known in the art. In phage display methods, functional antibody domains are displayed on the surface of a phage particle which carries polynucleotide sequences encoding them. Phages with a desired binding property are selected from a repertoire or combinatorial antibody library (e.g., human or murine) by selecting directly with an antigen, typically an antigen bound or captured to a solid surface or bead. Phages used in these methods are typically filamentous phage including fd and M13 with Fab, Fv or disulfide stabilized Fv antibody domains that are recombinantly fused to either the phage gene III or gene VIII protein. In addition, methods can be adapted for the construction of Fab expression libraries (See, e.g., Huse, et al., Science 246: 1275-1281, 1989) to allow rapid and effective identification of monoclonal Fab fragments with the desired specificity for a DLL3 polypeptide, e.g., a polypeptide or derivatives, fragments, analogs or homologs thereof. Other examples of phage display methods that can be used to make the antibodies of the present technology include those disclosed in Huston et al., Proc. Natl. Acad. Sci U.S.A., 85: 5879-5883, 1988; Chaudhary et al., Proc. Natl. Acad. Sci U.S.A., 87: 1066-1070, 1990; Brinkman et al., J. Immunol. Methods 182: 41-50, 1995; Ames et al., J. Immunol. Methods 184: 177-186, 1995; Kettleborough et al., Eur. J. Immunol. 24: 952-958, 1994; Persic et al., Gene 187: 9-18, 1997; Burton et al., Advances in Immunology 57: 191-280, 1994; PCT/GB91/01134; WO 90/02809; WO 91/10737; WO 92/01047; WO 92/18619; WO 93/11236; WO 95/15982; WO 95/20401; WO 96/06213; WO 92/01047 (Medical Research Council et al.); WO 97/08320 (Morphosys); WO 92/01047 (CAT/MRC); WO 91/17271 (Affymax); and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727 and 5,733,743. Methods useful for displaying polypeptides on the surface of bacteriophage particles by attaching the polypeptides via disulfide bonds have been described by Lohning, U.S. Pat. No. 6,753,136. As described in the above references, after phage selection, the antibody coding regions from the phage can be isolated and used to generate whole antibodies, including human antibodies, or any other desired antigen binding fragment, and expressed in any desired host including mammalian cells, insect cells, plant cells, yeast, and bacteria. For example, techniques to recombinantly produce Fab, Fab′ and F(ab′)₂ fragments can also be employed using methods known in the art such as those disclosed in WO 92/22324; Mullinax et al., BioTechniques 12: 864-869, 1992; and Sawai et al., AJRI 34: 26-34, 1995; and Better et al., Science 240: 1041-1043, 1988.

Generally, hybrid antibodies or hybrid antibody fragments that are cloned into a display vector can be selected against the appropriate antigen in order to identify variants that maintain good binding activity, because the antibody or antibody fragment will be present on the surface of the phage or phagemid particle. See, e.g., Barbas III et al., Phage Display, A Laboratory Manual (Cold Spring Harbor Laboratory Press, Cold Spring Harbor, N.Y, 2001). However, other vector formats could be used for this process, such as cloning the antibody fragment library into a lytic phage vector (modified T7 or Lambda Zap systems) for selection and/or screening.

Expression of Recombinant Anti-DLL3 Antibodies. As noted above, the antibodies of the present technology can be produced through the application of recombinant DNA technology. Recombinant polynucleotide constructs encoding an anti-DLL3 antibody of the present technology typically include an expression control sequence operably-linked to the coding sequences of anti-DLL3 antibody chains, including naturally-associated or heterologous promoter regions. As such, another aspect of the technology includes vectors containing one or more nucleic acid sequences encoding an anti-DLL3 antibody of the present technology. For recombinant expression of one or more of the polypeptides of the present technology, the nucleic acid containing all or a portion of the nucleotide sequence encoding the anti-DLL3 antibody is inserted into an appropriate cloning vector, or an expression vector (i.e., a vector that contains the necessary elements for the transcription and translation of the inserted polypeptide coding sequence) by recombinant DNA techniques well known in the art and as detailed below. Methods for producing diverse populations of vectors have been described by Lerner et al., U.S. Pat. Nos. 6,291,160 and 6,680,192.

In general, expression vectors useful in recombinant DNA techniques are often in the form of plasmids. In the present disclosure, “plasmid” and “vector” can be used interchangeably as the plasmid is the most commonly used form of vector. However, the present technology is intended to include such other forms of expression vectors that are not technically plasmids, such as viral vectors (e.g., replication defective retroviruses, adenoviruses and adeno-associated viruses), which serve equivalent functions. Such viral vectors permit infection of a subject and expression of a construct in that subject. In some embodiments, the expression control sequences are eukaryotic promoter systems in vectors capable of transforming or transfecting eukaryotic host cells. Once the vector has been incorporated into the appropriate host, the host is maintained under conditions suitable for high level expression of the nucleotide sequences encoding the anti-DLL3 antibody, and the collection and purification of the anti-DLL3 antibody, e.g., cross-reacting anti-DLL3 antibodies. See generally, U.S. 2002/0199213. These expression vectors are typically replicable in the host organisms either as episomes or as an integral part of the host chromosomal DNA. Commonly, expression vectors contain selection markers, e.g., ampicillin-resistance or hygromycin-resistance, to permit detection of those cells transformed with the desired DNA sequences. Vectors can also encode signal peptide, e.g., pectate lyase, useful to direct the secretion of extracellular antibody fragments. See U.S. Pat. No. 5,576,195.

The recombinant expression vectors of the present technology comprise a nucleic acid encoding a protein with DLL3 binding properties in a form suitable for expression of the nucleic acid in a host cell, which means that the recombinant expression vectors include one or more regulatory sequences, selected on the basis of the host cells to be used for expression that is operably-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, e.g., in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of polypeptide desired, etc. Typical regulatory sequences useful as promoters of recombinant polypeptide expression (e.g., anti-DLL3 antibody), include, e.g., but are not limited to, promoters of 3-phosphoglycerate kinase and other glycolytic enzymes. Inducible yeast promoters include, among others, promoters from alcohol dehydrogenase, isocytochrome C, and enzymes responsible for maltose and galactose utilization. In one embodiment, a polynucleotide encoding an anti-DLL3 antibody of the present technology is operably-linked to an ara B promoter and expressible in a host cell. See U.S. Pat. No. 5,028,530. The expression vectors of the present technology can be introduced into host cells to thereby produce polypeptides or peptides, including fusion polypeptides, encoded by nucleic acids as described herein (e.g., anti-DLL3 antibody, etc.).

Another aspect of the present technology pertains to anti-DLL3 antibody-expressing host cells, which contain a nucleic acid encoding one or more anti-DLL3 antibodies. The recombinant expression vectors of the present technology can be designed for expression of an anti-DLL3 antibody in prokaryotic or eukaryotic cells. For example, an anti-DLL3 antibody can be expressed in bacterial cells such as Escherichia coli, insect cells (using baculovirus expression vectors), fungal cells, e.g., yeast, yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, e.g., using T7 promoter regulatory sequences and T7 polymerase. Methods useful for the preparation and screening of polypeptides having a predetermined property, e.g., anti-DLL3 antibody, via expression of stochastically generated polynucleotide sequences has been previously described. See U.S. Pat. Nos. 5,763,192; 5,723,323; 5,814,476; 5,817,483; 5,824,514; 5,976,862; 6,492,107; 6,569,641.

Expression of polypeptides in prokaryotes is most often carried out in E. coli with vectors containing constitutive or inducible promoters directing the expression of either fusion or non-fusion polypeptides. Fusion vectors add a number of amino acids to a polypeptide encoded therein, usually to the amino terminus of the recombinant polypeptide. Such fusion vectors typically serve three purposes: (i) to increase expression of recombinant polypeptide; (ii) to increase the solubility of the recombinant polypeptide; and (iii) to aid in the purification of the recombinant polypeptide by acting as a ligand in affinity purification. Often, in fusion expression vectors, a proteolytic cleavage site is introduced at the junction of the fusion moiety and the recombinant polypeptide to enable separation of the recombinant polypeptide from the fusion moiety subsequent to purification of the fusion polypeptide. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding polypeptide, or polypeptide A, respectively, to the target recombinant polypeptide.

Examples of suitable inducible non-fusion E. coli expression vectors include pTrc (Amrann et al., (1988) Gene 69: 301-315) and pET 11d (Studier et al., GENE EXPRESSION TECHNOLOGY: METHODS IN ENZYMOLOGY 185, Academic Press, San Diego, Calif. (1990) 60-89). Methods for targeted assembly of distinct active peptide or protein domains to yield multifunctional polypeptides via polypeptide fusion has been described by Pack et al., U.S. Pat. Nos. 6,294,353; 6,692,935. One strategy to maximize recombinant polypeptide expression, e.g., an anti-DLL3 antibody, in E. coli is to express the polypeptide in host bacteria with an impaired capacity to proteolytically cleave the recombinant polypeptide. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in the expression host, e.g., E. coli (See, e.g., Wada, et al., 1992. Nucl. Acids Res. 20: 2111-2118). Such alteration of nucleic acid sequences of the present technology can be carried out by standard DNA synthesis techniques.

In another embodiment, the anti-DLL3 antibody expression vector is a yeast expression vector. Examples of vectors for expression in yeast Saccharomyces cerevisiae include pYepSec1 (Baldari, et al., 1987. EMBO J. 6: 229-234), pMFa (Kurjan and Herskowitz, Cell 30: 933-943, 1982), pJRY88 (Schultz et al., Gene 54: 113-123, 1987), pYES2 (Invitrogen Corporation, San Diego, Calif.), and picZ (Invitrogen Corp, San Diego, Calif.). Alternatively, an anti-DLL3 antibody can be expressed in insect cells using baculovirus expression vectors. Baculovirus vectors available for expression of polypeptides, e.g., anti-DLL3 antibody, in cultured insect cells (e.g., SF9 cells) include the pAc series (Smith, et al., Mol. Cell. Biol. 3: 2156-2165, 1983) and the pVL series (Lucklow and Summers, 1989. Virology 170: 31-39).

In yet another embodiment, a nucleic acid encoding an anti-DLL3 antibody of the present technology is expressed in mammalian cells using a mammalian expression vector. Examples of mammalian expression vectors include, e.g., but are not limited to, pCDM8 (Seed, Nature 329: 840, 1987) and pMT2PC (Kaufman, et al., EMBO J. 6: 187-195, 1987). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells that are useful for expression of the anti-DLL3 antibody of the present technology, see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y, 1989.

In another embodiment, the recombinant mammalian expression vector is capable of directing expression of the nucleic acid in a particular cell type (e.g., tissue-specific regulatory elements). Tissue-specific regulatory elements are known in the art. Non-limiting examples of suitable tissue-specific promoters include the albumin promoter (liver-specific; Pinkert, et al., Genes Dev. 1: 268-277, 1987), lymphoid-specific promoters (Calame and Eaton, Adv. Immunol. 43: 235-275, 1988), promoters of T cell receptors (Winoto and Baltimore, EMBO J. 8: 729-733, 1989) and immunoglobulins (Banerji, et al., 1983. Cell 33: 729-740; Queen and Baltimore, Cell 33: 741-748, 1983.), neuron-specific promoters (e.g., the neurofilament promoter; Byrne and Ruddle, Proc. Natl. Acad. Sci. USA 86: 5473-5477, 1989), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, Science 249: 374-379, 1990) and the α-fetoprotein promoter (Campes and Tilghman, Genes Dev. 3: 537-546, 1989).

Another aspect of the present methods pertains to host cells into which a recombinant expression vector of the present technology has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, an anti-DLL3 antibody can be expressed in bacterial cells such as E. coli, insect cells, yeast or mammalian cells. Mammalian cells are a suitable host for expressing nucleotide segments encoding immunoglobulins or fragments thereof. See Winnacker, From Genes To Clones, (VCH Publishers, NY, 1987). A number of suitable host cell lines capable of secreting intact heterologous proteins have been developed in the art, and include Chinese hamster ovary (CHO) cell lines, various COS cell lines, HeLa cells, L cells and myeloma cell lines. In some embodiments, the cells are non-human. Expression vectors for these cells can include expression control sequences, such as an origin of replication, a promoter, an enhancer, and necessary processing information sites, such as ribosome binding sites, RNA splice sites, polyadenylation sites, and transcriptional terminator sequences. Queen et al., Immunol. Rev. 89: 49, 1986. Illustrative expression control sequences are promoters derived from endogenous genes, cytomegalovirus, SV40, adenovirus, bovine papillomavirus, and the like. Co et al., J Immunol. 148: 1149, 1992. Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, electroporation, biolistics or viral-based transfection. Other methods used to transform mammalian cells include the use of polybrene, protoplast fusion, liposomes, electroporation, and microinjection (See generally, Sambrook et al., Molecular Cloning). Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y, 1989), and other laboratory manuals. The vectors containing the DNA segments of interest can be transferred into the host cell by well-known methods, depending on the type of cellular host.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding the anti-DLL3 antibody or can be introduced on a separate vector. Cells stably transfected with the introduced nucleic acid can be identified by drug selection (e.g., cells that have incorporated the selectable marker gene will survive, while the other cells die).

A host cell that includes an anti-DLL3 antibody of the present technology, such as a prokaryotic or eukaryotic host cell in culture, can be used to produce (i.e., express) recombinant anti-DLL3 antibody. In one embodiment, the method comprises culturing the host cell (into which a recombinant expression vector encoding the anti-DLL3 antibody has been introduced) in a suitable medium such that the anti-DLL3 antibody is produced. In another embodiment, the method further comprises the step of isolating the anti-DLL3 antibody from the medium or the host cell. Once expressed, collections of the anti-DLL3 antibody, e.g., the anti-DLL3 antibodies or the anti-DLL3 antibody-related polypeptides are purified from culture media and host cells. The anti-DLL3 antibody can be purified according to standard procedures of the art, including HPLC purification, column chromatography, gel electrophoresis and the like. In one embodiment, the anti-DLL3 antibody is produced in a host organism by the method of Boss et al., U.S. Pat. No. 4,816,397. Usually, anti-DLL3 antibody chains are expressed with signal sequences and are thus released to the culture media. However, if the anti-DLL3 antibody chains are not naturally secreted by host cells, the anti-DLL3 antibody chains can be released by treatment with mild detergent. Purification of recombinant polypeptides is well known in the art and includes ammonium sulfate precipitation, affinity chromatography purification technique, column chromatography, ion exchange purification technique, gel electrophoresis and the like (See generally Scopes, Protein Purification (Springer-Verlag, N.Y, 1982).

Polynucleotides encoding anti-DLL3 antibodies, e.g., the anti-DLL3 antibody coding sequences, can be incorporated in transgenes for introduction into the genome of a transgenic animal and subsequent expression in the milk of the transgenic animal. See, e.g., U.S. Pat. Nos. 5,741,957, 5,304,489, and 5,849,992. Suitable transgenes include coding sequences for light and/or heavy chains in operable linkage with a promoter and enhancer from a mammary gland specific gene, such as casein or β-lactoglobulin. For production of transgenic animals, transgenes can be microinjected into fertilized oocytes, or can be incorporated into the genome of embryonic stem cells, and the nuclei of such cells transferred into enucleated oocytes.

Single-Chain Antibodies. In one embodiment, the anti-DLL3 antibody of the present technology is a single-chain anti-DLL3 antibody. According to the present technology, techniques can be adapted for the production of single-chain antibodies specific to a DLL3 protein (See, e.g., U.S. Pat. No. 4,946,778). Examples of techniques which can be used to produce single-chain Fvs and antibodies of the present technology include those described in U.S. Pat. Nos. 4,946,778 and 5,258,498; Huston et al., Methods in Enzymology, 203: 46-88, 1991; Shu, L. et al., Proc. Natl. Acad. Sci. USA, 90: 7995-7999, 1993; and Skerra et al., Science 240: 1038-1040, 1988.

Chimeric and Humanized Antibodies. In one embodiment, the anti-DLL3 antibody of the present technology is a chimeric anti-DLL3 antibody. In one embodiment, the anti-DLL3 antibody of the present technology is a humanized anti-DLL3 antibody. In one embodiment of the present technology, the donor and acceptor antibodies are monoclonal antibodies from different species. For example, the acceptor antibody is a human antibody (to minimize its antigenicity in a human), in which case the resulting CDR-grafted antibody is termed a “humanized” antibody.

Recombinant anti-DLL3 antibodies, such as chimeric and humanized monoclonal antibodies, comprising both human and non-human portions, can be made using standard recombinant DNA techniques, and are within the scope of the present technology. For some uses, including in vivo use of the anti-DLL3 antibody of the present technology in humans as well as use of these agents in in vitro detection assays, it is possible to use chimeric or humanized anti-DLL3 antibodies. Such chimeric and humanized monoclonal antibodies can be produced by recombinant DNA techniques known in the art. Such useful methods include, e.g., but are not limited to, methods described in International Application No. PCT/US86/02269; U.S. Pat. No. 5,225,539; European Patent No. 184187; European Patent No. 171496; European Patent No. 173494; PCT International Publication No. WO 86/01533; U.S. Pat. Nos. 4,816,567; 5,225,539; European Patent No. 125023; Better, et al., 1988. Science 240: 1041-1043; Liu, et al., 1987. Proc. Natl. Acad. Sci. USA 84: 3439-3443; Liu, et al., 1987. J. Immunol. 139: 3521-3526; Sun, et al., 1987. Proc. Natl. Acad. Sci. USA 84: 214-218; Nishimura, et al., 1987. Cancer Res. 47: 999-1005; Wood, et al., 1985. Nature 314: 446-449; Shaw, et al., 1988. J. Natl. Cancer Inst. 80: 1553-1559; Morrison (1985) Science 229: 1202-1207; Oi, et al. (1986) BioTechniques 4: 214; Jones, et al., 1986. Nature 321: 552-525; Verhoeyan, et al., 1988. Science 239: 1534; Morrison, Science 229: 1202, 1985; Oi et al., BioTechniques 4: 214, 1986; Gillies et al., J. Immunol. Methods, 125: 191-202, 1989; U.S. Pat. No. 5,807,715; and Beidler, et al., 1988. J. Immunol. 141: 4053-4060. For example, antibodies can be humanized using a variety of techniques including CDR-grafting (EP 0 239 400; WO 91/09967; U.S. Pat. Nos. 5,530,101; 5,585,089; 5,859,205; 6,248,516; EP460167), veneering or resurfacing (EP 0 592 106; EP 0 519 596; Padlan E. A., Molecular Immunology, 28: 489-498, 1991; Studnicka et al., Protein Engineering 7: 805-814, 1994; Roguska et al., PNAS 91: 969-973, 1994), and chain shuffling (U.S. Pat. No. 5,565,332). In one embodiment, a cDNA encoding a murine anti-DLL3 monoclonal antibody is digested with a restriction enzyme selected specifically to remove the sequence encoding the Fc constant region, and the equivalent portion of a cDNA encoding a human Fc constant region is substituted (See Robinson et al., PCT/US86/02269; Akira et al., European Patent Application 184,187; Taniguchi, European Patent Application 171,496; Morrison et al., European Patent Application 173,494; Neuberger et al., WO 86/01533; Cabilly et al. U.S. Pat. No. 4,816,567; Cabilly et al., European Patent Application 125,023; Better et al. (1988) Science 240: 1041-1043; Liu et al. (1987) Proc. Natl. Acad. Sci. USA 84: 3439-3443; Liu et al. (1987) J Immunol 139: 3521-3526; Sun et al. (1987) Proc. Natl. Acad. Sci. USA 84: 214-218; Nishimura et al. (1987) Cancer Res 47: 999-1005; Wood et al. (1985) Nature 314: 446-449; and Shaw et al. (1988) J. Natl. Cancer Inst. 80: 1553-1559; U.S. Pat. Nos. 6,180,370; 6,300,064; 6,696,248; 6,706,484; 6,828,422.

In one embodiment, the present technology provides the construction of humanized anti-DLL3 antibodies that are unlikely to induce a human anti-mouse antibody (hereinafter referred to as “HAMA”) response, while still having an effective antibody effector function. As used herein, the terms “human” and “humanized”, in relation to antibodies, relate to any antibody which is expected to elicit a therapeutically tolerable weak immunogenic response in a human subject. In one embodiment, the present technology provides for a humanized anti-DLL3 antibodies, heavy and light chain immunoglobulins.

CDR-Grafted Antibodies. In some embodiments, the anti-DLL3 antibody of the present technology is an anti-DLL3 CDR-grafted antibody. Generally the donor and acceptor antibodies used to generate the anti-DLL3 CDR antibody are monoclonal antibodies from different species; typically the acceptor antibody is a human antibody (to minimize its antigenicity in a human), in which case the resulting CDR-grafted antibody is termed a “humanized” antibody. For detail, “humanized antibodies” refer to antibodies which comprise at least one chain comprising variable region framework residues from a human antibody chain and at least one complementarity determining region (CDR) from a non-human-antibody (e.g., mouse). The term “human antibody,” as used herein, is intended to include antibodies having variable and constant regions derived from human immunoglobulin sequences. However, the term “human antibody,” as used herein, is not intended to include antibodies in which CDR sequences derived from another mammalian species, such as a mouse, have been grafted onto human framework sequences. The graft may be of a single CDR (or even a portion of a single CDR) within a single V_{H} or V_{L} of the acceptor antibody, or can be of multiple CDRs (or portions thereof) within one or both of the V_{H} and V_{L}. Frequently, all three CDRs in all variable domains of the acceptor antibody will be replaced with the corresponding donor CDRs, though one needs to replace only as many as necessary to permit adequate binding of the resulting CDR-grafted antibody to DLL3 protein. Methods for generating CDR-grafted and humanized antibodies are taught by Queen et al. U.S. Pat. Nos. 5,585,089; 5,693,761; 5,693,762; and Winter U.S. Pat. No. 5,225,539; and EP 0682040. Methods useful to prepare V_{H} and V_{L} polypeptides are taught by Winter et al., U.S. Pat. Nos. 4,816,397; 6,291,158; 6,291,159; 6,291,161; 6,545,142; EP 0368684; EP0451216; and EP0120694.

After selecting suitable framework region candidates from the same family and/or the same family member, either or both the heavy and light chain variable regions are produced by grafting the CDRs from the originating species into the hybrid framework regions. Assembly of hybrid antibodies or hybrid antibody fragments having hybrid variable chain regions with regard to either of the above aspects can be accomplished using conventional methods known to those skilled in the art. For example, DNA sequences encoding the hybrid variable domains described herein (i.e., frameworks based on the target species and CDRs from the originating species) can be produced by oligonucleotide synthesis and/or PCR. The nucleic acid encoding CDR regions can also be isolated from the originating species antibodies using suitable restriction enzymes and ligated into the target species framework by ligating with suitable ligation enzymes. Alternatively, the framework regions of the variable chains of the originating species antibody can be changed by site-directed mutagenesis.

Since the hybrids are constructed from choices among multiple candidates corresponding to each framework region, there exist many combinations of sequences which are amenable to construction in accordance with the principles described herein. Accordingly, libraries of hybrids can be assembled having members with different combinations of individual framework regions. Such libraries can be electronic database collections of sequences or physical collections of hybrids.

This process typically does not alter the acceptor antibody's FRs flanking the grafted CDRs. However, one skilled in the art can sometimes improve antigen binding affinity of the resulting anti-DLL3 CDR-grafted antibody by replacing certain residues of a given FR to make the FR more similar to the corresponding FR of the donor antibody. Suitable locations of the substitutions include amino acid residues adjacent to the CDR, or which are capable of interacting with a CDR (See, e.g., U.S. Pat. No. 5,585,089, especially columns 12-16). Or one skilled in the art can start with the donor FR and modify it to be more similar to the acceptor FR or a human consensus FR. Techniques for making these modifications are known in the art. Particularly if the resulting FR fits a human consensus FR for that position, or is at least 90% or more identical to such a consensus FR, doing so may not increase the antigenicity of the resulting modified anti-DLL3 CDR-grafted antibody significantly compared to the same antibody with a fully human FR.

Bispecific Antibodies (BsAbs). A bispecific antibody is an antibody that can bind simultaneously to two targets that have a distinct structure, e.g., two different target antigens, two different epitopes on the same target antigen. BsAbs can be made, for example, by combining heavy chains and/or light chains that recognize different epitopes of the same or different antigen. In some embodiments, by molecular function, a bispecific binding agent binds one antigen (or epitope) on one of its two binding arms (one VH/VL pair), and binds a different antigen (or epitope) on its second arm (a different VH/VL pair). By this definition, a bispecific binding agent has two distinct antigen binding arms (in both specificity and CDR sequences), and is monovalent for each antigen to which it binds.

Bispecific antibodies (BsAb) and bispecific antibody fragments (BsFab) of the present technology have at least one arm that specifically binds to, for example, DLL3 and at least one other arm that specifically binds to a second target antigen. In certain embodiments, the BsAbs are capable of binding to tumor cells that express DLL3 antigen on the cell surface.

A variety of bispecific fusion proteins can be produced using molecular engineering. For example, BsAbs have been constructed that either utilize the full immunoglobulin framework (e.g., IgG), single chain variable fragment (scFv), or combinations thereof. In some embodiments, the bispecific fusion protein is divalent, comprising, for example, a scFv with a single binding site for one antigen and a Fab fragment with a single binding site for a second antigen. In some embodiments, the bispecific fusion protein is divalent, comprising, for example, an scFv with a single binding site for one antigen and another scFv fragment with a single binding site for a second antigen. In other embodiments, the bispecific fusion protein is tetravalent, comprising, for example, an immunoglobulin (e.g., IgG) with two binding sites for one antigen and two identical scFvs for a second antigen. BsAbs composed of two scFv units in tandem have been shown to be a clinically successful bispecific antibody format. In some embodiments, BsAbs comprise two single chain variable fragments (scFvs) in tandem have been designed such that an scFv that binds a tumor antigen (e.g., DLL3) is linked with an scFv that binds to a different target antigen.

Recent methods for producing BsAbs include engineered recombinant monoclonal antibodies which have additional cysteine residues so that they crosslink more strongly than the more common immunoglobulin isotypes. See, e.g., FitzGerald et al., Protein Eng. 10(10):1221-1225 (1997). Another approach is to engineer recombinant fusion proteins linking two or more different single-chain antibody or antibody fragment segments with the needed dual specificities. See, e.g., Coloma et al., Nature Biotech. 15:159-163 (1997). A variety of bispecific fusion proteins can be produced using molecular engineering.

Bispecific fusion proteins linking two or more different single-chain antibodies or antibody fragments are produced in a similar manner. Recombinant methods can be used to produce a variety of fusion proteins. In some certain embodiments, a BsAb according to the present technology comprises an immunoglobulin, which immunoglobulin comprises a heavy chain and a light chain, and an scFv. In some certain embodiments, the scFv is linked to the C-terminal end of the heavy chain of any DLL3 immunoglobulin disclosed herein. In some certain embodiments, scFvs are linked to the C-terminal end of the light chain of any DLL3 immunoglobulin disclosed herein. In various embodiments, scFvs are linked to heavy or light chains via a linker sequence. Appropriate linker sequences necessary for the in-frame connection of the heavy chain Fd to the scFv are introduced into the V_{L} and Vₖₐₚₚₐ domains through PCR reactions. The DNA fragment encoding the scFv is then ligated into a staging vector containing a DNA sequence encoding the CH1 domain. The resulting scFv-CH1 construct is excised and ligated into a vector containing a DNA sequence encoding the V_{H} region of a DLL3 antibody. The resulting vector can be used to transfect an appropriate host cell, such as a mammalian cell for the expression of the bispecific fusion protein.

In some embodiments, a linker is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more amino acids in length. In some embodiments, a linker is characterized in that it tends not to adopt a rigid three-dimensional structure, but rather provides flexibility to the polypeptide (e.g., first and/or second antigen binding sites). In some embodiments, a linker is employed in a BsAb described herein based on specific properties imparted to the BsAb such as, for example, an increase in stability. In some embodiments, a BsAb of the present technology comprises G₄S linker (SEQ ID NO: 82). In some certain embodiments, a BsAb of the present technology comprises a (G₄S)ₙ linker, wherein n is 1, 2, 3, 4, 5, 6, 7, 8, 9, 10, 11, 12, 13, 14, 15 or more (SEQ ID NO: 83).

Fc Modifications. In some embodiments, the anti-DLL3 antibodies of the present technology comprise a variant Fc region, wherein said variant Fc region comprises at least one amino acid modification relative to a wild-type Fc region (or the parental Fc region), such that said molecule has an altered affinity for an Fc receptor (e.g., an FcγR), provided that said variant Fc region does not have a substitution at positions that make a direct contact with Fc receptor based on crystallographic and structural analysis of Fc-Fc receptor interactions such as those disclosed by Sondermann et al., Nature, 406:267-273 (2000). Examples of positions within the Fc region that make a direct contact with an Fc receptor such as an FcγR, include amino acids 234-239 (hinge region), amino acids 265-269 (B/C loop), amino acids 297-299 (C7E loop), and amino acids 327-332 (F/G) loop.

In some embodiments, an anti-DLL3 antibody of the present technology has an altered affinity for activating and/or inhibitory receptors, having a variant Fc region with one or more amino acid modifications, wherein said one or more amino acid modification is a N297 substitution with alanine, or a K322 substitution with alanine.

Glycosylation Modifications. In some embodiments, anti-DLL3 antibodies of the present technology have an Fc region with variant glycosylation as compared to a parent Fc region. In some embodiments, variant glycosylation includes the absence of fucose; in some embodiments, variant glycosylation results from expression in GnT1-deficient CHO cells.

In some embodiments, the antibodies of the present technology, may have a modified glycosylation site relative to an appropriate reference antibody that binds to an antigen of interest (e.g., DLL3), without altering the functionality of the antibody, e.g., binding activity to the antigen. As used herein, “glycosylation sites” include any specific amino acid sequence in an antibody to which an oligosaccharide (i.e., carbohydrates containing two or more simple sugars linked together) will specifically and covalently attach.

Oligosaccharide side chains are typically linked to the backbone of an antibody via either N- or O-linkages. N-linked glycosylation refers to the attachment of an oligosaccharide moiety to the side chain of an asparagine residue. O-linked glycosylation refers to the attachment of an oligosaccharide moiety to a hydroxyamino acid, e.g., serine, threonine. For example, an Fc-glycoform (hDLL3-IgG1n) that lacks certain oligosaccharides including fucose and terminal N-acetylglucosamine may be produced in special CHO cells and exhibit enhanced ADCC effector function.

In some embodiments, the carbohydrate content of an immunoglobulin-related composition disclosed herein is modified by adding or deleting a glycosylation site. Methods for modifying the carbohydrate content of antibodies are well known in the art and are included within the present technology, see, e.g., U.S. Pat. No. 6,218,149; EP 0359096B1; U.S. Patent Publication No. US 2002/0028486; International Patent Application Publication WO 03/035835; U.S. Patent Publication No. 2003/0115614; U.S. Pat. Nos. 6,218,149; 6,472,511; all of which are incorporated herein by reference in their entirety. In some embodiments, the carbohydrate content of an antibody (or relevant portion or component thereof) is modified by deleting one or more endogenous carbohydrate moieties of the antibody. In some certain embodiments, the present technology includes deleting the glycosylation site of the Fc region of an antibody, by modifying position 297 from asparagine to alanine.

Engineered glycoforms may be useful for a variety of purposes, including but not limited to enhancing or reducing effector function. Engineered glycoforms may be generated by any method known to one skilled in the art, for example by using engineered or variant expression strains, by co-expression with one or more enzymes, for example N-acetylglucosaminyltransferase III (GnTIII), by expressing a molecule comprising an Fc region in various organisms or cell lines from various organisms, or by modifying carbohydrate(s) after the molecule comprising Fc region has been expressed. Methods for generating engineered glycoforms are known in the art, and include but are not limited to those described in Umana et al., 1999, Nat. Biotechnol. 17: 176-180; Davies et al., 2001, Biotechnol. Bioeng. 74:288-294; Shields et al., 2002, J. Biol. Chem. 277:26733-26740; Shinkawa et al., 2003, J. Biol. Chem. 278:3466-3473; U.S. Pat. No. 6,602,684; U.S. patent application Ser. No. 10/277,370; U.S. patent application Ser. No. 10/113,929; International Patent Application Publications WO 00/61739A1; WO 01/292246A1; WO 02/311140A1; WO 02/30954A1; POTILLEGENT™ technology (Biowa, Inc. Princeton, N.J.); GLYCOMAB™ glycosylation engineering technology (GLYCART biotechnology AG, Zurich, Switzerland); each of which is incorporated herein by reference in its entirety. See, e.g., International Patent Application Publication WO 00/061739; U.S. Patent Application Publication No. 2003/0115614; Okazaki et al., 2004, JMB, 336: 1239-49.

Fusion Proteins. In one embodiment, the anti-DLL3 antibody of the present technology is a fusion protein. The anti-DLL3 antibodies of the present technology, when fused to a second protein, can be used as an antigenic tag. Examples of domains that can be fused to polypeptides include not only heterologous signal sequences, but also other heterologous functional regions. The fusion does not necessarily need to be direct, but can occur through linker sequences. Moreover, fusion proteins of the present technology can also be engineered to improve characteristics of the anti-DLL3 antibodies. For instance, a region of additional amino acids, particularly charged amino acids, can be added to the N-terminus of the anti-DLL3 antibody to improve stability and persistence during purification from the host cell or subsequent handling and storage. Also, peptide moieties can be added to an anti-DLL3 antibody to facilitate purification. Such regions can be removed prior to final preparation of the anti-DLL3 antibody. The addition of peptide moieties to facilitate handling of polypeptides are familiar and routine techniques in the art. The anti-DLL3 antibody of the present technology can be fused to marker sequences, such as a peptide which facilitates purification of the fused polypeptide. In select embodiments, the marker amino acid sequence is a hexa-histidine peptide (SEQ ID NO: 84), such as the tag provided in a pQE vector (QIAGEN, Inc., Chatsworth, Calif), among others, many of which are commercially available. As described in Gentz et al., Proc. Natl. Acad. Sci. USA 86: 821-824, 1989, for instance, hexa-histidine (SEQ ID NO: 84) provides for convenient purification of the fusion protein. Another peptide tag useful for purification, the “HA” tag, corresponds to an epitope derived from the influenza hemagglutinin protein. Wilson et al., Cell 37: 767, 1984.

Thus, any of these above fusion proteins can be engineered using the polynucleotides or the polypeptides of the present technology. Also, in some embodiments, the fusion proteins described herein show an increased half-life in vivo.

Fusion proteins having disulfide-linked dimeric structures (due to the IgG) can be more efficient in binding and neutralizing other molecules compared to the monomeric secreted protein or protein fragment alone. Fountoulakis et al., J Biochem. 270: 3958-3964, 1995.

Similarly, EP-A-O 464 533 (Canadian counterpart 2045869) discloses fusion proteins comprising various portions of constant region of immunoglobulin molecules together with another human protein or a fragment thereof. In many cases, the Fc part in a fusion protein is beneficial in therapy and diagnosis, and thus can result in, e.g., improved pharmacokinetic properties. See EP-A 0232 262. Alternatively, deleting or modifying the Fc part after the fusion protein has been expressed, detected, and purified, may be desired. For example, the Fc portion can hinder therapy and diagnosis if the fusion protein is used as an antigen for immunizations. In drug discovery, e.g., human proteins, such as hIL-5, have been fused with Fc portions for the purpose of high-throughput screening assays to identify antagonists of hIL-5. Bennett et al., J. Molecular Recognition 8: 52-58, 1995; Johanson et al., J. Biol. Chem., 270: 9459-9471, 1995.

Preparation of antigen: The DLL3 antigen can be obtained by allowing host cells to produce a gene encoding the antigen protein according to genetic manipulation. Specifically, a vector capable of expressing the antigen gene is produced, and the vector is then introduced into host cells, so that the gene is expressed therein, and thereafter, the expressed antigen may be purified. The antibody can also be obtained by a method of immunizing an animal with the antigen-expressing cells based on the above-described genetic manipulation, or a cell line expressing the antigen.

Alternatively, the antibody can also be obtained, without the use of the antigen protein, by incorporating cDNA of the antigen protein into an expression vector, then administering the expression vector to an animal to be immunized, and expressing the antigen protein in the body of the animal thus immunized, so that an antibody against the antigen protein is produced therein.

The anti-DLL3 antibody used in the present invention is not particularly limited. For example, an antibody specified by an amino acid sequence shown in the sequence listing of the present application can be suitably used. The anti-DLL3 antibody used in the present invention is desirably an antibody having the following properties:
(1) an antibody having the following properties:
   (a) specifically binding to DLL3, and
   (b) having the activity of being internalized into DLL3-expressing cells by binding to DLL3; or
(2) the antibody according to the above (1), wherein the DLL3 is human DLL3.

The method for obtaining the antibody against DLL3 of the present invention is not particularly limited as long as an anti-DLL3 antibody can be obtained. It is preferred to use DLL3 retaining its conformation as an antigen.

One example of the method for obtaining the antibody can include a DNA immunization method. The DNA immunization method is an approach which involves transfecting an animal (e.g., mouse or rat) individual with an antigen expression plasmid, and then expressing the antigen in the individual to induce immunity against the antigen. The transfection approach includes a method of directly injecting the plasmid to the muscle, a method of injecting a transfection reagent such as a liposome or polyethylenimine to the vein, an approach using a viral vector, an approach of injecting gold particles attached with the plasmid using a gene gun, a hydrodynamic method of rapidly injecting a plasmid solution in a large amount to the vein, and the like. With regard to the transfection method of injecting the expression plasmid to the muscle, a technique called in vivo electroporation, which involves applying electroporation to the intramuscular injection site of the plasmid, is known as an approach for improving expression levels (Aihara H, Miyazaki J. Nat Biotechnol. 1998 September; 16 (9): 867-70 or Mir L M, Bureau M F, Gehl J, Rangara R, Rouy D, Caillaud J M, Delaere P, Branellec D, Schwartz B, Scherman D. Proc Natl Acad Sci USA. 1999 Apr. 13; 96 (8): 4262-7). This approach further improves the expression level by treating the muscle with hyaluronidase before the intramuscular injection of the plasmid (McMahon J M1, Signori E, Wells K E, Fazio V M, Wells D J., Gene Ther. 2001 August; 8 (16): 1264-70). Furthermore, the hybridoma production can be performed by a known method, and can also be performed using, for example, a Hybrimune Hybridoma Production System (Cyto Pulse Sciences, Inc.).

Specific examples of obtaining a monoclonal antibody can include the following procedures:
(a) immune response can be induced by incorporating DLL3 cDNA into an expression vector (e.g., pcDNA3.1; Thermo Fisher Scientific Inc.), and directly administering the vector to an animal (e.g., a rat or a mouse) to be immunized by a method such as electroporation or a gene gun, so as to express DLL3 in the body of the animal. The administration of the vector by electroporation or the like may be performed one or more times, preferably a plurality of times, if necessary for enhancing antibody titer;
(b) collection of tissue (e.g., a lymph node) containing antibody-producing cells from the aforementioned animal in which the immune response has been induced;
(c) preparation of myeloma cells (hereinafter, referred to as “myelomas”) (e.g., mouse myeloma SP2/0-ag14 cells);
(d) cell fusion between the antibody-producing cells and the myelomas;
(e) selection of a hybridoma group producing an antibody of interest;
(f) division into single cell clones (cloning);
(g) optionally, the culture of hybridomas for the mass production of monoclonal antibodies, or the breeding of animals into which the hybridomas are inoculated; and/or
(h) study of the physiological activity (internalization activity) and binding specificity of the monoclonal antibody thus produced, or examination of the properties of the antibody as a labeling reagent.

Examples of the method for measuring the antibody titer used herein can include, but are not limited to, flow cytometry and Cell-ELISA.

The antibody of the present invention also includes genetically recombinant antibodies that have been artificially modified for the purpose of reducing heterogenetic antigenicity to humans, such as a chimeric antibody, a humanized antibody and a human antibody, as well as the above-described monoclonal antibody against DLL3. These antibodies can be produced by known methods.

Example of the chimeric antibody can include antibodies in which a variable region and a constant region are heterologous to each other, such as a chimeric antibody formed by conjugating the variable region of a mouse- or rat-derived antibody to a human-derived constant region (see Proc. Natl. Acad. Sci. U.S.A., 81, 6851-6855, (1984)).

Examples of the humanized antibody can include an antibody formed by incorporating only complementarity determining regions (CDRs) into a human-derived antibody (see Nature (1986) 321, p. 522-525), an antibody formed by incorporating the amino acid residues from some frameworks, as well as CDR sequences, into a human antibody according to a CDR grafting method (International Publication No. WO90/07861), and an antibody formed by modifying the amino acid sequences of some CDRs while maintaining antigen-binding ability.

Further examples of the antibody of the present invention can include a human antibody binding to DLL3. The anti-DLL3 human antibody means a human antibody having only the gene sequence of an antibody derived from human chromosomes. The anti-DLL3 human antibody can be obtained by a method using a human antibody-producing mouse having a human chromosomal fragment comprising the heavy chain and light chain genes of a human antibody (see Tomizuka, K. et al., Nature Genetics (1997) 16, p. 133-143; Kuroiwa, Y et al., Nucl. Acids Res. (1998) 26, p. 3447-3448; Yoshida, H. et al., Animal Cell Technology: Basic and Applied Aspects vol. 10, p. 69-73 (Kitagawa, Y, Matsuda, T. and Iijima, S. eds.), Kluwer Academic Publishers, 1999; Tomizuka, K. et al., Proc. Natl. Acad. Sci. USA (2000) 97, p. 722-727; etc.).

Such a human antibody-producing mouse can be specifically produced by using a genetically modified animal, the gene loci of endogenous immunoglobulin heavy chain and light chain of which have been disrupted and instead the gene loci of human immunoglobulin heavy chain and light chain have been then introduced using a yeast artificial chromosome (YAC) vector or the like, then producing a knock-out animal and a transgenic animal from such a genetically modified animal, and then breeding such animals with one another.

Otherwise, the anti-DLL3 human antibody can also be obtained by transforming eukaryotic cells with cDNA encoding each of the heavy chain and light chain of such a human antibody, or preferably with a vector comprising the cDNA, according to genetic recombination techniques, and then culturing the transformed cells producing a genetically modified human monoclonal antibody, so that the antibody can be obtained from the culture supernatant.

In this context, eukaryotic cells, and preferably, mammalian cells such as CHO cells, lymphocytes or myelomas can, for example, be used as a host.

Furthermore, a method of obtaining a phage display-derived human antibody that has been selected from a human antibody library (see Wormstone, I. M. et al., Investigative Ophthalmology & Visual Science. (2002) 43 (7), p. 2301-2308; Carmen, S. et al., Briefings in Functional Genomics and Proteomics (2002), 1 (2), p. 189-203; Siriwardena, D. et al., Ophthalmology (2002) 109 (3), p. 427-431; etc.) is also known.

For example, a phage display method, which comprises allowing the variable regions of a human antibody to express as a single chain antibody (scFv) on the surface of phages, and then selecting a phage binding to an antigen, can be applied (Nature Biotechnology (2005), 23, (9), p. 1105-1116).

By analyzing the phage gene that has been selected because of its binding ability to the antigen, DNA sequences encoding the variable regions of a human antibody binding to the antigen can be determined.

Once the DNA sequence of scFv binding to the antigen is determined, an expression vector having the aforementioned sequence is produced, and the produced expression vector is then introduced into an appropriate host and can be allowed to express therein, thereby obtaining a human antibody (International Publication Nos. WO92/01047, WO92/20791, WO93/06213, WO93/11236, WO93/19172, WO95/01438, and WO95/15388, Annu. Rev. Immunol (1994) 12, p. 433-455, Nature Biotechnology (2005) 23 (9), p. 1105-1116).

The amino acid substitution in the present description is preferably a conservative amino acid substitution. The conservative amino acid substitution is a substitution occurring within an amino acid group associated with certain amino acid side chains. Preferred amino acid groups are the following: acidic group=aspartic acid and glutamic acid; basic group=lysine, arginine, and histidine; non-polar group=alanine, valine, leucine, isoleucine, proline, phenylalanine, methionine, and tryptophan; and uncharged polar family=glycine, asparagine, glutamine, cysteine, serine, threonine, and tyrosine. Other preferred amino acid groups are the following: aliphatic hydroxy group=serine and threonine; amide-containing group=asparagine and glutamine; aliphatic group=alanine, valine, leucine and isoleucine; and aromatic group=phenylalanine, tryptophan and tyrosine. Such amino acid substitution is preferably carried out without impairing the properties of a substance having the original amino acid sequence.

### V. Identification and Characterization of Anti-DLL3 Antibodies

Methods for identifying and/or screening the anti-DLL3 antibodies of the present technology. Methods useful to identify and screen antibodies against DLL3 polypeptides for those that possess the desired specificity to DLL3 protein (e.g., those that bind to the extracellular domain of DLL3) include any immunologically-mediated techniques known within the art. Components of an immune response can be detected in vitro by various methods that are well known to those of ordinary skill in the art. For example, (1) cytotoxic T lymphocytes can be incubated with radioactively labeled target cells and the lysis of these target cells detected by the release of radioactivity; (2) helper T lymphocytes can be incubated with antigens and antigen presenting cells and the synthesis and secretion of cytokines measured by standard methods (Windhagen A et al., Immunity, 2: 373-80, 1995); (3) antigen presenting cells can be incubated with whole protein antigen and the presentation of that antigen on MHC detected by either T lymphocyte activation assays or biophysical methods (Harding et al., Proc. Natl. Acad. Sci., 86: 4230-4, 1989); (4) mast cells can be incubated with reagents that cross-link their Fc-epsilon receptors and histamine release measured by enzyme immunoassay (Siraganian et al., TIPS, 4: 432-437, 1983); and (5) enzyme-linked immunosorbent assay (ELISA).

Similarly, products of an immune response in either a model organism (e.g., mouse) or a human subject can also be detected by various methods that are well known to those of ordinary skill in the art. For example, (1) the production of antibodies in response to vaccination can be readily detected by standard methods currently used in clinical laboratories, e.g., an ELISA; (2) the migration of immune cells to sites of inflammation can be detected by scratching the surface of skin and placing a sterile container to capture the migrating cells over scratch site (Peters et al., Blood, 72: 1310-5, 1988); (3) the proliferation of peripheral blood mononuclear cells (PBMCs) in response to mitogens or mixed lymphocyte reaction can be measured using 3H-thymidine; (4) the phagocytic capacity of granulocytes, macrophages, and other phagocytes in PBMCs can be measured by placing PBMCs in wells together with labeled particles (Peters et al., Blood, 72: 1310-5, 1988); and (5) the differentiation of immune system cells can be measured by labeling PBMCs with antibodies to CD molecules such as CD4 and CD8 and measuring the fraction of the PBMCs expressing these markers.

In one embodiment, anti-DLL3 antibodies of the present technology are selected using display of DLL3 peptides on the surface of replicable genetic packages. See, e.g., U.S. Pat. Nos. 5,514,548; 5,837,500; 5,871,907; 5,885,793; 5,969,108; 6,225,447; 6,291,650; 6,492,160; EP 585 287; EP 605522; EP 616640; EP 1024191; EP 589 877; EP 774 511; EP 844 306. Methods useful for producing/selecting a filamentous bacteriophage particle containing a phagemid genome encoding for a binding molecule with a desired specificity has been described. See, e.g., EP 774 511; U.S. Pat. Nos. 5,871,907; 5,969,108; 6,225,447; 6,291,650; 6,492,160.

In some embodiments, anti-DLL3 antibodies of the present technology are selected using display of DLL3 peptides on the surface of a yeast host cell. Methods useful for the isolation of scFv polypeptides by yeast surface display have been described by Kieke et al., Protein Eng. 1997 November; 10(11): 1303-10.

In some embodiments, anti-DLL3 antibodies of the present technology are selected using ribosome display. Methods useful for identifying ligands in peptide libraries using ribosome display have been described by Mattheakis et al., Proc. Natl. Acad. Sci. USA 91: 9022-26, 1994; and Hanes et al., Proc. Natl. Acad. Sci. USA 94: 4937-42, 1997.

In certain embodiments, anti-DLL3 antibodies of the present technology are selected using tRNA display of DLL3 peptides. Methods useful for in vitro selection of ligands using tRNA display have been described by Merryman et al., Chem. Biol., 9: 741-46, 2002.

In one embodiment, anti-DLL3 antibodies of the present technology are selected using RNA display. Methods useful for selecting peptides and proteins using RNA display libraries have been described by Roberts et al. Proc. Natl. Acad. Sci. USA, 94: 12297-302, 1997; and Nemoto et al., FEBS Lett., 414: 405-8, 1997. Methods useful for selecting peptides and proteins using unnatural RNA display libraries have been described by Frankel et al., Curr. Opin. Struct. Biol., 13: 506-12, 2003.

In some embodiments, anti-DLL3 antibodies of the present technology are expressed in the periplasm of gram negative bacteria and mixed with labeled DLL3 protein. See WO 02/34886. In clones expressing recombinant polypeptides with affinity for DLL3 protein, the concentration of the labeled DLL3 protein bound to the anti-DLL3 antibodies is increased and allows the cells to be isolated from the rest of the library as described in Harvey et al., Proc. Natl. Acad. Sci. 22: 9193-98 2004 and U.S. Pat. Publication No. 2004/0058403.

After selection of the desired anti-DLL3 antibodies, it is contemplated that said antibodies can be produced in large volume by any technique known to those skilled in the art, e.g., prokaryotic or eukaryotic cell expression and the like. The anti-DLL3 antibodies which are, e.g., but not limited to, anti-DLL3 hybrid antibodies or fragments can be produced by using conventional techniques to construct an expression vector that encodes an antibody heavy chain in which the CDRs and, if necessary, a minimal portion of the variable region framework, that are required to retain original species antibody binding specificity (as engineered according to the techniques described herein) are derived from the originating species antibody and the remainder of the antibody is derived from a target species immunoglobulin which can be manipulated as described herein, thereby producing a vector for the expression of a hybrid antibody heavy chain.

Measurement of DLL3 Binding. In some embodiments, a DLL3 binding assay refers to an assay format wherein DLL3 protein and an anti-DLL3 antibody are mixed under conditions suitable for binding between the DLL3 protein and the anti-DLL3 antibody and assessing the amount of binding between the DLL3 protein and the anti-DLL3 antibody. The amount of binding is compared with a suitable control, which can be the amount of binding in the absence of the DLL3 protein, the amount of the binding in the presence of a non-specific immunoglobulin composition, or both. The amount of binding can be assessed by any suitable method. Binding assay methods include, e.g., ELISA, radioimmunoassays, scintillation proximity assays, fluorescence energy transfer assays, liquid chromatography, membrane filtration assays, and the like. Biophysical assays for the direct measurement of DLL3 protein binding to anti-DLL3 antibody are, e.g., nuclear magnetic resonance, fluorescence, fluorescence polarization, surface plasmon resonance (BIACORE chips), biolayer interferometry, and the like. Specific binding is determined by standard assays known in the art, e.g., radioligand binding assays, ELISA, FRET, immunoprecipitation, SPR, NMR (2D-NMR), mass spectroscopy and the like. If the specific binding of a candidate anti-DLL3 antibody is at least 1 percent greater than the binding observed in the absence of the candidate anti-DLL3 antibody, the candidate anti-DLL3 antibody is useful as an anti-DLL3 antibody of the present technology.

By combining together sequences showing a high identity to the above-described heavy chain amino acid sequences and light chain amino acid sequences, it is possible to select an antibody having a biological activity equivalent to that of each of the above-described antibodies. Such an identity is an identity of generally 80% or more, preferably 90% or more, more preferably 95% or more, and most preferably 99% or more. Moreover, also by combining amino acid sequences of a heavy chain and a light chain comprising a substitution, deletion or addition of one or several amino acid residues thereof with respect to the amino acid sequence of a heavy chain or a light chain, it is possible to select an antibody having a biological activity equivalent to that of each of the above-described antibodies.

The identity between two types of amino acid sequences can be determined by aligning the sequences using the default parameters of Clustal W version 2 (Larkin M A, Blackshields G, Brown N P, Chenna R, McGettigan P A, McWilliam H, Valentin F, Wallace I M, Wilm A, Lopez R, Thompson J D, Gibson T J and Higgins D G (2007), “Clustal W and Clustal X version 2.0”, Bioinformatics. 23 (21): 2947-2948).

If a newly produced human antibody binds to a partial peptide or a partial three-dimensional structure to which any one rat anti-human DLL3 antibody, chimeric anti-human DLL3 antibody or humanized anti-human DLL3 antibody described in the present description binds, it can be determined that the human antibody binds to the same epitope to which the rat anti-human DLL3 antibody, the chimeric anti-human DLL3 antibody or the humanized anti-human DLL3 antibody binds. Alternatively, by confirming that the human antibody competes with the rat anti-human DLL3 antibody, the chimeric anti-human DLL3 antibody or the humanized anti-human DLL3 antibody described in the present description in the binding of the antibody to DLL3, it can be determined that the human antibody binds to the same epitope to which the rat anti-human DLL3 antibody, the chimeric anti-human DLL3 antibody or the humanized anti-human DLL3 antibody described in the present description binds, even if the specific sequence or structure of the epitope has not been determined. In the present description, when it is determined by at least one of these determination methods that the human antibody “binds to the same epitope”, it is concluded that the newly prepared human antibody “binds to the same epitope” as that for the rat anti-human DLL3 antibody, the chimeric anti-human DLL3 antibody or the humanized anti-human DLL3 antibody described in the present description. When it is confirmed that the human antibody binds to the same epitope, then it is expected that the human antibody should have a biological activity equivalent to that of the rat anti-human DLL3 antibody, the chimeric anti-human DLL3 antibody or the humanized anti-human DLL3 antibody.

The chimeric antibodies, the humanized antibodies, or the human antibodies obtained by the above-described methods are evaluated for their binding activity against the antigen according to a known method, etc., so that a preferred antibody can be selected.

One example of another indicator for comparison of the properties of antibodies can include the stability of an antibody. A differential scanning calorimeter (DSC) is an apparatus capable of promptly and exactly measuring a thermal denaturation midpoint (Tm) serving as a good indicator for the relative structural stability of a protein. By using DSC to measure Tm values and making a comparison regarding the obtained values, differences in thermal stability can be compared. It is known that the preservation stability of an antibody has a certain correlation with the thermal stability of the antibody (Lori Burton, et al., Pharmaceutical Development and Technology (2007) 12, p. 265-273), and thus, a preferred antibody can be selected using thermal stability as an indicator. Other examples of the indicator for selection of an antibody can include high yield in suitable host cells and low agglutination in an aqueous solution. For example, since an antibody with the highest yield does not always exhibit the highest thermal stability, it is necessary to select an antibody most suitable for administration to a human by comprehensively determining it based on the aforementioned indicators.

The obtained antibody can be purified to a homogenous state. For separation and purification of the antibody, separation and purification methods used for ordinary proteins may be used. For example, column chromatography, filtration, ultrafiltration, salting-out, dialysis, preparative polyacrylamide gel electrophoresis, and isoelectric focusing are appropriately selected and combined with one another, so that the antibody can be separated and purified (Strategies for Protein Purification and Characterization: A Laboratory Course Manual, Daniel R. Marshak et al. eds., Cold Spring Harbor Laboratory Press (1996); and Antibodies: A Laboratory Manual. Ed Harlow and David Lane, Cold Spring Harbor Laboratory (1988)), though examples of the separation and purification methods are not limited thereto.

Examples of the chromatography can include affinity chromatography, ion exchange chromatography, hydrophobic chromatography, gel filtration chromatography, reverse phase chromatography, and absorption chromatography.

These chromatographic techniques can be carried out using liquid chromatography such as HPLC or FPLC.

Examples of the column used in the affinity chromatography can include a Protein A column and a Protein G column. Examples of the column involving the use of Protein A can include Hyper D, POROS, and Sepharose F. F. (Pharmacia).

Also, using an antigen-immobilized carrier, the antibody can be purified by utilizing the binding activity of the antibody to the antigen.

### VI. Anti-DLL3 Antibody-Drug Conjugate (ADC)

The anti-DLL3 antibodies described herein (e.g., 2-C8-A, 6-G23-F, and 10-O18-A or human antibody: H2-C8-A, H2-C8-A-2, H2-C8-A-3, H6-G23-F, H6-G23-F-2, H6-G23-F-3, H10-O18-A, H10-O18-A-2, and H10-O18-A-3) can be conjugated to a drug via a linker structure moiety to prepare an anti-DLL3 antibody-drug conjugate (ADC). The drug is not particularly limited as long as it has a substituent or a partial structure that can be connected to a linker structure. The anti-DLL3 antibody-drug conjugate (ADC) can be used for various purposes according to the conjugated drug. Examples of such a drug can include substances having antitumor activity, substances effective for blood diseases, substances effective for autoimmune diseases, anti-inflammatory substances, antimicrobial substances, antifungal substances, antiparasitic substances, antiviral substances, and anti-anesthetic substances.

The antibody-drug conjugate of the present invention is represented by the following formula:

Ab [symbol] L-D]ₘ_{¹} [Formula 36]

m₁ represents the number of conjugated drug molecules per antibody molecule in the antibody-drug conjugate, Ab represents an antibody or a functional fragment of the antibody, L represents a linker linking Ab and D, and D represents a drug.

A. Drug

Drug D conjugated in the antibody-drug conjugate of the present invention is described herein. Drug D of the present invention is preferably an antitumor compound. The antitumor compound develops antitumor effect, when a part or the entire of the linker is cleaved in a tumor cell and the antitumor compound moiety is released. When the linker and the drug are cleaved apart at the bonding part, the antitumor compound in the original structure is released and the original antitumor effect is exerted.

The antitumor compound in the antibody-drug conjugate of the present invention is a pyrrolobenzodiazepine derivative (PBD derivative) represented by general formula (V):

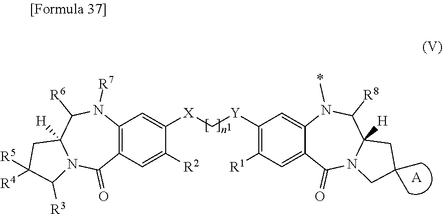

The variable groups of the Formula are described in more detail below.

The asterisk represents bonding to linker L.

n¹ represents an integer of 2 to 8, and is preferably an integer of 2 to 6, and more preferably an integer of 3 to 5.

The alkyl chain with the subscript n¹ being an integer of 2 to 8, preferably an integer of 2 to 6, and more preferably an integer of 3 to 5, may include a double bond.

A represents a spiro-bonded three- to five-membered saturated hydrocarbon ring or a three- to five-membered saturated heterocycle, and is preferably a three- to five-membered saturated hydrocarbon ring (cyclopropane, cyclobutane, or cyclopentane), more preferably cyclopropane or cyclobutane, and most preferably cyclopropane.

The spiro-bonded three- to five-membered saturated hydrocarbon ring may be substituted with one to four halogen atoms, and may be preferably substituted with one or two fluorine atoms (e.g., 2,2-difluorocyclopropane).

R¹ and R² each independently represent a C1 to C6 alkoxy group, a C1 to C6 alkyl group, a hydrogen atom, a hydroxy group, a thiol group, a C1 to C6 alkylthio group, a halogen atom, or —NR′R″, and are each preferably a C1 to C6 alkoxy group, a C1 to C6 alkyl group, or a hydroxy group, more preferably a C1 to C3 alkoxy group, and most preferably a methoxy group.

R³, R⁴, and R⁵ are as described in any of the following (i) to (iii).

### i. Compound Embodiment One

If R³ and R⁴ are combined together with the carbon atoms to which R³ and R⁴ are bound to form a double bond as shown in the following:

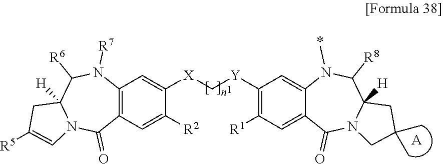

wherein R⁵ represents an aryl group or heteroaryl group optionally having one or more substituents selected from group 1 or a C1 to C6 alkyl group optionally having one or more substituents selected from group 2, and is preferably an aryl group optionally having one or more substituents selected from group 1.

“Aryl group” in “aryl group or heteroaryl group optionally having one or more substituents selected from group 1” for R⁵ is preferably a phenyl group or a naphthyl group, and more preferably a phenyl group.

“Heteroaryl group” in “aryl group or heteroaryl group optionally having one or more substituents selected from group 1” for R⁵ is preferably a thienyl group, a pyridyl group, a pyrimidyl group, a quinolyl group, a quinoxalyl group, or a benzothiophenyl group, more preferably a 2-thienyl group, a 3-thienyl group, a 2-pyridyl group, a 3-pyridyl group, or a 4-pyridyl group, and even more preferably a 3-pyridyl group or a 3-thienyl group.

Examples of substituents of the aryl group or heteroaryl group for R⁵ may include, but are not limited to, the following a) to j):
a) a C1 to C6 alkoxy group optionally substituted with one to three halogen atoms,
b) a C1 to C6 alkyl group optionally substituted with any one selected from one to three halogen atoms, a hydroxy group, —OCOR′, —NR′R″, —C(═NR′)—NR″R′″, and —NHC(═NR′)—NR″R′″,
c) a halogen atom,
d) a C3 to C5 cycloalkoxy group,
e) a C1 to C6 alkylthio group,
f) —NR′R″,
g) —C(═NR′)—NR″R′″,
h) —NHC(═NR′)—NR″R′″,
i) —NHCOR′, and
j) a hydroxy group.

Here, R′, R″, and R′″ in b) and f) to i) each independently represent a hydrogen atom or a C1 to C6 alkyl group, and are preferably each independently a hydrogen atom or a C1 to C3 alkyl group.

a) to j) are preferably as follows:
a) a C1 to C3 alkoxy group optionally substituted with one to three halogen atoms, more preferably a methoxy group, an ethoxy group, an n-propoxy group, an i-propoxy group, or a trifluoromethoxy, even more preferably a methoxy group, an ethoxy group, or a trifluoromethoxy group, and most preferably a methoxy group;
b) a C1 to C3 alkyl group optionally substituted with one to three halogen atoms, a hydroxy group, —OCOR′, —C(═NR′)—NR″R′″, or —NHC(═NR′)—NR″R′″, wherein R′, R″, and R′″ are each independently a hydrogen atom or a C1 to C3 alkyl group, more preferably a C1 to C3 alkyl group optionally substituted with any selected from one to three halogen atoms, a hydroxy group, —OCOR′, —C(═NR′)—NR″R′″, and —NHC(═NR′)—NR″R′″, wherein R′, R″, and R′″ are each independently a hydrogen atom or a methyl group, even more preferably a methyl group, an ethyl group, a n-propyl group, an i-propyl group, a fluoromethyl group, a difluoromethyl group, a trifluoromethyl group, a hydroxymethyl group, —CH₂OCOMe, —CH₂—NHC(═NH)—NH₂, or —CH₂—NHC(═NMe)-NH₂;
c) a halogen atom, preferably a fluorine atom or a chlorine atom;
d) a C3 to C5 cycloalkoxy group, more preferably a cyclopropoxy group;
e) a C1 to C3 alkylthio group, more preferably a methylthio group or an ethylthio group;
f) —NR′R″, wherein R′ and R″ are each independently a hydrogen atom or a C1 to C3 alkyl group, more preferably —NH₂, —NHMe, —NMe₂, —NHEt, or -NEt₂;
g) —C(═NR′)—NR″R′″, wherein R′, R″, and R′″ are each independently a hydrogen atom or a C1 to C3 alkyl group, more preferably —C(═NH)—NH₂ or —C(═NMe)-NH₂;
h) —NHC(═NR′)—NR″R′″, wherein R′, R″, and R′″ are each independently a hydrogen atom or a C1 to C3 alkyl group, more preferably —NHC(═NH)—NH₂ or —NHC(═NMe)-NH₂;
i) —NHCOR′, wherein R′ is a hydrogen atom or a C1 to C3 alkyl group, more preferably —NHCOMe or -NHCOEt; and
j) a hydroxy group.

The aryl group (preferably, a phenyl group) or heteroaryl group (preferably, a pyridyl group) for R⁵ may have at least one substituent at any position. If a plurality of substituents is present, the substituents may be the same or different.

If R⁵ is an aryl group, each substituent is preferably a), b), d), g), h), or j), and more preferably a), b), d), or j).

If R⁵ is a phenyl group, R5 may have a substituent at any position and may have a plurality of substituents, and preferably one or two substituents are present at the 3-position and/or the 4-position, and more preferably one substituent is present at the 4-position. If R⁵ is a naphthyl group, R⁵ may have a substituent at any position and may have a plurality of substituents, and preferably one substituent is present at the 6-position.

If R⁵ is a phenyl group, R⁵ is more preferably a phenyl group, a 4-methoxyphenyl group, a 3-methoxyphenyl group, a 4-ethoxyphenyl group, a 4-(n-propoxy)-phenyl group, a 4-(i-propoxy)-phenyl group, a 4-cyclopropoxy-phenyl group, a 4-trifluoromethylphenyl group, a 4-hydroxymethyl-phenyl group, a 4-acetoxymethyl-phenyl group, or a 4-carbamimidamidomethyl-phenyl group, and even more preferably a phenyl group, a 4-methoxyphenyl group, a 3-methoxyphenyl group, a 4-cyclopropoxy-phenyl group, a 4-hydroxymethyl-phenyl group, a 4-acetoxymethyl-phenyl group, a 4-carbamimidamidomethyl-phenyl group, or a 4-trifluoromethylphenyl group.

If R⁵ is a naphthyl group, R⁵ is more preferably a naphthyl group or a 6-methoxy-2-naphthyl group.

The most preferred is a 4-methoxyphenyl group.

If R⁵ is a heteroaryl group, each substituent is preferably a), b), d), g), h), or j), and more preferably a) or b).

If R⁵ is a heteroaryl group, R⁵ may have at least one substituent at any position. If R⁵ is a 3-pyridyl group, its substituent(s) is preferably present at the 6-position and/or the 5-position. If R⁵ is 2-pyridyl, its substituent(s) is preferably present at the 5-position and/or the 4-position, or at the 5-position and/or the 6-position. If R⁵ is 4-pyridyl, its substituent(s) is preferably present at the 2-position and/or the 6-position.

If R⁵ is a heteroaryl group, R⁵ may have a plurality of substituents, and preferably has one or two substituents, and preferably has one substituent.

If R⁵ is a pyridyl group, R⁵ is preferably a 6-methoxy-3-pyridyl group or a 6-methyl-3-pyridyl group.

If R⁵ is a 3-thienyl group or a 6-quinoxalyl group, R⁵ is preferably unsubstituted.

“C1 to C6 alkyl group” in “C1 to C6 alkyl group optionally having one or more substituents selected from group 2” for R⁵ is preferably a C1 to C3 alkyl group, and more preferably a methyl group or an ethyl group.

The substituents in “C1 to C6 alkyl group optionally having one or more substituents selected from group 2” for R⁵ are each a halogen atom, a hydroxy group, or a C1 to C6 alkoxy group (preferably, a C1 to C3 alkoxy group), preferably a hydroxy group, a methoxy group, or an ethoxy group, and more preferably a hydroxy group.

### ii. Compound Embodiment Two

If R³ represents a hydrogen atom, R⁴ and R⁵ are combined, together with the carbon atom to which R⁴ and R⁵ are bound, to form a three- to five-membered saturated hydrocarbon ring or three- to five-membered saturated heterocycle, or CH₂═ as shown in the following:

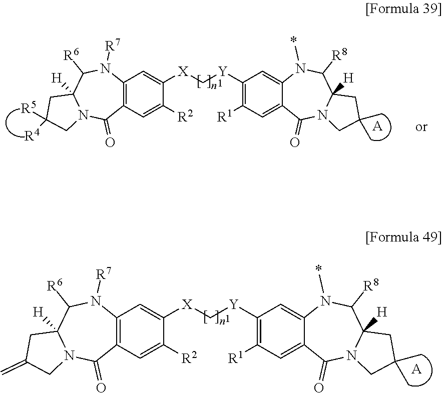

The three- to five-membered saturated hydrocarbon ring may be substituted with one to four halogen atoms, and may be preferably substituted with one or two fluorine atoms.

R⁴ and R⁵ are preferably combined to form a three- to five-membered saturated hydrocarbon ring or CH₂═, more preferably to form cyclopropane, cyclobutane, or CH₂═(exomethylene group), and even more preferably to form cyclopropane.

If R⁴ and R⁵ are combined to form a three- to five-membered saturated hydrocarbon ring or three- to five-membered saturated heterocycle, the three- to five-membered saturated hydrocarbon ring or three- to five-membered saturated heterocycle is preferably the same as A. More preferably, A is a three- to five-membered saturated hydrocarbon ring and R⁴ and R⁵ are combined to form a three- to five-membered saturated hydrocarbon ring, and even more preferably A is a cyclopropane ring and R⁴ and R⁵ are combined to form a cyclopropane ring.

### iii. Compound Embodiment Three

R³, R⁴, and R⁵ are combined, together with the carbon atom to which R³ is bound and the carbon atom to which R⁴ and R⁵ are bound, to form a benzene ring or six-membered heterocycle optionally having one or more substituents selected from group 3.

The following formula shows the case in which R³ and R⁴ are combined to form a benzene ring optionally having one or more substituents:

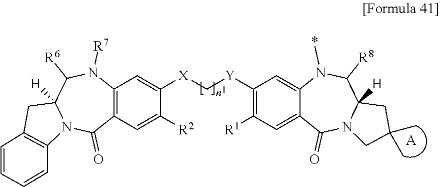

The benzene ring or heterocycle may have at least one substituent at any position. If a plurality of substituents is present, the substituents may be the same or different.

Each substituent of the benzene ring or the heterocycle is a halogen atom, a C1 to C6 alkyl group optionally substituted with one to three halogen atoms, or a C1 to C6 alkoxy group, preferably a halogen atom, a C1 to C3 alkyl group optionally substituted with one to three halogen atoms, or a C1 to C3 alkoxy, and more preferably a halogen atom, a methyl group, or a methoxy group.

“Benzene ring or six-membered heterocycle optionally having one or more substituents” is preferably an unsubstituted benzene ring.

R³, R⁴ and R⁵ most preferably satisfy the above from subsection (i) “Compound Embodiment One”.

R⁶ and R⁷ each represent a hydrogen atom, or R⁶ and R⁷ are combined to represent an imine bond (C═N).

R⁸ is a hydroxy group or a C1 to C3 alkoxy group, preferably a hydroxy group or a methoxy group, and more preferably a hydroxy group. R⁸ may be a hydrogensulfite adduct (OSO₃M, wherein M is a metal cation).

Since R⁸ bonds to an asymmetric carbon atom, a steric configuration represented by partial structure (Va) or (Vb) below is provided. Each wavy line represents bonding to Y in general formula (V), and each asterisk represents bonding to L.

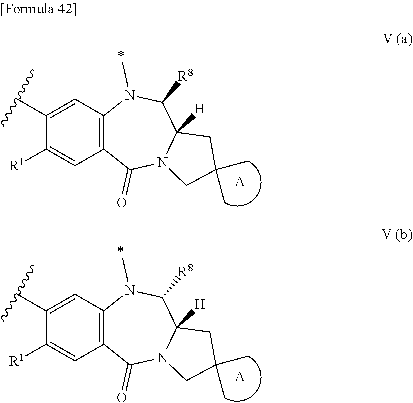

X and Y are each independently an oxygen atom, a nitrogen atom, or a sulfur atom, and preferably an oxygen atom.

Drug D of the present invention is preferably any one compound selected from the following group:

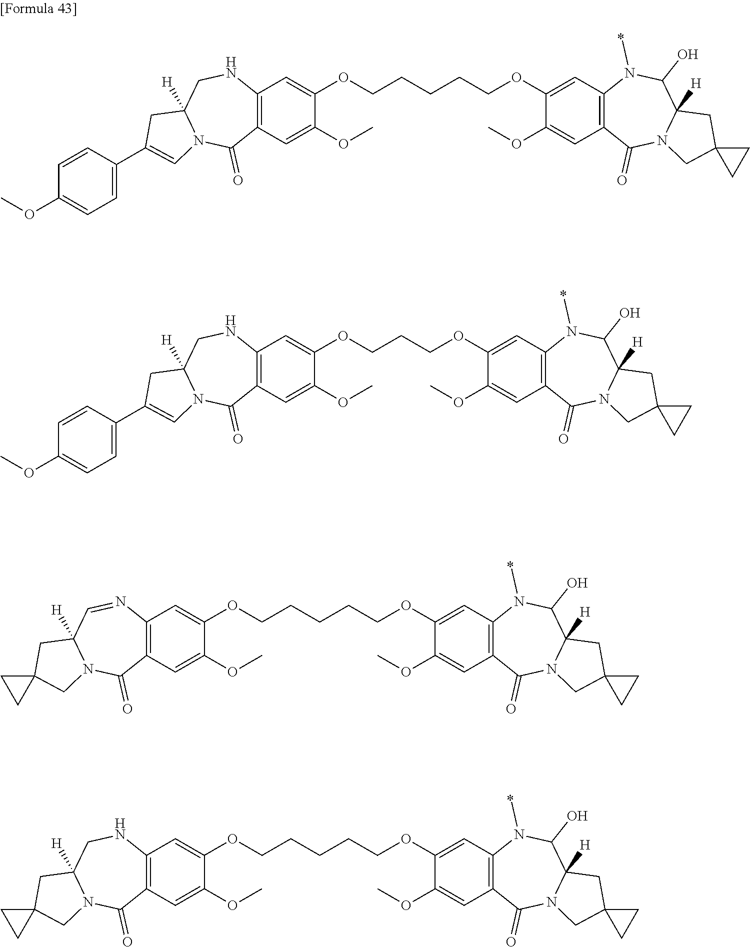 [SMILES: COc1ccc(C2=CN3C(=O)c4cc(OC)c(OCCCCCOc5cc6c(cc5OC)C(=O)N5CC7(CC7)C[C@H]5C(O)N6C)cc4NC[C@@H]3C2)cc1 ; COc1ccc(C2=CN3C(=O)c4cc(OC)c(OCCCOc5cc6c(cc5OC)C(=O)N5CC7(CC7)C[C@H]5C(O)N6C)cc4NC[C@@H]3C2)cc1 ; COc1cc2c(cc1OCCCCCOc1cc3c(cc1OC)C(=O)N1CC4(CC4)C[C@H]1C(O)N3C)N=C[C@@H]1CC3(CC3)CN1C2=O ; COc1cc2c(cc1OCCCCCOc1cc3c(cc1OC)C(=O)N1CC4(CC4)C[C@H]1C(O)N3C)NC[C@@H]1CC3(CC3)CN1C2=O]

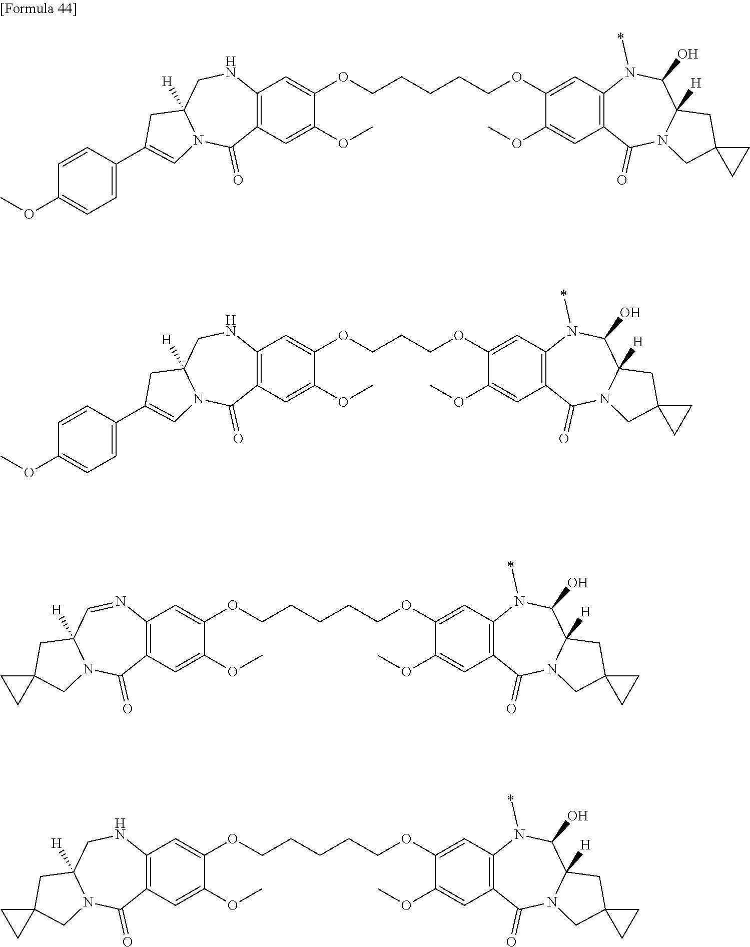 [SMILES: COc1ccc(C2=CN3C(=O)c4cc(OC)c(OCCCCCOc5cc6c(cc5OC)C(=O)N5CC7(CC7)C[C@H]5[C@H](O)N6C)cc4NC[C@@H]3C2)cc1 ; COc1ccc(C2=CN3C(=O)c4cc(OC)c(OCCCOc5cc6c(cc5OC)C(=O)N5CC7(CC7)C[C@H]5[C@H](O)N6C)cc4NC[C@@H]3C2)cc1 ; COc1cc2c(cc1OCCCCCOc1cc3c(cc1OC)C(=O)N1CC4(CC4)C[C@H]1[C@H](O)N3C)N=C[C@@H]1CC3(CC3)CN1C2=O ; COc1cc2c(cc1OCCCCCOc1cc3c(cc1OC)C(=O)N1CC4(CC4)C[C@H]1[C@H](O)N3C)NC[C@@H]1CC3(CC3)CN1C2=O]

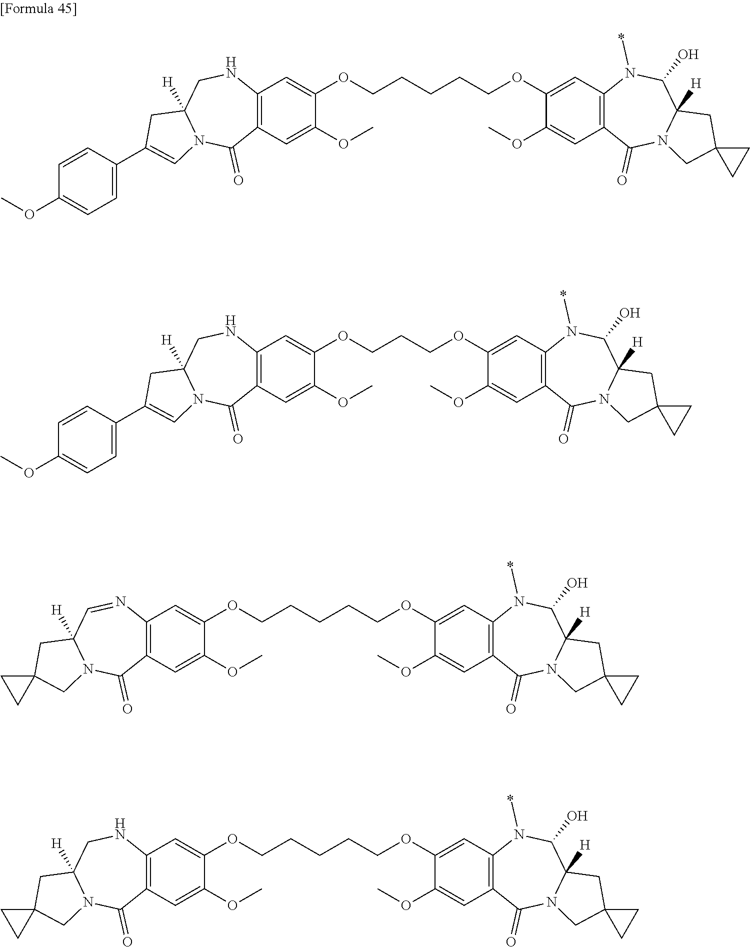 [SMILES: COc1ccc(C2=CN3C(=O)c4cc(OC)c(OCCCCCOc5cc6c(cc5OC)C(=O)N5CC7(CC7)C[C@H]5[C@@H](O)N6C)cc4NC[C@@H]3C2)cc1 ; COc1ccc(C2=CN3C(=O)c4cc(OC)c(OCCCOc5cc6c(cc5OC)C(=O)N5CC7(CC7)C[C@H]5[C@@H](O)N6C)cc4NC[C@@H]3C2)cc1 ; COc1cc2c(cc1OCCCCCOc1cc3c(cc1OC)C(=O)N1CC4(CC4)C[C@H]1[C@@H](O)N3C)N=C[C@@H]1CC3(CC3)CN1C2=O ; COc1cc2c(cc1OCCCCCOc1cc3c(cc1OC)C(=O)N1CC4(CC4)C[C@H]1[C@@H](O)N3C)NC[C@@H]1CC3(CC3)CN1C2=O]

wherein each asterisk represents where the drug is bonded to the linker structure (L).

In some embodiments, —OH is at the position 11′.

B. Linker Structure

The linker structure to bond the antitumor drug to the antibody in the antibody-drug conjugate of the present invention will be described.

Linker L is represented by the following formula:

-Lb-La-Lp-NH—B—CH₂—O(C═O)—*

wherein the asterisk represents bonding to the nitrogen atom at the N10′-position of drug D, Lb represents a spacer which connects La to a glycan or remodeled glycan of Ab.

B represents a phenyl group or a heteroaryl group, and is preferably a 1,4-phenyl group, a 2,5-pyridyl group, a 3,6-pyridyl group, a 2,5-pyrimidyl group, or a 2,5-thienyl group, and more preferably a 1,4-phenyl group.

Lp represents a linker consisting of an amino acid sequence cleavable in vivo or in a target cell. Lp is, for example, cleaved by the action of an enzyme such as esterase and peptidase.

Lp is a peptide residue composed of two to seven (preferably, two to four) amino acids. That is, Lp is composed of an oligopeptide residue in which two to seven amino acids are connected via peptide bonding.

Lp is bound at the N terminal to a carbonyl group of La in Lb-La—, and forms at the C terminal an amide bond with the amino group (—NH—) of the part —NH—B—CH₂—O(C═O)— of the linker. The bond between the C terminal of Lp and —NH— is cleaved by the enzyme such as esterase.

The amino acids constituting Lp are not limited to particular amino acids, and, for example are L- or D-amino acids, and preferably L-amino acids. The amino acids may be not only α-amino acids, but may include an amino acid with structure, for example, of β-alanine, ε-aminocaproic acid, or γ-aminobutyric acid, and may further include a non-natural amino acid such as an N-methylated amino acid.

The amino acid sequence of Lp is not limited to a particular amino acid sequence, and examples of amino acids that constitute Lp may include, but are not limited to, glycine (Gly; G), valine (Val; V), alanine (Ala; A), phenylalanine (Phe; F), glutamic acid (Glu; E), isoleucine (Ile; I), proline (Pro; P), citrulline (Cit), leucine (Leu; L), serine (Ser; S), lysine (Lys; K), and aspartic acid (Asp; D). Preferred among them are glycine (Gly; G), valine (Val; V), alanine (Ala; A), and citrulline (Cit).

Any of these amino acids may appear multiple times, and Lp has an amino acid sequence including arbitrarily selected amino acids. Drug release pattern may be controlled via amino acid type.

Specific of linker Lp may include, but are not limited to, -GGVA- (SEQ ID NO: 85), -GG-(D-)VA-, -VA-, -GGFG- (SEQ ID NO: 86), -GGPI- (SEQ ID NO: 87), -GGVCit- (SEQ ID NO: 88), -GGVK- (SEQ ID NO: 89), -GG(D-)PI-, -GGPL- (SEQ ID NO: 90), -EGGVA (SEQ ID NO: 91), -PI-, -GGF-, DGGF- (SEQ ID NO: 92), (D-)D-GGF-, -EGGF- (SEQ ID NO: 93), -SGGF- (SEQ ID NO: 94), -KGGF- (SEQ ID NO: 95), -DGGFG- (SEQ ID NO: 96), -GGFGG- (SEQ ID NO: 97), -DDGGFG- (SEQ ID NO: 98), -KDGGFG- (SEQ ID NO: 99), and -GGFGGGF- (SEQ ID NO: 100).

Here, “(D-)V” indicates D-valine, “(D)-P” indicates D-proline, and “(D-)D” indicates D-aspartic acid.

Linker Lp is preferably any of the following:

(SEQ ID NO: 85)
-GGVA-,
-GG-(D-)VA-,
-VA-,
(SEQ ID NO: 86)
-GGFG-,
(SEQ ID NO: 87)
-GGPI-,
(SEQ ID NO: 88)
-GGVCit-,
(SEQ ID NO: 89)
-GGVK-,
-GG(D-)PI-,
and
(SEQ ID NO: 90)
-GGPL-.

Linker Lp is more preferably any of the following:

(SEQ ID NO: 85)
-GGVA-,
(SEQ ID NO: 88)
-GGVCit-,
and
-VA-.

Lb represents: a spacer which connects La to a glycan or remodeled glycan of Ab. In some embodiments, Lb represents any one selected from the following group: —C(═O)—(CH₂CH₂)n²-C(═O)—, —C(═O)—(CH₂CH₂)n²-C(═O)—NH—(CH₂CH₂)n³-C(═O)—, —C(═O)—(CH₂CH₂)n²-C(═O)—NH—(CH₂CH₂O)n³-CH₂—C(═O)—, —C(═O)—(CH₂CH₂)n²-NH—C(═O)—(CH₂CH₂O)n³-CH₂CH₂—C(═O)—, —(CH₂)n⁴-O—C(═O)— wherein, n² represents an integer of 1 to 3 (preferably, 1 or 2), n³ represents an integer of 1 to 5 (preferably, an integer of 2 to 4, more preferably, 2 or 4), and n⁴ represents an integer of 0 to 2 (preferably, 0 or 1).

In some embodiments, La preferably represents any one selected from the following group:
—C(═O)—CH₂CH₂—C(═O)—, —C(═O)—(CH₂CH₂)₂—C(═O)—,
—C(═O)—CH₂CH₂—C(═O)—NH—(CH₂CH₂)₂—C(═O)—
—C(═O)—CH₂CH₂—C(═O)—NH—(CH₂CH₂O)₂—CH₂—C(═O)—,
—C(═O)—CH₂CH₂—NH—C(═O)—(CH₂CH₂O)₄—CH₂CH₂—C(═O)—,
—CH₂—OC(═O)—, and —OC(═O)—, and
La is more preferably —C(═O)—CH₂CH₂—C(═O)— or —C(═O)—(CH₂CH₂)₂—C(═O)—.

Spacer Lb is not limited to a particular spacer, and examples thereof may include, but are not limited to, a spacer represented by the following formulas.

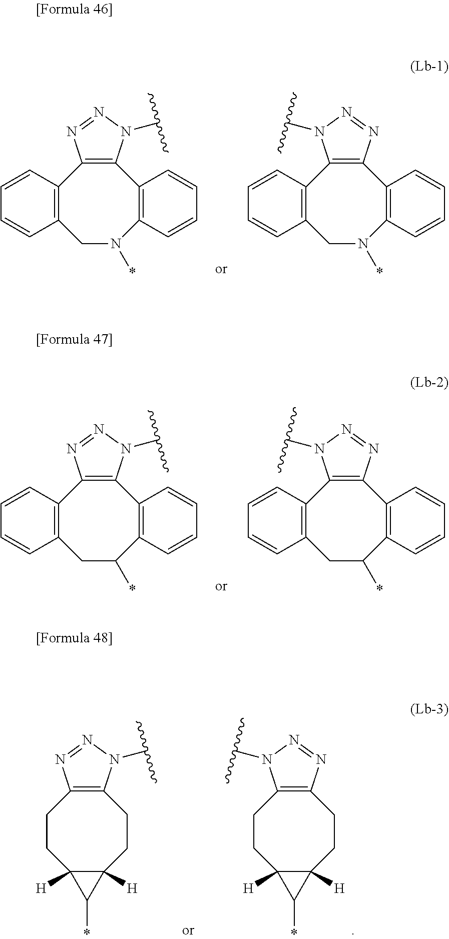 [SMILES: *N1Cc2ccccc2-c2nnn(*)c2-c2ccccc21 ; *N1Cc2ccccc2-c2c(nnn2*)-c2ccccc21 ; *C1Cc2ccccc2-c2nnn(*)c2-c2ccccc21 ; *C1Cc2ccccc2-c2c(nnn2*)-c2ccccc21 ; *C1[C@H]2CCc3c(nnn3*)CC[C@@H]12 ; *C1[C@H]2CCc3nnn(*)c3CC[C@@H]12]

In the structural formulas for Lb shown above, each asterisk represents bonding to —(C═O) or —(CH₂)n⁴ at the left end of La, and each wavy line represents bonding to a glycan or remodeled glycan of Ab.

In each structural formula for Lb (Lb-1, Lb-2, or Lb-3) shown above, the triazole ring site formed through click reaction of an azide group and DBCO provides structures of geometric isomers, and molecules of Lb exist as any one of the two structures or as a mixture of both of them. There exist m¹ “-L-D” moieties per molecule of the antibody-drug conjugate of the present invention, and either one of the two structures exist or both of them coexist as Lb (Lb-1, Lb-2, or Lb-3) in L of each of the m¹ “-L-D” moieties.

In some embodiments, L is preferably represented by -Lb-La-Lp-NH—B—CH₂—O(C═O)—*, wherein:
B is a 1,4-phenyl group,
Lp represents any one selected from the following group:

(SEQ ID NO: 85)
-GGVA-,
-GG-(D-)VA-,
-VA-,
(SEQ ID NO: 86)
-GGFG-,
(SEQ ID NO: 87)
-GGPI-,
(SEQ ID NO: 88)
-GGVCit-,
(SEQ ID NO: 89)
-GGVK-,
-GG(D-)PI-,
and
(SEQ ID NO: 90)
-GGPL-,

La represents any one selected from the following group:
—C(═O)—CH₂CH₂—C(═O)—, —C(═O)—(CH₂CH₂)₂—C(═O)—,
—C(═O)—CH₂CH₂—C(═O)—NH—(CH₂CH₂)₂—C(═O)—,
—C(═O)—CH₂CH₂—C(═O)—NH—(CH₂CH₂O)₂—CH₂—C(═O)—,
—C(═O)—CH₂CH₂—NH—C(═O)—(CH₂CH₂O)₄—CH₂CH₂—C(═O)—, —CH₂—OC(═O)—, —OC(═O)—, and
Lb represents any of the structural formulas above for Lb.

In some embodiments, L is more preferably any one selected from the following group:
—Z¹—C(═O)—CH₂CH₂—C(═O)-GGVA-NH—B—CH₂—OC(═O)— (“GGVA” disclosed as SEQ ID NO: 85),
—Z¹—C(═O)—CH₂CH₂—C(═O)-GG-(D-)VA-NH—B—CH₂—OC(═O)—,
—Z¹—C(═O)—CH₂CH₂—C(═O)—VA-NH—B—CH₂—OC(═O)—,
—Z¹—C(═O)—(CH₂CH₂)₂—C(═O)—VA-NH—B—CH₂—OC(═O)—,
—Z¹—C(═O)—CH₂CH₂—C(═O)-GGPI-NH—B—CH₂—OC(═O)— (“GGPI” disclosed as SEQ ID NO: 87),
—Z¹—C(═O)—CH₂CH₂—C(═O)-GGFG-NH—B—CH₂—OC(═O)— (“GGFG” disclosed as SEQ ID NO: 86),
—Z¹—C(═O)—CH₂CH₂—C(═O)-GGVCit-NH—B—CH₂—OC(═O)— (“GGVCit” disclosed as SEQ ID NO: 88),
—Z¹—C(═O)—CH₂CH₂—C(═O)-GGVK-NH—B—CH₂—OC(═O)— (“GGVK” disclosed as SEQ ID NO: 89),
—Z¹—C(═O)—CH₂CH₂—C(═O)-GGPL-NH—B—CH₂—OC(═O)— (“GGPL” disclosed as SEQ ID NO: 90),
—Z¹—C(═O)—CH₂CH₂—C(═O)—NH—(CH₂CH₂)₂—C(═O)—VA-NH—B—CH₂—OC(═O)—,
—Z¹—C(═O)—CH₂CH₂—C(═O)—NH—(CH₂CH₂O)₂—CH₂—C(═O)—VA-NH—B—CH₂—OC(═O)—,
—Z¹—C(═O)—CH₂CH₂—NH—C(═O)—(CH₂CH₂O)₄—CH₂CH₂—C(═O)—VA-NH—B—CH₂—OC(═O)—,
—Z²—OC(═O)-GGVA-NH—B—CH₂—OC(═O)— (“GGVA” disclosed as SEQ ID NO: 85),
—Z³—CH₂—OC(═O)-GGVA-NH—B—CH₂—OC(═O)— (“GGVA” disclosed as SEQ ID NO: 85);
wherein Z¹ represents the following structural formula as described for Lb:

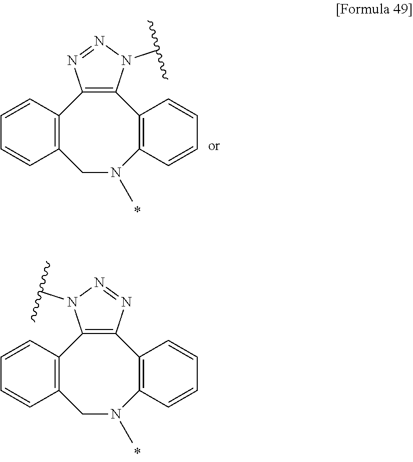 [SMILES: *n1nnc2c1-c1ccccc1N(C)Cc1ccccc1-2 ; *n1nnc2c1-c1ccccc1CN(C)c1ccccc1-2]

Z² represents the following structural formula as described for Lb:

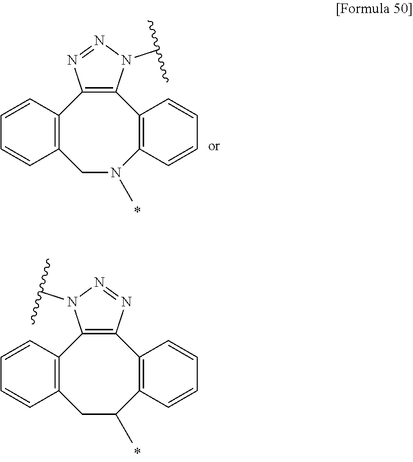 [SMILES: *n1nnc2c1-c1ccccc1N(C)Cc1ccccc1-2 ; *n1nnc2c1-c1ccccc1CC(C)c1ccccc1-2]

Z³ represents the following structural formula as described for Lb:

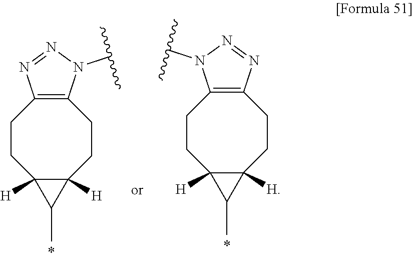

and B is a 1,4-phenyl group.

L is most preferably any of the following:
—Z¹—C(═O)—CH₂CH₂—C(═O)-GGVA-NH—B—CH₂—OC(═O)— (“GGVA” disclosed as SEQ ID NO: 85),
—Z¹—C(═O)—CH₂CH₂—C(═O)—VA-NH—B—CH₂—OC(═O)—,
—Z¹—C(═O)—(CH₂CH₂)₂—C(═O)—VA-NH—B—CH₂—OC(═O)—,
—Z¹—C(═O)—CH₂CH₂—C(═O)-GGVCit-NH—B—CH₂—OC(═O)— (“GGVCit” disclosed as SEQ ID NO: 88),
—Z¹—C(═O)—CH₂CH₂—C(═O)—NH—(CH₂CH₂)₂—C(═O)—VA-NH—B—CH₂—OC(═O)—,
—Z¹—C(═O)—CH₂CH₂—C(═O)—NH—(CH₂CH₂O)₂—CH₂—C(═O)—VA-NH—B—CH₂—OC(═O)—, and —Z¹—C(═O)—CH₂CH₂—NH—C(═O)—(CH₂CH₂O)₄—CH₂CH₂—C(═O)—VA-NH—B—CH₂—OC(═O)—, wherein
B is a 1,4-phenyl group, and Z¹ represents the following structural formula as described for Lb:

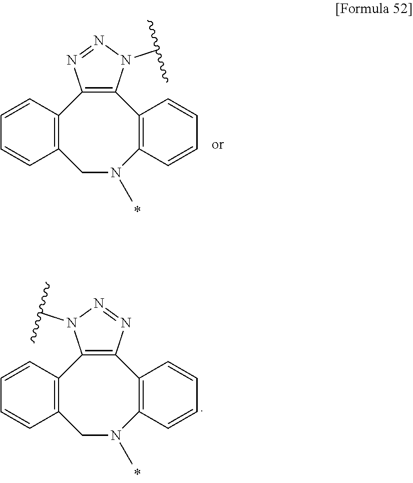 [SMILES: *n1nnc2c1-c1ccccc1N(C)Cc1ccccc1-2 ; *n1nnc2c1-c1ccccc1CN(C)c1ccccc1-2]

The antibody-drug conjugate of the present invention is inferred to exhibit antitumor activity through a process in which most molecules of the antibody-drug conjugate migrate into tumor cells, and a linker portion (e.g., Lp) is then cleaved by an enzyme or the like to activate the antibody-drug conjugate, which releases the portion of drug D (hereinafter, referred to as a free drug (described later)).

Therefore, it is preferable that the antibody-drug conjugate of the present invention is stable outside of tumor cells.

C. Antibody

The antibodies to be used in the disclosed antibody-drug conjugates (ADCs) are anti-DLL3 antibodies (e.g., 2-C8-A, 6-G23-F, and 10-O18-A, or human antibody H2-C8-A, H6-G23-F, H10-O18-A, H2-C8-A-3, and H10-O18-A-3). The disclosed antibodies that may be incorporated into the disclosed ADCs are detailed in Section III “Anti-DLL3 Antibodies” above.

Briefly, in some embodiments, the anti-DLL3 antibody of the ADC may comprise a heavy chain immunoglobulin variable domain (V_{H}) and a light chain immunoglobulin variable domain (V_{L}), wherein (a) the V_{H} comprises a V_{H}-CDR1 sequence, a V_{H}-CDR2 sequence, and a V_{H}-CDR3 sequence selected from the group consisting of (i) SEQ ID NO: 3, SEQ ID NO: 4, and SEQ ID NO: 5, respectively; (ii) SEQ ID NO: 13, SEQ ID NO: 14, and SEQ ID NO: 15, respectively; (iii) SEQ ID NO: 23, SEQ ID NO: 24, and SEQ ID NO: 25, respectively; and (iv) SEQ ID NO: 33, SEQ ID NO: 34, and SEQ ID NO: 35, respectively; and/or (b) the V_{L} comprises a V_{L}-CDR1 sequence, a V_{L}—CDR2 sequence, and a V_{L}-CDR3 sequence selected from the group consisting of (i) SEQ ID NO: 8, SEQ ID NO: 9, and SEQ ID NO: 10, respectively; (ii) SEQ ID NO: 18, SEQ ID NO: 19, and SEQ ID NO: 20, respectively; (iii) SEQ ID NO: 28, SEQ ID NO: 29, and SEQ ID NO: 30, respectively; and (iv) SEQ ID NO: 38, SEQ ID NO: 39, and SEQ ID NO: 40, respectively.

In some embodiments, the anti-DLL3 antibody of the ADC may comprise one or more of the following characteristics: (a) a light chain immunoglobulin variable domain sequence that is at least 80%, at least 85%, at least 90%, at least 95%, or at least 99% identical to the light chain immunoglobulin variable domain sequence present in any one of SEQ ID NOs: 7, 17, 27, 37, 62, 66, or 70; and/or (b) a heavy chain immunoglobulin variable domain sequence that is at least 80%, at least 85%, at least 90%, at least 95%, or at least 99% identical to the heavy chain immunoglobulin variable domain sequence present in any one of SEQ ID NOs: 2, 12, 22, 32, 59, 60, 61, 63, 64, 65, 67, 68, or 69.

In some embodiments, the anti-DLL3 antibody of the ADC may comprise a heavy chain immunoglobulin variable domain (V_{H}) or heavy chain and a light chain immunoglobulin variable domain (V_{L}) or light chain, wherein: (a) the V_{H} or heavy chain comprises an amino acid sequence selected from the group consisting of SEQ ID NO: 2, SEQ ID NO: 12, SEQ ID NO: 22, SEQ ID NO: 32, SEQ ID NO: 59, SEQ ID NO: 60, SEQ ID NO: 61, SEQ ID NO: 63, SEQ ID NO: 64, SEQ ID NO: 65, SEQ ID NO: 67, SEQ ID NO: 68, and SEQ ID NO: 69; and/or (b) the V_{L} or light chain comprises an amino acid sequence selected from the group consisting of SEQ ID NO: 7, SEQ ID NO: 17, SEQ ID NO: 27, SEQ ID NO: 37, SEQ ID NO 62, SEQ ID NO: 66, and SEQ ID NO: 70. In some embodiments, the V_{H} or heavy chain amino acid sequence and the V_{L} or light chain amino acid sequence is selected from the group consisting of: SEQ ID NO: 2 and SEQ ID NO: 7 (7-I1-B), respectively; SEQ ID NO: 12 and SEQ ID NO: 17 (2-C8-A), respectively; SEQ ID NO: 59 and SEQ ID NO: 62 (H2-C8-A), respectively; SEQ ID NO: 60 and SEQ ID NO: 62 (H2-C8-A-2), respectively; SEQ ID NO: 61 and SEQ ID NO: 62 (H2-C8-A-3), respectively; SEQ ID NO: 22 and SEQ ID NO: 27 (10-O18-A), respectively; SEQ ID NO: 67 and SEQ ID NO: 70 (H10-O18-A), respectively; SEQ ID NO: 68 and SEQ ID NO: 70 (H10-O18-A-2), respectively; SEQ ID NO: 69 and SEQ ID NO: 70 (H10-O18-A-3); SEQ ID NO: 32 and SEQ ID NO: 37 (6-G23-F), respectively; SEQ ID NO: 63 and SEQ ID NO: 66 (H6-G23-F), respectively; SEQ ID NO: 64 and SEQ ID NO: 66 (H6-G23-F-2), respectively; and SEQ ID NO: 65 and SEQ ID NO: 66 (H6-G23-F-3), respectively.

In some embodiments, the anti-DLL3 antibody of the ADC may be glycan remodeled or glycan modified, as discussed in more detail below.

The functional fragment of the present invention includes a functional fragment that has well conserved asparagine (Asn297) to be modified with an N-linked glycan in the IgG heavy chain Fc region and amino acids around Asn297, while retains binding activity to an antigen.

i. Glycan Remodeling of the Disclosed Anti-DLL3 Antibodies:

Recently has been reported a method for remodeling heterogeneous glycoprotein of an antibody by enzymatic reaction or the like to homogeneously introduce a glycan having a functional group (ACS Chemical Biology 2012, 7, 110, ACS Medicinal Chemistry Letters 2016, 7, 1005). An attempt with use of this glycan remodeling technique has been made to site-specifically introduce a drug to synthesize a homogeneous ADC (Bioconjugate Chemistry 2015, 26, 2233, Angew. Chem. Int. Ed. 2016, 55, 2361-2367, US 2016361436).

In the glycan remodeling of the present invention, using hydrolase, heterogeneous glycans added to a protein (e.g., an antibody) are cleaved off to leave only GlcNAc at each terminus thereby producing a homogenous protein moiety with GlcNAc (hereinafter, referred to as an “acceptor”). Subsequently, an arbitrary glycan separately prepared (hereinafter, referred to as a “donor”) is provided, and the acceptor and the donor are linked together by using transglycosidase. Thereby, a homogeneous glycoprotein with arbitrary glycan structure can be synthesized.

In the present invention, a “glycan” refers to a structural unit of two or more monosaccharides bonded together via glycosidic bonds. Specific monosaccharides and glycans are occasionally abbreviated, for example, as “GlcNAc-”, “MSG-”, and so on. When any of these abbreviations is used in a structural formula, the abbreviation is shown with an intention that an oxygen atom or nitrogen atom involved in a glycosidic bond at the reducing terminal to another structural unit is not included in the abbreviation indicating the glycan, unless specifically defined.

In the present invention, a monosaccharide as a basic unit of a glycan is indicated for convenience so that in the ring structure, the position of a carbon atom bonding to an oxygen atom constituting the ring and directly bonding to a hydroxy group (or an oxygen atom involved in a glycosidic bond) is defined as the 1-position (the 2-position only for sialic acids), unless otherwise specified. The names of compounds in Examples are each provided in view of the chemical structure as a whole, and that rule is not necessarily applied.

When a glycan is indicated as a sign (e.g., GLY, SG, MSG, GlcNAc) in the present invention, the sign is intended, unless otherwise defined, to include carbon atoms ranging to the reducing terminal and not to include N or O involved in an N- or O-glycosidic bond.

In the present invention, unless specifically stated, a partial structure when a glycan is linking to a side chain of an amino acid is indicated in such a manner that the side chain portion is indicated in parentheses, for example, “(SG-)Asn”.

D. Antibody-Drug Conjugate (ADC)

In some embodiments, the anti-DLL3 antibody-drug conjugate of the present invention is represented by the following formula:

Ab [symbol] L-D]ₘ_{¹} [Formula 53]

wherein antibody Ab or a functional fragment of the antibody may bond from a side chain of an amino acid residue thereof (e.g., cysteine, lysine) directly to L, or bond via a glycan or remodeled glycan of Ab to L, and preferably bonds via a glycan or remodeled glycan of Ab to L, and more preferably bonds via a remodeled glycan of Ab to L.

Glycans in Ab of the present invention are N-linked glycans or O-linked glycans, and preferably N-linked glycans. N-linked glycans and O-linked glycans bond to an amino acid side chain of an antibody via an N-glycosidic bond and an O-glycosidic bond, respectively.

In some embodiments, the antibody (Ab) of the present invention is an IgG, and preferably IgG1, IgG2, or IgG4. In some embodiments, the antibody (Ab) is 2-C8-A, 6-G23-F, 10-O18-A, H2-C8-A, H2-C8-A-2, H2-C8-A-3, H6-G23-F, H6-G23-F-2, H6-G23-F-3, H10-O18-A, H10-O18-A-2, or H10-O18-A-3.

IgG has a well conserved N-linked glycan on an asparagine residue at the 297-position of the Fc region of the heavy chain (hereinafter, referred to as “Asn297 or N297”), and the N-linked glycan is known to contribute to the activity and kinetics of the antibody molecule. (Biotechnol. Prog., 2012, 28, 608-622, Sanglier-Cianferani, S., Anal. Chem., 2013, 85, 715-736)

The amino acid sequence in the constant region of IgG is well conserved, and each amino acid is specified by Eu index numbering in Edelman et al. (Proc. Natl. Acad. Sci. U.S.A., Vol. 63, No. 1 (May 15, 1969), pp. 78-85). For example, Asn297, to which an N-linked glycan is added in the Fc region, corresponds to the 297-position in Eu index numbering, and each amino acid is uniquely specified by Eu index numbering, even if the actual position of the amino acid has varied through fragmentation of the molecule or deletion of a region.

In some embodiments of the antibody-drug conjugate of the present invention, the antibody or functional fragment of the antibody may bond to L via a glycan bonding to a side chain of Asn297 thereof (hereinafter, referred to as “N297 glycan”), and the antibody or functional fragment of the antibody even more preferably bonds via the N297 glycan to L, wherein the N297 glycan is a remodeled glycan.

The following formula illustrates the situation that the antibody-drug conjugate of the present invention or a functional fragment of the antibody bonds via the N297 glycan to L.

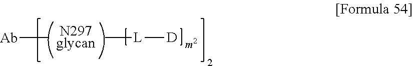

An antibody having the remodeled glycan is referred to as a glycan-remodeled antibody.

SGP, an abbreviation for sialyl glycopeptide, is a representative N-linked complex glycan. SGP can be separated/purified from the yolk of a hen egg, for example, by using a method described in WO 2011/0278681. Purified products of SGP are commercially available (Tokyo Chemical Industry Co., Ltd., FUSHIMI Pharmaceutical Co., Ltd.), and may be purchased. For example, disialooctasaccharide (Tokyo Chemical Industry Co., Ltd.), a glycan formed by deleting one GlcNAc at the reducing terminal in the glycan moiety of SG (hereinafter, referred to as “SG (10)”, is commercially available.

In the present invention, a glycan structure formed by deleting a sialic acid at a non-reducing terminal only in either one of the branched chains of β-Man in SG (10) refers to MSG (9), and a structure having a sialic acid only in the 1-3 branched chain is called as MSG1, and a structure having a sialic acid only in the 1-6 branched chain is called as MSG2.

The remodeled glycan of the present invention is N297-(Fuc)MSG1, N297-(Fuc)MSG2, or a mixture of N297-(Fuc)MSG1 and N297-(Fuc)MSG2, or N297-(Fuc)SG, and is preferably N297-(Fuc)MSG1, N297-(Fuc)MSG2, or N297-(Fuc)SG, and is more preferably N297-(Fuc)MSG1 or N297-(Fuc)MSG2.

N297-(Fuc)MSG1 is represented by the following structural formula or sequence formula:

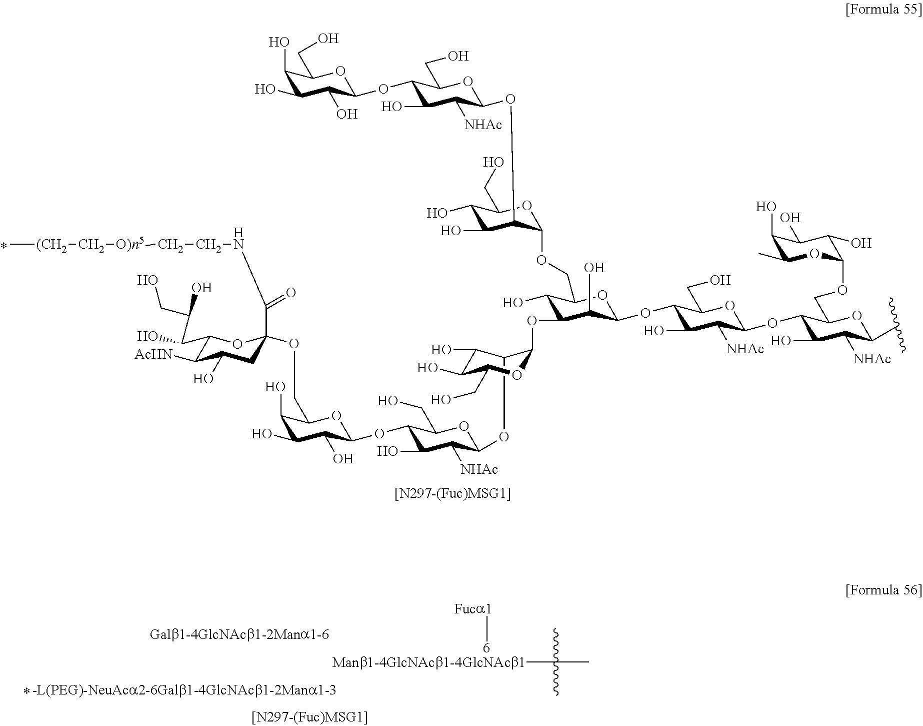

In the formulas above.
each wavy line represents bonding to Asn297 of the antibody,
L(PEG) represents —(CH₂CH₂—O)n⁵-CH₂CH₂—NH—, wherein the amino group at the right end represents amide-bonding to carboxylic acid at the 2-position of a sialic acid at the non-reducing terminal in the 1-3 branched chain of β-Man in the N297 glycan,
each asterisk represents bonding to linker L, in particular, a nitrogen atom at the 1- or 3-position of the 1,2,3-triazole ring of Lb in linker L, and
n⁵ is an integer of 2 to 10, and preferably an integer of 2 to 5.

N297-(Fuc)MSG2 is represented by the following structural formula or sequence formula:

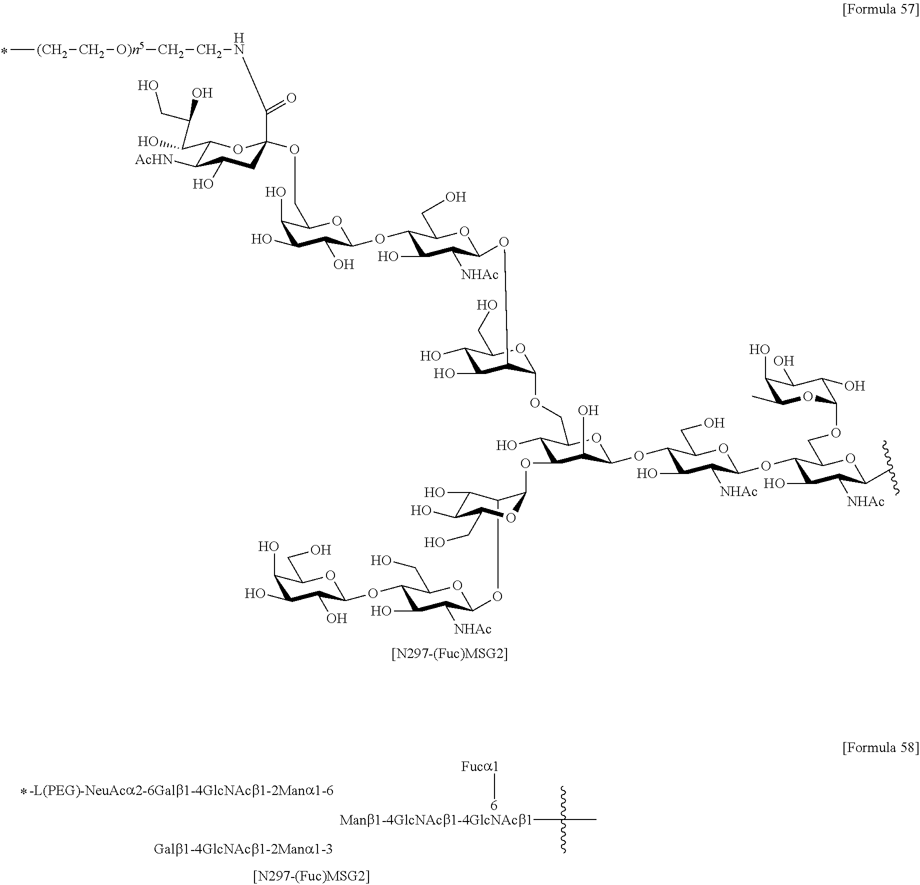

In the formulas above:
each wavy line represents bonding to Asn297 of the antibody,
L(PEG) represents —(CH₂CH₂—O)n⁵-CH₂CH₂—NH—, wherein the amino group at the right end represents amide-bonding to carboxylic acid at the 2-position of a sialic acid at the non-reducing terminal in the 1-6 branched chain of β-Man in the N297 glycan,
each asterisk represents bonding to linker L, in particular, a nitrogen atom at the 1- or 3-position of the 1,2,3-triazole ring of Lb in linker L, and
n⁵ is an integer of 2 to 10, and preferably an integer of 2 to 5.

N297-(Fuc)SG is represented by the following structural formula or sequence formula:

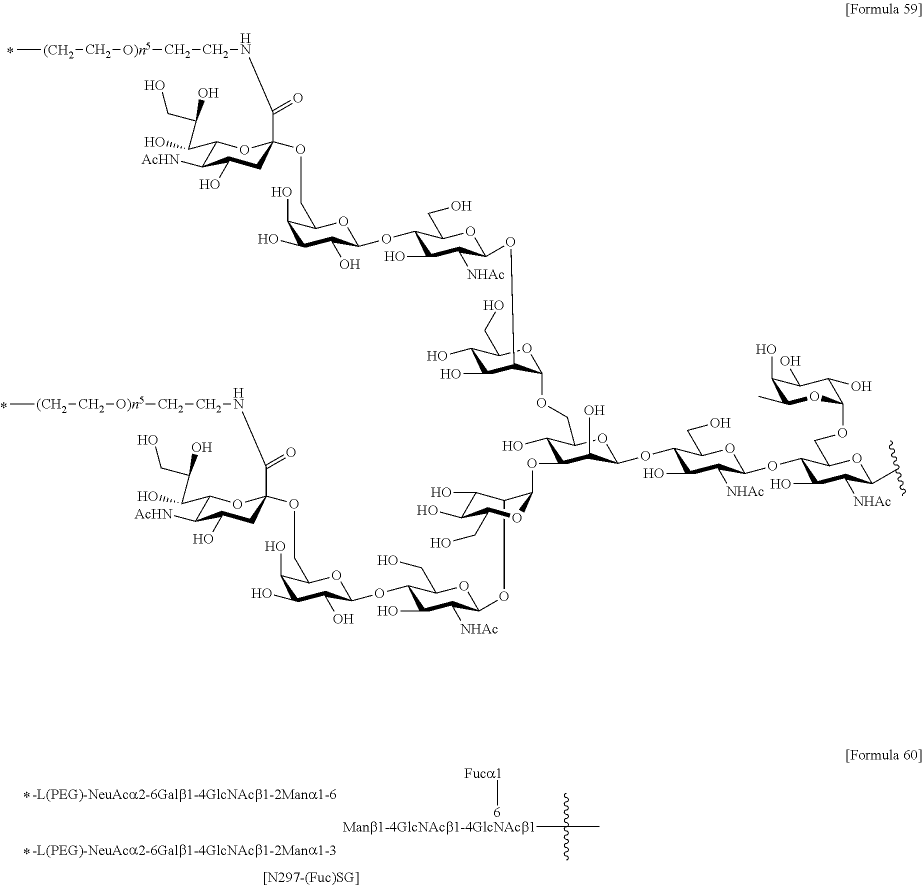

In the formulas above:
each wavy line represents bonding to Asn297 of the antibody,
L(PEG) represents —(CH₂CH₂—O)n⁵-CH₂CH₂—NH—, wherein the amino group at the right end represents amide-bonding to carboxylic acid at the 2-position of a sialic acid at the non-reducing terminal in each of the 1-3 and 1-6 branched chains of β-Man in the N297 glycan, each asterisk represents bonding to linker L, in particular, a nitrogen atom at the 1- or 3-position of the 1,2,3-triazole ring of Lb in linker L, and n5 is an integer of 2 to 10, and preferably an integer of 2 to 5.

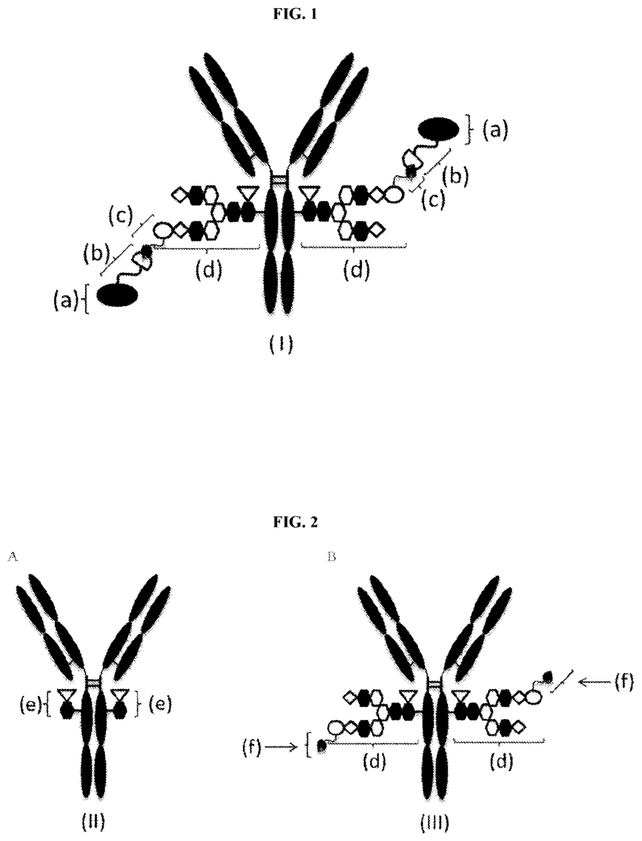

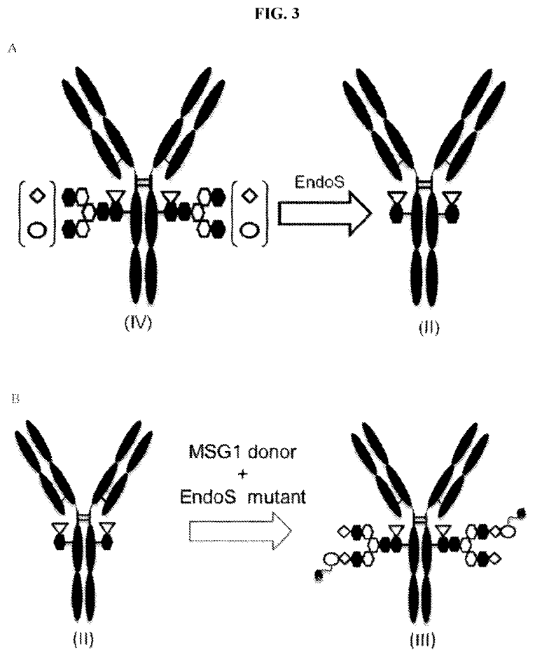

If N297 glycan of the antibody in the antibody-drug conjugate of the present invention is N297-(Fuc)MSG1, N297-(Fuc)MSG2, or a mixture of them, the antibody-drug conjugate is a molecule to which two molecules of linker L and two molecules of drug D have been conjugated (m²=1) since the antibody is a dimer (see FIG. 1).

If N297 glycan of the antibody in the antibody-drug conjugate of the present invention is N297-(Fuc)SG, the antibody-drug conjugate is a molecule to which four molecules of linker L and four molecules of drug D have been conjugated (m²=2). N297 glycan is preferably N297-(Fuc)MSG1, N297-(Fuc)MSG2, or N297-(Fuc)SG, and more preferably N297-(Fuc)MSG1 or N297-(Fuc)MSG2.

If N297 glycan of the antibody in the antibody-drug conjugate of the present invention is N297-(Fuc)MSG1, N297-(Fuc)MSG2, or N297-(Fuc)SG, a homogeneous ADC can be obtained.

The antibody-drug conjugate of the present invention is the most preferably one antibody-drug conjugate selected from the following group:

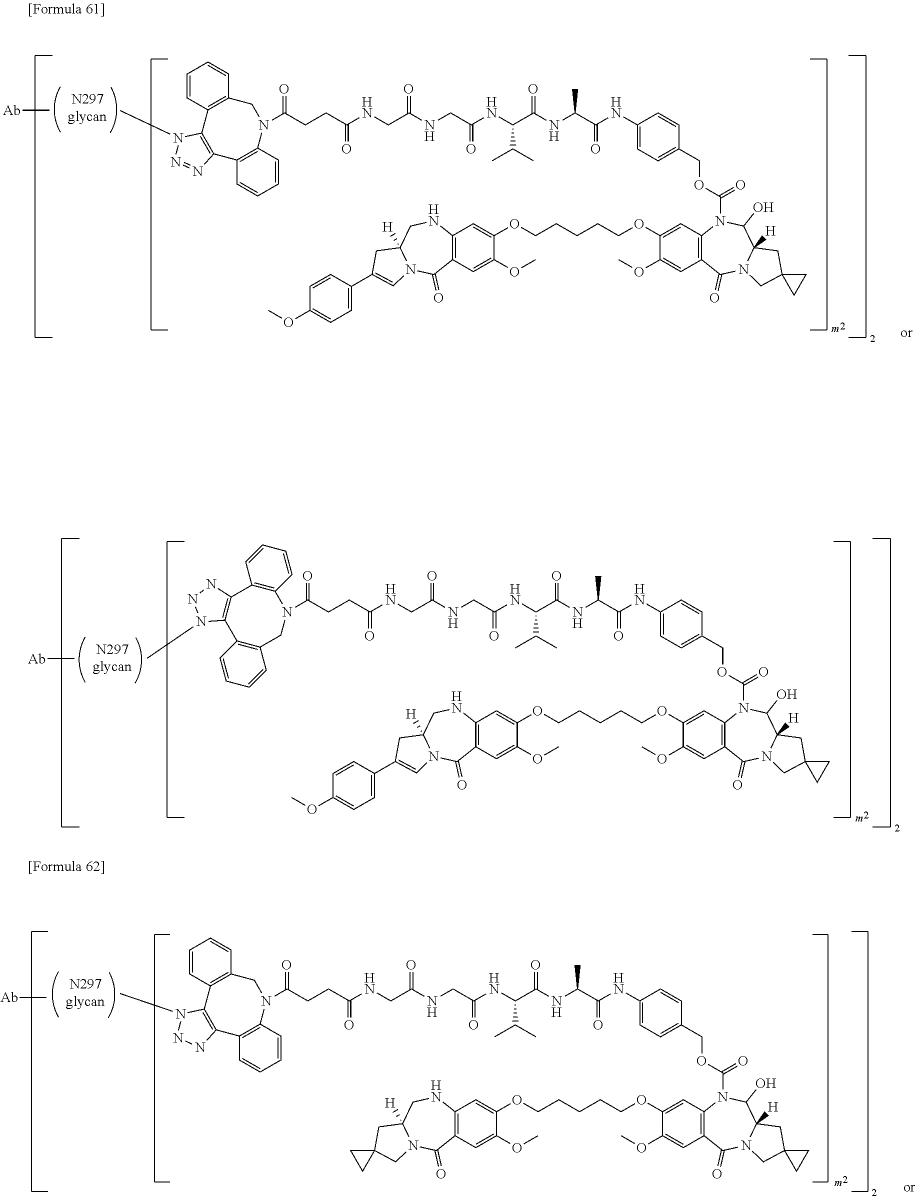

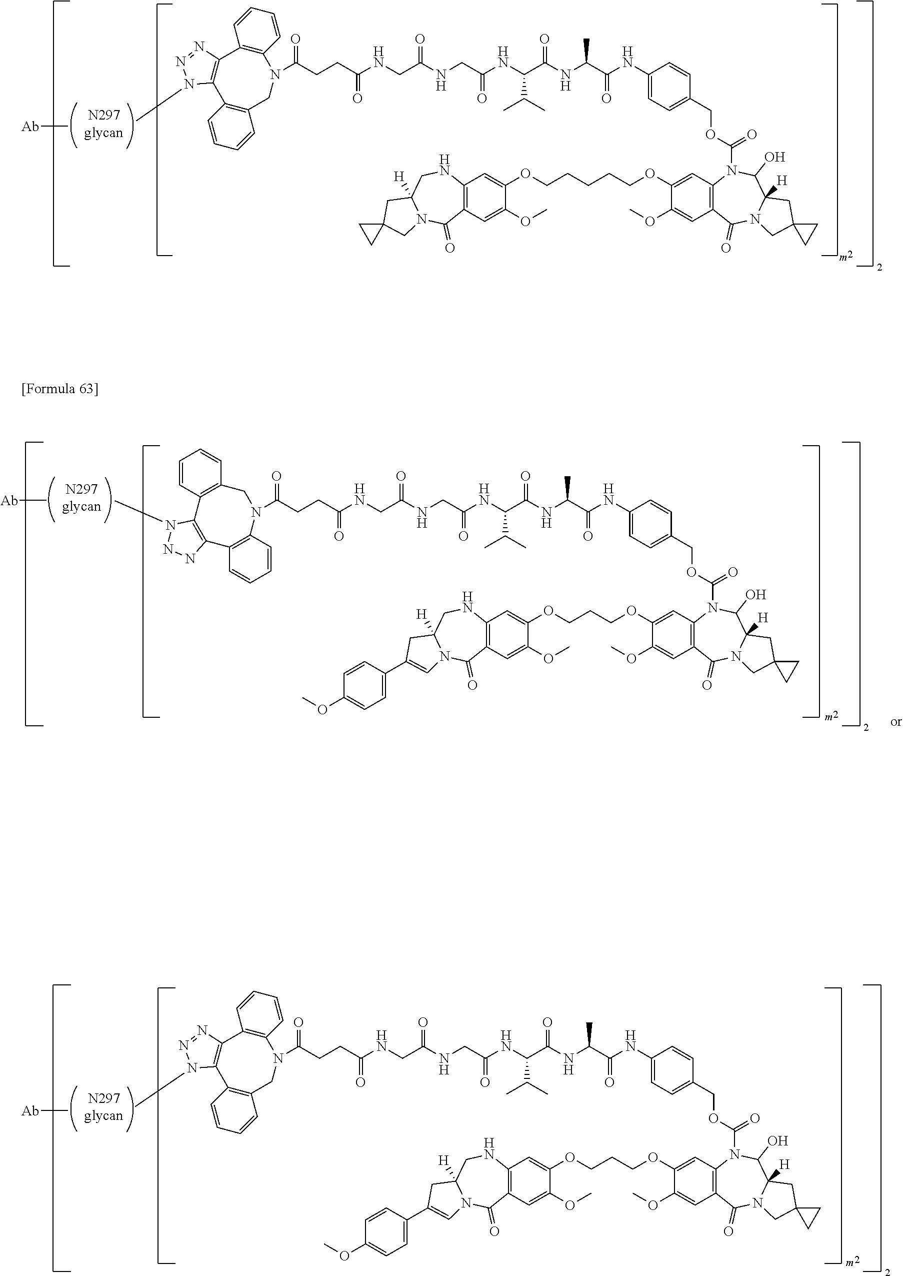

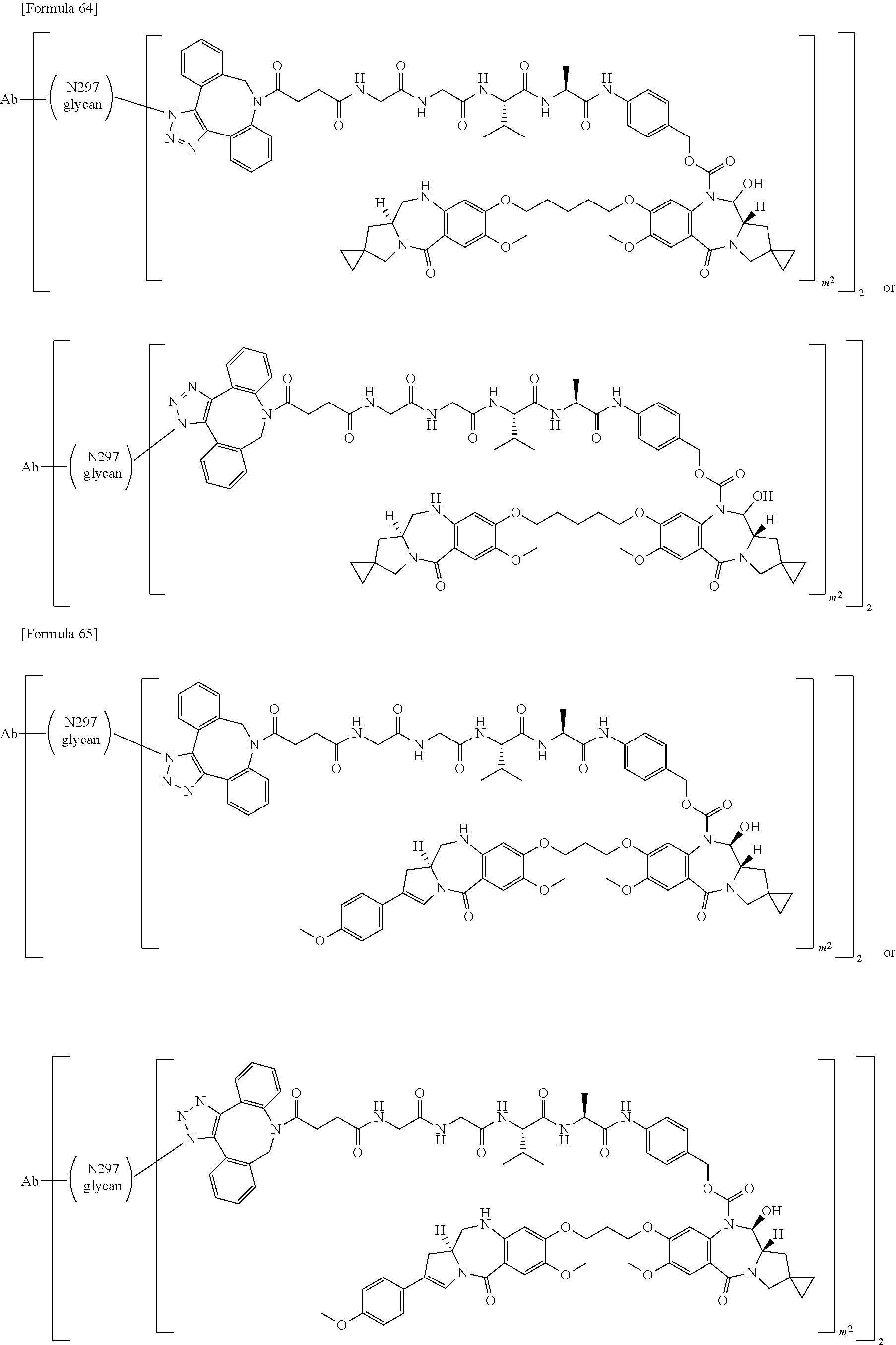

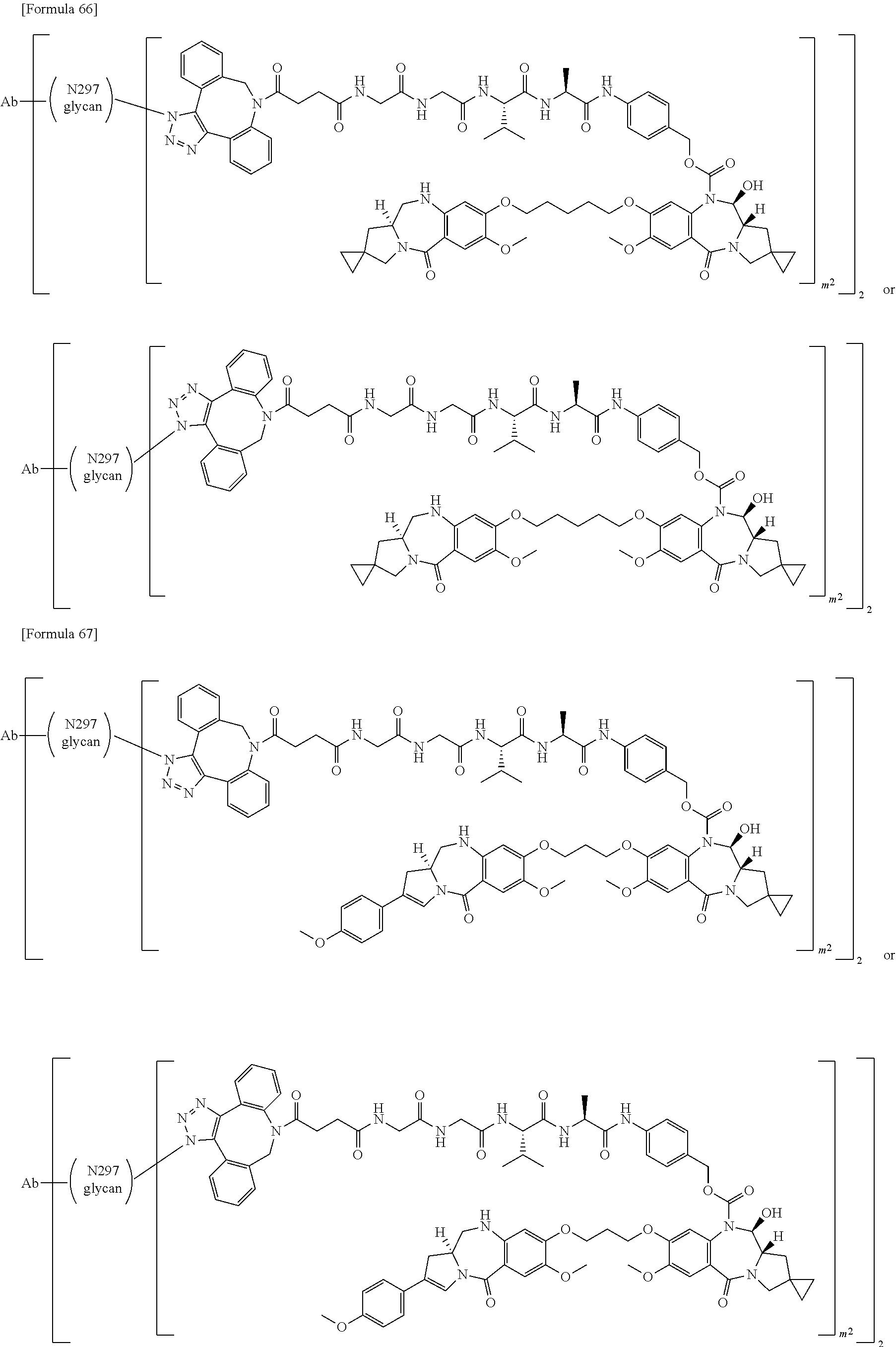

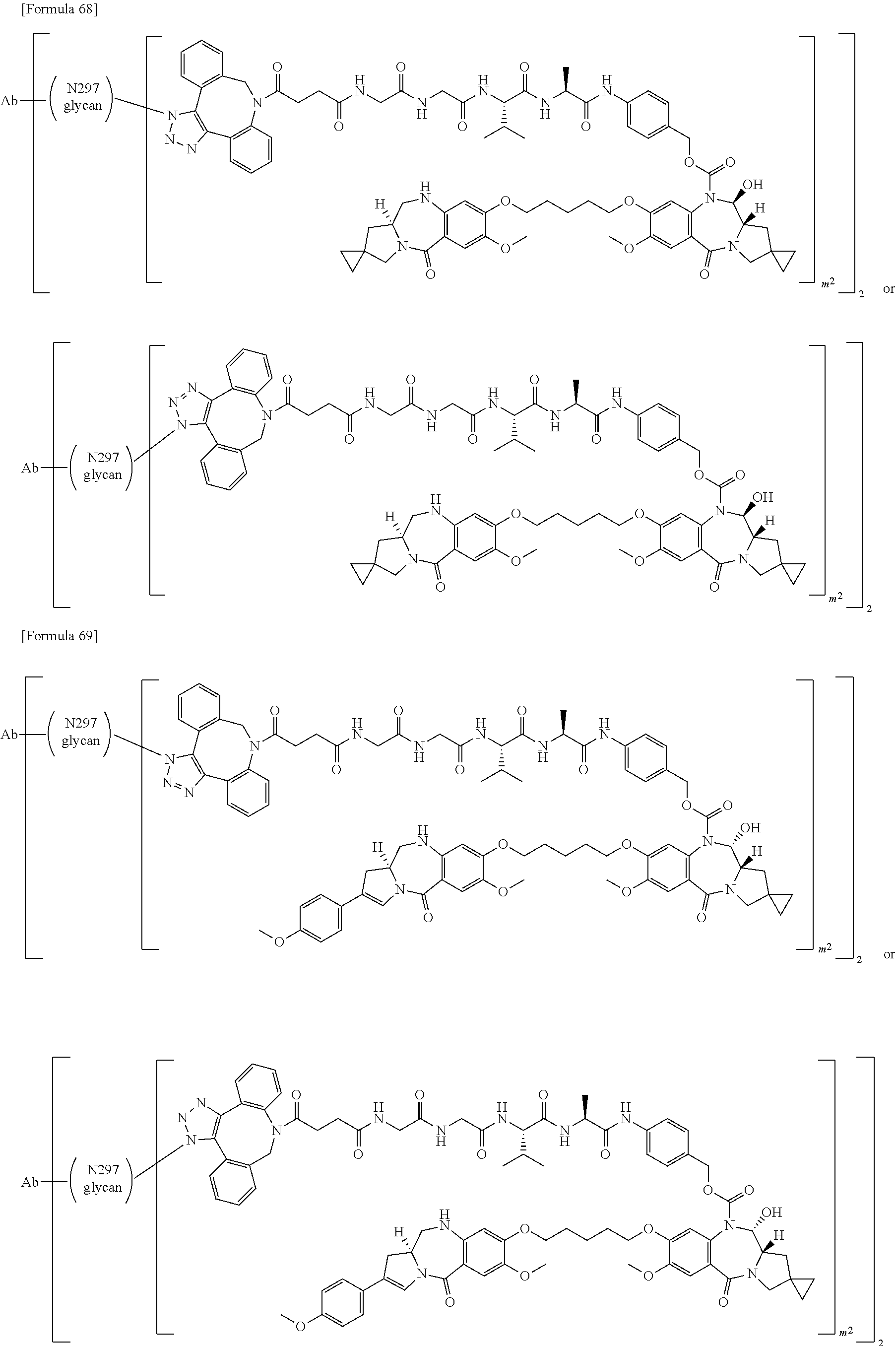

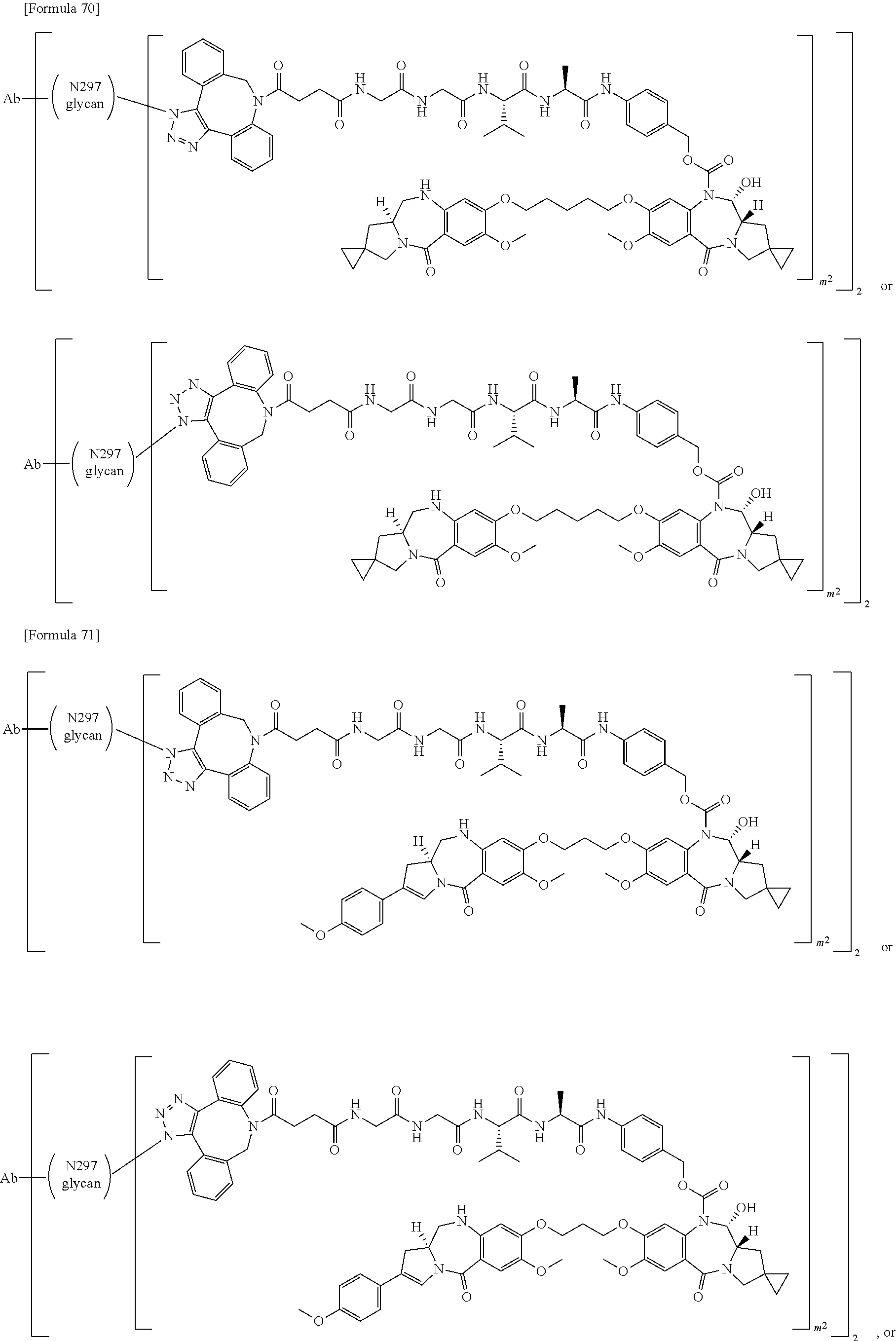

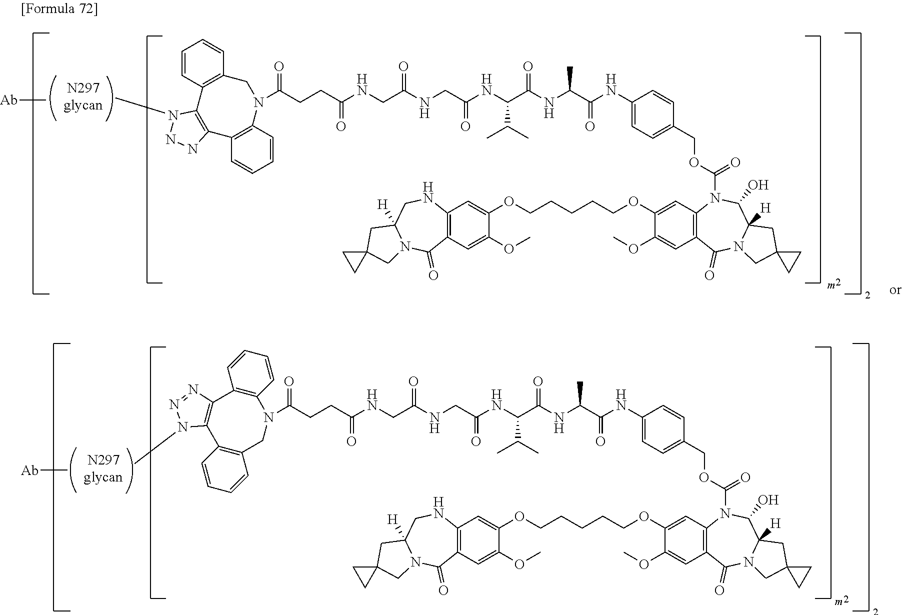

In each of the structural formulas above,
m² represents 1 or 2 (preferably, m² is 1),
antibody Ab is an anti-DLL3 IgG antibody (preferably, IgG1, IgG2, or IgG4, more preferably, IgG1), or a functional fragment of the antibody,
N297 glycan represents any one of N297-(Fuc)MSG1, N297-(Fuc)MSG2, and a mixture of them, and N297-(Fuc)SG (preferably, N297-(Fuc)MSG1),
L(PEG) represents —NH—CH₂CH₂—(O—CH₂CH₂)₃—*, wherein the amino group at the left end represents amide-bonding to carboxylic acid at the 2-position of a sialic acid at the non-reducing terminal of each or either one of the 1-3 and 1-6 branched chains (preferably, the 1-3 branched chain) of β-Man in N297 glycan, and the asterisk represents bonding to a nitrogen atom at the 1- or 3-position of the triazole ring of Lb in linker L. In some embodiments, the anti-DLL3 antibody is, for example, 2-C8-A, 6-G23-F, 10-O18-A, H2-C8-A, H2-C8-A-2, H2-C8-A-3, H6-G23-F, H6-G23-F-2, H6-G23-F-3, H10-O18-A, H10-O18-A-2, or H10-O18-A-3.

Although structures with two or four units (m²=1 or 2) of “-(N297 glycan)-L-D” in each of which N297 glycan bonds to the nitrogen atom at the 1-position of the triazole ring of Lb in L in one conjugate molecule (“(N297 glycan)-(N1Lb)L-D”) or structures with two or four units (m²=1 or 2) of “-(N297 glycan)-L-D” in each of which N297 glycan bonds to the nitrogen atom at the 3-position of the triazole ring of Lb in Lin one conjugate molecule (“(N297 glycan)-(N3Lb)L-D”) are illustrated as the most preferred antibody-drug conjugate for convenience, antibody-drug conjugates having both “(N297 glycan)-(N1Lb)L-D” (if m²=1, then one unit, if m²=2, then one, two, or three units) and “(N297 glycan)-(N3Lb)L-D” (if m²=1, then one unit, if m²=2, then three, two, or one unit) in one conjugate molecule are also included. In other words, either one of “(N297 glycan)-(N1Lb)L-D” and “(N297 glycan)-(N3Lb)L-D” exists or both of them coexist in one conjugate molecule.

The antibody-drug conjugate of the present invention and the anti-DLL3 antibody-drug conjugate of the present invention exhibit strong tumor activity (in vivo antitumor activity, in vitro anticellular activity) and satisfactory in vivo kinetics and physical property, and have high safety, and hence are useful as a pharmaceutical.

There may exist stereoisomers, optical isomers due to an asymmetric carbon atom, geometric isomers, tautomers, or optical isomers such as d-forms, 1-forms and atropisomers for the antibody-drug conjugate of the present invention, and a free drug or production intermediate of the antibody-drug conjugate, and these isomers, optical isomers, and mixtures of them are all included in the present invention. PBD derivative (V) or (VI) of the present invention has an asymmetric carbon at the 11′-position, and thus there exist optical isomers. Herein, these isomers and mixtures of these isomers are all represented by a single formula, namely, general formula (V) or (VI). Accordingly, (V) or (VI) includes all the optical isomers and mixtures of the optical isomers at any ratio. The absolute steric configuration at the 11′-position of (V) or (VI) can be determined through X-ray crystal structure analysis or NMR such as a Mosher method for its crystalline product or intermediate, or a derivative thereof. Then, the absolute steric configuration may be determined by using a crystalline product or intermediate derivatized with a reagent having an asymmetric center whose steric configuration is known. As desired, stereoisomers of the synthesized compound according to the present invention may be obtained by isolating with a common optical resolution method or separation method.

The number of conjugated drug molecules per antibody molecule is an important factor having influence on efficacy and safety for the antibody-drug conjugate of the present invention. Antibody-drug conjugates are produced with reaction conditions, such as the amounts of raw materials and reagents to be reacted, specified so as to give a constant number of conjugated drug molecules, but, in contrast to chemical reaction of low-molecular-weight compounds, a mixture with different numbers of conjugated drug molecules is typically obtained. Numbers of conjugated drug molecules per antibody molecule are specified as the average value, namely, the average number of conjugated drug molecules (DAR: Drug to Antibody Ratio). The number of pyrrolobenzodiazepine derivative molecules conjugated to an antibody molecule is controllable, and 1 to 10 pyrrolobenzodiazepine derivative molecules can be conjugated as the average number of conjugated drug molecules per antibody molecule (DAR), but preferably the number is one to eight, and more preferably one to five.

If the antibody bonds via a remodeled glycan of the antibody to L in the antibody-drug conjugate of the present invention, the number of conjugated drug molecules per antibody molecule in the antibody-drug conjugate, m², is an integer of 1 or 2. If the glycan is N297 glycan and the glycan is N297-(Fuc)MSG1, N297-(Fuc)MSG2, or a mixture of N297-(Fuc)MSG1 and N297-(Fuc)MSG2, m² is 1, and DAR is in the range of 1 to 3 (preferably, in the range of 1.0 to 2.5, more preferably, in the range of 1.2 to 2.2). If the N297 glycan is N297-(Fuc)SG, m² is 2, and DAR is in the range of 3 to 5 (preferably, in the range of 3.2 to 4.8, more preferably, in the range of 3.5 to 4.2).

In any of the foregoing embodiments, the anti-DLL3 antibody (i.e., “the antibodiy” or “Ab”), can be selected from 2-C8-A, 6-G23-F, 10-O18-A, H2-C8-A, H2-C8-A-2, H2-C8-A-3, H6-G23-F, H6-G23-F-2, H6-G23-F-3, H10-O18-A, H10-O18-A-2, or H10-O18-A-3. In some embodiments, the anti-DLL3 antibody of the ADC may comprise a heavy chain immunoglobulin variable domain (V_{H}) and a light chain immunoglobulin variable domain (V_{L}), wherein (a) the V_{H} comprises a V_{H}-CDR1 sequence, a V_{H}-CDR2 sequence, and a V_{H}-CDR3 sequence selected from the group consisting of (i) SEQ ID NO: 3, SEQ ID NO: 4, and SEQ ID NO: 5, respectively; (ii) SEQ ID NO: 13, SEQ ID NO: 14, and SEQ ID NO: 15, respectively; (iii) SEQ ID NO: 23, SEQ ID NO: 24, and SEQ ID NO: 25, respectively; and (iv) SEQ ID NO: 33, SEQ ID NO: 34, and SEQ ID NO: 35, respectively; and/or (b) the V_{L} comprises a V_{L}-CDR1 sequence, a V_{L}-CDR2 sequence, and a V_{L}-CDR3 sequence selected from the group consisting of (i) SEQ ID NO: 8, SEQ ID NO: 9, and SEQ ID NO: 10, respectively; (ii) SEQ ID NO: 18, SEQ ID NO: 19, and SEQ ID NO: 20, respectively; (iii) SEQ ID NO: 28, SEQ ID NO: 29, and SEQ ID NO: 30, respectively; and (iv) SEQ ID NO: 38, SEQ ID NO: 39, and SEQ ID NO: 40, respectively. In some embodiments, the anti-DLL3 antibody of the ADC may comprise one or more of the following characteristics: (a) a light chain immunoglobulin variable domain sequence that is at least 80%, at least 85%, at least 90%, at least 95%, or at least 99% identical to the light chain immunoglobulin variable domain sequence or light chain sequence present in any one of SEQ ID NOs: 7, 17, 27, 37, 62, 66, or 70; and/or (b) a heavy chain immunoglobulin variable domain sequence that is at least 80%, at least 85%, at least 90%, at least 95%, or at least 99% identical to the heavy chain immunoglobulin variable domain sequence or heavy chain sequence present in any one of SEQ ID NOs: 2, 12, 22, In some embodiments, the anti-DLL3 antibody of the ADC may comprise a heavy chain immunoglobulin variable domain (V_{H}) or heavy chain and a light chain immunoglobulin variable domain (V_{L}) or light chain, wherein: (a) the V_{H} or heavy chain comprises an amino acid sequence selected from the group consisting of SEQ ID NO: 2, SEQ ID NO: 12, SEQ ID NO: 22, SEQ ID NO: 32, SEQ ID NO: 59, SEQ ID NO: 60, SEQ ID NO: 61, SEQ ID NO: 63, SEQ ID NO: 64, SEQ ID NO: 65, SEQ ID NO: 67, SEQ ID NO: 68, and SEQ ID NO: 69; and/or (b) the V_{L} comprises an amino acid sequence selected from the group consisting of SEQ ID NO: 7, SEQ ID NO: 17, SEQ ID NO: 27, SEQ ID NO: 37, SEQ ID NO 62, SEQ ID NO: 66, and SEQ ID NO: 70. In some embodiments, the V_{H} or heavy chain amino acid sequence and the V_{L} or light chain amino acid sequence is selected from the group consisting of: SEQ ID NO: 2 and SEQ ID NO: 7 (7-I1-B), respectively; SEQ ID NO: 12 and SEQ ID NO: 17 (2-C8-A), respectively; SEQ ID NO: 59 and SEQ ID NO: 62 (H2-C8-A), respectively; SEQ ID NO: 60 and SEQ ID NO: 62 (H2-C8-A-2), respectively; SEQ ID NO: 61 and SEQ ID NO: 62 (H2-C8-A-3), respectively; SEQ ID NO: 22 and SEQ ID NO: 27 (10-O18-A), respectively; SEQ ID NO: 67 and SEQ ID NO: 70 (H10-O18-A), respectively; SEQ ID NO: 68 and SEQ ID NO: 70 (H10-O18-A-2), respectively; SEQ ID NO: 69 and SEQ ID NO: 70 (H10-O18-A-3); SEQ ID NO: 32 and SEQ ID NO: 37 (6-G23-F), respectively; SEQ ID NO: 63 and SEQ ID NO: 66 (H6-G23-F), respectively; SEQ ID NO: 64 and SEQ ID NO: 66 (H6-G23-F-2), respectively; and SEQ ID NO: 65 and SEQ ID NO: 66 (H6-G23-F-3), respectively.

Those skilled in the art could engineer the reaction method to conjugate a required number of drug molecules to each antibody molecule on the basis of the description in Examples herein, and obtain an antibody with a controlled number of conjugated pyrrolobenzodiazepine derivative molecules.

The antibody-drug conjugate, free drug, or production intermediate of the present invention may absorb moisture, allow adhesion of adsorbed water, or become a hydrate when being left to stand in the atmosphere or recrystallized, and such compounds and salts containing water are also included in the present invention.

The antibody-drug conjugate, free drug, or production intermediate of the present invention may be converted into a pharmaceutically acceptable salt, as desired, if having a basic group such as an amino group. Examples of such salts may include, but not limited to, hydrogen halide salts such as hydrochlorides and hydroiodides; inorganic acid salts such as nitrates, perchlorates, sulfates, and phosphates; lower alkanesulfonates such as methanesulfonates, trifluoromethanesulfonates, and ethanesulfonates; arylsufonates such as benzenesulfonates and p-toluenesulfonates; organic acid salts such as formates, acetates, malates, fumarates, succinates, citrates, tartrates, oxalates, and maleates; and amino acid salts such as ornithinates, glutamates, and aspartates.

If the antibody-drug conjugate, free drug, or production intermediate of the present invention has an acidic group such as a carboxy group, a base addition salt can be generally formed. Examples of pharmaceutical acceptable salts may include, but not limited to, alkali metal salts such as sodium salts, potassium salts, and lithium salts; alkali earth metal salts such as calcium salts and magnesium salts; inorganic salts such as ammonium salts; and organic amine salts such as dibenzylamine salts, morpholine salts, phenylglycine alkyl ester salts, ethylenediamine salts, N-methylglucamates, diethylamine salts, triethylamine salts, cyclohexylamine salts, dicyclohexylamine salts, N,N′-dibenzylethylenediamine salts, diethanolamine salts, N-benzyl-N-(2-phenylethoxy)amine salts, piperazine salts, tetramethylammonium salts, and tris(hydroxymethyl)aminomethane salts.

The antibody-drug conjugate, free drug, or production intermediate of the present invention may exist as a hydrate, for example, by absorbing moisture in the air. The solvate of the present invention is not limited to a particular solvate and may be any pharmaceutically acceptable solvate, and specifically hydrates, ethanol solvates, 2-propanol solvates, and so on are preferred. The antibody-drug conjugate, free drug, or production intermediate of the present invention may be its N-oxide form if a nitrogen atom is present therein, and these solvates and N-oxide forms are included in the scope of the present invention.

The present invention includes compounds labeled with various radioactive or nonradioactive isotopes. The antibody-drug conjugate, free drug, or production intermediate of the present invention may contain one or more constituent atoms with non-natural ratios of atomic isotopes. Examples of atomic isotopes may include, but not limited to, deuterium (²H), tritium (³H), iodine-125 (¹²⁵I), and carbon-14 (¹⁴C). The compound of the present invention may be radiolabeled with a radioactive isotope such as tritium (³H), iodine-125 (¹²⁵I), and carbon-14 (¹⁴C). The radiolabeled compound is useful as a therapeutic or prophylactic agent, a reagent for research such as an assay reagent, and a diagnostic agent such as a diagnostic agent for in vivo imaging. Isotopic variants of the antibody-drug conjugate of the present invention are all included in the scope of the present invention, regardless of whether they are radioactive or not.

E. Production Methods

Disclosed herein are representative methods for producing the antibody-drug conjugate of the present invention and free drugs or production intermediates thereof will be described. In the following, compound numbers shown in reaction formulas are used to identify compounds from each other. Specifically, reference in the form of “compound of formula (1)”, “compound (1)”, and so on will be made. Compounds with the other numbers will be indicated in the same manner.

Scheme R: Preparation of Antibody

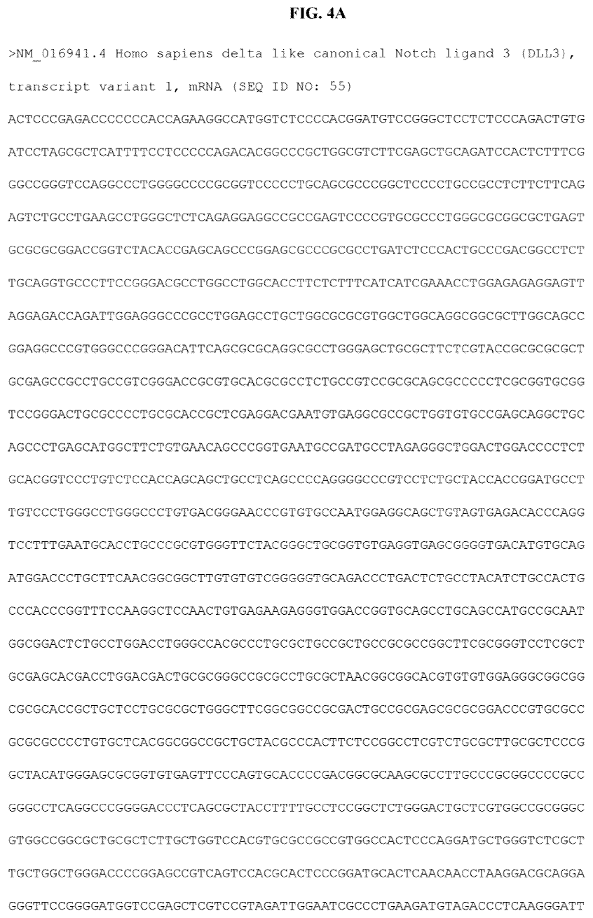

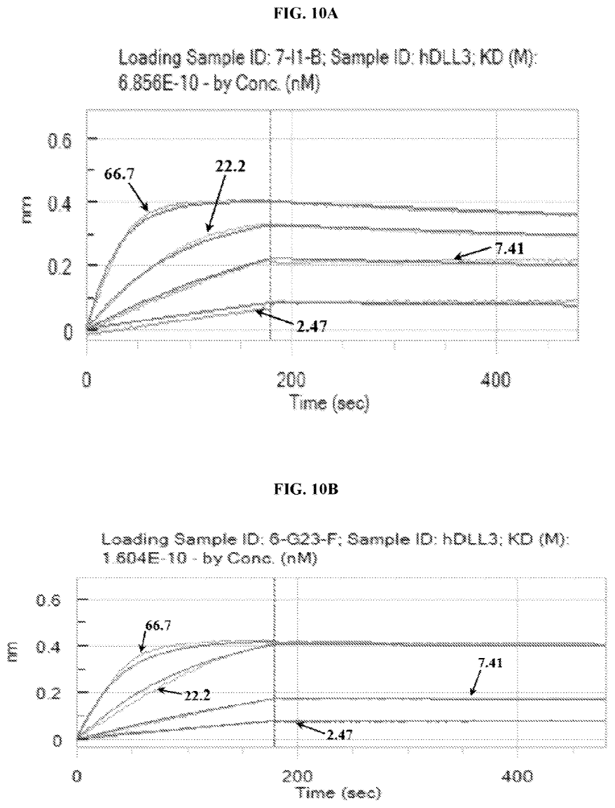

A glycan-remodeled antibody may be produced by using a method as illustrated in FIG. 3, for example, according to a method described in WO 2013/120066. The following descriptions of the Steps R1 and R2 of the synthesis are made in relation to FIG. 12 and the scheme below.

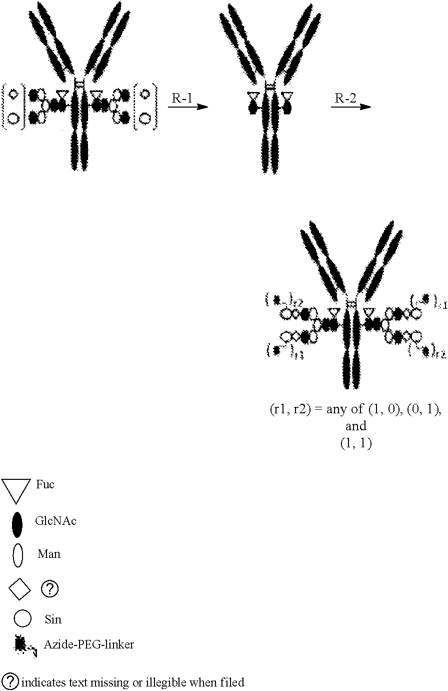

Step R-1: Hydrolysis of Glycosidic Bond at GlcNAcβ1-4GlcNAc of Chitobiose Structure at Reducing Terminal

The step is a step of preparing a glycan-truncated antibody by cleaving N-linked glycan bonding to asparagine at the 297-position of the amino acid sequence of a targeted antibody (N297-linked glycan) with use of a known enzymatic reaction.

A targeted antibody (20 mg/mL) in buffer solution (e.g., 50 mM phosphate buffer solution) is subjected to hydrolysis reaction of the glycosidic bond between GlcNAc31 and 4GlcNAc in the chitobiose structure at the reducing terminal with use of hydrolase such as the enzyme EndoS at 0° C. to 40° C. The reaction time is 10 minutes to 72 hours, and preferably 1 hour to 6 hours. The amount of the wild-type enzyme EndoS to be used is 0.1 to 10 mg, preferably 0.1 to 3 mg, to 100 mg of the antibody. After the completion of the reaction, purification with affinity chromatography and/or purification with a hydroxyapatite column, each described later, are/is carried out to produce a (Fucα1,6)GlcNAc antibody with the glycan hydrolyzed between GlcNAc31 and 4GlcNAc.

Step R-2: Transglycosylation Reaction

The step is a step of producing a glycan-remodeled antibody by bonding the (Fucα1,6)GlcNAc antibody to MSG- (MSG1-, MSG2-) or SG-type glycan oxazoline form (hereinafter, referred to as “azide glycan oxazoline form”) having a PEG linker including an azide group with use of enzymatic reaction.

The glycan-truncated antibody in buffer solution (e.g., phosphate buffer solution) is subjected to transglycosylation reaction by reacting with an azide glycan oxazoline form in the presence of a catalytic amount of transglycosidase such as EndoS (D233Q/Q303L) at 0° C. to 40° C. The reaction time is 10 minutes to 72 hours, and preferably 1 hour to 6 hours. The amount of the enzyme EndoS (D233Q/Q303L) to be used is 1 to 10 mg, preferably 1 to 3 mg, to 100 mg of the antibody, and the amount of the azide glycan oxazoline form to be used is 2 equivalents to an excessive equivalent, preferably 2 equivalents to 20 equivalents.

After the completion of the reaction, purification with affinity chromatography and purification with a hydroxyapatite column are carried out to afford a purified glycan-remodeled antibody.

The azide glycan oxazoline (ex.[N3-PEG (3)]-MSG1-Ox, Example 56 in WO19065964) form may be prepared according to methods described in Examples 55 to 57 in WO19065964. By using a reaction known in the field of synthetic organic chemistry (e.g., condensation reaction), N₃—(CH₂CH₂—O)n⁵-CH₂CH₂—NH₂, a PEG linker including an azide group (N₃-L(PEG)), may be introduced to MSG (MSG1, MSG2) or disialooctasaccharide (Tokyo Chemical Industry Co., Ltd.). Specifically, carboxylic acid at the 2-position of a sialic acid and the amino group at the right end of N₃—(CH₂CH₂—O)n⁵-CH₂CH₂—NH2 undergo condensation reaction to form an amide bond.

Examples of the condensing agent in using condensation reaction may include, but not limited to, N,N′-dicyclohexylcarbodiimide (DCC), 1-ethyl-3-(3-dimethylaminopropyl)carbodiimide (EDCI), carbonyldiimidazole (CDI), 2-(2H-benzotriazol-2-yl)-4-(1,1,3,3-tetramethylbutyl)phenol (BOP), 1H-benzotriazol-1-yloxytripyrrolidinophosphonium hexafluorophosphate (PyBOP), and O-(7-azabenzotriazol-1-yl)-N,N,N′,N′-tetramethyluronium hexafluorophosphate (HATU), and examples of the solvent for the reaction may include, but not limited to, dichloromethane, DMF, THF, ethyl acetate, and mixed solvent thereof.

The reaction temperature is typically −20° C. to 100° C. or the boiling point of the solvent, and preferably in the range of −5° C. to 50° C. As necessary, an organic base such as triethylamine, diisopropylethylamine, N-methylmorpholine, and 4-dimethylaminopyridine or an inorganic base such as potassium carbonate, sodium carbonate, potassium hydrogen carbonate, and sodium hydrogen carbonate may be added. Further, for example, 1-hydroxybenzotriazole or N-hydroxysuccinimide may be added as a reaction accelerator.

MSG, MSG1, or MSG2 may be obtained by hydrolysis of the (MSG-)Asn or separated/purified (MSG1-)Asn or (MSG2-)Asn with hydrolase such as EndoM.

Oxazolination may be prepared from GlcNAc at the reducing terminal of MSG-(MSG1-, MSG2-) or SG-type glycan according to a known article (J. Org Chem., 2009, 74(5), 2210-2212. Helv. Chim. Acta, 2012, 95, 1928-1936.).

Step R-3: Transglycosylation Reaction

Instead of Step R-2, the following step can be applied to produce the above glycan-remodeled antibody along with the method using two enzymes described in WO 2018/003983. The (Fucα1,6)GlcNAc antibody resulted from Step R-1 is applied to reaction with a glycan donor molecule which is (SG-)Asn, (MSG-)Asn and the like with an azide group introduced in sialic acid thereof, in the presence of two glycosyltransferases (enzyme A and enzyme B) to synthesize a SG type glycan-remodeled Fc-containing molecule with the azide group introduced in the sialic acid. Examples of enzyme A can include EndoM, EndoOm, and EndoCC, and EndoM mutants, EndoOm mutants, and EndoCC mutants that exhibit reduced hydrolyzing activity. Enzyme A is preferably EndoM N175Q, EndoCC N180H, or EndoOm N194Q. Examples of enzyme B can include EndoS and EndoS2 (EndoS49), and EndoS mutants and EndoS2 (EndoS49) mutants that exhibit reduced hydrolyzing activity. Enzyme B is preferably EndoS D233Q, EndoS D233Q/Q303L, EndoS D233Q/E350A, EndoS D233Q/E350Q, EndoS D233Q/E350D, EndoS D233Q/E350N, EndoS D233Q/D405A, EndoS2 D184M, EndoS2 T138Q or the like. In preparing the glycan-remodeled antibody, concentration of an aqueous solution of an antibody, measurement of concentration, and buffer exchange may be carried out according to common operations A to C in the following.

i. Common Operation A: Concentration of Aqueous Solution of Antibody

A solution of an antibody or antibody-drug conjugate was placed in a container of an Amicon Ultra (30,000 to 50,000 MWCO, Millipore Corporation), and the solution of an antibody or antibody-drug conjugate, which is described later, was concentrated through a centrifugation operation (centrifugation at 2000 G to 4000 G for 5 to 20 minutes) using a centrifuge (Allegra X-15R, Beckman Coulter, Inc.).

ii. Common Operation B: Measurement of Antibody Concentration

Measurement of antibody concentration was carried out by using a UV measurement apparatus (Nanodrop 1000, Thermo Fisher Scientific Inc.) according to a method specified by the manufacturer. Then, 280 nm absorption coefficients, being different among antibodies (1.3 mL mg⁻¹ cm⁻¹ to 1.8 mL mg⁻¹ cm⁻¹), were used.

iii. Common Operation C: Buffer Exchange for Antibody

A buffer solution (e.g., phosphate buffered saline (pH 6.0), phosphate buffer (pH 6.0)) was added to an aqueous solution of an antibody, which was concentrated according to common operation A. This operation was carried out several times, and the antibody concentration was then measured by using common operation B, and adjusted to 10-20 mg/mL with a buffer solution (e.g., phosphate buffered saline (pH 6.0), phosphate buffer (pH 6.0)).

Scheme S: Conjugation

The production method is a method for producing an antibody-drug conjugate by conjugating the above-described glycan-remodeled antibody to production intermediate (2) through SPAAC (strain-promoted alkyne azide cycloaddition: J. AM. CHEM. SOC. 2004, 126, 15046-15047) reaction. In the formula, Ab represents the glycan-remodeled antibody.

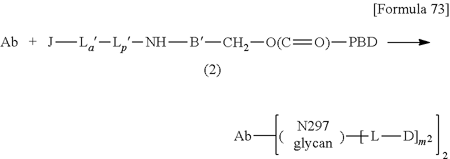

SPAAC reaction proceeds by mixing a buffer solution (sodium acetate solution, sodium phosphate, sodium borate solution, or the like, or a mixture thereof) of antibody Ab and a solution dissolving compound (2) in an appropriate solvent (dimethyl sulfoxide (DMSO), dimethylformamide (DMF), dimethylacetamide (DMA), N-methyl-2-pyridone (NMP), propylene glycol (PG), or the like, or a mixture thereof).

The amount of moles of compound (2) to be used is 2 mol to an excessive amount of moles, preferably 1 mol to 30 mol, per mole of the antibody, and the ratio of the organic solvent is preferably 1 to 200% v/v to the buffer of the antibody. The reaction temperature is 0° C. to 37° C., and preferably 10° C. to 25° C., and the reaction time is 1 to 150 hours, and preferably 6 hours to 100 hours. The pH in the reaction is preferably 5 to 9.

Antibody-drug conjugate compounds (ADCs) can be identified from each other through buffer exchange, purification, and measurement of antibody concentration and average number of conjugated drug molecules per antibody molecule according to common operations A to C described above and common operations D to F described later.

iv. Common Operation D: Purification of Antibody-Drug Conjugate

An NAP-25 column (GE Healthcare) was equilibrated with acetic acid buffer solution (10 mM, pH 5.5; herein, referred to as ABS) containing commercially available sorbitol (5%). To this NAP-25 column, an aqueous reaction solution of an antibody-drug conjugate (about 1.5 to 2.5 mL) was applied, and eluted with a buffer in an amount specified by the manufacturer to separate and collect an antibody fraction. The fraction separated and collected was again applied to the NAP-25 column, and a gel filtration purification operation to elute with a buffer was repeated twice or three times in total to afford the antibody-drug conjugate with an unbound drug-linker, dimethyl sulfoxide, and propylene glycol removed. As necessary, the concentration of the solution of the antibody-drug conjugate was adjusted through common operations A to C.

v. Common Operation E: Measurement of Antibody Concentration of Antibody-Drug Conjugate

The concentration of the conjugated drug in an antibody-drug conjugate can be calculated by using the Lambert-Beer's law shown below. Expression (I) using the Lambert-Beer's law is as follows.

$$\begin{matrix}[ Expression I ] & \\ \underline{{A}_{280}} = \underline{{ε}_{280} ( L \cdot {mol}^{- 1} \cdot {cm}^{- 1} )} \cdot \underline{C ( mol \cdot {L}^{- 1} )} \cdot \underline{l ( cm )} & Expression ( I )\end{matrix}$$

$$Absorbance = molar absorption coefficient \times Molarity \times Optical path length$$

Here, A280 denotes absorbance of an aqueous solution of an antibody-drug conjugate at 280 nm, 8280 denotes the molar absorption coefficient of an antibody-drug conjugate at 280 nm, and C (mol·L-1) denotes the molarity of an antibody-drug conjugate. From expression (I), the molarity of an antibody-drug conjugate, C (mol·L⁻¹), can be determined by using expression (II) below.

$$\begin{matrix}[ Expression II ] & \\ C ( mol \cdot {L}^{- 1} ) = \frac{{A}_{280}}{{ε}_{280} ( L \cdot {mol}^{- 1} \cdot {cm}^{- 1} ) \cdot l ( cm )} & Expression ( II )\end{matrix}$$

Further, the both sides are multiplied by the molar mass of the antibody-drug conjugate, MW (g·mol-1), to determine the weight concentration of the antibody-drug conjugate, C′ (mg·mL-1) (expression (III)).

$$\begin{matrix}[ Expression III ] & \\ {C}^{*} ( mg \cdot {mL}^{- 1} ) = M W ( g \cdot {mol}^{- 1} ) \cdot C ( mol \cdot {L}^{- 1} ) = \frac{{A}_{280} \cdot M W ( g \cdot {mol}^{- 1} )}{{ε}_{280} ( L \cdot {mol}^{- 1} \cdot {cm}^{- 1} ) \cdot l ( cm )} & Expression ( III )\end{matrix}$$

Values used for the expression and applied to Examples will be described.

The absorbance A280 used was a measured value of UV absorbance of an aqueous solution of an antibody-drug conjugate at 280 nm. For molar mass, MW (g·mol⁻¹), an estimated value of the molecular weight of an antibody was calculated from the amino acid sequence of the antibody, and used as an approximate value of the molar mass of an antibody-drug conjugate. The optical path length, 1 (cm), used in measurement was 1 cm.

The molar absorption coefficient, 8280, of the antibody-drug conjugate can be determined by using expression (IV) below.

$$\begin{matrix}[ Expression IV ] & \\ {ε}_{280} = \begin{matrix}molar absorption \\ coefficient of antibody\end{matrix} {ε}_{Ab , 280} + \begin{matrix}molar absorption \\ coefficient of drug\end{matrix} {ε}_{DL , 280} \times \begin{matrix}\begin{matrix}Number of \\ conjugated drug\end{matrix} \\ molecules\end{matrix} & Expression ( IV )\end{matrix}$$

Here, ε_{Ab, 280} denotes the molar absorption coefficient of an antibody at 280 nm, and ε_{DL, 280} denotes the molar absorption coefficient of a drug at 280 nm.

By using a known calculation method (Protein Science, 1995, vol. 4, 2411-2423), ε_{Ab, 280} can be estimated from the amino acid sequence of an antibody. In Examples, the molar absorption coefficient of the DLL3 antibody (H2-C8-A) used was ε_{Ab, 280}=220378 (calculated estimated value), the molar absorption coefficient of the DLL3 antibody (H6-G23-F) used was ε_{Ab, 280}=215353 (calculated estimated value), the molar absorption coefficient of the DLL3 antibody (H10-O18-A) used was ε_{Ab, 280}=215424 (calculated estimated value), the molar absorption coefficient of the LPS antibody used was ε_{Ab, 280}=230280 (calculated estimated value), the molar absorption coefficient of the DLL3 antibody (H2-C8-A-3) used was ε_{Ab, 280}=220420 (calculated estimated value), and the molar absorption coefficient of the DLL3 antibody (H10-O18-A-3) used was ε_{Ab, 280}=215380 (calculated estimated value).

ε_{DL, 280} was calculated for use from a measured value obtained in each UV measurement. Specifically, the absorbance of a solution dissolving a conjugate precursor (drug) with a certain molarity was measured, and expression (I), the Lambert-Beer's law, was applied thereto, and the resulting value was used.

vi. Common Operation F: Measurement of Average Number of Conjugated Drug Molecules Per Antibody Molecule in Antibody-Drug Conjugate

The average number of conjugated drug molecules per antibody molecule in an antibody-drug conjugate can be determined through high-performance liquid chromatography (HPLC) with the following method.

[F-1. Preparation of Sample for HPLC Analysis (Reduction of Antibody-Drug Conjugate)]

A solution of an antibody-drug conjugate (about 1 mg/mL, 60 μL) is mixed with an aqueous solution of dithiothreitol (DTT) (100 mM, 15 μL). The mixture is incubated at 37° C. for 30 minutes to prepare a sample in which the disulfide bond between the L chain and H chain of the antibody-drug conjugate cleaved, and this sample is used for HPLC analysis.

[F-2. HPLC Analysis]

HPLC analysis is carried out under the following conditions.
HPLC system: Agilent 1290 HPLC system (Agilent Technologies)
Detector: Ultraviolet absorption spectrometer (measurement wavelength: 280 nm, 329 nm)
Column: BEH Phenyl (2.1×50 mm, 1.7 μm, Waters Acquity)
Column temperature: 75° C.
Mobile phase A: 0.1% trifluoroacetic acid (TFA)-15% isopropyl alcohol aqueous solution
Mobile phase B: 0.075% TFA-15% isopropyl alcohol acetonitrile solution
Gradient program 1: 14%-36% (0 min to 15 min), 36%-80% (15 min to 17 min), 80%-14% (17 min to 17.1 min), 14%-14% (17.1 min to 23 min)
Gradient program 2: 14%-50% (0 min to 15 min), 50%-80% (15 min to 17 min), 80%-14% (17 min to 17.1 min), 14%-14% (17.1 min to 23 min)

Sample injection volume: 5 μL

[F-3. Data Analysis]

[F-3-1] An L chain with a conjugated drug molecule (L chain with one conjugated drug molecule: L₁) and H chain with a conjugated drug molecule(s) (H chain with one conjugated drug molecule: H₁, H chain with two conjugated drug molecules: H₂, H chain with three conjugated drug molecules: H₃) have hydrophobicity increased in proportion to the number of conjugated drug molecules and have longer retention time as compared to the L chain (L₀) and H chain (H₀) of an antibody without any conjugated drug molecule, and hence L₀, L₁, H₀, H₁, H₂, and H₃ are eluted in the presented order. While the order of L₁ and H₀ is inversed in some cases, H₀, which has no conjugated drug molecule, does not absorb at a wavelength of 329 nm characteristic to drugs. Therefore, L₁ and H₀ can be distinguished by checking absorption at a wavelength of 329 nm. Through comparison of retention time between L₀ and H₀, each peak detected can be assigned to L₀, L₁, H₀, H₁, H₂, or H₃.

[F-3-2] Since each drug-linker absorbs UV, peak area values are corrected by using the following expression with the molar absorption coefficients of an L chain, H chain, and drug-linker according to the number of conjugated drug-linker molecules.

$$\begin{matrix}Corrected L chain peak \\ area ( Li )\end{matrix} = Peak area \times \frac{Molar absorption coefficient of L chain}{\begin{matrix}\begin{matrix}Molar absorption \\ coefficient of L chain\end{matrix} + \begin{matrix}\begin{matrix}Number of \\ conjugated\end{matrix} \\ druge molecules\end{matrix} \times \\ \begin{matrix}Molar absorption coefficient \\ of drug - linker\end{matrix}\end{matrix}} \qquad [ Expression V ]$$

$$\begin{matrix}Corrected H chain peak \\ area ( Hi )\end{matrix} = Peak area \times \frac{Molar absorption coefficient of H chain}{\begin{matrix}\begin{matrix}Molar absorption \\ coefficient of H chain\end{matrix} + \begin{matrix}\begin{matrix}Number of \\ conjugated\end{matrix} \\ druge molecules\end{matrix} \times \\ \begin{matrix}Molar absorption coefficient \\ of drug - linker\end{matrix}\end{matrix}}$$

Here, for the molar absorption coefficients (280 nm) of the L chain and H chain of each antibody, values estimated from the amino acid sequences of the L chain and H chain of the antibody by using a known calculation method (Protein Science, 1995, vol. 4, 2411-2423) may be used. In the case of the DLL3 antibody (H2-C8-A), 26123 was used as the molar absorption coefficient of the L chain estimated from the amino acid sequence, and 84150 was used as the molar absorption coefficient of the H chain estimated from the amino acid sequence. In the case of the DLL3 antibody (H6-G23-F), 30105 was used as the molar absorption coefficient of the L chain estimated from the amino acid sequence, and 77423 was used as the molar absorption coefficient of the H chain estimated from the amino acid sequence. In the case of the DLL3 antibody (H10-O18-A), 26166 was used as the molar absorption coefficient of the L chain estimated from the amino acid sequence, and 81340 was used as the molar absorption coefficient of the H chain estimated from the amino acid sequence. In the case of the DLL3 antibody (H2-C8-A-3), 26212 was used as the molar absorption coefficient of the L chain estimated from the amino acid sequence, and 83993 was used as the molar absorption coefficient of the H chain estimated from the amino acid sequence. In the case of the DLL3 antibody (H10-O18-A-3), 26212 was used as the molar absorption coefficient of the L chain estimated from the amino acid sequence, and 81478 was used as the molar absorption coefficient of the H chain estimated from the amino acid sequence.

[F-3-3] The peak area ratio (%) of each chain to the total of corrected peak areas is calculated by using the following expression.

$$L chain peak area ratio = \frac{{A}_{Li}}{{A}_{L 0} + {A}_{L 1}} \times 100 \qquad [ Expression VI ]$$

$$H chain peak area ratio = \frac{{A}_{Hi}}{{A}_{H 0} + {A}_{H 1} + {A}_{H 2} + {A}_{H 3}} \times 100$$

$${A}_{Li} , {A}_{Hi} ; Li , Hi Respective corrected peak areas$$

[F-3-4] The average number of conjugated drug molecules per antibody molecule in an antibody-drug conjugate is calculated by using the following expression.

Average number of conjugated drug molecules=(L₀ peak area ratio×0+L₀ peak area ratio×1+H₀ peak area ratio×0+H₁ peak area ratio×1+H₂ peak area ratio×2+H₃ peak area ratio×3)/100×2.

### VII. Free Drug and Production Intermediate

The intermediate and free drug of the antibody-drug conjugate of the present invention is represented by the following formula:

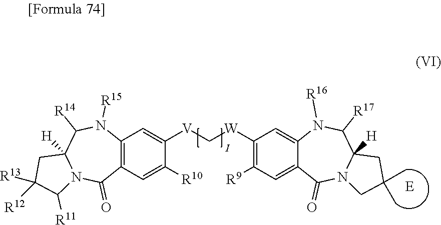

This will be described in the following.

The free drug of the present invention is generated through a process in which the antibody-drug conjugate migrates into tumor cells and the portion of linker L in the antibody-drug conjugate is then cleaved.

The antibody-drug conjugate of the present invention is produced by using the production intermediate.

The free drug for the antibody-drug conjugate of the present invention corresponds to the case in which (a) R¹⁶ and R¹⁷ are combined to form an imine bond (N═C).

The production intermediate for the antibody-drug conjugate of the present invention corresponds to the case in which (b) R¹⁶ is represented by J-La′-Lp′—NH—B′—CH₂—O(C═O)—*.

Accordingly, 1 and n¹, E and A, R⁹ and R¹, R¹⁰ and R², R¹¹ and R³, R¹² and R⁴, R¹³ and R⁵, R¹⁴ and R⁶, R¹⁵ and R⁷, V and X, W and Y, group 7 and group 1, group 8 and group 2, group 9 and group 3, group 10 and group 4, group 11 and group 5, and group 12 and group 6 in the formulas are respectively synonymous.

l represents an integer of 2 to 8, and is preferably an integer of 2 to 6, and more preferably an integer of 3 to 5. The alkyl chain with 1 being an integer of 2 to 8, preferably an integer of 2 to 6, and more preferably an integer of 3 to 5, may include a double bond.

E represents a spiro-bonded three- to five-membered saturated hydrocarbon ring or a three- to five-membered saturated heterocycle, and is preferably a three- to five-membered saturated hydrocarbon ring (cyclopropane, cyclobutane, or cyclopentane), more preferably cyclopropane or cyclobutane, and most preferably cyclopropane. The spiro-bonded three- to five-membered saturated hydrocarbon ring may be substituted with one to four halogen atoms, and may be preferably substituted with one or two fluorine atoms (e.g., 2,2-difluorocyclopropane).

R⁹ and R¹⁰ each independently represent a C1 to C6 alkoxy group, a C1 to C6 alkyl group, a hydrogen atom, a hydroxy group, a thiol group, a C1 to C6 alkylthio group, a halogen atom, or —NR′R″, and are each preferably a C1 to C6 alkoxy group, a C1 to C6 alkyl group, or a hydroxy group, more preferably a C1 to C3 alkoxy group, and most preferably a methoxy group.

R″, R¹², and R¹³ are as described in any of the following (i) to (iii).

### A. Compound Embodiment One

If R″ and R¹² are combined together with the carbon atoms to which R³ and R⁴ are bound to form a double bond, R¹³ represents an aryl group or heteroaryl group optionally having one or more substituents selected from group 7 or a C1 to C6 alkyl group optionally having one or more substituents selected from group 8, and is preferably an aryl group optionally having one or more substituents selected from group 7.

“Aryl group” in “aryl group or heteroaryl group optionally having one or more substituents selected from group 7” for R¹³ is preferably a phenyl group or a naphthyl group, and more preferably a phenyl group.

“Heteroaryl group” in “aryl group or heteroaryl group optionally having one or more substituents selected from group 7” for R¹³ is preferably a thienyl group, a pyridyl group, a pyrimidyl group, a quinolyl group, a quinoxalyl group, or a benzothiophenyl group, more preferably a 2-thienyl group, a 3-thienyl group, a 2-pyridyl group, a 3-pyridyl group, or a 4-pyridyl group, and even more preferably a 3-pyridyl group or a 3-thienyl group.

Examples of substituents of the aryl group or heteroaryl group for R¹³ may include, but are not limited to, the following a) to j):
a) a C1 to C6 alkoxy group optionally substituted with one to three halogen atoms,
b) a C1 to C6 alkyl group optionally substituted with any one selected from one to three halogen atoms, a hydroxy group, —OCOR′, —NR′R″, —C(═NR′)—NR″R′″, and —NHC(═NR′)—NR″R′″,
c) a halogen atom,
d) a C3 to C5 cycloalkoxy group,
e) a C1 to C6 alkylthio group,
f) —NR′R″,
g) —C(═NR′)—NR″R′″,
h) —NHC(═NR′)—NR″R′″,
i) —NHCOR′, and
j) a hydroxy group,

Here, R′, R″, and R′″ in b) and f) to i) each independently represent a hydrogen atom or a C1 to C6 alkyl group, and are preferably each independently a hydrogen atom or a C1 to C3 alkyl group.

a) to j) are preferably as follows:
a) a C1 to C3 alkoxy group optionally substituted with one to three halogen atoms, more preferably a methoxy group, an ethoxy group, a n-propoxy group, an i-propoxy group, or a trifluoromethoxy group, even more preferably a methoxy group, an ethoxy group, or a trifluoromethoxy group, most preferably a methoxy group;
b) a C1 to C3 alkyl group optionally substituted with any selected from one to three halogen atoms, a hydroxy group, —OCOR′, —C(═NR′)—NR″R′″, and —NHC(═NR′)—NR″R′″, wherein R′, R″, and R′″ are each independently a hydrogen atom or a C1 to C3 alkyl group, more preferably a C1 to C3 alkyl group optionally substituted with any selected from one to three halogen atoms, a hydroxy group, —OCOR′, —C(═NR′)—NR″R′″, and —NHC(═NR′)—NR″R′″, wherein R′, R″, and R′″ are each independently a hydrogen atom or a methyl group, even more preferably a methyl group, an ethyl group, a n-propyl group, an i-propyl group, a fluoromethyl group, a difluoromethyl group, a trifluoromethyl group, a hydroxymethyl group, —CH₂OCOMe, —CH₂—NHC(═NH)—NH₂, or —CH₂—NHC(═NMe)-NH₂;
c) a halogen atom, preferably a fluorine atom or a chlorine atom;
d) a C3 to C5 cycloalkoxy group, more preferably a cyclopropoxy group;
e) a C1 to C3 alkylthio group, more preferably a methylthio group or an ethylthio group;
f) —NR′R″, wherein R′ and R″ are each independently a hydrogen atom or a C1 to C3 alkyl group, more preferably —NH₂, —NHMe, —NMe₂, —NHEt, or -NEt₂;
g) —C(═NR′)—NR″R′″, wherein R′, R″, and R′″ are each independently a hydrogen atom or a C1 to C3 alkyl group, more preferably —C(═NH)—NH₂ or —C(═NMe)-NH₂;
h) —NHC(═NR′)—NR″R′″, wherein R′, R″, and R′″ are each independently a hydrogen atom or a C1 to C3 alkyl group, more preferably —NHC(═NH)—NH₂ or —NHC(═NMe)-NH₂;
i) —NHCOR′, wherein R′ is a hydrogen atom or a C1 to C3 alkyl group, more preferably —NHCOMe or -NHCOEt; and
j) a hydroxy group.

The aryl group (preferably, a phenyl group) or heteroaryl group (preferably, a pyridyl group) for R¹³ may have at least one substituent at any position. If a plurality of substituents is present, the substituents may be the same or different.

If R¹³ is an aryl group, each substituent is preferably a), b), d), g), h), or j), and more preferably a), b), d), or j).

If R¹³ is a phenyl group, R¹³ may have a substituent at any position and may have a plurality of substituents, and preferably one or two substituents are present at the 3-position and/or the 4-position, and more preferably one substituent is present at the 4-position.

If R⁵ is a naphthyl group, R⁵ may have a substituent at any position and may have a plurality of substituents, and preferably one substituent is present at the 6-position.

If R¹³ is a phenyl group, R¹³ is more preferably a phenyl group, a 4-methoxyphenyl group, a 3-methoxyphenyl group, a 4-ethoxyphenyl group, a 4-(n-propoxy)-phenyl group, a 4-(i-propoxy)-phenyl group, a 4-cyclopropoxy-phenyl group, a 4-trifluoromethylphenyl group, a 4-hydroxymethyl-phenyl group, a 4-acetoxymethyl-phenyl group, or a 4-carbamimidamidomethyl-phenyl group, and even more preferably a phenyl group, a 4-methoxyphenyl group, a 3-methoxyphenyl group, a 4-cyclopropoxy-phenyl group, a 4-hydroxymethyl-phenyl group, a 4-acetoxymethyl-phenyl group, a 4-carbamimidamidomethyl-phenyl group, or a 4-trifluoromethylphenyl group.

If R¹³ is a naphthyl group, R¹³ is more preferably a naphthyl group or a 6-methoxy-2-naphthyl group. The most preferred is a 4-methoxyphenyl group.

If R¹³ is a heteroaryl group, each substituent is preferably a), b), d), g), h), or j), and more preferably a) or b).

If R¹³ is a heteroaryl group, R13 may have at least one substituent at any position. If R¹³ is a 3-pyridyl group, its substituent(s) is preferably present at the 6-position and/or the 5-position. If R¹³ is 2-pyridyl, its substituent(s) is preferably present at the 5-position and/or the 4-position or at the 5-position and/or the 6-position. If R¹³ is 4-pyridyl, its substituent is preferably present at the 2-position and/or the 6-position.

If R¹³ is a heteroaryl group, R¹³ may have a plurality of substituents, and preferably has one or two substituents, and preferably has one substituent.

If R¹³ is a pyridyl group, R¹³ is preferably a 6-methoxy-3-pyridyl group or a 6-methyl-3-pyridyl group.

If R¹³ is a 3-thienyl group or a 6-quinoxalyl group, R¹³ is preferably unsubstituted.

“C1 to C6 alkyl group” in “C1 to C6 alkyl group optionally having one or more substituents selected from group 8” for R¹³ is preferably a C1 to C3 alkyl group, and more preferably a methyl group or an ethyl group.

The substituents in “C1 to C6 alkyl group optionally having one or more substituents selected from group 8” for R¹³ are each a halogen atom, a hydroxy group, or a C1 to C6 alkoxy group (preferably, a C1 to C3 alkoxy group), preferably a hydroxy group, a methoxy group, or an ethoxy group, and more preferably a hydroxy group.

### B. Compound Embodiment Two

If R¹¹ represents a hydrogen atom, R¹² and R¹³ are combined, together with the carbon atom to which R¹² and R¹³ are bound, to form a three- to five-membered saturated hydrocarbon ring or a three- to five-membered saturated heterocycle, or CH₂═.

The three- to five-membered saturated hydrocarbon ring may be substituted with one to four halogen atoms, and may be preferably substituted with one or two fluorine atoms.

R¹² and R¹³ are preferably combined to form a three- to five-membered saturated hydrocarbon ring or CH₂═, more preferably to form cyclopropane, cyclobutane, or CH₂═(exomethylene group), and even more preferably to form cyclopropane.

If R¹² and R¹³ are combined to form a three- to five-membered saturated hydrocarbon ring or a three- to five-membered saturated heterocycle, the three- to five-membered saturated hydrocarbon ring or a three- to five-membered saturated heterocycle is preferably the same as E. More preferably, E is a three- to five-membered saturated hydrocarbon ring and R¹² and R¹³ are combined to form a three- to five-membered saturated hydrocarbon ring, and even more preferably E is a cyclopropane ring and R¹² and R¹³ are combined to form a cyclopropane ring.

### C. Compound Embodiment Three

R¹¹, R¹², and R¹³ are combined, together with the carbon atom to which R″ is bound and the carbon atom to which R¹² and R¹³ are bound, to form a benzene ring or six-membered heterocycle optionally having one or more substituents selected from group 9.

The benzene ring or heterocycle may have at least one substituent at any position. If a plurality of substituents is present, the substituents may be the same or different.

Each substituent of the benzene ring or heterocycle is a halogen atom, a C1 to C6 alkyl group optionally substituted with one to three halogen atoms, or a C1 to C6 alkoxy group, preferably a halogen atom, a C1 to C3 alkyl group optionally substituted with one to three halogen atoms, or a C1 to C3 alkoxy, and more preferably a halogen atom, a methyl group, or a methoxy group.

“Benzene ring or six-membered heterocycle optionally having one or more substituents” is preferably an unsubstituted benzene ring.

R¹¹, R¹² and R¹³ most preferably satisfy the above (i).

### D. Other Variable Groups and Embodiments

R¹⁴ and R¹⁵ each represent a hydrogen atom, or R¹⁴ and R¹⁵ are combined to represent an imine bond (C═N).

V and W are each independently an oxygen atom, a nitrogen atom, or a sulfur atom, and preferably an oxygen atom.

R¹⁶ and R¹⁷ are such that:
(a) R¹⁶ and R¹⁷ are combined to form an imine bond (N═C); or
(b) R¹⁶ represents J-La′-Lp′—NH—B′—CH₂—O(C═O)—* and R¹⁷ represents a hydroxy group or a C1 to C3 alkoxy group.

In the case of (b) R¹⁶ is J-La′-Lp′—NH—B′—CH₂—O(C═O)—*, the asterisk in the formula represents bonding to the N10′-position of the pyrrolobenzodiazepine ring represented by the above formula.

B′ represents a phenyl group or a heteroaryl group, and is preferably a 1,4-phenyl group, a 2,5-pyridyl group, a 3,6-pyridyl group, a 2,5-pyrimidyl group, or a 2,5-thienyl group, and more preferably a 1,4-phenyl group.

Lp′ represents a linker consisting of an amino acid sequence cleavable in vivo or in a target cell. Lp is, for example, cleaved by the action of an enzyme such as esterase and peptidase.

Specific examples of linker Lp′ may include, but are not limited to, -GGVA- (SEQ ID NO: 85), -GG-(D-)VA-, -VA-, -GGFG- (SEQ ID NO: 86), -GGPI- (SEQ ID NO: 87), -GGVCit- (SEQ ID NO: 88), -GGVK- (SEQ ID NO: 89), -GG(D-)PI-, -GGPL- (SEQ ID NO: 90), -EGGVA (SEQ ID NO: 91), -PI-, -GGF-, DGGF- (SEQ ID NO: 92), (D-)D-GGF-, -EGGF- (SEQ ID NO: 93), -SGGF- (SEQ ID NO: 94), -KGGF- (SEQ ID NO: 95), -DGGFG- (SEQ ID NO: 96), -GGFGG- (SEQ ID NO: 97), -DDGGFG- (SEQ ID NO: 98), -KDGGFG- (SEQ ID NO: 99), and -GGFGGGF- (SEQ ID NO: 100).

Here, “(D-)V” indicates D-valine, “(D)-P” indicates D-proline, and “(D-)D” indicates D-aspartic acid.

Linker Lp′ is preferably as follows:

(SEQ ID NO: 85)
-GGVA-,
-GG-(D-)VA-,
-VA-,
(SEQ ID NO: 86)
-GGFG-,
(SEQ ID NO: 87)
-GGPI-,
(SEQ ID NO: 88)
-GGVCit-,
(SEQ ID NO: 89)
-GGVK-,
-GG(D-)PI-,
or
(SEQ ID NO: 90)
-GGPL-.

More preferred examples are -GGVA- (SEQ ID NO: 85), -GGVCit- (SEQ ID NO: 88), and -VA-.

La′ represents any one selected from the following group: —C(═O)—(CH₂CH₂)n⁶-C(═O)—, —C(═O)—(CH₂CH₂)n⁶-C(═O)—NH—(CH₂CH₂)n⁷-C(═O)—, —C(═O)—(CH₂CH₂)n⁶-C(═O)—NH—(CH₂CH₂O)n⁷-CH₂—C(═O)—, —C(═O)—(CH₂CH₂)n⁶-NH—C(═O)—(CH₂CH₂O)n⁷-CH₂CH₂—C(═O)—, —(CH₂)n⁸-O—C(═O)—, —(CH₂)n¹²-C(═O)—, and, —(CH₂CH₂)n¹³-C(═O)—NH—(CH₂CH₂O)n¹⁴-CH₂CH₂—C(═O)—

In the formulas, n⁶ represents an integer of 1 to 3 (preferably, 1 or 2), n⁷ represents an integer of 1 to 5 (preferably, an integer of 2 to 4, more preferably, 2 or 4), n⁸ represents an integer of 0 to 2 (preferably, 0 or 1), n¹² represents an integer of 2 to 7 (preferably, an integer of 2 to 5, more preferably, 2 or 5), n¹³ represents an integer of 1 to 3 (preferably, 1), and n14 represents an integer of 6 to 10 (preferably, 8).

La′ preferably represents any one selected from the following group: —C(═O)—CH₂CH₂—C(═O)—, —C(═O)—(CH₂CH₂)₂—C(═O)—, —C(═O)—CH₂CH₂—C(═O)—NH—(CH₂CH₂)₂—C(═O)—, —C(═O)—CH₂CH₂—C(═O)—NH—(CH₂CH₂O)₂—CH₂—C(═O)—, —C(═O)—CH₂CH₂—NH—C(═O)—(CH₂CH₂O)₄—CH₂CH₂—C(═O)—, —CH₂—OC(═O)—, —OC(═O)—, —(CH₂)₂—C(═O)—, —(CH₂)₅—C(═O)—, and —CH₂CH₂—C(═O)—NH—(CH₂CH₂O)₈—CH₂CH₂—C(═O)—.

La′ is more preferably —C(═O)—CH₂CH₂—C(═O)—, —C(═O)—(CH₂CH₂)₂—C(═O)—, or —(CH₂)₅—C(═O)—.

J is not limited to a particular structure and may be any cyclic structure including an alkyne structure that reacts with an azide group to form a 1,2,3-triazole ring, and examples thereof may include, but are not limited to, compounds represented by the following formulas:

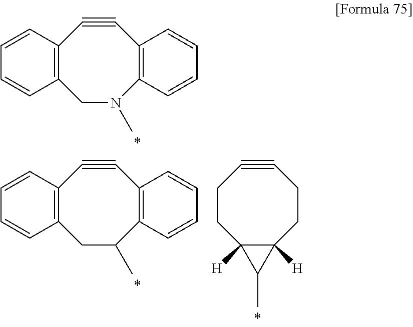

In the structural formulas for J shown above, each asterisk represents bonding to —(C═O) or —(CH₂)n⁸ at the left end of La′.

Alternatively, J may be a compound that bonds to a side chain of an amino acid residue (e.g., cysteine, or lysine) of antibody Ab, or a halogen atom, and examples of J may include, but are not limited to, a maleimidyl group represented by the following formula:

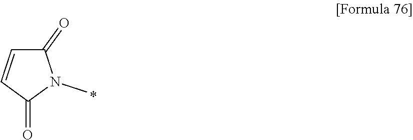

In the maleimidyl group shown above, the asterisk represents bonding to —(CH₂)n¹² or —(CH₂CH₂)n¹³ at the left end of La′.

R¹⁶ is preferably represented by J-La′-Lp′—NH—B′—CH₂—O(C═O)—*, wherein
B′ is a 1,4-phenyl group;
Lp′ represents any one selected from the following group:

(SEQ ID NO: 85)
-GGVA-,
-GG-(D-)VA-,
-VA-,
(SEQ ID NO: 86)
-GGFG-,
(SEQ ID NO: 87)
-GGPI-,
(SEQ ID NO: 88)
-GGVCit-,
(SEQ ID NO: 89)
-GGVK-,
GG(D-)PI-,
and
(SEQ ID NO: 90)
-GGPL-;

La′ represents any one selected from the following group: —C(═O)—CH₂CH₂—C(═O)—, —C(═O)—(CH₂CH₂)₂—C(═O)—, —C(═O)—CH₂CH₂—C(═O)—NH—(CH₂CH₂)₂—C(═O)—, —C(═O)—CH₂CH₂—C(═O)—NH—(CH₂CH₂O)₂—CH₂—C(═O)—, —C(═O)—CH₂CH₂—NH—C(═O)—(CH₂CH₂O)₄—CH₂CH₂—C(═O)—, —OC(═O)—, —CH₂—OC(═O)—, —(CH₂)₅—C(═O)—, and —CH₂CH₂—C(═O)—NH—(CH₂CH₂O)₈—CH₂CH₂—C(═O)—; and
J represents any of the structural formulas:

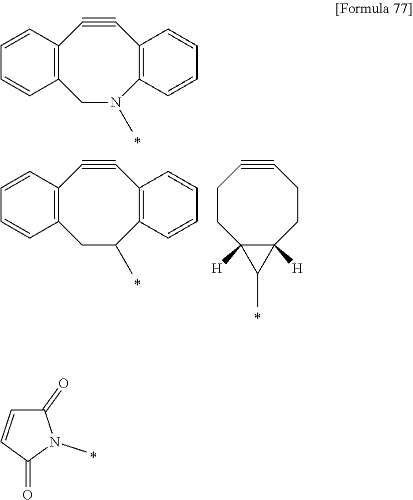

wherein, in the structural formulas for J,
each asterisk represents bonding to La′.

R¹⁶ is more preferably any one selected from the following group:
J¹-C(═O)—CH₂CH₂—C(═O)-GGVA-NH—B′—CH₂—OC(═O)— (“GGVA” disclosed as SEQ ID NO: 85),
J¹-C(═O)—CH₂CH₂—C(═O)-GG-(D-)VA-NH—B′—CH₂—OC(═O)—,
J¹-C(═O)—CH₂CH₂—C(═O)—VA-NH—B′—CH₂—OC(═O)—,
J¹-C(═O)—(CH₂CH₂)₂—C(═O)—VA-NH—B′—CH₂—OC(═O)—,
J¹-C(═O)—CH₂CH₂—C(═O)-GGPI-NH—B′—CH₂—OC(═O)— (“GGPI” disclosed as SEQ ID NO: 87),
J¹-C(═O)—CH₂CH₂—C(═O)-GGFG-NH—B′—CH₂—OC(═O)— (“GGFG” disclosed as SEQ ID NO: 86),
J¹-C(═O)—CH₂CH₂—C(═O)-GGVCit-NH—B′—CH₂—OC(═O)— (“GGVCit” disclosed as SEQ ID NO: 88),
J¹-C(═O)—CH₂CH₂—C(═O)-GGVK-NH—B′—CH₂—OC(═O)— (“GGVK” disclosed as SEQ ID NO: 89),
J¹-C(═O)—CH₂CH₂—C(═O)-GGPL-NH—B′—CH₂—OC(═O)— (“GGPL” disclosed as SEQ ID NO: 90),
J¹-C(═O)—CH₂CH₂—C(═O)—NH—(CH₂CH₂)₂—C(═O)—VA-NH—B′—CH₂—OC(═O)—,
J¹-C(═O)—CH₂CH₂—C(═O)—NH—(CH₂CH₂O)₂—CH₂—C(═O)—VA-NH—B′—CH₂—OC(═O)—,
J¹-C(═O)—CH₂CH₂—NH—C(═O)—(CH₂CH₂O)₄—CH₂CH₂—C(═O)—VA-NH—B′—CH₂—OC(═O)—,
J²-OC(═O)-GGVA-NH—B′—CH₂—OC(═O)— (“GGVA” disclosed as SEQ ID NO: 85), J³-CH₂—OC(═O)-GGVA-NH—B′—CH₂—OC(═O)— (“GGVA” disclosed as SEQ ID NO: 85),
J⁴-(CH₂)₅—C(═O)-GGVA-NH—B′—CH₂—OC(═O)— (“GGVA” disclosed as SEQ ID NO: 85),
J⁴-(CH₂)₅—C(═O)—VA-NH—B′—CH₂—OC(═O)—, and
J⁴-CH₂CH₂—C(═O)—NH—(CH₂CH₂O)₈—CH₂CH₂—C(═O)—VA-NH—B′—CH₂—OC(═O)—
   wherein J¹, n, J³, and J⁴ represent structural formulas represented by the following:

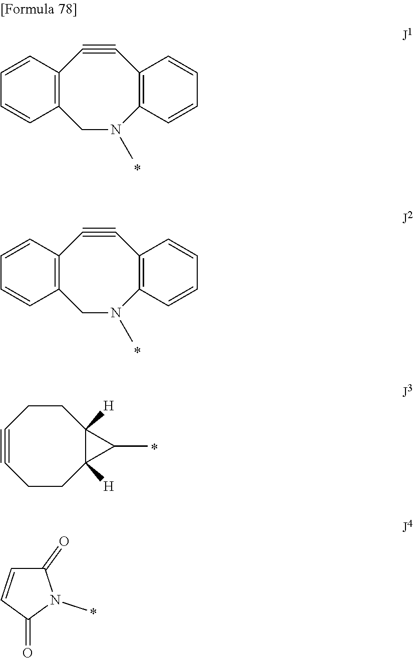

wherein, in the structural formulas for J¹, J², J³ and J⁴,
each asterisk represents bonding to a group neighboring to J¹, J², J³, or J⁴, and B′ is a 1,4-phenyl group.

R¹⁶ is most preferably any of the following:
J¹-C(═O)—CH₂CH₂—C(═O)-GGVA-NH—B′—CH₂—OC(═O)— (“GGVA” disclosed as SEQ ID NO: 85),
J¹-C(═O)—CH₂CH₂—C(═O)—VA-NH—B′—CH₂—OC(═O)—,
J¹-C(═O)—(CH₂CH₂)₂—C(═O)—VA-NH—B′—CH₂—OC(═O)—,
J¹-C(═O)—CH₂CH₂—C(═O)-GGVCit-NH—B′—CH₂—OC(═O)— (“GGVCit” disclosed as SEQ ID NO: 88),
J¹-C(═O)—CH₂CH₂—C(═O)—NH—(CH₂CH₂)₂—C(═O)—VA-NH—B′—CH₂—OC(═O)—,
J¹-C(═O)—CH₂CH₂—C(═O)—NH—(CH₂CH₂O)₂—CH₂—C(═O)—VA-NH—B′—CH₂—OC(═O)—,
J¹-C(═O)—CH₂CH₂—NH—C(═O)—(CH₂CH₂O)₄—CH₂CH₂—C(═O)—VA-NH—B′—CH₂—OC(═O)—,
J⁴-(CH₂)₅—C(═O)—VA-NH—B′—CH₂—OC(═O)—
   wherein
B′ is a 1,4-phenyl group, and
J¹ and J⁴ are represented by the following structural formulas for J:

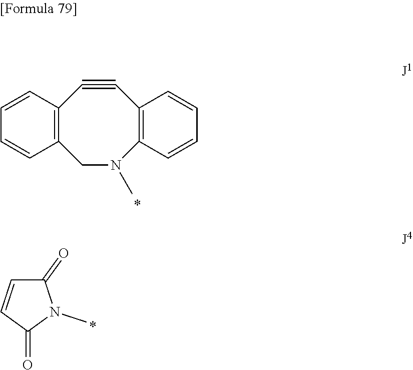

wherein, in the structural formulas for J¹ and J⁴,
each asterisk represents bonding to a group neighboring to J¹ or J⁴.
   R¹⁷ is a hydroxy group or a C1 to C3 alkoxy group, and preferably a hydroxy group or a methoxy group. R17 may be hydrogensulfite adduct (OSO3M, wherein M is a metal cation). Since R17 bonds to an asymmetric carbon atom, a steric configuration represented by partial structure (VIa) or (VIb) below is provided. Each wavy line represents bonding to W in the intermediate and free drug represented by general formula (VI).

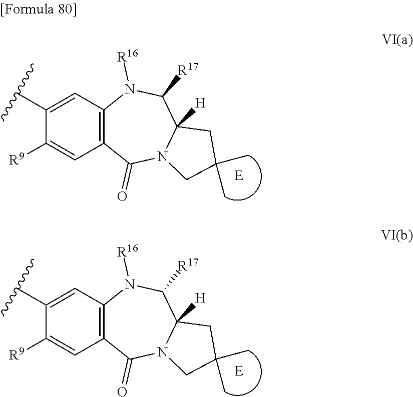

The free drug is preferably one compound selected from the following group:

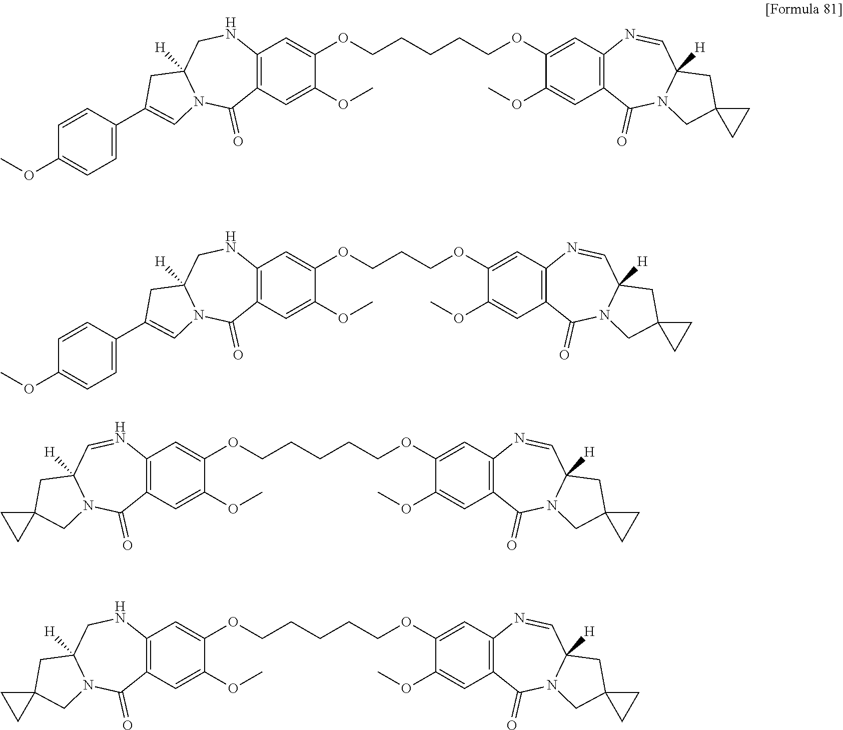

The free drug is in some cases released in tumor cells with a part of linker L bonded, but is a superior drug that exerts superior antitumor effect even in such state. The free drug, after migrating to tumor cells, is in some cases further oxidized to cause dehydrogenation of R¹⁶ and R¹⁷, but exerts superior antitumor effect even in such state.

The production intermediate may also be one compound selected from the following group:

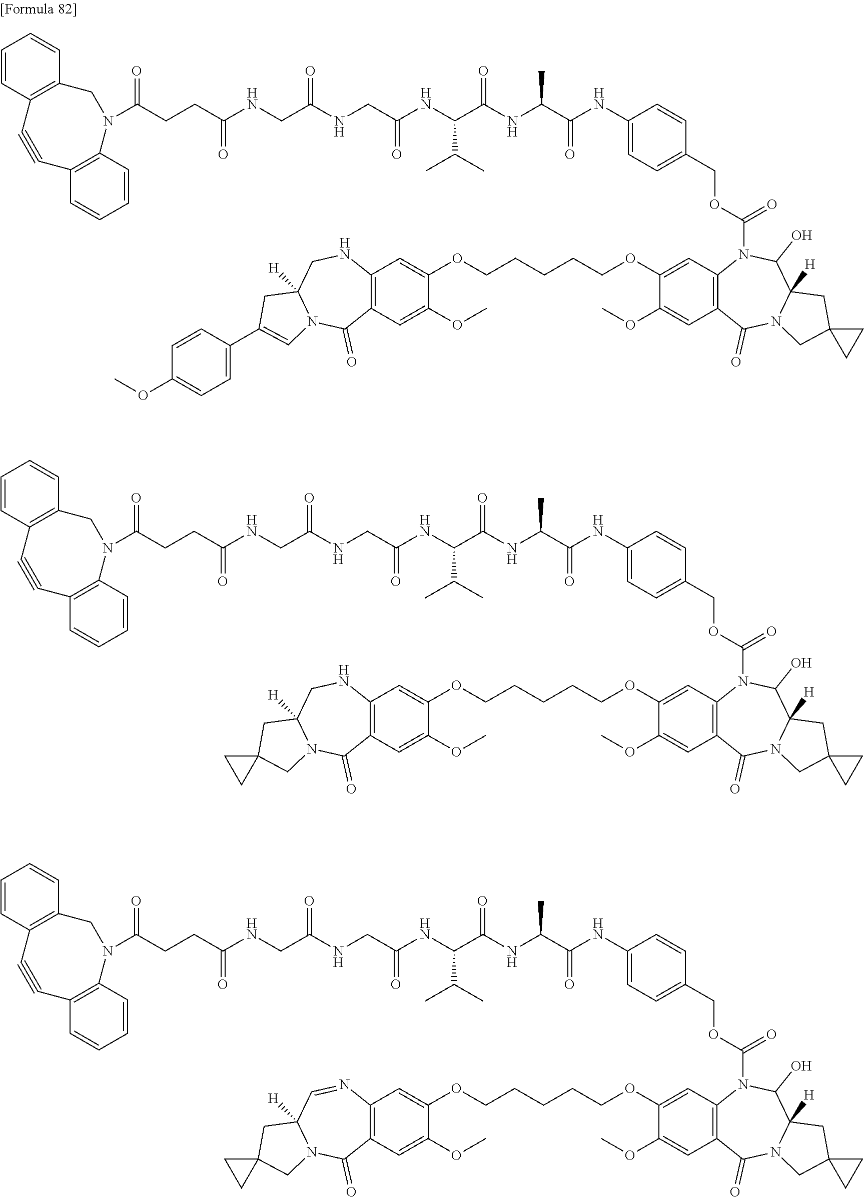

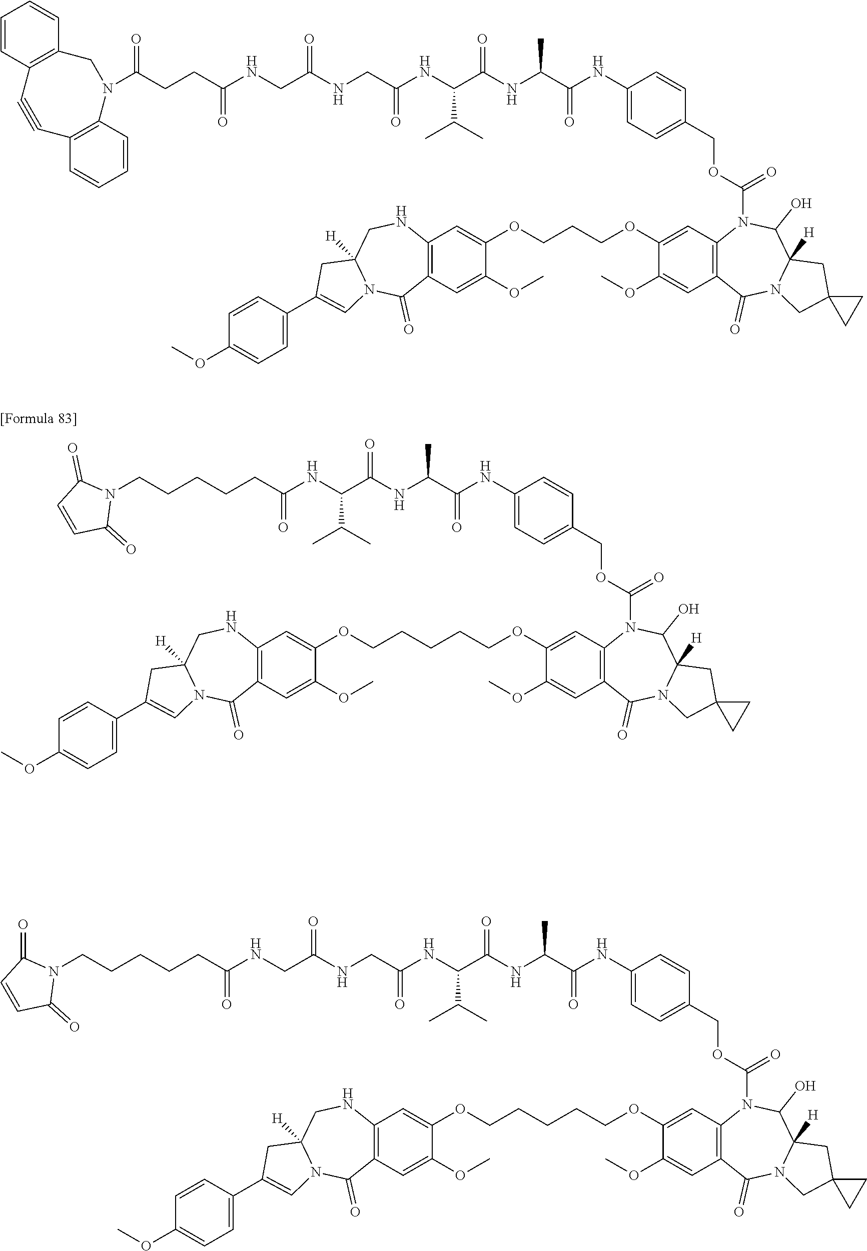

The production intermediate may also be one compound selected from the following group:

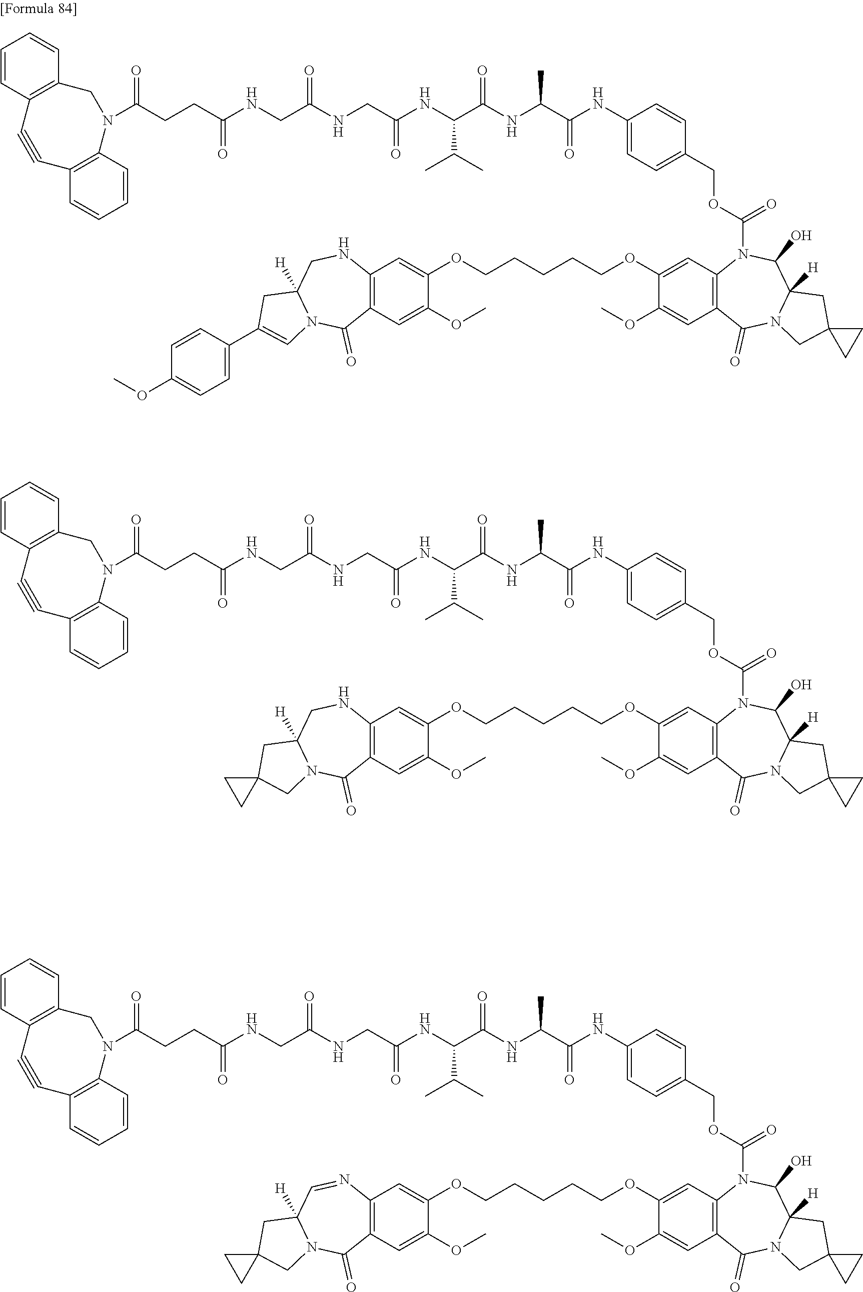

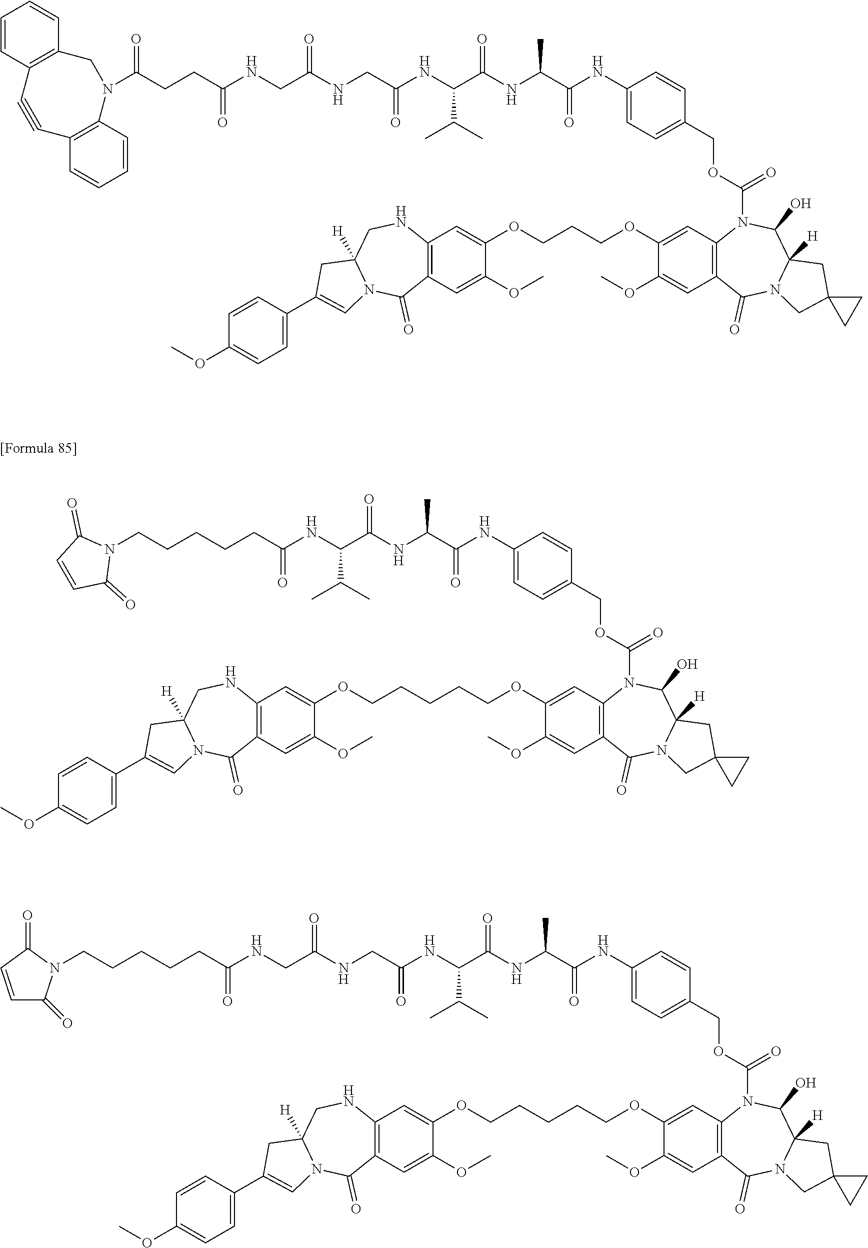

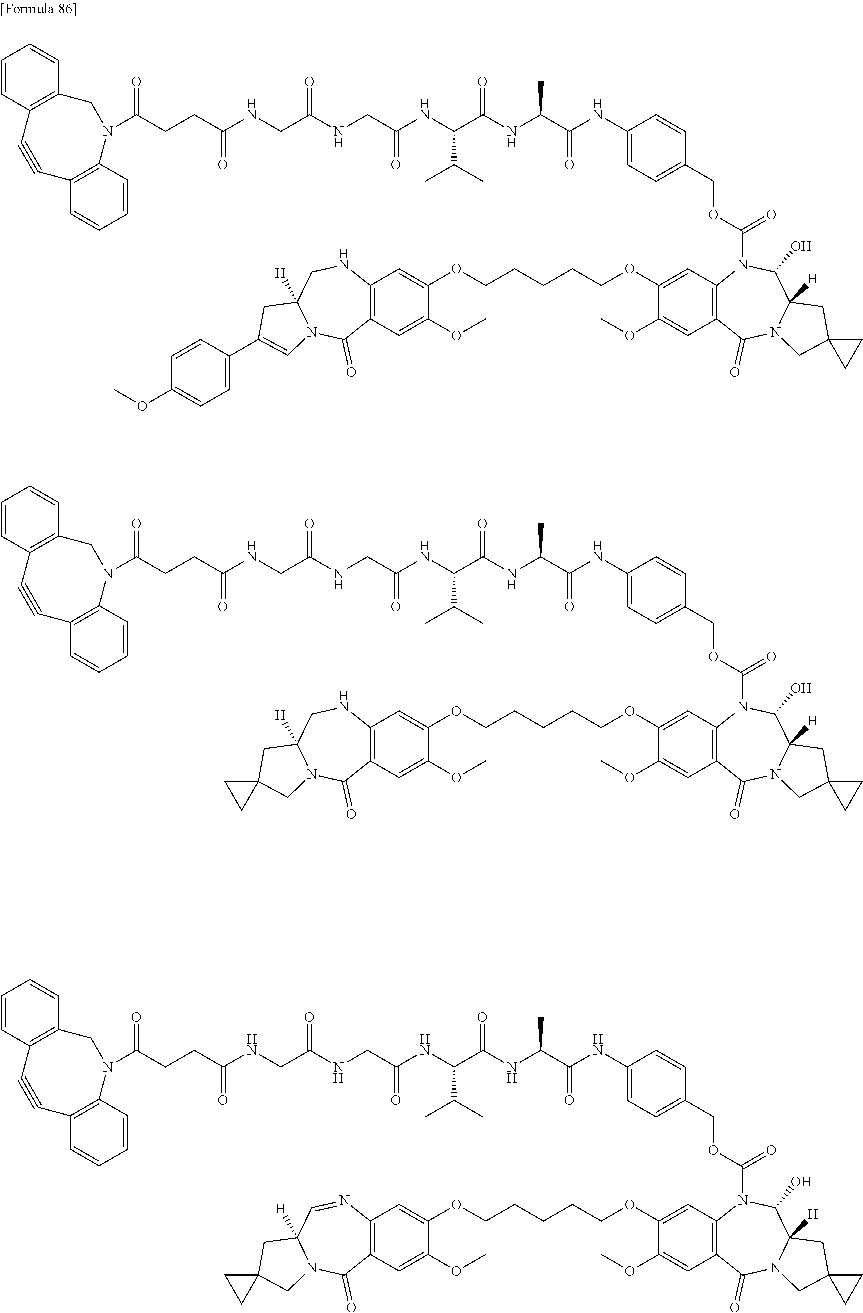

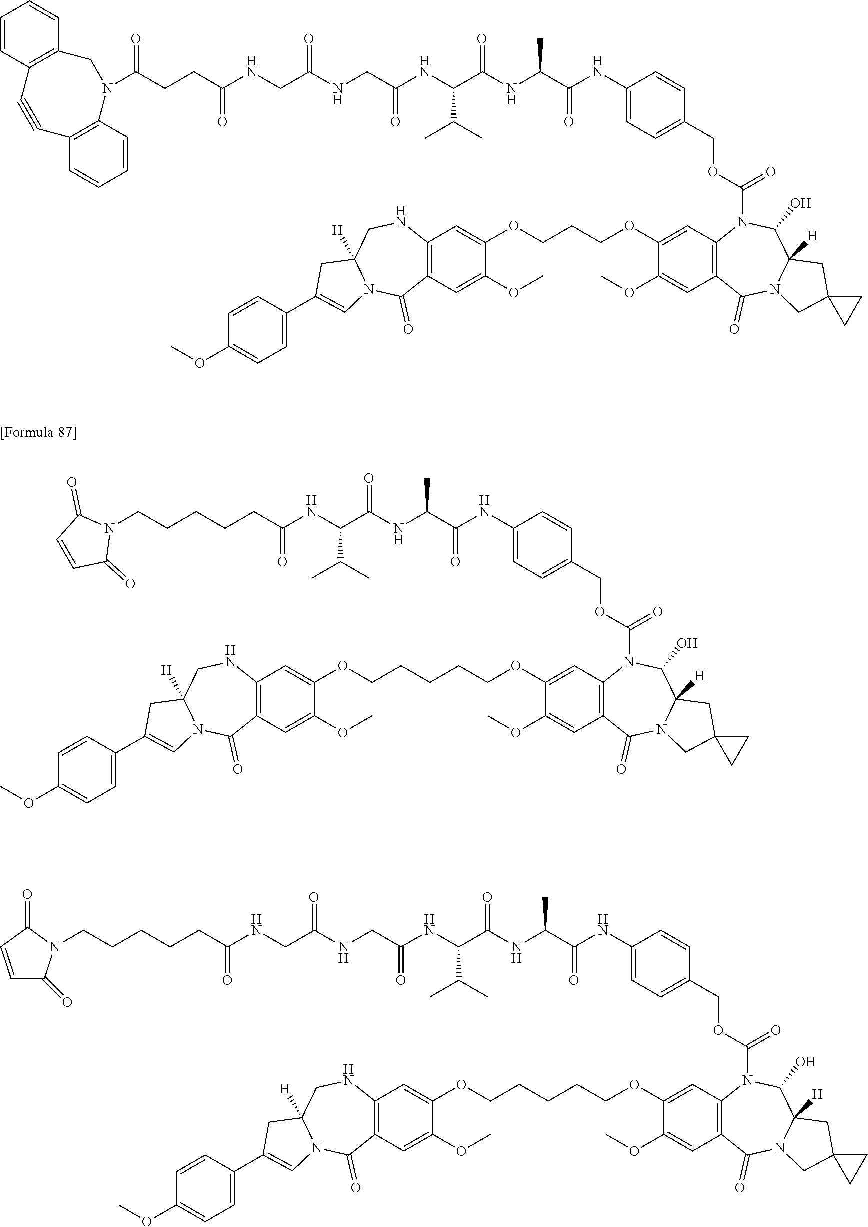

### VIII. Medicament

The antibody-drug conjugate of the present invention exhibits cellular cytotoxic activity to cancer cells, and hence may be used as a medicine, in particular, a therapeutic agent and/or prophylactic agent for cancer. In some embodiments, the cancer expresses DLL3 or overexpresses DLL3.

Examples of cancers to which the antibody-drug conjugate of the present invention is applied may include small cell lung cancer (SCLC), large cell neuroendocrine carcinoma (LCNEC), neuroendocrine tumors of various tissues including kidney, genitourinary tract (bladder, prostate, ovary, cervix, and endometrium), gastrointestinal tract (stomach, colon), thyroid (medullary thyroid cancer), pancreas and lung, gliomas or pseudo neuroendocrine tumors (pNETs), but are not limited thereto as long as cancer cells as a therapeutic target are expressing protein recognizable for the antibody in the antibody-drug conjugate.

The antibody-drug conjugate of the present invention can be preferably administered to mammals, and are more preferably administered to humans.

Substances used in a pharmaceutical composition containing the antibody-drug conjugate of the present invention may be suitably selected and applied from formulation additives or the like that are generally used in the field in view of the dose or concentration for administration.

The antibody-drug conjugate of the present invention may be administered as a pharmaceutical composition containing one or more pharmaceutically applicable components. For example, the pharmaceutical composition typically contains one or more pharmaceutical carriers (e.g., sterilized liquid (including water and oil (petroleum oil and oil of animal origin, plant origin, or synthetic origin (such as peanut oil, soybean oil, mineral oil, and sesame oil)))). Water is a more typical carrier when the pharmaceutical composition above is intravenously administered. Saline solution, an aqueous dextrose solution, and an aqueous glycerol solution can be also used as a liquid carrier, in particular, for an injection solution. Suitable pharmaceutical vehicles are known in the art. If desired, the composition above may also contain a trace amount of a moisturizing agent, an emulsifying agent, or a pH buffering agent. Examples of suitable pharmaceutical carriers are disclosed in “Remington's Pharmaceutical Sciences” by E. W. Martin. The formulations correspond to the administration mode.

The type of cancer to which the anti-DLL3 antibody-drug conjugate of the present invention is applied is not particularly limited as long as the cancer expresses DLL3 in cancer cells to be treated. Examples thereof can include small cell lung cancer (SCLC), large cell neuroendocrine carcinoma (LCNEC), neuroendocrine tumors of various tissues including kidney, genitourinary tract (bladder, prostate, ovary, cervix, and endometrium), gastrointestinal tract (stomach, colon), thyroid (medullary thyroid cancer), pancreas and lung, gliomas or pseudo neuroendocrine tumors (pNETs). However the cancer is not limited thereto as long as the cancer expresses DLL3. More preferred examples of the cancer can include small cell lung cancer (SCLC), large cell neuroendocrine carcinoma (LCNEC), and neuroendocrine tumors of various tissues.

The anti-DLL3 antibody-drug conjugate of the present invention can preferably be administered to a mammal, and more preferably to a human.

Various delivery systems are known and they may be used for administering the antibody-drug conjugate of the present invention. Examples of the administration route may include, but not limited to, intradermal, intramuscular, intraperitoneal, intravenous, and subcutaneous routes. The administration may be made by injection or bolus injection, for example. According to a specific preferred embodiment, the administration of the above ligand-drug conjugate form is done by injection. Parenteral administration is a preferred administration route.

According to a representative embodiment, the pharmaceutical composition is prescribed, as a pharmaceutical composition suitable for intravenous administration to humans, according to conventional procedures. The composition for intravenous administration is typically a solution in a sterile and isotonic aqueous buffer. If necessary, the medicine may contain a solubilizing agent and a local anesthetic to alleviate pain at an injection site (e.g., lignocaine). Generally, the ingredients above are provided either individually as a dried lyophilized powder or an anhydrous concentrate contained in each container which is obtained by sealing in an ampoule or a sachet with indication of the amount of the active agent, or as a mixture in a unit dosage form. When the pharmaceutical composition is to be administered by injection, it may be administered from an injection bottle containing water or saline of sterile pharmaceutical grade. When the medicine is administered by injection, an ampoule of sterile water or saline for injection may be provided so that the aforementioned ingredients are admixed with each other before administration.

The pharmaceutical composition of the present invention may be a pharmaceutical composition containing only the present antibody-drug conjugate, or a pharmaceutical composition containing the antibody-drug conjugate and at least one cancer treating agent other than the antibody-drug conjugate. The antibody-drug conjugate of the present invention may be administered in combination with other cancer treating agents, and thereby the anti-cancer effect may be enhanced. Other anti-cancer agents used for such purpose may be administered to an individual simultaneously with, separately from, or subsequently to the antibody-drug conjugate, and may be administered while varying the administration interval for each. Examples of such cancer treating agents may include abraxane, carboplatin, cisplatin, gemcitabine, irinotecan (CPT-11), paclitaxel, pemetrexed, sorafenib, vinblastin, agents described in International Publication No. WO2003/038043, LH-RH analogues (e.g., leuprorelin, goserelin), estramustine phosphate, estrogen antagonists (e.g., tamoxifen, raloxifene), and aromatase inhibitors (e.g., anastrozole, letrozole, exemestane), but are not limited thereto as long as they are agents having an antitumor activity.

The pharmaceutical composition can be formulated into a lyophilization formulation or a liquid formulation as a formulation having the selected composition and required purity. When formulated as a lyophilization formulation, it may be a formulation containing suitable formulation additives that are used in the art. Also for a liquid formulation, it may be formulated as a liquid formulation containing various formulation additives that are used in the art.

The composition and concentration of the pharmaceutical composition may vary depending on the administration method. However, the antibody-drug conjugate contained in the pharmaceutical composition of the present invention can exhibit a pharmaceutical effect even at a small dosage when the antibody-drug conjugate has a higher affinity for an antigen, that is, a higher affinity (lower Kd value) in terms of the dissociation constant (Kd value) for the antigen. Thus, for determining the dosage of the antibody-drug conjugate, the dosage may be set in view of the situation relating to the affinity of the antibody-drug conjugate with the antigen. When the antibody-drug conjugate of the present invention is administered to a human, for example, about 0.001 to 100 mg/kg can be administered once or administered in several portions with intervals of 1 to 180 days.

In sum, the disclosed immunoglobulin-related compositions (e.g., antibodies or antigen binding fragments thereof, such as 2-C8-A, 6-G23-F, 10-O18-A, H2-C8-A, H6-G23-F, or H10-O18-A) and antibody-drug conjugates thereof are useful for the treatment of DLL3-associated cancers. Such treatment can be used in patients identified as having pathologically high levels of the DLL3 (e.g., those diagnosed by the methods described herein) or in patients diagnosed with a disease known to be associated with such pathological levels. In one aspect, the present disclosure provides a method for treating a DLL3-associated cancer in a subject in need thereof, comprising administering to the subject an effective amount of an antibody (or antigen binding fragment thereof) of the present technology. Examples of cancers that can be treated by the antibodies of the present technology include, but are not limited to: small cell lung cancer (SCLC), large cell neuroendocrine carcinoma (LCNEC), neuroendocrine tumors of various tissues including kidney, genitourinary tract (bladder, prostate, ovary, cervix, and endometrium), gastrointestinal tract (stomach, colon), thyroid (medullary thyroid cancer), pancreas and lung, gliomas or pseudo neuroendocrine tumors (pNETs). The compositions of the present technology may optionally be administered as a single bolus to a subject in need thereof. Alternatively, the dosing regimen may comprise multiple administrations performed at various times after the appearance of tumors. Administration can be carried out by any suitable route, including orally, intranasally, parenterally (intravenously, intramuscularly, intraperitoneally, or subcutaneously), rectally, intracranially, intratumorally, intrathecally, or topically. Administration includes self-administration and the administration by another. It is also to be appreciated that the various modes of treatment of medical conditions as described are intended to mean “substantial”, which includes total but also less than total treatment, and wherein some biologically or medically relevant result is achieved.

### EXAMPLES

The present technology is further illustrated by the following Examples, which should not be construed as limiting in any way. The following Examples demonstrate the preparation, characterization, and use of illustrative anti-DLL3 antibodies and antibody-drug conjugates (ADC) of the present technology. However, these examples are not intended to limit the scope of the present invention. Furthermore, these examples should not be construed in a limited manner by any means. It is to be noted that, in the following examples, unless otherwise specified, individual operations regarding genetic manipulation have been carried out according to the method described in “Molecular Cloning” (Sambrook, J., Fritsch, E. F. and Maniatis, T., published by Cold Spring Harbor Laboratory Press in 1989) or other methods described in experimental manuals used by persons skilled in the art, or when commercially available reagents or kits have been used, the examples have been carried out in accordance with the instructions included in the commercially available products. In the present description, reagents, solvents and starting materials are readily available from commercially available sources, unless otherwise specified.

### Example 1-Generation of Monoclonal Antibodies

The extracellular domain (ECD) of DLL3 (GenBank accession number Q9NY J7-1) corresponding to amino acids Ala27-Ala479 with a C-terminal 6×His tag (SEQ ID NO: 84) produced in HEK293T cells stably expressing full length DLL3 were used as immunogens. Ablexis AlivaMAb Kappa Mice (Ablexis, San Diego, CA) harboring a human immunoglobulin repertoire were immunized either with soluble DLL3-ECD or stable cells following standard immunization techniques over a period of 3 weeks. Splenocytes and draining lymph node cells from mice with high serum titers specific for DLL3 were harvested and fused with mouse myeloma cells to generate hybridomas using electrofusion. These hybridomas were then screened to identify the presence of antibodies that bound specifically to soluble DLL3-ECD by ELISA and full-length DLL3 protein on stably expressing 293 cells by flow cytometry versus parental 293 cells. Hybridomas were selected for further investigation by ranking in flow cytometry for staining intensity on 293 DLL3 transfectants along with 4° C./37° C. staining as described below.

### Example 2—The 4/37 Internalization Assay with the Monoclonal Antibodies 6-G23-F, 2-C8-A, 7-I1-B and 10-O18-A

The four monoclonal antibodies (6-G23-F, 2-C8-A, 7-I1-B and 10-O18-A) were compared for their ability to internalize DLL3 by comparing the staining at 4° C. with that at 37° C. A reference monoclonal antibody SC16, which is known to internalize via DLL3 and to have ADC activity, and which was previously reported in the literature, was used as the positive control for internalization. NCI-H82 cells in exponential growth were harvested with trypsin/EDTA, washed once in RPMI containing 10% fetal calf serum (FCS) and resuspended in DMEM supplemented with 10% FCS at 2×107 cells/ml. 100 μl (2×106 cells) were added to U bottom 96 well plates. The test monoclonal antibodies (6-G23-F, 2-C8-A, 7-I1-B or 10-O18-A) or the reference monoclonal antibody were added to separate wells to give a final concentration of 10 μg/ml in duplicate plates. Both plates were held at 4° C. for 30 minutes after which both plates were washed 2× with cold RPMI supplemented with 10% FCS and resuspended in RPMI supplemented with 10% FCS. One plate was held at 4° C. (the control plate) and the other plate was incubated at 37° C. in a CO2 incubator (the experimental plate). After 4 hours incubation at 37° C. in a CO2 incubator for the experimental plate and at 4° C. for the control plate, the cells were washed 3 times at 4° C. with cold wash buffer (PBS containing 0.5% BSA). Then the samples were re-suspended in cold wash buffer+R-Phycoerythrin-AffiniPure F(ab′)2 Fragment Goat Anti-Mouse IgG (Jackson 115-116-071) at a final concentration of 7 μg/ml in wash buffer. After 30 minutes incubation at 4° C. the cells were washed three times in cold wash buffer and either fixed in PBS 0.5% paraformaldehyde and analyzed by flow cytometry within 48 hours. The mean fluorescent intensity (MFI) ratios calculated by dividing the MFI obtained from the control plate (which incubated with the antibodies at 4° C.) by the corresponding MFI obtained from the experimental plate (which incubated with the antibodies at 37° C.) was taken as a relative measure of internalization. A high value indicated greater internalization. As shown in the Table below, all of the monoclonal antibodies (6-G23-F, 2-C8-A, 7-I1-B and 10-O18-A) were able to internalize on binding DLL3 but not to the extent of the reference monoclonal antibody.

| Clone | MFI Ratio |
|---|---|
| 6-G23-F | 1.67 |
| 2-C8-A | 1.76 |
| 7-I1-B | 1.68 |
| 10-O18-A | 1.56 |
| Reference Monoclonal antibody | 2.66 |
| Mouse IgG1 | 1.28 |

These results demonstrate that the immunoglobulin-related compositions of the present technology undergo internalization via binding to DLL3. Accordingly, the immunoglobulin-related compositions disclosed herein are useful for delivering therapeutic agents to DLL3-positive cancer cells.

### Example 3—The Quenching Internalization Assay for Monoclonal Antibodies 6-G23-F, 2-C8-A, 7-I1-B and 10-O18-A

To rank the monoclonal antibodies with respect to internalization, a Quenching Internalization Assay was used. This method reflects internalization and entry into the endosome/lysosome pathway. A goat anti-mouse IgG1 F(ab) (Jackson Immunoresearch 115-007-185) was doubly labelled with Dy light Dy650 NHS ester (Thermofisher 02206) and a LICOR TRDye QC1 NHS ester (LICOR 929-7030) (the doubly labelled antibody is referred to as “F(ab) Dy650-QC1” herein). The principle of this assay is as follows: F(ab) Dy650-QC1 is not fluorescent because the Dy light Dy650 fluorescence is quenched by IRDye QC1. However, upon internalization, the F(ab) Dy650-QC1 is degraded via the endosome/lysosome pathway, and resultant release of the IRDye QC1 makes the fluorescence of Dy light Dy650 observable. Accordingly, the Dy light Dy650 fluorescence signal was taken as a measure of internalization via the lysosomes. Briefly, NCI-H82 cells in exponential growth were harvested with trypsin/EDTA, washed once in growth media RPMI supplemented with 10% FCS and re-suspended in of growth media and 1.25×10⁶ cell (80 μl) were added per well. Monoclonal DLL3 antibodies at 200 μg/ml concentration were mixed with the goat anti-mouse IgG1 Dy650 QC1 at 200 μg/ml at room temperature for 20 minutes, and 20 μl of the mixture added to the cells. After 30 minutes incubation at 4° C. the cells were washed twice with the growth media, resuspended in the growth media and transferred to 37° C. in a CO₂ incubator for 4 hours to allow internalization. The cells were then washed 2× with ice cold PBS containing 0.5% BSA and analyzed on by flow cytometry, and the Mean Fluorescent Intensity was determined. The Mean Fluorescent Intensity of the control reference monoclonal was set at 100% internalization. As shown in the Table below, all four monoclonal antibodies demonstrated internalization and entry into the endosome/lysosome pathway.

| | Dy light Dy658 Fluorescence (% of the |
|---|---|
| Clone | Control Reference Monoclonal Antibody |
| 6-G23-F (10 μg/ml) | 76.5 |
| 7-I1-B (10 μg/ml) | 70.1 |
| 2-C8-A (10 μg/ml) | 62.9 |
| 10-O18-A (10 μg/ml) | 62.2 |
| Isotype Control | 20 |

These results demonstrate that the immunoglobulin-related compositions of the present technology undergo internalization via binding to DLL3, and enter the phagosome/lysosome compartment of the cells. Accordingly, the immunoglobulin-related compositions disclosed herein are useful for delivering therapeutic agents to DLL3-positive cancer cells.

### Example 4—The Fab ZAP Assay for Anti-DLL3 Monoclonal Antibodies

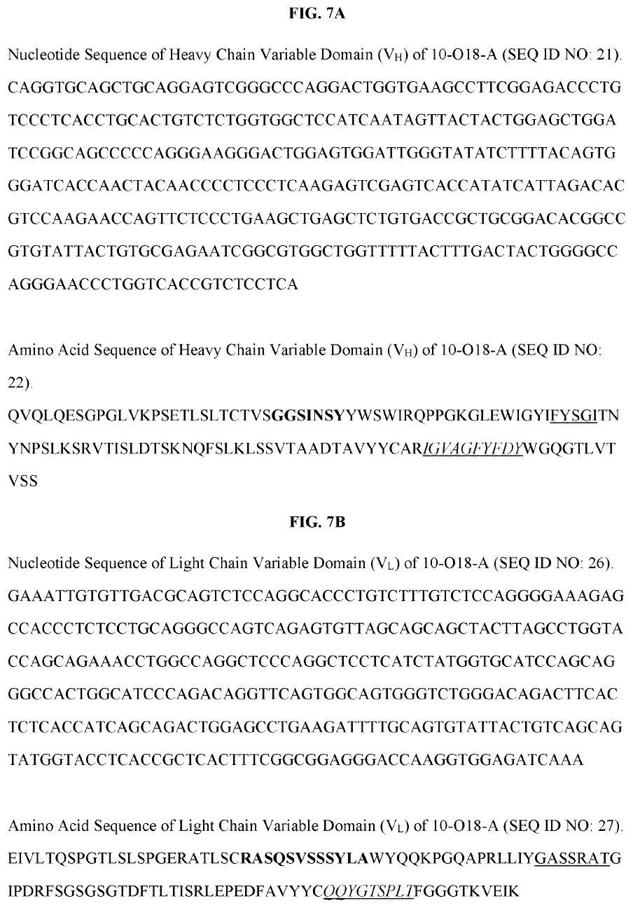

A Fab ZAP assay was used as another way to measure internalization. The Fab ZAP assay measures the delivery of a toxin to a cell via internalization of the anti-DLL3 monoclonal antibody. The Fab ZAP assay uses the saporin toxin conjugated F(ab) anti-mouse heavy and Light Chain to tag the monoclonal antibodies with toxin. A kit from Advanced Targeting Systems was used, and the Fab ZAP assay protocol was followed to characterize the panel of anti-DLL3 monoclonal antibodies. Briefly, NCI-H82 cells in exponential growth are harvested with trypsin/EDTA, washed once in RMPI supplemented with 10% FCS and plated at 5000 cells/well in 96 well white solid plates in 100 μl RPMI supplemented with 10% FCS. The next day, 25 μl of the purified monoclonal antibodies (G23-F, 2-C8-A, 7-I1-B or 10-O18-A) or the reference monoclonal monoclonal antibody, were added at a starting concentration of 10 μg/ml, and serial three fold dilutions performed. The saponin conjugated F(ab) anti-mouse Ig HL (Fab ZAP) was added in 25 μl to give a final concentration of 4.4 nM. After 3-4 days an equal volume of Cell Titre Glow (Promega G7571) was added to the plate, which was shaken on an orbital shaker for 2 minutes and after a further 10 minutes at room temperature the luminescence was read using a plate reader. All of the monoclonal antibodies were tested as full titrations in order to negate the prozone effect. As shown in FIG. 9 and the Table below, all of the monoclonal antibodies exhibited cytotoxic activity, comparable to the reference monoclonal antibody. In other experiments with these monoclonal antibodies, a mouse IgG1 control monoclonal antibody did not exhibit cytotoxic activity. Accordingly, these results demonstrate that the cytotoxic activity is mediated through recognition of DLL3 and not through FcRs.

| | FAB ZAP % SC16 |
|---|---|
| | Max Killing |
| 6-G23-F (10 μg/ml) | 94.0 |
| 7-I1-B (10 μg/ml) | 97.0 |
| 10-O18-A (10 μg/ml) | 88.0 |
| 2-C8-A (10 μg/ml) | 89.0 |

These results demonstrate that the immunoglobulin-related compositions of the present technology can deliver therapeutic agents to tumors that express DLL3 on their cell surface. Accordingly, the immunoglobulin-related compositions disclosed herein are useful for delivering therapeutic agents to DLL3-positive cancer cells.

### Example 5—Epitope Binding of Anti-DLL3 Antibodies

The panel of purified anti-DLL3 monoclonal antibodies and the reference monoclonal antibody were subjected to pairwise epitope binning on a Carterra® array surface plasmon resonance (SPR) assay platform (Carterra® Inc., Salt Lake City UT) where each monoclonal antibody was tested for the capture of Histidine-tagged DLL3 antigen (DLL3-His), and also for competing with every other antibody in the panel for the binding to DLL3-His. The antibodies were immobilized on a HC200M chip (ligand) through standard amine coupling techniques by the print array method. Then in each cycle antigen was injected across the entire array followed by a single antibody (analyte). At the end of each cycle the surface was regenerated to remove antigen and analyte before a new cycle started. As shown in the Table below, three different bins were identified with the panel with 7-I1-B and 2-C8-A mapping to bin 2 while 6-G23-F was in bin 3 and 10-O18-A was in bin 1.

| Monoclonal | Epitope Bin |
|---|---|
| 10-O18-A | 1 |
| 7-I1-B | 2 |
| 2-C8-A | 2 |
| 6-G23-F | 3 |

These results demonstrate that the immunoglobulin-related compositions of the present technology bind to three distinct epitopes present in DLL3 protein. Accordingly, the immunoglobulin-related compositions disclosed herein may be used in combination with each other for delivering multiple therapeutic agents to tumor cells expressing DLL3.

### Example 6—Affinity Measurement

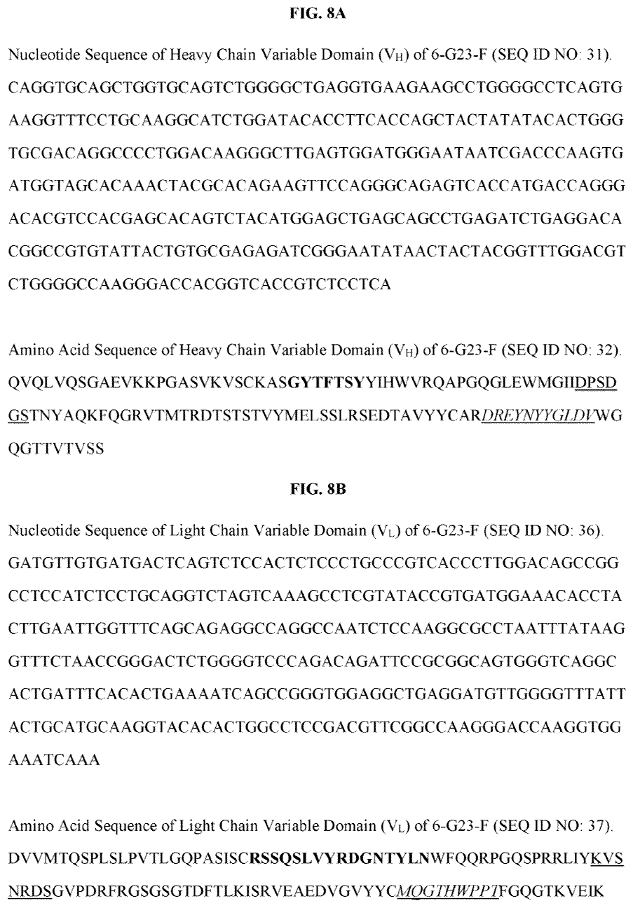

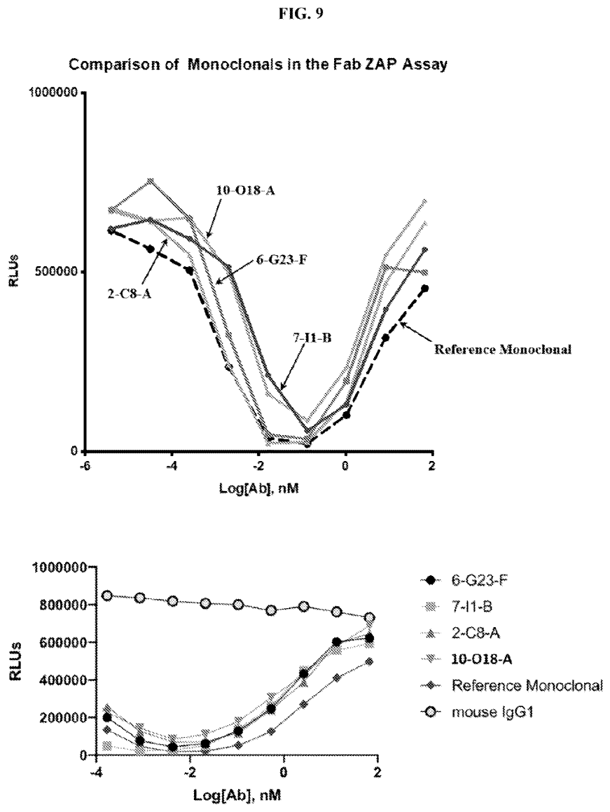

Binding affinities of the four monoclonal antibodies (6-G23-F, 2-C8-A, 7-I1-B and 10-O18-A) were determined by biolayer interferometry (BLI) using the Octet HTX instrument at 25° C. using PBS 0.1% BSA 0.02% Tween 20 as the binding buffer and 10 mM Glycine pH 1.7 as the regeneration buffer. The four purified monoclonal antibodies (5 μg/mL each) were loaded onto anti-mouse Fc sensors. Loaded sensors were dipped into a serial dilutions of Recombinant Human DLL3 Protein, (amino acids Ala27-Ala479, cat #9749-DL, R&D Systems) at a 200 nM starting concentration, with 7 serial 1:3 dilutions. As shown in FIGS. 10A-10D, binding was concentration-dependent. Dissociation constants (KD) were calculated using a monovalent (1:1) binding model. As shown in FIG. 10E, all the monoclonal antibodies had affinities in the sub-nanomolar range. FIG. 11 shows that the 6-G23-F, 10-O18-A, and 2-C8-A monoclonal antibodies (mAbs) selectively bind DLL3, but not DLL1 or DLL4. The 7-I1-B mAb binds both DLL3 and DLL4, but not DLL1.

These results demonstrate that the immunoglobulin-related compositions of the present technology specifically bind DLL3 with high affinity. Accordingly, the immunoglobulin-related compositions of the present technology are useful in methods for detecting DLL3 protein in a biological sample.

### Example 7—Binding of Monoclonal Antibodies to Transfected and Primary Cells

The four monoclonal antibodies (6-G23-F, 2-C8-A, 7-I1-B and 10-O18-A) were tested for their ability to bind to mouse and cynomolgous DLL3 as well as endogenous human DLL3 by flow cytometry. For this purpose, HEK293 cells were transfected with plasmid DNA encoding full-length human, mouse or cynomolgous DLL3 and used in the experiment. Briefly 106 transfected HEK293 cells or NCI-H82 primary cells were added in FACS buffer PBS 0.5% BSA to the wells of a 96 well U bottom plate and purified monoclonal added to 10 μg/ml. After 30 minutes incubation at 4° C. the cells were washed 3 times in FACS buffer and incubated with a PE labelled F(ab)2 anti-mouse IgG H and L 2nd stage. After another 30 minutes incubation at 4° C. the cells were washed three times in FACS buffer and analyzed on the flow cytometer. Data are presented at the ratio of the Mean Fluorescent Intensity for the monoclonal divided by the background staining with the 2nd stage. As shown in the Table below, all of the monoclonal antibodies cross reacted with cynomolgous DLL3 and detected endogenous DLL3 on NCI H82 cells but only 6-G23-F and 7-I1-B bound to mouse DLL3.

| | 293 | 293 cyno | 293 | 293 (negative | |
|---|---|---|---|---|---|
| Monoclonal | hDLL3 | DLL3 | mDLL3 | control) | NCI-H82 |
| 6-G23-F | 6.33 | 13.06 | 3.97 | 1.32 | 11.10 |
| 7-I1-B | 9.74 | 18.67 | 4.70 | 1.46 | 19.21 |
| 10-O18-A | 4.74 | 9.85 | 0.99 | 0.60 | 9.40 |
| 2-C8-A | 7.71 | 15.03 | 1.50 | 0.59 | 11.89 |

These results demonstrate that the immunoglobulin-related compositions of the present technology are useful in methods for detecting DLL3 protein in a biological sample.

### Example 8—Sequencing

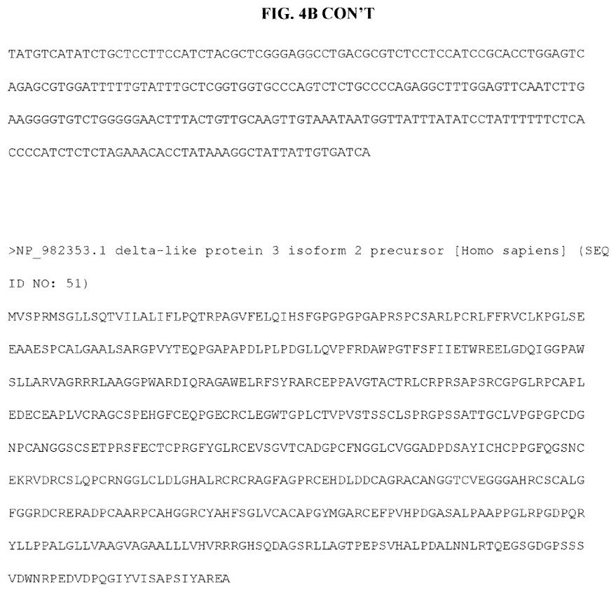

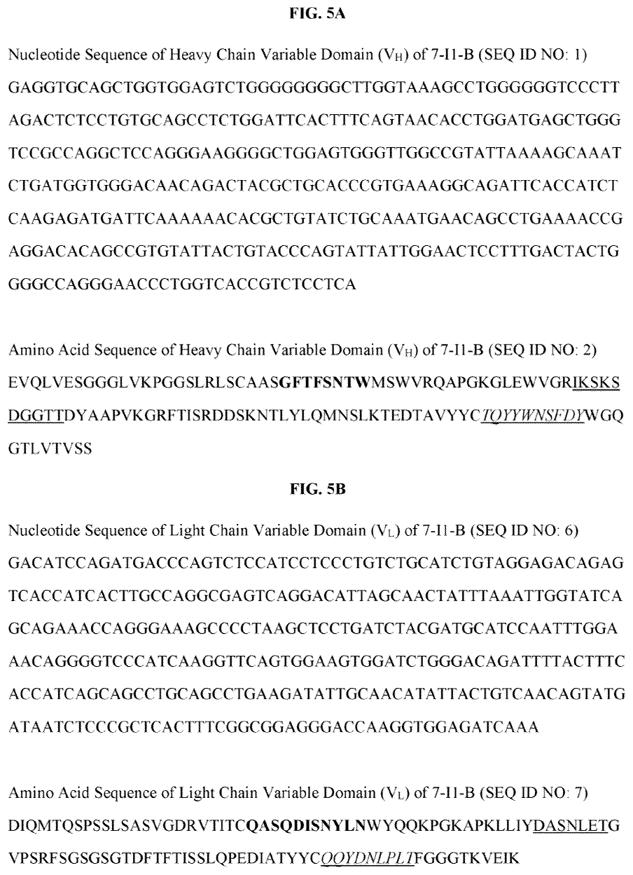

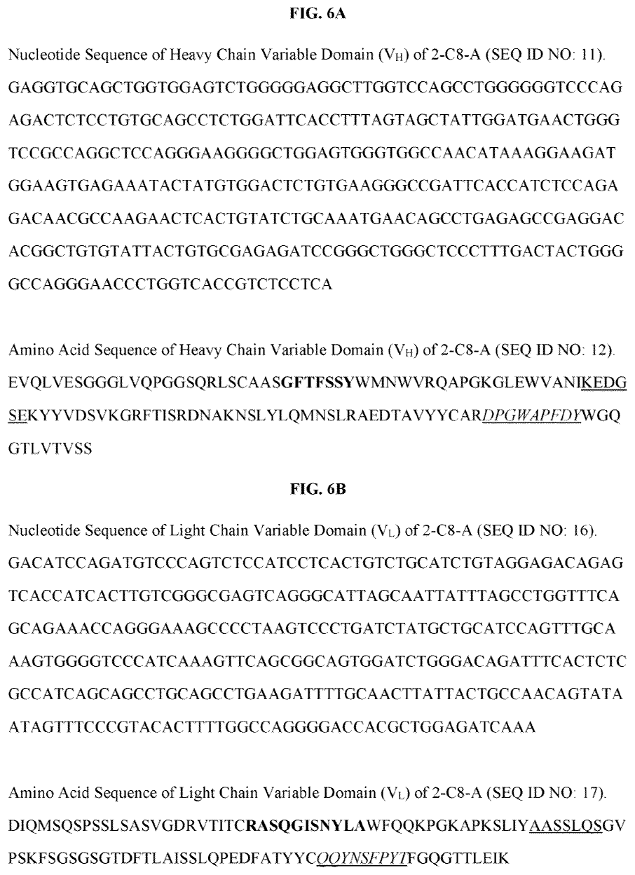

Variable Heavy and Variable Light chains of the four monoclonal antibodies were isolated from the corresponding hybridomas for 7-I1-B, 6-G23-F, 2-C8-A and 10-O18-A by RACE (Rapid amplification of cDNA ends). RNA was isolated from lysed hybridoma with a RNAEasy kit (Qiagen). The mRNA was isolated for cDNA synthesis and PCR products were generated using the RACE kit. The PCR products were then cloned into a TOPO vector, PCR amplified, and subsequently gel isolated for sequencing. The nucleotide and amino acid sequences of heavy chain variable domain (VH) and light chain variable domain (VL) are shown in the Table below and FIGS. 5A-5D (7-I1-B), FIGS. 6A-6D (2-C8-A), FIGS. 7A-7D (10-O18-A), and FIGS. 8A-8D (6-G23-F).

| SEQ ID NO: | Description | Sequence |
|---|---|---|
| SEQ ID NO: 1 | Nucleotide Sequence of | GAGGTGCAGCTGGTGGAGTCTGGGGGGGGC |
| | V_{H} of 7-11-B | TTGGTAAAGCCTGGGGGGTCCCTTAGACTCT |
| | | CCTGTGCAGCCTCTGGATTCACTTTCAGTAA |
| | | CACCTGGATGAGCTGGGTCCGCCAGGCTCC |
| | | AGGGAAGGGGCTGGAGTGGGTTGGCCGTAT |
| | | TAAAAGCAAATCTGATGGTGGGACAACAGA |
| | | CTACGCTGCACCCGTGAAAGGCAGATTCAC |
| | | CATCTCAAGAGATGATTCAAAAAACACGCT |
| | | GTATCTGCAAATGAACAGCCTGAAAACCGA |
| | | GGACACAGCCGTGTATTACTGTACCCAGTAT |
| | | TATTGGAACTCCTTTGACTACTGGGGCCAGG |
| | | GAACCCTGGTCACCGTCTCCTCA |
| SEQ ID NO: 2 | Amino acid Sequence of | EVQLVESGGGLVKPGGSLRLSCAASGFTFSNT |
| | V_{H} of 7-11-B | WMSWVRQAPGKGLEWVGRIKSKSDGGTTDY |
| | | AAPVKGRFTISRDDSKNTLYLQMNSLKTEDTA |
| | | VYYCTQYYWNSFDYWGQGTLVTVSS |
| SEQ ID NO: 3 | Amino acid Sequence of | GFTFSNTW |
| | V_{H} CDR1 of 7-11-B | |
| SEQ ID NO: 4 | Amino acid Sequence of | IKSKSDGGTT |
| | V_{H} CDR2 of 7-11-B | |
| SEQ ID NO: 5 | Amino acid Sequence of | TQYYWNSFDY |
| | V_{H} CDR3 of 7-11-B | |
| SEQ ID NO: 6 | Nucleotide Sequence of | GACATCCAGATGACCCAGTCTCCATCCTCCC |
| | V_{L} of 7-11-B | TGTCTGCATCTGTAGGAGACAGAGTCACCAT |
| | | CACTTGCCAGGCGAGTCAGGACATTAGCAA |
| | | CTATTTAAATTGGTATCAGCAGAAACCAGG |
| | | GAAAGCCCCTAAGCTCCTGATCTACGATGC |
| | | ATCCAATTTGGAAACAGGGGTCCCATCAAG |
| | | GTTCAGTGGAAGTGGATCTGGGACAGATTTT |
| | | ACTTTCACCATCAGCAGCCTGCAGCCTGAAG |
| | | ATATTGCAACATATTACTGTCAACAGTATGA |
| | | TAATCTCCCGCTCACTTTCGGCGGAGGGACC |
| | | AAGGTGGAGATCAAA |
| SEQ ID NO: 7 | Amino acid Sequence of | DIQMTQSPSSLSASVGDRVTITCQASQDISNY |
| | V_{L} of 7-11-B | NWYQQKPGKAPKLLIYDASNLETGVPSRFSGS |
| | | GSGTDFTFTISSLQPEDIATYYCQQYDNLPLTF |
| | | GGGTKVEIK |
| SEQ ID NO: 8 | Amino acid Sequence of | QASQDISNYLN |
| | V_{L} CDR1 of 7-11-B | |
| SEQ ID NO: 9 | Amino acid Sequence of | DASNLET |
| | V_{L} CDR2 of 7-11-B | |
| SEQ ID NO: 10 | Amino acid Sequence of | QQYDNLPLT |
| | V_{L} CDR3 of 7-11-B | |
| SEQ ID NO: 11 | Nucleotide Sequence of | GAGGTGCAGCTGGTGGAGTCTGGGGGAGGC |
| | V_{H} of 2-C8-A | TTGGTCCAGCCTGGGGGGTCCCAGAGACTCT |
| | | CCTGTGCAGCCTCTGGATTCACCTTTAGTAG |
| | | CTATTGGATGAACTGGGTCCGCCAGGCTCCA |
| | | GGGAAGGGGCTGGAGTGGGTGGCCAACATA |
| | | AAGGAAGATGGAAGTGAGAAATACTATGTG |
| | | GACTCTGTGAAGGGCCGATTCACCATCTCCA |
| | | GAGACAACGCCAAGAACTCACTGTATCTGC |
| | | AAATGAACAGCCTGAGAGCCGAGGACACGG |
| | | CTGTGTATTACTGTGCGAGAGATCCGGGCTG |
| | | GGCTCCCTTTGACTACTGGGGCCAGGGAAC |
| | | CCTGGTCACCGTCTCCTCA |
| SEQ ID NO: 12 | Amino acid Sequence of | EVQLVESGGGLVQPGGSQRLSCAASGFTFSSY |
| | V_{H} of 2-C8-A | WMNWVRQAPGKGLEWVANIKEDGSEKYYV |
| | | DSVKGRFTISRDNAKNSLYLQMNSLRAEDTA |
| | | VYYCARDPGWAPFDYWGQGTLVTVSS |
| SEQ ID NO: 13 | Amino acid Sequence of | GFTFSSY |
| | V_{H} CDR1 of 2-C8-A | |
| SEQ ID NO: 14 | Amino acid Sequence of | KEDGSE |
| | V_{H} CDR2 of 2-C8-A | |
| SEQ ID NO: 15 | Amino acid Sequence of | DPGWAPFDY |
| | V_{H} CDR3 of 2-C8-A | |
| SEQ ID NO: 16 | Nucleotide Sequence of | GACATCCAGATGTCCCAGTCTCCATCCTCAC |
| | V_{L} of 2-C8-A | TGTCTGCATCTGTAGGAGACAGAGTCACCAT |
| | | CACTTGTCGGGCGAGTCAGGGCATTAGCAA |
| | | TTATTTAGCCTGGTTTCAGCAGAAACCAGGG |
| | | AAAGCCCCTAAGTCCCTGATCTATGCTGCAT |
| | | CCAGTTTGCAAAGTGGGGTCCCATCAAAGTT |
| | | CAGCGGCAGTGGATCTGGGACAGATTTCAC |
| | | TCTCGCCATCAGCAGCCTGCAGCCTGAAGAT |
| | | TTTGCAACTTATTACTGCCAACAGTATAATA |
| | | GTTTCCCGTACACTTTTGGCCAGGGGACCAC |
| | | GCTGGAGATCAAA |
| SEQ ID NO: 17 | Amino acid Sequence of | DIQMSQSPSSLSASVGDRVTITCRASQGISNYL |
| | V_{L} of 2-C8-A | AWFQQKPGKAPKSLIYAASSLQSGVPSKFSGS |
| | | GSGTDFTLAISSLQPEDFATYYCQQYNSFPYTF |
| | | GQGTTLEIK |
| SEQ ID NO: 18 | Amino acid Sequence of | RASQGISNYLA |
| | V_{L} CDR1 of 2-C8-A | |
| SEQ ID NO: 19 | Amino acid Sequence of | AASSLQS |
| | V_{L} CDR2 of 2-C8-A | |
| SEQ ID NO: 20 | Amino acid Sequence of | QQYNSFPYT |
| | V_{L} CDR3 of 2-C8-A | |
| SEQ ID NO: 21 | Nucleotide Sequence of | CAGGTGCAGCTGCAGGAGTCGGGCCCAGGA |
| | V_{H} of 10-018-A | CTGGTGAAGCCTTCGGAGACCCTGTCCCTCA |
| | | CCTGCACTGTCTCTGGTGGCTCCATCAATAG |
| | | TTACTACTGGAGCTGGATCCGGCAGCCCCCA |
| | | GGGAAGGGACTGGAGTGGATTGGGTATATC |
| | | TTTTACAGTGGGATCACCAACTACAACCCCT |
| | | CCCTCAAGAGTCGAGTCACCATATCATTAGA |
| | | CACGTCCAAGAACCAGTTCTCCCTGAAGCTG |
| | | AGCTCTGTGACCGCTGCGGACACGGCCGTG |
| | | TATTACTGTGCGAGAATCGGCGTGGCTGGTT |
| | | TTTACTTTGACTACTGGGGCCAGGGAACCCT |
| | | GGTCACCGTCTCCTCA |
| SEQ ID NO: 22 | Amino acid Sequence of | QVQLQESGPGLVKPSETLSLTCTVSGGSINSYY |
| | V_{H} of 10-O18-A | WSWIRQPPGKGLEWIGYIFYSGITNYNPSLKSR |
| | | VTISLDTSKNQFSLKLSSVTAADTAVYYCARI |
| | | GVAGFYFDYWGQGTLVTVSS |
| SEQ ID NO: 23 | Amino acid Sequence of | GGSINSY |
| | V_{H} CDR1 of 10-O18-A | |
| SEQ ID NO: 24 | Amino acid Sequence of | FYSGI |
| | V_{H} CDR2 of 10-O18-A | |
| SEQ ID NO: 25 | Amino acid Sequence of | IGVAGFYFDY |
| | V_{H} CDR3 of 10-O18-A | |
| SEQ ID NO: 26 | Nucleotide Sequence of | GAAATTGTGTTGACGCAGTCTCCAGGCACCC |
| | V_{L} of 10-O18-A | TGTCTTTGTCTCCAGGGGAAAGAGCCACCCT |
| | | CTCCTGCAGGGCCAGTCAGAGTGTTAGCAG |
| | | CAGCTACTTAGCCTGGTACCAGCAGAAACC |
| | | TGGCCAGGCTCCCAGGCTCCTCATCTATGGT |
| | | GCATCCAGCAGGGCCACTGGCATCCCAGAC |
| | | AGGTTCAGTGGCAGTGGGTCTGGGACAGAC |
| | | TTCACTCTCACCATCAGCAGACTGGAGCCTG |
| | | AAGATTTTGCAGTGTATTACTGTCAGCAGTA |
| | | TGGTACCTCACCGCTCACTTTCGGCGGAGGG |
| | | ACCAAGGTGGAGATCAAA |
| SEQ ID NO: 27 | Amino acid Sequence of | EIVLTQSPGTLSLSPGERATLSCRASQSVSSSYL |
| | V_{L} of 10-O18-A | AWYQQKPGQAPRLLIYGASSRATGIPDRESGS |
| | | GSGTDFTLTISRLEPEDFAVYYCQQYGTSPLTF |
| | | GGGTKVEIK |
| SEQ ID NO: 28 | Amino acid Sequence of | RASQSVSSSYLA |
| | V_{L} CDR1 of 10-O18-A | |
| SEQ ID NO: 29 | Amino acid Sequence of | GASSRAT |
| | V_{L} CDR2 of 10-O18-A | |
| SEQ ID NO: 30 | Amino acid Sequence of | QQYGTSPLT |
| | V_{L} CDR3 of 10-O18-A | |
| SEQ ID NO: 31 | Nucleotide Sequence of | CAGGTGCAGCTGGTGCAGTCTGGGGCTGAG |
| | V_{H} of 6-G23-F | GTGAAGAAGCCTGGGGCCTCAGTGAAGGTT |
| | | TCCTGCAAGGCATCTGGATACACCTTCACCA |
| | | GCTACTATATACACTGGGTGCGACAGGCCC |
| | | CTGGACAAGGGCTTGAGTGGATGGGAATAA |
| | | TCGACCCAAGTGATGGTAGCACAAACTACG |
| | | CACAGAAGTTCCAGGGCAGAGTCACCATGA |
| | | CCAGGGACACGTCCACGAGCACAGTCTACA |
| | | TGGAGCTGAGCAGCCTGAGATCTGAGGACA |
| | | CGGCCGTGTATTACTGTGCGAGAGATCGGG |
| | | AATATAACTACTACGGTTTGGACGTCTGGGG |
| | | CCAAGGGACCACGGTCACCGTCTCCTCA |
| SEQ ID NO: 32 | Amino acid Sequence of | QVQLVQSGAEVKKPGASVKVSCKASGYTFTS |
| | V_{H} of 6-G23-F | YYIHWVRQAPGQGLEWMGIIDPSDGSTNYAQ |
| | | KFQGRVTMTRDTSTSTVYMELSSLRSEDTAV |
| | | YYCARDREYNYYGLDVWGQGTTVTVSS |
| SEQ ID NO: 33 | Amino acid Sequence of | GYTFTSY |
| | V_{H} CDR1 of 6-G23-F | |
| SEQ ID NO: 34 | Amino acid Sequence of | DPSDGS |
| | V_{H} CDR2 of 6-G23-F | |
| SEQ ID NO: 35 | Amino acid Sequence of | DREYNYYGLDV |
| | V_{H} CDR3 of 6-G23-F | |
| SEQ ID NO: 36 | Nucleotide Sequence of | GATGTTGTGATGACTCAGTCTCCACTCTCCC |
| | V_{L} of 6-G23-F | TGCCCGTCACCCTTGGACAGCCGGCCTCCAT |
| | | CTCCTGCAGGTCTAGTCAAAGCCTCGTATAC |
| | | CGTGATGGAAACACCTACTTGAATTGGTTTC |
| | | AGCAGAGGCCAGGCCAATCTCCAAGGCGCC |
| | | TAATTTATAAGGTTTCTAACCGGGACTCTGG |
| | | GGTCCCAGACAGATTCCGCGGCAGTGGGTC |
| | | AGGCACTGATTTCACACTGAAAATCAGCCG |
| | | GGTGGAGGCTGAGGATGTTGGGGTTTATTA |
| | | CTGCATGCAAGGTACACACTGGCCTCCGAC |
| | | GTTCGGCCAAGGGACCAAGGTGGAAATCAA |
| | | A |
| SEQ ID NO: 37 | Amino acid Sequence of | DVVMTQSPLSLPVTLGQPASISCRSSQSLVYR |
| | V_{L} of 6-G23-F | DGNTYLNWFQQRPGQSPRRLIYKVSNRDSGV |
| | | PDRFRGSGSGTDFTLKISRVEAEDVGVYYCM |
| | | QGTHWPPTFGQGTKVEIK |
| SEQ ID NO: 38 | Amino acid Sequence of | RSSQSLVYRDGNTYLN |
| | V_{L} CDR1 of 6-G23-F | |
| SEQ ID NO: 39 | Amino acid Sequence of | KVSNRDS |
| | V_{L} CDR2 of 6-G23-F | |
| SEQ ID NO: 40 | Amino acid Sequence of | MQGTHWPPT |
| | V_{L} CDR3 of 6-G23-F | |

### Example 8-1 Production of Recombinant Anti-DLL3 Antibodies H2-C8-A, H2-C8-A-2, and H2-C8-A-3

Construction of the heavy chain: The heavy chain of the anti-DLL3 antibody H2-C8-A (SEQ ID NO: 59) was constructed by connecting the variable region obtained in Example 8 (SEQ ID NO: 12) with the human gamma chain constant region of IgG1 (SEQ ID: 42). The heavy chains of the anti-DLL3 antibody H2-C8-A-2 (SEQ ID NO: 60) and H2-C8-A-3 (SEQ ID NO: 61) were also constructed by connecting the variable region obtained in Example 8 (SEQ ID NO: 12) with the human gamma chain constant region of IgG1 variant (SEQ ID NOs: 57 and 58).

(SEQ ID NO: 59)
EVQLVESGGGLVQPGGSQRLSCAASGFTFSSYWMNWVRQAPGKGLEWVA
NIKEDGSEKYYVDSVKGRFTISRDNAKNSLYLQMNSLRAEDTAVYYCAR
DPGWAPFDYWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKS
LSLSPGK
(SEQ ID NO: 60)
EVQLVESGGGLVQPGGSQRLSCAASGFTFSSYWMNWVRQAPGKGLEWVA
NIKEDGSEKYYVDSVKGRFTISRDNAKNSLYLQMNSLRAEDTAVYYCAR
DPGWAPFDYWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKS
LSLSPGK
(SEQ ID NO: 61)
EVQLVESGGGLVQPGGSQRLSCAASGFTFSSYWMNWVRQAPGKGLEWVA
NIKEDGSEKYYVDSVKGRFTISRDNAKNSLYLQMNSLRAEDTAVYYCAR
DPGWAPFDYWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKS
LSLSPGK

Construction of the light chain: The light chain of the anti-DLL3 antibody H2-C8-A was constructed, and the light chain of the anti-DLL3 antibody H2-C8-A-2 and H2-C8-A-3 can be constructed (SEQ ID NO: 62) by connecting the variable region obtained in Example 8 with the human kappa chain constant region of IgG1 (SEQ ID NOs: 17 and 49).

(SEQ ID NO: 62)
DIQMSQSPSSLSASVGDRVTITCRASQGISNYLAWFQQKPGKAPKSLIY
AASSLQSGVPSKFSGSGSGTDFTLAISSLQPEDFATYYCQQYNSFPYTF
GQGTTLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

Construction of the expression vector pCMA-G1: Construct of the expression vector pCMA-G1 comprising the human heavy chain signal sequence and a DNA sequence encoding a human gamma chain constant region, was described in Patent Appl. No. WO2017/051888.

Construction of the expression vector pCMA-LK: Construct of the expression vector pCMA-LK comprising the human light chain signal sequence and a DNA sequence encoding a human kappa chain constant region, was described in Patent Appl. No. WO2017/051888.

Construction of the expression vector pCMA-G1-1: A DNA fragment (SEQ ID No: 75) was synthesized (Eurofins Genomics, artificial gene synthesis service). The DNA fragment was digested with restriction enzymes of XbaI and PmeI. The resulted 1.1 kb fragment was separated by agarose gel electrophoresis and purified with Wizard SV Gel and PCR Clean-Up System (Promega). The expression vector of pCMA-G1 was also digested with restriction enzymes of XbaI and PmeI to remove the human heavy chain signal sequence and a DNA sequence encoding a human gamma chain constant region by agarose gel electrophoresis. Resulted 3.4 kb of XbaI/PmeI fragment of pCMA-G1 was also purified with Wizard SV Gel and PCR Clean-Up System. The purified 1.1 kb and 3.4 kb XbaI/PmeI fragments were ligated with Ligation High (Toyobo) to construct the expression vector pCMA-G1-1.

(SEQ ID NO: 75)
GGGTCTAGAGCCACCATGAAACACCTGTGGTTCTTCCTCCTGCTGGTGG
CAGCTCCCAGATGGGTGCTGAGCCAGGTGCAATTGTGCAGGCGGTTAGC
TCAGCCTCCACCAAGGGCCCAAGCGTCTTCCCCCTGGCACCCTCCTCCA
AGAGCACCTCTGGCGGCACAGCCGCCCTGGGCTGCCTGGTCAAGGACTA
CTTCCCCGAACCCGTGACCGTGAGCTGGAACTCAGGCGCCCTGACCAGC
GGCGTGCACACCTTCCCCGCTGTCCTGCAGTCCTCAGGACTCTACTCCC
TCAGCAGCGTGGTGACCGTGCCCTCCAGCAGCTTGGGCACCCAGACCTA
CATCTGCAACGTGAATCACAAGCCCAGCAACACCAAGGTGGACAAGAAG
GTTGAGCCCAAATCTTGTGACAAAACTCACACATGCCCACCCTGCCCAG
CACCTGAACTCCTGGGGGGACCCTCAGTCTTCCTCTTCCCCCCAAAACC
CAAGGACACCCTCATGATCTCCCGGACCCCTGAGGTCACATGCGTGGTG
GTGGACGTGAGCCACGAAGACCCTGAGGTCAAGTTCAACTGGTACGTGG
ACGGCGTGGAGGTGCATAATGCCAAGACAAAGCCCCGGGAGGAGCAGTA
CAACAGCACGTACCGGGTGGTCAGCGTCCTCACCGTCCTGCACCAGGAC
TGGCTGAATGGCAAGGAGTACAAGTGCAAGGTCTCCAACAAAGCCCTCC
CAGCCCCCATCGAGAAAACCATCTCCAAAGCCAAAGGCCAGCCCCGGGA
ACCACAGGTGTACACCCTGCCCCCATCCCGGGAGGAGATGACCAAGAAC
CAGGTCAGCCTGACCTGCCTGGTCAAAGGCTTCTATCCCAGCGACATCG
CCGTGGAGTGGGAGAGCAATGGCCAGCCCGAGAACAACTACAAGACCAC
CCCTCCCGTGCTGGACTCCGACGGCTCCTTCTTCCTCTACAGCAAGCTC
ACCGTGGACAAGAGCAGGTGGCAGCAGGGCAACGTCTTCTCATGCTCCG
TGATGCATGAGGCTCTGCACAACCACTACACCCAGAAGAGCCTCTCCCT
GTCTCCCGGCAAATGAGATATCGGGCCCGTTTAAACGGG

Construction of the expression vector of the anti-DLL3 antibody H2-C8-A-2 heavy chain: DNA fragment consisting the nucleotides at nucleotide positions 58 to 405 (in SEQ ID NO: 76) with flanking recombination sites for In-Fusion reaction both at the 5′-site (AGCTCCCAGATGGGTGCTGAGC; nucleotides 36-57 of SEQ ID NO: 76) and at the 3′-site (AGCTCAGCCTCCACCAAGGGCCC; nucleotides 406-428 of SEQ ID NO: 76) was synthesized (GENEART, artificial gene synthesis service). Using an In-Fusion HD PCR cloning kit (Takara Bio USA), the synthesized DNA fragment was inserted into a site of pCMA-G1-1 that had been cleaved with the restriction enzyme BIpI, resulted in constructing the expression vector of the anti-DLL3 antibody H2-C8-A-2 heavy chain.

(SEQ ID NO: 76)
ATGAAACACCTGTGGTTCTTCCTCCTGCTGGTGGCAGCTCCCAGATGGG
TGCTGAGCGAGGTGCAGCTGGTTGAATCTGGCGGAGGACTGGTTCAGCC
TGGCGGATCTCAGAGACTGTCTTGTGCCGCCAGCGGCTTCACCTTCAGC
AGCTACTGGATGAACTGGGTCCGACAGGCCCCTGGCAAAGGCCTTGAAT
GGGTCGCCAACATCAAAGAGGACGGCAGCGAGAAGTACTACGTGGACAG
CGTGAAGGGCAGATTCACCATCTCCAGAGACAACGCCAAGAACAGCCTG
TACCTGCAGATGAACTCCCTGAGAGCCGAGGACACCGCCGTGTACTACT
GTGCCAGAGATCCTGGCTGGGCCCCTTTCGATTATTGGGGCCAGGGCAC
ACTGGTCACCGTTAGCTCAGCCTCCACCAAGGGCCCAAGCGTCTTCCCC
CTGGCACCCTCCTCCAAGAGCACCTCTGGCGGCACAGCCGCCCTGGGCT
GCCTGGTCAAGGACTACTTCCCCGAACCCGTGACCGTGAGCTGGAACTC
AGGCGCCCTGACCAGCGGCGTGCACACCTTCCCCGCTGTCCTGCAGTCC
TCAGGACTCTACTCCCTCAGCAGCGTGGTGACCGTGCCCTCCAGCAGCT
TGGGCACCCAGACCTACATCTGCAACGTGAATCACAAGCCCAGCAACAC
CAAGGTGGACAAGAAGGTTGAGCCCAAATCTTGTGACAAAACTCACACA
TGCCCACCCTGCCCAGCACCTGAACTCCTGGGGGGACCCTCAGTCTTCC
TCTTCCCCCCAAAACCCAAGGACACCCTCATGATCTCCCGGACCCCTGA
GGTCACATGCGTGGTGGTGGACGTGAGCCACGAAGACCCTGAGGTCAAG
TTCAACTGGTACGTGGACGGCGTGGAGGTGCATAATGCCAAGACAAAGC
CCCGGGAGGAGCAGTACAACAGCACGTACCGGGTGGTCAGCGTCCTCAC
CGTCCTGCACCAGGACTGGCTGAATGGCAAGGAGTACAAGTGCAAGGTC
TCCAACAAAGCCCTCCCAGCCCCCATCGAGAAAACCATCTCCAAAGCCA
AAGGCCAGCCCCGGGAACCACAGGTGTACACCCTGCCCCCATCCCGGGA
GGAGATGACCAAGAACCAGGTCAGCCTGACCTGCCTGGTCAAAGGCTTC
TATCCCAGCGACATCGCCGTGGAGTGGGAGAGCAATGGCCAGCCCGAGA
ACAACTACAAGACCACCCCTCCCGTGCTGGACTCCGACGGCTCCTTCTT
CCTCTACAGCAAGCTCACCGTGGACAAGAGCAGGTGGCAGCAGGGCAAC
GTCTTCTCATGCTCCGTGATGCATGAGGCTCTGCACAACCACTACACCC
AGAAGAGCCTCTCCCTGTCTCCCGGCAAA

Construction of the expression vector of the anti-DLL3 antibody H2-C8-A-3 heavy chain: DNA fragment consisting the nucleotides at nucleotide positions 1 to 1401 (in SEQ ID NO: 77) with flanking recombination sites for In-Fusion reaction both at the 5′-site outside of SEQ ID NO: 77 (CCAGCCTCCGGACTCTAGAGCCACC; SEQ ID NO: 101) and at the 3′-site outside of SEQ ID NO: 77 (TGAGTTTAAACGGGGGAGGCTAACT; SEQ ID NO: 102) was synthesized (GENEART, artificial gene synthesis service). Using an In-Fusion HD PCR cloning kit, the amplified DNA fragment was inserted into a site of pCMA-LK that had been cleaved with the restriction enzyme XbaI/PmeI, resulted in constructing the expression vector of the anti-DLL3 antibody H2-C8-A-3 heavy chain.

(SEQ ID NO: 77)
ATGAAGCACCTGTGGTTCTTTCTGCTGCTGGTGGCCGCTCCTAGATGGG
TGCTGTCTGAAGTGCAGCTGGTGGAATCTGGCGGAGGACTGGTTCAACC
TGGCGGCTCTCAGAGACTGTCTTGTGCCGCCAGCGGCTTCACCTTCAGC
AGCTACTGGATGAACTGGGTCCGACAGGCCCCTGGCAAAGGCCTTGAAT
GGGTCGCCAACATCAAAGAGGACGGCAGCGAGAAGTACTACGTGGACAG
CGTGAAGGGCAGATTCACCATCTCCAGAGACAACGCCAAGAACAGCCTG
TACCTGCAGATGAACTCCCTGCGCGCCGAAGATACCGCCGTGTACTACT
GTGCCAGAGATCCTGGCTGGGCCCCTTTCGATTATTGGGGCCAGGGAAC
CCTGGTCACCGTGTCATCTGCCTCCACCAAGGGCCCAAGCGTCTTCCCC
CTGGCACCCTCCTCCAAGAGCACCTCTGGCGGCACAGCCGCCCTGGGCT
GCCTGGTCAAGGACTACTTCCCCGAACCCGTGACCGTGAGCTGGAACTC
AGGCGCCCTGACCAGCGGCGTGCACACCTTCCCCGCTGTCCTGCAGTCC
TCAGGACTCTACTCCCTCAGCAGCGTGGTGACCGTGCCCTCCAGCAGCT
TGGGCACCCAGACCTACATCTGCAACGTGAATCACAAGCCCAGCAACAC
CAAGGTGGACAAGAAGGTTGAGCCCAAATCTTGTGACAAAACTCACACA
TGCCCACCCTGCCCAGCACCTGAAGCCGCGGGGGGACCCTCAGTCTTCC
TCTTCCCCCCAAAACCCAAGGACACCCTCATGATCTCCCGGACCCCTGA
GGTCACATGCGTGGTGGTGGACGTGAGCCACGAAGACCCTGAGGTCAAG
TTCAACTGGTACGTGGACGGCGTGGAGGTGCATAATGCCAAGACAAAGC
CCCGGGAGGAGCAGTACAACAGCACGTACCGGGTGGTCAGCGTCCTCAC
CGTCCTGCACCAGGACTGGCTGAATGGCAAGGAGTACAAGTGCAAGGTC
TCCAACAAAGCCCTCCCAGCCCCCATCGAGAAAACCATCTCCAAAGCCA
AAGGCCAGCCCCGGGAACCACAGGTGTACACCCTGCCCCCATCCCGGGA
GGAGATGACCAAGAACCAGGTCAGCCTGACCTGCCTGGTCAAAGGCTTC
TATCCCAGCGACATCGCCGTGGAGTGGGAGAGCAATGGCCAGCCCGAGA
ACAACTACAAGACCACCCCTCCCGTGCTGGACTCCGACGGCTCCTTCTT
CCTCTACAGCAAGCTCACCGTGGACAAGAGCAGGTGGCAGCAGGGCAAC
GTCTTCTCATGCTCCGTGATGCATGAGGCTCTGCACAACCACTACACCC
AGAAGAGCCTCTCCCTGTCTCCCGGCAAA

Construction of the expression vector of the anti-DLL3 antibody H2-C8-A-2 and H2-C8-A-3 light chains: DNA fragment consisting the nucleotides at nucleotide positions 61 to 381 (in SEQ ID NO: 80) with flanking recombination sites for In-Fusion reaction both at the 5′-site (CTGTGGATCTCCGGCGCGTACGGC; nucleotides 37-60 of SEQ ID NO: 80) and at the 3′-site (CGTACGGTGGCCGCCCCCTCC; nucleotides 382-402 of SEQ ID NO: 80) was synthesized (GENEART, artificial gene synthesis service). Using an In-Fusion HD PCR cloning kit (Takara Bio USA), the synthesized DNA fragment was inserted into a site of pCMA-LK that had been cleaved with the restriction enzyme BsiWI, resulted in constructing the expression vector of the anti-DLL3 antibody H2-C8-A-2 and H2-C8-A-3 light chains.

(SEQ ID NO: 80)
ATGGTGCTGCAGACCCAGGTGTTCATCTCCCTGCTGCTGTGGATCTCCG
GCGCGTACGGCGACATCCAGATGTCTCAGAGCCCTAGCAGCCTGTCTGC
CAGCGTGGGAGACAGAGTGACCATCACCTGTAGAGCCAGCCAGGGCATC
AGCAACTACCTGGCCTGGTTCCAGCAGAAGCCTGGCAAGGCCCCTAAGA
GCCTGATCTATGCCGCTAGCTCTCTGCAGTCTGGCGTGCCCTCTAAGTT
TAGCGGCTCTGGCAGCGGCACCGATTTCACACTGGCCATATCTAGCCTG
CAGCCTGAGGACTTCGCCACCTACTACTGCCAGCAGTACAACAGCTTCC
CCTACACCTTCGGCCAGGGCACCACACTGGAAATCAAGCGTACGGTGGC
CGCCCCCTCCGTGTTCATCTTCCCCCCCTCCGACGAGCAGCTGAAGTCC
GGCACCGCCTCCGTGGTGTGCCTGCTGAATAACTTCTACCCCAGAGAGG
CCAAGGTGCAGTGGAAGGTGGACAACGCCCTGCAGTCCGGGAACTCCCA
GGAGAGCGTGACCGAGCAGGACAGCAAGGACAGCACCTACAGCCTGAGC
AGCACCCTGACCCTGAGCAAAGCCGACTACGAGAAGCACAAGGTGTACG
CCTGCGAGGTGACCCACCAGGGCCTGAGCTCCCCCGTCACCAAGAGCTT
CAACAGGGGGGAGTGT

Expression and purification: The expression vectors coding the corresponding DNA sequence of the heavy chain and the light chain of H2-C8-A were constructed (Transfection-grade plasmids, Genscript) and transfected to the HEK293 cell (HD 293F, Genscript). Followed by the culture and the harvest, the antibody was purified from the obtained supernatant by Protein A affinity chromatography.

Alternatively, regarding H2-C8-A-3, in accordance with the manual, FreeStyle 293F cells (Thermo Fisher Scientific) were cultured and passaged. FreeStyle 293F cells in the logarithmic growth phase were seeded on a 3-L Erlenmeyer Flask (CORNING), and were diluted with FreeStyle293 expression medium (Thermo Fisher Scientific) at 2.0-2.4×10⁶ cells/mL to a total volume of 580 mL. Meanwhile, 300 μg of the heavy chain expression vector of H2-C8-A-3, 300 μg of the light chain expression vector of H2-C8-A-3 and 1.8 mg of Polyethyleneimine (Polyscience) were added to 20 mL of Opti-Pro SFM medium (Thermo Fisher Scientific), and the obtained mixture was gently stirred. After incubation for 5 minutes, the mixture was added to the FreeStyle 293F cells. The cells were incubated in an incubator (37° C., 8% CO₂) with shaking at 95 rpm for 4 hours, and thereafter, 480 mL of BalanCD® HEK293 (FUJIFILM Irvine Scientific) including 4 mM GlutaMAX Supplement I (Thermo Fisher Scientific) and 120 mL of BalanCD® HEK293 Feed (FUJIFILM Irvine Scientific) including 4 mM GlutaMAX Supplement I were added to the culture. The cells were further incubated in an incubator (37° C., 8% CO₂) with shaking at 95 rpm for 6 days. The culture supernatant was harvested and filtrated with a 500-mL Filter System (Thermo Fisher Scientific).

On the other hand, regarding H2-C8-A-2, in accordance with the manual, FreeStyle 293F cells were cultured and passaged in a spinner flask with Middle Scale Bioreactor BCP (Biott) at 37° C., 8% CO₂. Transfection and cultivation of FreeStyle 293F cells were carried out with WAVE BIOREACTOR (GE healthcare). 2.5 L of FreeStyle 293F cells at 2.0-2.4×10⁶ cells/mL in the logarithmic growth phase were seeded on a WAVE CELLBAG10L (Cytiva). Meanwhile, 1.25 mg of the heavy chain expression vector of H2-C8-A-2, 1.25 mg of the light chain expression vector of H2-C8-A-2 and 7.5 mg of Polyethyleneimine (Polyscience) were added to 160 mL of Opti-Pro SFM medium (Thermo Fisher Scientific), and the obtained mixture was gently stirred. After incubation for 5 minutes, the mixture was added to the FreeStyle 293F cells in the WAVE CELLBAG10L. The cells were cultivated in the WAVE CELLBAG10L (37° C., 8% CO₂) with rocking for 4 hours, and thereafter, 1.92 L of BalanCD® HEK293 including 4 mM GlutaMAX Supplement I and 480 mL of BalanCD® HEK293 Feed including 4 mM GlutaMAX Supplement I were added to the culture. The cells were further cultivated in the WAVE CELLBAG10L (37° C., 8% CO₂) with rocking for 6 days. The culture supernatant was harvested, centrifuged and filtrated with the CAPSULE CARTRIDGE FILTER (Pore size: 0.45 □m, ADVANTEC).

Purification of anti-DLL3 antibodies: The filtrated culture supernatant was purified by a two-step process of rProtein A affinity chromatography and ceramic hydroxyapatite. Detail of the purification method was described in Patent Appl. No. WO2020/013170.

### Example 8-2—MAB2-Production of Recombinant Anti-DLL3 Antibodies H6-G23-F, H6-G23-F-2, and H6-G23-F-3

Construction of the heavy chain: The heavy chains of the anti-DLL3 antibody H6-G23-F (SEQ ID NO: 63) was constructed by connecting the variable region obtained in Example 8 (SEQ ID NO: 32) with the human gamma chain constant region of IgG1 (SEQ ID: 42). The heavy chains of the anti-DLL3 antibody H6-G23-F-2 and H6-G23-F-3 (SEQ ID NOs: 64 and 65, respectively) were also constructed by connecting the variable region obtained in Example 8 (SEQ ID NO: 32) with the human gamma chain constant region of IgG1 variant (SEQ ID NOs: 57 and 58).

(SEQ ID NO: 63)
QVQLVQSGAEVKKPGASVKVSCKASGYTFTSYYIHWVRQAPGQGLEWMG
IIDPSDGSTNYAQKFQGRVTMTRDTSTSTVYMELSSLRSEDTAVYYCAR
DREYNYYGLDVWGQGTTVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQ
KSLSLSPGK
(SEQ ID NO: 64)
QVQLVQSGAEVKKPGASVKVSCKASGYTFTSYYIHWVRQAPGQGLEWMG
IIDPSDGSTNYAQKFQGRVTMTRDTSTSTVYMELSSLRSEDTAVYYCAR
DREYNYYGLDVWGQGTTVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQ
KSLSLSPGK
(SEQ ID NO: 65)
QVQLVQSGAEVKKPGASVKVSCKASGYTFTSYYIHWVRQAPGQGLEWMG
IIDPSDGSTNYAQKFQGRVTMTRDTSTSTVYMELSSLRSEDTAVYYCAR
DREYNYYGLDVWGQGTTVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSCDKTHTCPPCPAPEAAGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQ
KSLSLSPGK

Construction of the light chain: The light chain of the anti-DLL3 antibody H6-G23-F was constructed and the light chain of the anti-DLL3 antibody H6-G23-F-2 and H6-G23-F-3 can be constructed (SEQ ID NO: 66) by connecting the variable region obtained in Example 8 with the human kappa chain constant region of IgG1 (SEQ ID NOs: 37 and 49).

(SEQ ID NO: 66)
DVVMTQSPLSLPVTLGQPASISCRSSQSLVYRDGNTYLNWFQQRPGQSP
RRLIYKVSNRDSGVPDRFRGSGSGTDFTLKISRVEAEDVGVYYCMQGTH
WPPTFGQGTKVEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC

Expression and purification: The expression vectors coding the corresponding DNA sequence of the heavy chain and the light chain of H6-G23-F describe above were prepared (Transfection-grade plasmids, Genscript) and transfected to the HEK293 cell (HD 293F, Genscript). Followed by the culture and the harvest, the antibody was purified from the obtained supernatant by Protein A affinity chromatography.

### Example 8-3—MAB3-Production of Recombinant Anti-DLL3 Antibodies H10-O18-A, H10-O18-A-2, and H10-O18-A-3

Construction of the heavy chain: The heavy chains of the anti-DLL3 antibody H10-O18-A was constructed (SEQ ID NO: 67) by connecting the variable region obtained in Example 8 (SEQ ID NO: 22) with the human gamma chain constant region of IgG1 (SEQ ID NO: 42). The heavy chains of the anti-DLL3 antibody H10-O18-A-2 and H10-O18-A-3 (SEQ ID NOs:68 and 69, respectively) can be constructed by connecting the variable region obtained in Example 8 (SEQ ID NO: 22) with the human gamma chain constant region of IgG1 variant (SEQ ID NOs: 57 and 58)

(SEQ ID NO: 67)
QVQLQESGPGLVKPSETLSLTCTVSGGSINSYYWSWIRQPPGKGLEWIG
YIFYSGITNYNPSLKSRVTISLDTSKNQFSLKLSSVTAADTAVYYCARI
GVAGFYFDYWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKS
LSLSPGK
(SEQ ID NO: 68)
QVQLQESGPGLVKPSETLSLTCTVSGGSINSYYWSWIRQPPGKGLEWIG
YIFYSGITNYNPSLKSRVTISLDTSKNQFSLKLSSVTAADTAVYYCARI
GVAGFYFDYWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKS
LSLSPGK
(SEQ ID NO: 69)
QVQLQESGPGLVKPSETLSLTCTVSGGSINSYYWSWIRQPPGKGLEWIG
YIFYSGITNYNPSLKSRVTISLDTSKNQFSLKLSSVTAADTAVYYCARI
GVAGFYFDYWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKS
LSLSPGK

Construction of the light chain: The light chain of the anti-DLL3 antibody H10-O18-A was constructed, and the light chain of the anti-DLL3 antibody H10-O18-A-2 and H10-O18-A-3 can be constructed (SEQ ID NO: 70) by connecting the variable region obtained in Example 8 with the human kappa chain constant region of IgG1 (SEQ ID NOs: 27 and 49). EIVLTQSPGTLSL SPGERATL SCRASQSVSSSYLAWYQQKPGQAPRLLIYGASSRATGI PDRFSGSGSGTDFTLTISRLEPEDFAVYYCQQYGTSPLTFGGGTKVEIKRTVAAPSVFIF PPSDEQLKSGTASVVCLLNNFYPREAKVQWKVDNALQSGNSQESVTEQDSKDSTYS LSSTLTLSKADYEKHKVYACEVTHQGLSSPVTKSFNRGEC (SEQ ID NO:70)

Construction of the expression vector of the anti-DLL3 antibody H10-O18-A-2 heavy chain: DNA fragment consisting the nucleotides at nucleotide positions 58 to 405 (in SEQ ID NO: 78) with flanking recombination sites for In-Fusion reaction both at the 5′-site (AGCTCCCAGATGGGTGCTGAGC; nucleotides 36-57 of SEQ ID NO: 78) and at the 3′-site (AGCTCAGCCTCCACCAAGGGCCC; nucleotides 406-428 of SEQ ID NO: 78) was synthesized (GENEART, artificial gene synthesis service). Using an In-Fusion HD PCR cloning kit (Takara Bio USA), the synthesized DNA fragment was inserted into a site of pCMA-G1-1 that had been cleaved with the restriction enzyme BIpI, resulted in constructing the expression vector of the anti-DLL3 antibody H10-O18-A-2 heavy chain.

(SEQ ID NO: 78)
ATGAAACACCTGTGGTTCTTCCTCCTGCTGGTGGCAGCTCCCAGATGGG
TGCTGAGCCAGGTTCAGCTGCAAGAGTCTGGCCCTGGCCTGGTCAAGCC
TAGCGAAACACTGAGCCTGACCTGTACCGTGTCTGGCGGCAGCATCAAC
AGCTACTACTGGTCCTGGATCCGGCAGCCTCCTGGCAAAGGACTGGAAT
GGATCGGCTACATCTTCTACAGCGGCATCACCAACTACAACCCCAGCCT
GAAGTCCAGAGTGACCATCAGCCTGGACACCAGCAAGAACCAGTTCTCC
CTGAAGCTGAGCAGCGTGACAGCCGCCGATACAGCCGTGTACTACTGTG
CCAGAATCGGCGTGGCCGGCTTCTACTTCGATTATTGGGGCCAGGGCAC
CCTGGTCACAGTTAGCTCAGCCTCCACCAAGGGCCCAAGCGTCTTCCCC
CTGGCACCCTCCTCCAAGAGCACCTCTGGCGGCACAGCCGCCCTGGGCT
GCCTGGTCAAGGACTACTTCCCCGAACCCGTGACCGTGAGCTGGAACTC
AGGCGCCCTGACCAGCGGCGTGCACACCTTCCCCGCTGTCCTGCAGTCC
TCAGGACTCTACTCCCTCAGCAGCGTGGTGACCGTGCCCTCCAGCAGCT
TGGGCACCCAGACCTACATCTGCAACGTGAATCACAAGCCCAGCAACAC
CAAGGTGGACAAGAAGGTTGAGCCCAAATCTTGTGACAAAACTCACACA
TGCCCACCCTGCCCAGCACCTGAACTCCTGGGGGGACCCTCAGTCTTCC
TCTTCCCCCCAAAACCCAAGGACACCCTCATGATCTCCCGGACCCCTGA
GGTCACATGCGTGGTGGTGGACGTGAGCCACGAAGACCCTGAGGTCAAG
TTCAACTGGTACGTGGACGGCGTGGAGGTGCATAATGCCAAGACAAAGC
CCCGGGAGGAGCAGTACAACAGCACGTACCGGGTGGTCAGCGTCCTCAC
CGTCCTGCACCAGGACTGGCTGAATGGCAAGGAGTACAAGTGCAAGGTC
TCCAACAAAGCCCTCCCAGCCCCCATCGAGAAAACCATCTCCAAAGCCA
AAGGCCAGCCCCGGGAACCACAGGTGTACACCCTGCCCCCATCCCGGGA
GGAGATGACCAAGAACCAGGTCAGCCTGACCTGCCTGGTCAAAGGCTTC
TATCCCAGCGACATCGCCGTGGAGTGGGAGAGCAATGGCCAGCCCGAGA
ACAACTACAAGACCACCCCTCCCGTGCTGGACTCCGACGGCTCCTTCTT
CCTCTACAGCAAGCTCACCGTGGACAAGAGCAGGTGGCAGCAGGGCAAC
GTCTTCTCATGCTCCGTGATGCATGAGGCTCTGCACAACCACTACACCC
AGAAGAGCCTCTCCCTGTCTCCCGGCAAA

Construction of the expression vector of the anti-DLL3 antibody H10-O18-A-3 heavy chain: DNA fragment consisting the nucleotides at nucleotide positions 1 to 1401 (in SEQ ID NO: 79) with flanking recombination sites for In-Fusion reaction both at the 5′-site outside of SEQ ID NO: 79 (CCAGCCTCCGGACTCTAGAGCCACC; SEQ ID NO: 101) and at the 3′-site outside of SEQ ID NO: 79 (TGAGTTTAAACGGGGGAGGCTAACT; SEQ ID NO: 102) was synthesized (GENEART, artificial gene synthesis service). Using an In-Fusion HD PCR cloning kit, the amplified DNA fragment was inserted into a site of pCMA-LK that had been cleaved with the restriction enzyme PmeI/XbaI, resulted in constructing the expression vector of the anti-DLL3 antibody H10-O18-A-3 heavy chain.

(SEQ ID NO: 79)
ATGAAGCACCTGTGGTTCTTTCTGCTGCTGGTGGCCGCTCCTAGATGGG
TGCTGTCTCAGGTTCAGCTGCAAGAGTCTGGCCCTGGCCTGGTCAAGCC
TAGCGAAACACTGAGCCTGACCTGTACCGTGTCTGGCGGCAGCATCAAC
AGCTACTACTGGTCCTGGATCCGGCAGCCTCCTGGCAAAGGACTGGAAT
GGATCGGCTACATCTTCTACAGCGGCATCACCAACTACAACCCCAGCCT
GAAGTCCAGAGTGACCATCAGCCTGGACACCAGCAAGAACCAGTTCTCC
CTGAAGCTGAGCAGCGTGACAGCCGCCGATACAGCCGTGTACTACTGTG
CCAGAATCGGCGTGGCCGGCTTCTACTTCGATTATTGGGGCCAGGGCAC
CCTGGTCACCGTTTCTTCTGCCTCCACCAAGGGCCCAAGCGTCTTCCCC
CTGGCACCCTCCTCCAAGAGCACCTCTGGCGGCACAGCCGCCCTGGGCT
GCCTGGTCAAGGACTACTTCCCCGAACCCGTGACCGTGAGCTGGAACTC
AGGCGCCCTGACCAGCGGCGTGCACACCTTCCCCGCTGTCCTGCAGTCC
TCAGGACTCTACTCCCTCAGCAGCGTGGTGACCGTGCCCTCCAGCAGCT
TGGGCACCCAGACCTACATCTGCAACGTGAATCACAAGCCCAGCAACAC
CAAGGTGGACAAGAAGGTTGAGCCCAAATCTTGTGACAAAACTCACACA
TGCCCACCCTGCCCAGCACCTGAAGCCGCGGGGGGACCCTCAGTCTTCC
TCTTCCCCCCAAAACCCAAGGACACCCTCATGATCTCCCGGACCCCTGA
GGTCACATGCGTGGTGGTGGACGTGAGCCACGAAGACCCTGAGGTCAAG
TTCAACTGGTACGTGGACGGCGTGGAGGTGCATAATGCCAAGACAAAGC
CCCGGGAGGAGCAGTACAACAGCACGTACCGGGTGGTCAGCGTCCTCAC
CGTCCTGCACCAGGACTGGCTGAATGGCAAGGAGTACAAGTGCAAGGTC
TCCAACAAAGCCCTCCCAGCCCCCATCGAGAAAACCATCTCCAAAGCCA
AAGGCCAGCCCCGGGAACCACAGGTGTACACCCTGCCCCCATCCCGGGA
GGAGATGACCAAGAACCAGGTCAGCCTGACCTGCCTGGTCAAAGGCTTC
TATCCCAGCGACATCGCCGTGGAGTGGGAGAGCAATGGCCAGCCCGAGA
ACAACTACAAGACCACCCCTCCCGTGCTGGACTCCGACGGCTCCTTCTT
CCTCTACAGCAAGCTCACCGTGGACAAGAGCAGGTGGCAGCAGGGCAAC
GTCTTCTCATGCTCCGTGATGCATGAGGCTCTGCACAACCACTACACCC
AGAAGAGCCTCTCCCTGTCTCCCGGCAAA

Construction of the expression vector of the anti-DLL3 antibody H10-O18-A-2 and H10-O18-A-3 light chains: DNA fragment consisting the nucleotides at nucleotide positions 61 to 384 (in SEQ ID NO: 81) with flanking recombination sites for In-Fusion reaction both at the 5′-site (CTGTGGATCTCCGGCGCGTACGGC; nucleotides 37-60 of SEQ ID NO: 81) and at the 3′-site (CGTACGGTGGCCGCCCCCTCC; nucleotides 385-405 of SEQ ID NO: 81) was synthesized (GENEART, artificial gene synthesis service). Using an In-Fusion HD PCR cloning kit (Takara Bio USA), the synthesized DNA fragment was inserted into a site of pCMA-LK that had been cleaved with the restriction enzyme BsiWI, resulted in constructing the expression vector of the anti-DLL3 antibody H10-O18-A-2 and H10-O18-A-3 light chains.

(SEQ ID NO: 81)
ATGGTGCTGCAGACCCAGGTGTTCATCTCCCTGCTGCTGTGGATCTCCG
GCGCGTACGGCGAGATCGTGCTGACACAGAGCCCTGGCACACTGTCACT
GTCTCCAGGCGAAAGAGCCACACTGAGCTGTAGAGCCAGCCAGAGCGTG
TCCAGCTCTTACCTGGCTTGGTATCAGCAGAAGCCCGGACAGGCTCCCA
GACTGCTGATCTATGGCGCCTCTTCTAGAGCCACAGGCATCCCCGATAG
ATTCAGCGGCTCTGGCAGCGGCACCGATTTCACCCTGACAATCAGCAGA
CTGGAACCCGAGGACTTCGCCGTGTACTACTGTCAGCAGTACGGCACAA
GCCCTCTGACCTTTGGCGGCGGAACAAAGGTGGAAATCAAGCGTACGGT
GGCCGCCCCCTCCGTGTTCATCTTCCCCCCCTCCGACGAGCAGCTGAAG
TCCGGCACCGCCTCCGTGGTGTGCCTGCTGAATAACTTCTACCCCAGAG
AGGCCAAGGTGCAGTGGAAGGTGGACAACGCCCTGCAGTCCGGGAACTC
CCAGGAGAGCGTGACCGAGCAGGACAGCAAGGACAGCACCTACAGCCTG
AGCAGCACCCTGACCCTGAGCAAAGCCGACTACGAGAAGCACAAGGTGT
ACGCCTGCGAGGTGACCCACCAGGGCCTGAGCTCCCCCGTCACCAAGAG
CTTCAACAGGGGGGAGTGT

Expression and purification: The expression vectors coding the corresponding DNA sequence of the heavy chain and the light chain of H10-O18-A were constructed (Transfection-grade plasmids, Genscript) and transfected to the HEK293 cell (HD 293F, Genscript). Followed by the culture and the harvest, the antibody was purified from the obtained supernatant by Protein A affinity chromatography.

Alternatively, regarding H10-O18-A-3, in accordance with the manual, FreeStyle 293F cells (Thermo Fisher Scientific) were cultured and passaged. FreeStyle 293F cells in the logarithmic growth phase were seeded on a 3-L Erlenmeyer Flask (CORNING), and were diluted with FreeStyle293 expression medium (Thermo Fisher Scientific) at 2.0-2.4×10⁶ cells/mL to a total volume of 580 mL. Meanwhile, 300 μg of the heavy chain expression vector of H10-O18-A-3, 300 μg of the light chain expression vector of H10-O18-A-3 and 1.8 mg of Polyethyleneimine (Polyscience) were added to 20 mL of Opti-Pro SFM medium (Thermo Fisher Scientific), and the obtained mixture was gently stirred. After incubation for 5 minutes, the mixture was added to the FreeStyle 293F cells. The cells were incubated in an incubator (37° C., 8% CO₂) with shaking at 95 rpm for 4 hours, and thereafter, 480 mL of BalanCD® HEK293 (FUJIFILM Irvine Scientific) including 4 mM GlutaMAX Supplement I (Thermo Fisher Scientific) and 120 mL of BalanCD® HEK293 Feed (FUJIFILM Irvine Scientific) including 4 mM GlutaMAX Supplement I were added to the culture. The cells were further incubated in an incubator (37° C., 8% CO₂) with shaking at 95 rpm for 6 days. The culture supernatant was harvested and filtrated with a 500-mL Filter System (Thermo Fisher Scientific). On the other hand, regarding H10-O18-A-2, in accordance with the manual, FreeStyle 293F cells were cultured and passaged in a spinner flask with Middle Scale Bioreactor BCP (Biott) at 37° C., 8% CO₂. Transfection and cultivation of FreeStyle 293F cells were carried out with WAVE BIOREACTOR (GE healthcare). 2.5 L of FreeStyle 293F cells at 2.0-2.4×10⁶ cells/mL in the logarithmic growth phase were seeded on a WAVE CELLBAG10L (Cytiva). Meanwhile, 1.25 mg of the heavy chain expression vector of H10-O18-A-2, 1.25 mg of the light chain expression vector of H10-O18-A-2 and 7.5 mg of Polyethyleneimine (Polyscience) were added to 160 mL of Opti-Pro SFM medium (Thermo Fisher Scientific), and the obtained mixture was gently stirred. After incubation for 5 minutes, the mixture was added to the FreeStyle 293F cells in the WAVE CELLBAG10L. The cells were cultivated in the WAVE CELLBAG10L (37° C., 8% CO₂) with rocking for 4 hours, and thereafter, 1.92 L of BalanCD® HEK293 including 4 mM GlutaMAX Supplement I and 480 mL of BalanCD® HEK293 Feed including 4 mM GlutaMAX Supplement I were added to the culture. The cells were further cultivated in the WAVE CELLBAG10L (37° C., 8% CO₂) with rocking for 6 days. The culture supernatant was harvested, centrifuged and filtrated with the CAPSULE CARTRIDGE FILTER (Pore size: 0.45 μm, ADVANTEC)

Purification of anti-DLL3 antibodies: The filtrated culture supernatant was purified by a two-step process of rProtein A affinity chromatography and ceramic hydroxyapatite. Detail of the purification method was described in Patent Appl. No. WO2020/013170.

### Example 9—Glycan Remodeling (H2-C8-A Antibody-[MSG1-N₃]₂)

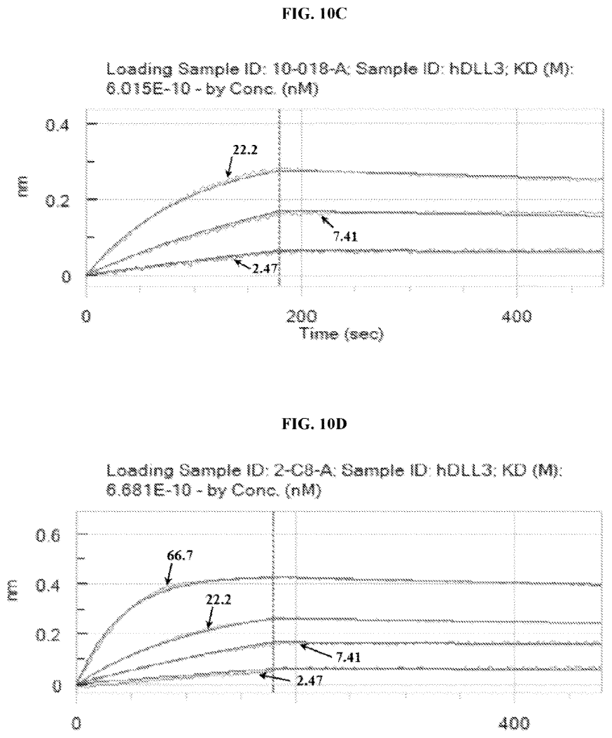

The glycan remodeling of anti-DLL3 antibody H2-C8-A was performed, as shown in the scheme in FIG. 13. This figure shows a scheme in which a linker structure in which an azide group has been introduced to a sialic acid at the non-reducing terminal of an MSG1-type N297 glycan. The linker structures of intermediates formed by introducing an azide group to an N297 glycan are all the same as the structure represented by the formula.

Step 1: Preparation of (Fucα1,6)GlcNAc-H2-C8-A antibody

The H2-C8-A antibody solution (8.02 mg/mL in PBS (pH 7.2), 12.0 mL) was subjected to buffer exchange to 50 mM phosphate buffer (pH 6.0) according to common operation C. To the resulting H2-C8-A antibody solution (20.2 mg/mL in 50 mM phosphate buffer (pH 6.0), 2.50 mL), 0.0335 mL of wild-type EndoS solution (7.52 mg/mL in PBS) was added, and the solutions was incubated at 37° C. for 4 hours. The progress of the reaction was checked by Microchip electrophoresis system (Bioanalyzer2100, Agilent). After the completion of the reaction, purification by affinity chromatography and purification with a hydroxyapatite column were performed in accordance with the following procedure.

(1) Purification Apparatus: AKTA Pure150 (Produced by GE Healthcare)
Column: HiTrap rProtein A FF (5 mL) (produced by GE Healthcare)
Flow rate: 5 mL/min (1.25 mL/min in charging)

3 CV of binding buffer (20 mM phosphate buffer (pH 6.0)) was flowed at 1.25 mL/min and 5 CV thereof was further flowed at 5 mL/min. In intermediate washing, 15 CV of washing solution (20 mM phosphate buffer (pH 7.0), 0.5 M sodium chloride solution) was flowed. In elution, 6 CV of elution buffer (ImmunoPure IgG Elution buffer, produced by Pierce) was flowed. The eluate was immediately neutralized with 1 M Tris buffer (pH 9.0). Fractions containing the desired compound were subjected to buffer exchange to 5 mM phosphate buffer/50 mM 2-morpholinoethanesulfonic acid (MES) solution (pH 6.8) by using common operation C.

(2) Purification by Hydroxyapatite Chromatography
Purification apparatus: AKTA avant25 (produced by GE Healthcare)
Column: Bio-Scale Mini CHT Type I cartridge (5 mL) (produced by Bio-Rad Laboratories, Inc.)
Flow rate: 5 mL/min (1.25 mL/min in charging)

The solution obtained in step 1 was added to the upper part of the column, and 3 CV of solution (5 mM phosphate buffer, 50 mM 2-morpholinoethanesulfonic acid (MES) solution (pH 6.8)) was flowed at 1.25 mL/min and 3 CV thereof was further flowed at 5 mL/min. Thereafter, elution was performed with solution A and solution B (5 mM phosphate buffer/50 mM 2-morpholinoethanesulfonic acid (MES) solution (pH 6.8), 2 M sodium chloride solution). The elution conditions were solution A: solution B=100:0 to 0:100 (15 CV). Further, 5 CV of washing solution (500 mM phosphate buffer (pH 6.5)) was flowed. Fractions containing the desired compound were subjected to buffer exchange by using common operation C to afford a (Fucα1,6)GlcNAc-H2-C8-A antibody solution (19.4 mg/mL in 50 mM phosphate buffer (pH 6.0), 2.10 mL).

Step 2: Preparation of H2-C8-A Antibody-[MSG1-N₃]₂

To the (Fucα1,6)GlcNAc-H2-C8-A antibody solution (19.4 mg/mL in 50 mM phosphate buffer (pH 6.0), 2.10 mL) obtained in step 1, a solution of the oxazoline (6.46 mg), [N3-PEG (3)]-MSG1-Ox (Example 56 in WO19065964), in 50 mM phosphate buffer (pH 6.0) (0.129 mL) and 0.170 mL of EndoS D233Q/Q303L solution (4.80 mg/mL in PBS) were added, and the mixture was incubated at 30° C. for 4.5 hours. The progress of the reaction was checked by Microchip electrophoresis system (Bioanalyzer2100, Agilent). After the completion of the reaction, purification by affinity chromatography and purification by hydroxyapatite chromatography were performed as in step 1, and fractions containing the desired compound were then subjected to buffer exchange to 10 mM Acetate Buffer, 5% Sorbitol (pH5.5) by using common operation C to afford a H2-C8-A antibody-[MSG1-N₃]₂ solution (11.1 mg/mL in 10 mM Acetate Buffer, 5% Sorbitol (pH5.5), 3.00 mL).

### Example 10—Glycan Remodeling (H6-G23-F Antibody-[MSG1-N₃]₂)

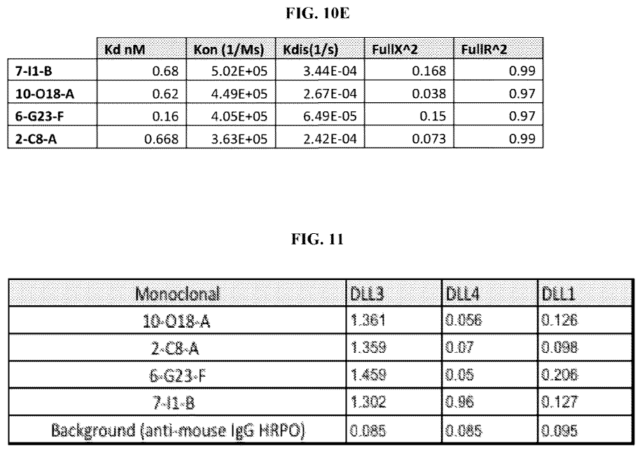

The glycan remodeling of anti-DLL3 antibody H6-G23-F was performed, as shown in the scheme in FIG. 14. This figure shows a scheme in which a linker structure in which an azide group has been introduced to a sialic acid at the non-reducing terminal of an MSG1-type N297 glycan. The linker structures of intermediates formed by introducing an azide group to an N297 glycan are all the same as the structure represented by the formula.

Step 1: Preparation of (Fucα1,6)GlcNAc-H6-G23-F Antibody

The H6-G23-F antibody solution (6.22 mg/mL in PBS (pH 7.2), 16.5 mL) was subjected to buffer exchange to 50 mM phosphate buffer (pH 6.0) according to common operation C. Using the resulting H6-G23-F antibody solution (19.9 mg/mL in 50 mM phosphate buffer (pH 6.0), 2.50 mL), the operations same as in step 1 of Example 9 were performed to afford a (Fucα1,6)GlucNAc-H6-G23-F antibody solution (19.9 mg/mL in PB (pH6.0), 2.00 mL).

Step 2: Preparation of H6-G23-F Antibody-[MSG1-N₃]₂

The operations same as in step 2 of Example 9 were performed using (Fucα1,6)GlucNAc-H6-G23-F antibody solution (19.9 mg/mL in PB (pH6.0), 2.00 mL) obtained in the above step 1 to afford a H6-G23-F Antibody-[MSG1-N₃]₂ solution (9.24 mg/mL in 10 mM Acetate Buffer, 5% Sorbitol (pH5.5), 3.50 mL).

### Example 11—Glycan Remodeling (H10-O18-A Antibody-[MSG1-N₃]₂)

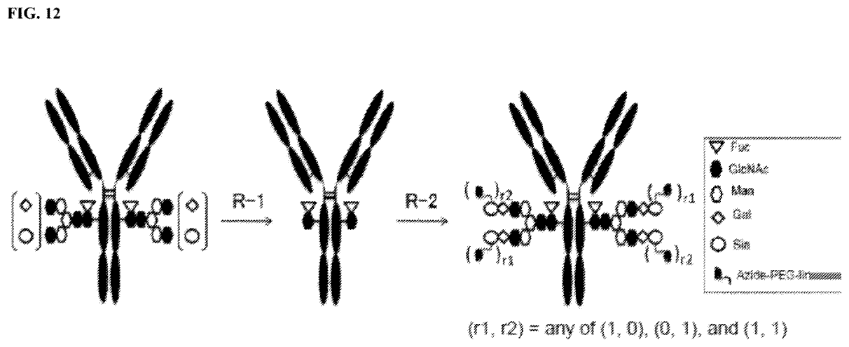

The glycan remodeling of anti-DLL3 antibody H10-O18-A was performed, as shown in the scheme in FIG. 15. This figure shows a scheme in which a linker structure in which an azide group has been introduced to a sialic acid at the non-reducing terminal of an MSG1-type N297 glycan. The linker structures of intermediates formed by introducing an azide group to an N297 glycan are all the same as the structure represented by the formula.

Step 1: Preparation of (Fucα1,6)GlcNAc-H10-O18-A antibody

The H10-O18-A antibody solution (6.87 mg/mL in PBS (pH 7.2), 16.0 mL) was subjected to buffer exchange to 50 mM phosphate buffer (pH 6.0) according to common operation C. Using the resulting H10-O18-A antibody solution (20.1 mg/mL in 50 mM phosphate buffer (pH 6.0), 2.50 mL), the operations same as in step 1 of Example 9 were performed to afford a (Fucα1,6)GlucNAc-H10-O18-A antibody solution (21.0 mg/mL in PB (pH6.0), 2.00 mL).

Step 2: Preparation of H10-O18-A Antibody-[MSG1-N₃]₂

The operations same as in step 2 of Example 9 were performed using (Fucα1,6)GlucNAc-H10-O18-A antibody solution (21.0 mg/mL in PB (pH 6.0), 2.00 mL) obtained in step 1 to afford a H10-O18-A Antibody-[MSG1-N₃]₂ solution (10.7 mg/mL in 10 mM Acetate Buffer, 5% Sorbitol (pH5.5), 3.50 mL).

### Example 12—Synthesis of H2-C8-A-Conjugate

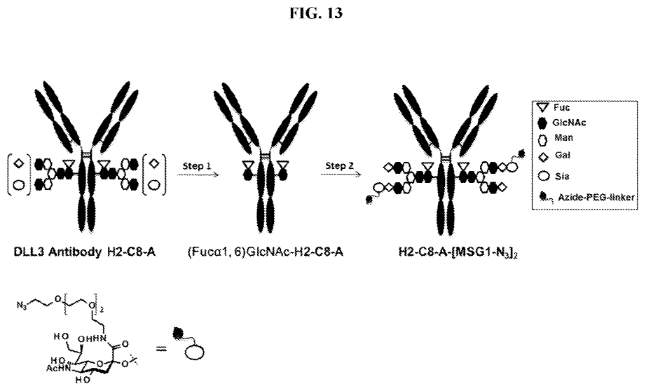

ADCs were synthesized, as illustrated in the following reaction formula, by conjugating the antibody obtained in step 2 of Example 9 and a drug-linker synthesized according to WO 2019/065964. The first step of the synthesis is shown in FIG. 16. The triazole ring to be formed in step 1 has geometric isomerization, and the compound obtained in step 1 has a linker as a mixture of the two structures shown as R in FIG. 16.

Step 1: Conjugation of Antibody H2-C8-A-[MSG1-N₃]₂ and drug-linker

To a 10 mM Acetate Buffer, 5% Sorbitol (pH 5.5) solution of the H2-C8-A antibody-(MSG1-N₃)₂ (11.1 mg/mL, 1.5 mL) obtained in step 2 of Example 9, 1,2-propanediol (1.43 mL) and a 10 mM dimethyl sulfoxide solution of N-[4-(11,12-didehydrodibenzo[b,f]azocin-5(6H)-yl)-4-oxobutanoyl]glycylglycyl-L-valyl-N-[4-({[(11′S,11′aS)-11′-hydroxy-7′-methoxy-8′-[(5-{[(11aS)-7-methoxy-2-(4-methoxyphenyl)-5-oxo-5,10,11,11a-tetrahydro-1H-pyrrolo[2,1-c][1,4]benzodiazepin-8-yl]oxy}pentyl)oxy]-5′-oxo-11′,11′a-dihydro-1′H,3′H-spiro[cyclopropane-1,2′-pyrrolo[2,1-c][1,4]benzodiazepine]-10′(5′H)-carbonyl]oxy}methyl)phenyl]-L-alaninamide (“GGVA” disclosed as SEQ ID NO: 85) prepared according to the procedure of Example 3 in WO 2019/065964 (0.0690 mL; 6 equivalents per antibody molecule) were added at room temperature, and the resultant was reacted using a tube rotator (MTR-103, AS ONE Corporation) at room temperature for 48 hours.

Purification operation: The solution was purified by using common operation D to afford 7.0 mL of a solution of the desired compound.

Characterization: The following characteristic values were obtained by using common operations E (using εA, 280=220378, εA, 329=0, ε D, 280=23155 and 8 D, 329=19492) and F (gradient program 1).

Antibody concentration: 1.77 mg/mL, antibody yield: 12.4 mg (74%), average number of conjugated drug molecules per antibody molecule (n): 1.8.

### Example 13—Synthesis of H6-G23-F-Conjugate

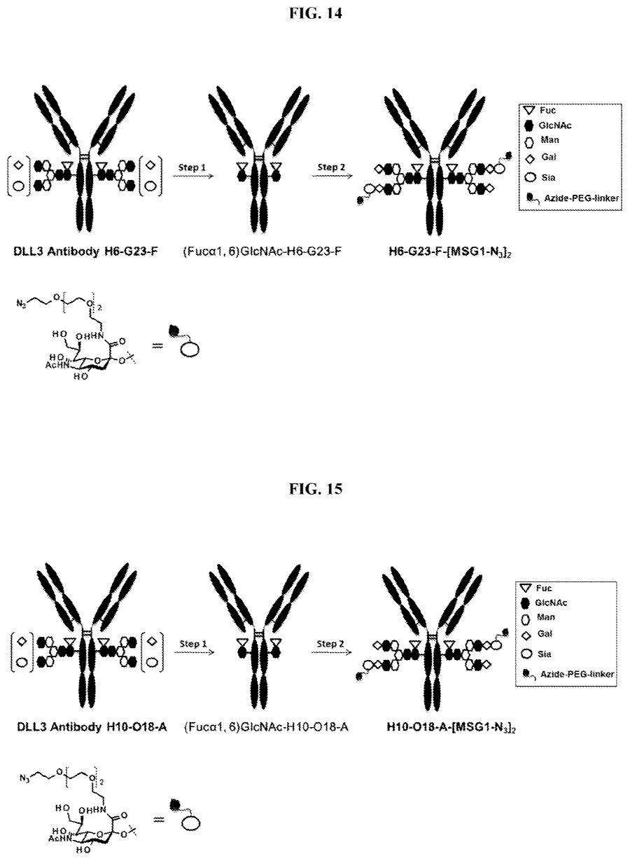

ADCs were synthesized, as illustrated in the following reaction formula, by conjugating the antibody obtained in step 2 of Example 10 and N-[4-(11,12-didehydrodibenzo[b,f]azocin-5(6H)-yl)-4-oxobutanoyl]glycylglycyl-L-valyl-N-[4-({[(11′S,11′aS)-11′-hydroxy-7′-methoxy-8′-[(5-{[(11aS)-7-methoxy-2-(4-methoxyphenyl)-5-oxo-5,10,11,11a-tetrahydro-1H-pyrrolo[2,1-c][1,4]benzodiazepin-8-yl]oxy}pentyl)oxy]-5′-oxo-11′,11′a-dihydro-1′H,3′H-spiro[cyclopropane-1,2′-pyrrolo[2,1-c][1,4]benzodiazepine]-10′(5′H)-carbonyl]oxy}methyl)phenyl]-L-alaninamide (“GGVA” disclosed as SEQ ID NO: 85) prepared according to the procedure of Example 3 in WO 2019/065964. The first step of the synthesis is shown in FIG. 17. The triazole ring to be formed in step 1 has geometric isomerization, and the compound obtained in step 1 has a linker as a mixture of the two structures shown as R in FIG. 17.

Step 1: Conjugation of Antibody H6-G23-F-[MSG1-N₃]₂ and Drug-Linker

The operations same as Example 12 were performed using a H6-G23-F-[MSG1-N₃]₂ solution (9.24 mg/mL in 10 mM Acetate Buffer, 5% Sorbitol (pH 5.5), 1.1 mL). As the result, H6-G23-F ADC solution (6.0 mL) was obtained.

Characterization: The following characteristic values were obtained by using common operations E (using εA, 280=215353, εA, 329=0, ε D, 280=23155 and 8 D, 329=19492) and F (gradient program 1).

Antibody concentration: 1.38 mg/mL, antibody yield: 8.30 mg (82%), average number of conjugated drug molecules per antibody molecule (n): 1.8

### Example 14—Synthesis of H10-O18-A-Conjugate

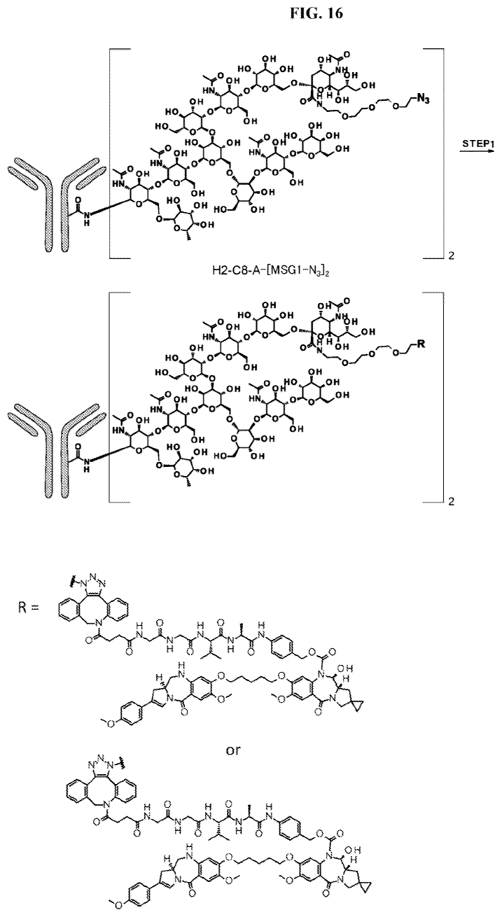

ADCs were synthesized, as illustrated in the following reaction formula, by conjugating the antibody obtained in step 2 of Example 11 and N-[4-(11,12-didehydrodibenzo[b,f]azocin-5(6H)-yl)-4-oxobutanoyl]glycylglycyl-L-valyl-N-[4-({[(11′S,11′aS)-11′-hydroxy-7′-methoxy-8′-[(5-{[(11aS)-7-methoxy-2-(4-methoxyphenyl)-5-oxo-5,10,11,11a-tetrahydro-1H-pyrrolo[2,1-c][1,4]benzodiazepin-8-yl]oxy}pentyl)oxy]-5′-oxo-11′,11′a-dihydro-1′H,3′H-spiro[cyclopropane-1,2′-pyrrolo[2,1-c][1,4]benzodiazepine]-10′(5′H)-carbonyl]oxy}methyl)phenyl]-L-alaninamide (“GGVA” disclosed as SEQ ID NO: 85) prepared according to the procedure of Example 3 in WO 2019/065964. The first step of the synthesis is shown in FIG. 18. The triazole ring to be formed in step 1 has geometric isomerization, and the compound obtained in step 1 has a linker as a mixture of the two structures shown as R in FIG. 18.

Step 1: Conjugation of Antibody H10-O18-A-[MSG1-N₃]₂ and Drug-Linker

The operations same as Example 12 were performed using a H10-O18-A-[MSG1-N₃]₂ solution (10.7 mg/mL in 10 mM Acetate Buffer, 5% Sorbitol (pH 5.5), 1.0 mL). As the result, H10-O18-AADC solution (5.0 mL) was obtained.

Characterization: The following characteristic values were obtained by using common operations E (using εA, 280=215424, εA, 329=0, ε D, 280=23155 and 8 D, 329=19492) and F (gradient program 2).

Antibody concentration: 1.68 mg/mL, antibody yield: 8.41 mg (79%), average number of conjugated drug molecules per antibody molecule (n): 1.8

### Example 15—Synthesis of Anti-LPS ADC (Anti-LPS antibody-Conjugate)

The anti-LPS antibody-conjugate is antibody-drug conjugates produced from human IgG recognizing an antigen unrelated to DLL3, and was used as negative controls.

The anti-LPS antibody was produced with reference to the heavy chain full-length and light chain full-length amino acid sequences as shown in SEQ ID NOs: 73 and 74 below (which correspond to SEQ ID NO: 57 and NO: 67 in International Publication No. WO 2015/046505) of h #1G5-H1L1 described in International Publication No. WO 2015/046505. With reference to Example 21 and 36 in WO 2019/065964, glycan remodeling and conjugation reaction was performed to afford the anti-LPS ADC (anti-LPS antibody-Conjugate).

(LPS_Heavy Chain; SEQ ID NO: 73)
QVQLVQSGAEVKKPGASVKVSCKASGYTFTSYWINWVRQAPGQGLEWMG
NIYPGSSSINYNEKFKSRVTITADTSTSTAYMELSSLRSEDTAVYYCAR
TIYNYGSSGYNYAMDYWGQGTLVTVSSASTKGPSVFPLAPSSKSTSGGT
AALGCLVKDYFPEPVTVSWNSGALTSGVHTFPAVLQSSGLYSLSSVVTV
PSSSLGTQTYICNVNHKPSNTKVDKRVEPKSCDKTHTCPPCPAPELLGG
PSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKT
ISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALH
NHYTQKSLSLSPGK
(LPS_Light Chain; SEQ ID NO: 74)
DIVMTQSPDSLAVSLGERATINCKASENVGNSVSWYQQKPGQPPKLLIY
GASNRYTGVPDRFSGSGSGTDFTLTISSLQAEDVAVYYCGQSYSYPYTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

### Example 16—Production of Anti-DLL3 ADC, SC16LD6.5

The anti-DLL3 ADC, SC16LD6.5, was prepared by following steps; The anti-DLL3 antibody, hSC16.56 was produced with reference to WO 2017/031458 A2. The amino acid sequences of the light chain and heavy chain of hSC16.56 (SC16) are represented of SEQ ID NOs: 71 and 72 below (which correspond to SEQ ID NO: 7 and NO: 8 in International Publication No. WO 2017/031458). The drug linker, SG3249, was synthesized according to the previously reported procedure (Med. Chem. Lett. 2016, 7, 983-987). hSC16.56 (SC16) was conjugated with SG3249 according to the reported procedure, Example 7 (a) Maleimide conjugation in WO 2014/130879 A2, to afford SC16LD6.5 (ADC1).

(SEQ ID NO: 71)
QVQLVQSGAEVKKPGASVKVSCKASGYTFTNYGMNWVRQAPGQGLEWMG
WINTYTGEPTYADDFKGRVTMTTDTSTSTAYMELRSLRSDDTAVYYCAR
IGDSSPSDYWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKS
LSLSPGK
(SEQ ID NO: 72)
EIVMTQSPATLSVSPGERATLSCKASQSVSNDVVWYQQKPGQAPRLLIY
YASNRYTGIPARFSGSGSGTEFTLTISSLQSEDFAVYYCQQDYTSPWTF
GQGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

### Example 17—In Vivo Anti-Tumor Effect of Antibody-Drug Conjugate

The antitumor effects of the antibody-drug conjugates were evaluated using animal models derived from immunodeficient mice by the inoculation of DLL3-positive human tumor cell line cells. 4- to 5-week-old BALB/c nude mice (CAnN. Cg-Foxn1 [nu]/CrlCrlj [Foxn1nu/Foxn1nu], Charles River Laboratories Japan inc.) were acclimatized for 3 days or longer under SPF conditions before use in the experiment. The mice were fed with a sterilized solid diet (FR-2, Funabashi Farms Co., Ltd) and given sterilized tap water (which had been prepared by adding a 5 to 15 ppm sodium hypochlorite solution to tap water). The long diameter and short diameter of the inoculated tumor were measured twice a week using electronic digital calipers (CD-15CX, Mitutoyo Corp.), and the volume of the tumor was then calculated according to the following expression.

Tumor volume (mm³=½×Long diameter (mm)×[Short diameter (mm)]²

Each antibody-drug conjugate was diluted with ABS buffer (10 mM acetate buffer, 5% sorbitol, pH 5.5) (Nacalai Tesque, Inc.), and the dilution was intravenously administered at a dose shown in each study to the tail of each mouse. ABS buffer was administered in the same manner as above to a control group (vehicle group). Six mice per group were used in the experiment. The group with the over 3000 mm³ estimated tumor volume were euthanized at that time.

17)-1 Antitumor Effect—(1)

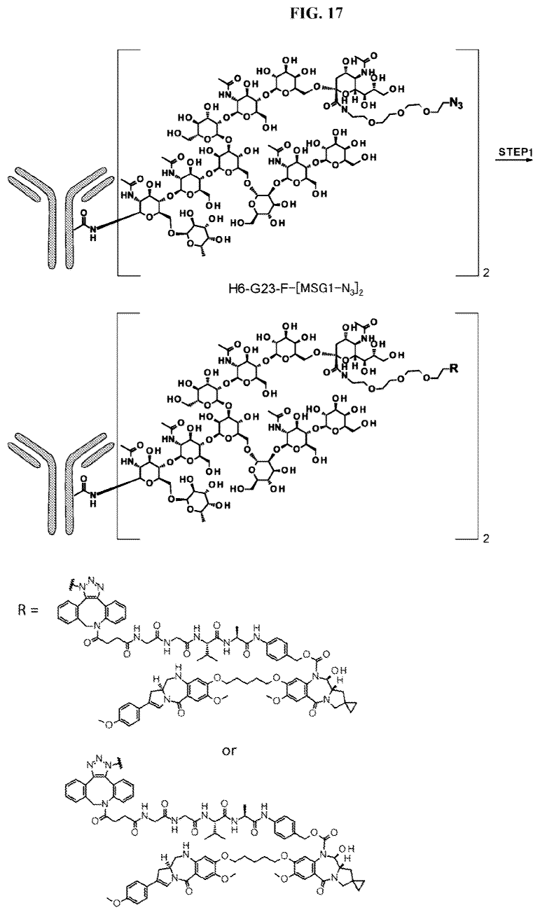

The DLL3-positive human small cell lung cancer cell line NCI-H209 (ATCC) was suspended in Matrigel (Corning Inc.), and the cell suspension was subcutaneously inoculated at a dose of 4×10⁶ cells to the right flank region of each female nude mouse (Day 0). On Day 11, the mice were randomly grouped. On the day of grouping, each of the 3 antibody-drug conjugates (H2-C8-A-Conjugate, H6-G23-F-Conjugate, H10-O18-A-Conjugate), or anti-LPS antibody-Conjugate was intravenously administered at a dose of 0.4 mg/kg to the tail of each mouse. The results are shown in FIG. 19. The abscissa depicts the days after inoculation, and the ordinate depicts estimated tumor volume. The error range depicts a SE value. Arrows indicate the date of administration.

Anti-LPS antibody-Conjugate exhibited no meaningful antitumor effect in this tumor model. All the 3 antibody-drug conjugates (H2-C8-A-Conjugate, H6-G23-F-Conjugate, H10-O18-A-Conjugate) decreased tumor volume after administration, exerted significant tumor regression, and sustained the tumor regression effect for 28 days after administration (FIG. 19). In addition, mice treated with each antibody-drug conjugate showed no significant signs of weight loss, suggesting that the 3 antibody-drug conjugates (H2-C8-A-Conjugate, H6-G23-F-Conjugate, H10-O18-A-Conjugate) are low toxic and safe. The Vehicle and anti-LPS antibody-Conjugate group were euthanized when the estimated tumor volume of at least one of the mice of the groups exceeded 3000 mm³.

17)-2 Antitumor Effect—(2)

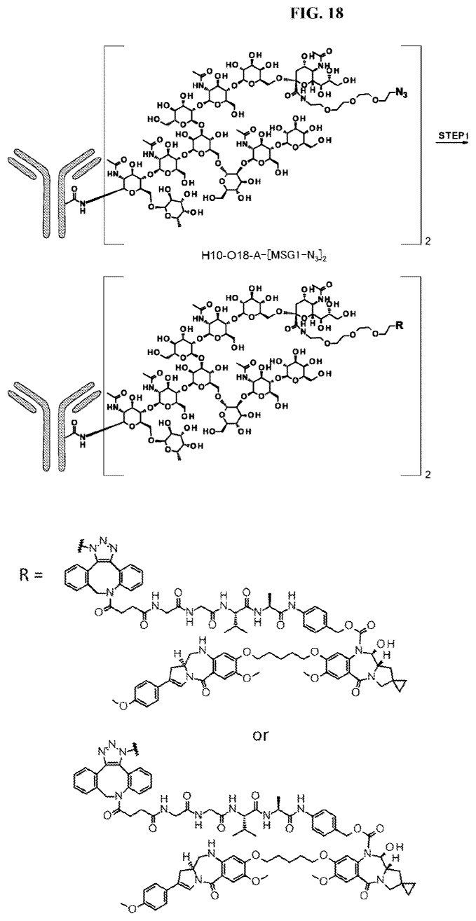

The DLL3-positive human small cell lung cancer cell line NCI-H524 (ATCC) was suspended in Matrigel (Corning Inc.), and the cell suspension was subcutaneously inoculated at a dose of 2.5×10⁶ cells to the right flank region of each female nude mouse (Day 0). On Day 13, the mice were randomly grouped. On the day of grouping, each of the 3 antibody-drug conjugates (H2-C8-A-Conjugate, H6-G23-F-Conjugate, H10-O18-A-Conjugate), or anti-LPS antibody-Conjugate was intravenously administered at a dose of 0.4 mg/kg to the tail of each mouse. The results are shown in FIG. 20. The abscissa depicts the days after inoculation, and the ordinate depicts estimated tumor volume. The error range depicts a SE value. Arrows indicate the date of administration.

Anti-LPS antibody-Conjugate exhibited no antitumor effect in this tumor model. All the 3 antibody-drug conjugates (H2-C8-A-Conjugate, H6-G23-F-Conjugate, H10-O18-A-Conjugate) decreased tumor volume after administration, exerted tumor regression, and H2-C8-A-Conjugate and H10-O18-A-Conjugate sustained the tumor regression effect for 29 days after administration (FIG. 20). In addition, mice treated with each antibody-drug conjugate showed no significant signs of weight loss, suggesting that the 3 antibody-drug conjugates (H2-C8-A-Conjugate, H6-G23-F-Conjugate, H10-O18-A-Conjugate) are low toxic and safe. The Vehicle and anti-LPS antibody-Conjugate group were euthanized when the estimated tumor volume of at least one of the mice of the groups exceeded 3000 mm³.

17)-3 Antitumor Effect—(3)

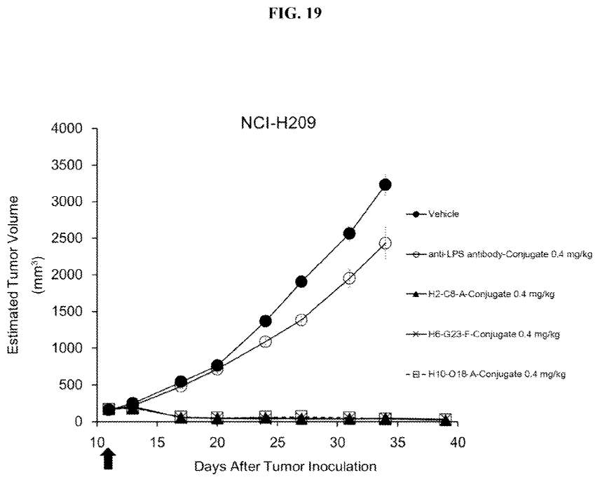

The DLL3-positive human small cell lung cancer cell line NCI-H510A (ATCC) was suspended in Matrigel (Corning Inc.), and the cell suspension was subcutaneously inoculated at a dose of 2.5×10⁶ cells to the right flank region of each female nude mouse (Day 0). On Day 15, the mice were randomly grouped. On the day of grouping, each of the 3 antibody-drug conjugates (H2-C8-A-Conjugate, H6-G23-F-Conjugate, H10-O18-A-Conjugate) or the reference anti-DLL3 antibody-Conjugate (SC16LD6.5) was intravenously administered at a dose of 0.2 mg/kg to the tail of each mouse. The results are shown in FIG. 21. The abscissa depicts the days after inoculation, and the ordinate depicts estimated tumor volume. The error range depicts a SE value. Arrows indicate the date of administration.

The reference anti-DLL3-drug conjugate (SC16LD6.5) exhibited some tumor growth inhibition without tumor regression effect in this tumor model. On the other hand, H2-C8-A-Conjugate, and H10-O18-A-Conjugate decreased tumor volume after administration, exerted tumor regression, and sustained the tumor regression effect for 27 days after administration. H2-C8-A-Conjugate and H10-O18-A-Conjugate further decrease tumor volume than SC16LD6.5. Thus, two antibody-drug conjugates (H2-C8-A-Conjugate, H10-O18-A-Conjugate) of the present invention are superior as antibody-drug conjugates acting as antitumor agents as compared with the SC16LD6.5 antibody drug conjugate. (FIG. 21). In addition, mice treated with each antibody-drug conjugate showed no significant signs of weight loss, suggesting that the three antibody-drug conjugates (H2-C8-A-Conjugate, H6-G23-F-Conjugate, H10-O18-A-Conjugate) are low toxic and safe.

### Example 18—Glycan Remodeling (H2-C8-A-3 Antibody-[MSG1-N₃]₂)

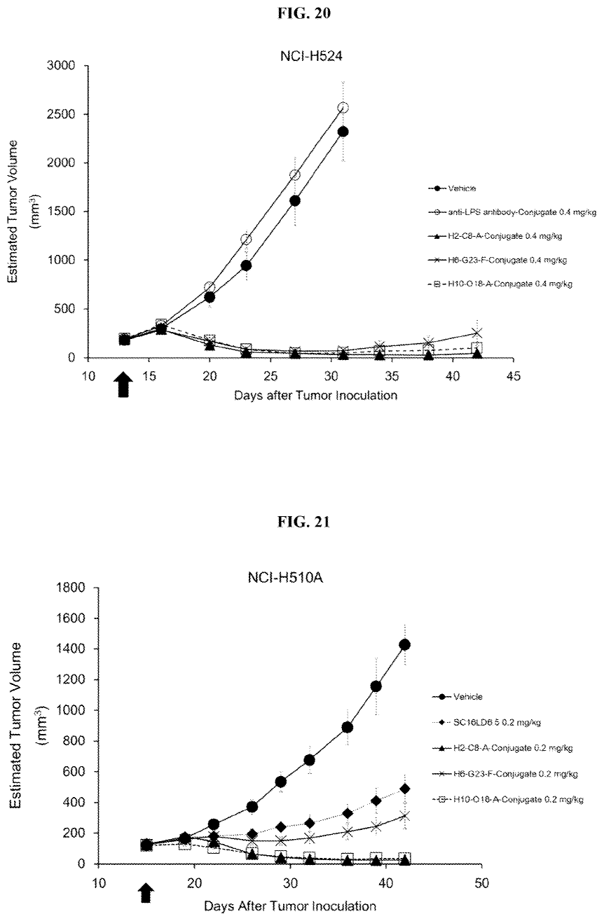

The glycan remodeling of anti-DLL3 antibody H2-C8-A-3 was performed, as shown in the scheme in FIG. 22. This figure shows a scheme in which a linker structure in which an azide group has been introduced to a sialic acid at the non-reducing terminal of an MSG1-type N297 glycan. The linker structures of intermediates formed by introducing an azide group to an N297 glycan are all the same as the structure represented by the formula.

Step 1: Preparation of (Fucα1,6)GlcNAc-H2-C8-A-3 antibody

The H2-C8-A-3 antibody solution (30.0 mg/mL in HBSor (Histidine Buffer Sorbitol), 3.43 mL) was subjected to buffer exchange to 50 mM phosphate buffer (pH 6.0) according to common operation C. Using the resulting H2-C8-A-3 antibody solution (20.0 mg/mL in 50 mM phosphate buffer (pH 6.0), 5.00 mL), the operations same as in step 1 of Example 9 were performed to afford a (Fucα1,6)GlcNAc-H2-C8-A-3 antibody solution (19.8 mg/mL in PB (pH6.0), 4.95 mL).

Step 2: Preparation of H2-C8-A-3 Antibody-[MSG1-N₃]₂

The operations same as in step 2 of Example 9 were performed using (Fucα1,6)GlucNAc-H2-C8-A-3 antibody solution (19.8 mg/mL in PB (pH6.0), 4.95 mL) obtained in the above step 1 to afford a H2-C8-A-3 Antibody-[MSG1-N₃]₂ solution (9.88 mg/mL in 10 mM Acetate Buffer, 5% Sorbitol (pH5.5), 9.30 mL).

### Example 19—Glycan Remodeling (H10-O18-A-3 Antibody-[MSG1-N₃]₂)

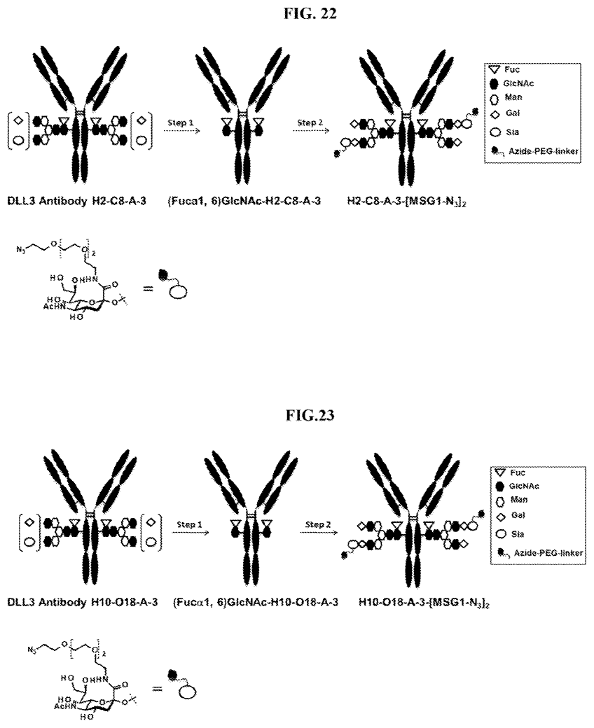

The glycan remodeling of anti-DLL3 antibody H10-O18-A-3 was performed, as shown in the scheme in FIG. 23. This figure shows a scheme in which a linker structure in which an azide group has been introduced to a sialic acid at the nonreducing terminal of an MSG1-type N297 glycan. The linker structures of intermediates formed by introducing an azide group to an N297 glycan are all the same as the structure represented by the formula.

Step 1: Preparation of (Fucα1,6)GlcNAc-H10-O18-A-3 Antibody

The H10-O18-A-3 antibody solution (37.8 mg/mL in HBSor, 2.20 mL) was subjected to buffer exchange to 50 mM phosphate buffer (pH 6.0) according to common operation C. Using the resulting H10-O18-A-3 antibody solution (20.0 mg/mL in 50 mM phosphate buffer (pH 6.0), 3.86 mL), the operations same as in step 1 of Example 9 were performed to afford a (Fucα1,6)GlucNAc-H10-O18-A-3 antibody solution (19.5 mg/mL in PB (pH6.0), 3.77 mL).

Step 2: Preparation of H10-O18-A-3 Antibody-[MSG1-N₃]₂

The operations same as in step 2 of Example 9 were performed using (Fucα1,6)GlucNAc-H10-O18-A-3 antibody solution (19.5 mg/mL in PB (pH 6.0), 3.77 mL) obtained in step 1 to afford a H10-O18-A-3 Antibody-[MSG1-N₃]₂ solution (10.6 mg/mL in 10 mM Acetate Buffer, 5% Sorbitol (pH5.5), 6.50 mL).

### Example 20—Synthesis of H2-C8-A-3-Conjugate

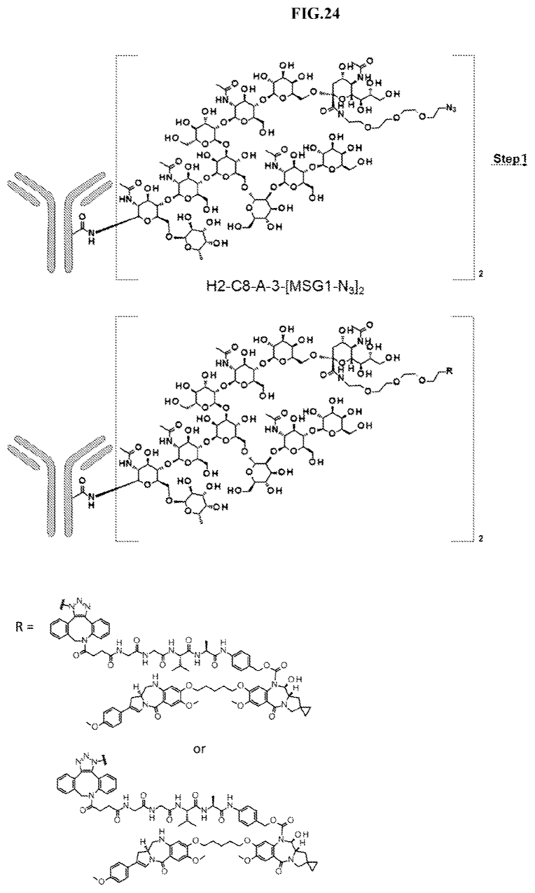

ADCs were synthesized, as illustrated in the following reaction formula, by conjugating the antibody obtained in step 2 of Example 18 and N-[4-(11,12-didehydrodibenzo[b,f]azocin-5(6H)-yl)-4-oxobutanoyl]glycylglycyl-L-valyl-N-[4-({[(11′S,11′aS)-11′-hydroxy-7′-methoxy-8′-[(5-{[(11aS)-7-methoxy-2-(4-methoxyphenyl)-5-oxo-5,10,11,11a-tetrahydro-1H-pyrrolo[2,1-c][1,4]benzodiazepin-8-yl]oxy}pentyl)oxy]-5′-oxo-11′,11′a-dihydro-1′H,3′H-spiro[cyclopropane-1,2′-pyrrolo[2,1-c][1,4]benzodiazepine]-10′(5′H)-carbonyl]oxy}methyl)phenyl]-L-alaninamide (“GGVA” disclosed as SEQ ID NO: 85) prepared according to the procedure of Example 3 in WO 2019/065964. The first step of the synthesis is shown in FIG. 24. The triazole ring to be formed in step 1 has geometric isomerization, and the compound obtained in step 1 has a linker as a mixture of the two structures shown as R in FIG. 24.

Step 1: Conjugation of Antibody H2-C8-A-3-[MSG1-N₃]₂ and Drug-Linker

The operations same as Example 12 were performed using a H2-C8-A-3-[MSG1-N₃]₂ solution (9.88 mg/mL in 10 mM Acetate Buffer, 5% Sorbitol (pH 5.5), 3.50 mL). As the result, H2-C8-A-3 ADC solution (17.5 mL) was obtained and further stable over time.

Characterization: The following characteristic values were obtained by using common operations E (using εA, 280=220420, εA, 329=0, ED, 280=23155 and ED, 329=19492) and F (gradient program 1).

Antibody concentration: 1.65 mg/mL, antibody yield: 29.0 mg (84%), average number of conjugated drug molecules per antibody molecule (n): 1.9 Example 21—Synthesis of H10-O18-A-3-Conjugate

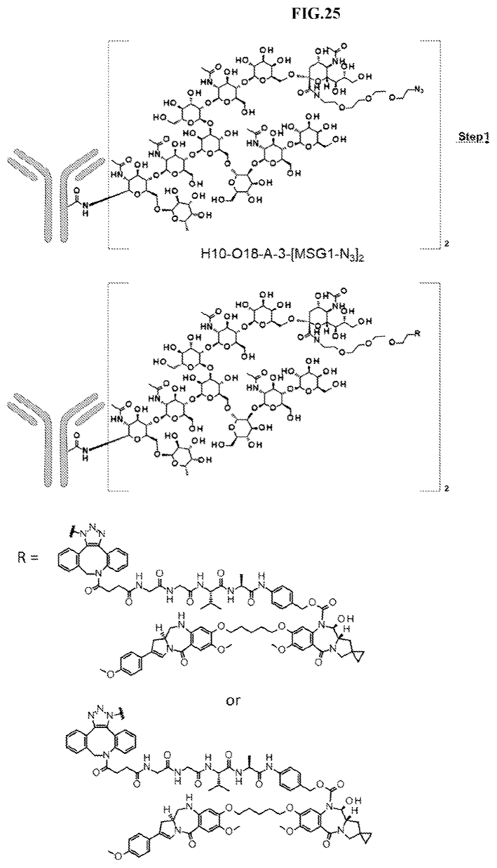

ADCs were synthesized, as illustrated in the following reaction formula, by conjugating the antibody obtained in step 2 of Example 19 and N-[4-(11,12-didehydrodibenzo[b,f]azocin-5(6H)-yl)-4-oxobutanoyl]glycylglycyl-L-valyl-N-[4-({[(11′S,11′aS)-11′-hydroxy-7′-methoxy-8′-[(5-{[(11aS)-7-methoxy-2-(4-methoxyphenyl)-5-oxo-5,10,11,11a-tetrahydro-1H-pyrrolo[2,1-c][1,4]benzodiazepin-8-yl]oxy}pentyl)oxy]-5′-oxo-11′,11′a-dihydro-1′H,3′H-spiro[cyclopropane-1,2′-pyrrolo[2,1-c][1,4]benzodiazepine]-10′(5′H)-carbonyl]oxy}methyl)phenyl]-L-alaninamide (“GGVA” disclosed as SEQ ID NO: 85) prepared according to the procedure of Example 3 in WO 2019/065964. The first step of the synthesis is shown in FIG. 25. The triazole ring to be formed in step 1 has geometric isomerization, and the compound obtained in step 1 has a linker as a mixture of the two structures shown as R in FIG. 25.

Step 1: Conjugation of Antibody H10-O18-A-3-[MSG1-N₃]₂ and Drug-Linker

The operations same as Example 12 were performed using a H10-O18-A-3-[MSG1-N₃]₂ solution (10.6 mg/mL in 10 mM Acetate Buffer, 5% Sorbitol (pH 5.5), 3.50 mL). As the result, H10-O18-A-3 ADC solution (17.5 mL) was obtained and further stable over time.

Characterization: The following characteristic values were obtained by using common operations E (using εA, 280=215380, εA, 329=0, ED, 280=23155 and ED, 329=19492) and F (gradient program 2).

Antibody concentration: 1.79 mg/mL, antibody yield: 31.3 mg (84%), average number of conjugated drug molecules per antibody molecule (n): 1.9 Example 22—In vivo anti-tumor effect of antibody-drug conjugate

Substantially the same protocol as in Example 17 was used.

22)-1 Antitumor Effect—(1)

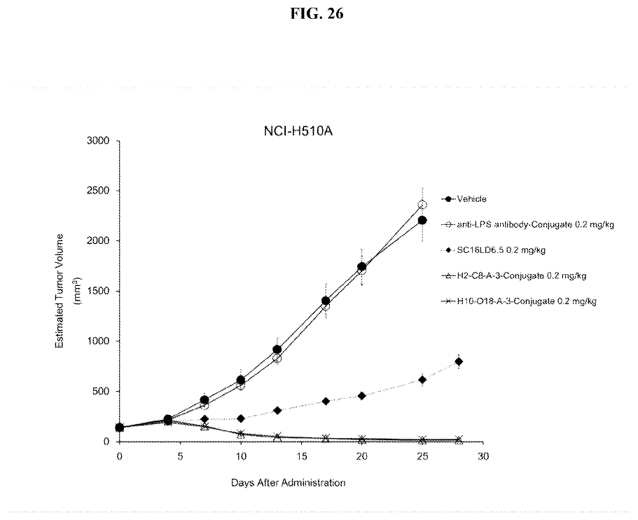

The DLL3-positive human small cell lung cancer cell line NCI-H510A (ATCC) was suspended in Matrigel (Corning Inc.), and the cell suspension was subcutaneously inoculated at a dose of 2.3×10⁶ cells to the right flank region of each female nude mouse. After the tumors were established, the mice were randomly grouped (6 mice per group). On the day of grouping, each of the 2 antibody-drug conjugates (H2-C8-A-3-Conjugate, H10-O18-A-3-Conjugate), the reference anti-DLL3 antibody-Conjugate (SC16LD6.5), or anti-LPS antibody-Conjugate was intravenously administered at a dose of 0.2 mg/kg to the tail of each mouse. The results are shown in FIG. 26. The abscissa depicts the days after administration, and the ordinate depicts estimated tumor volume. The error range depicts a SE value.

Anti-LPS antibody-Conjugate exhibited no meaningful antitumor effect in this tumor model. The reference anti-DLL3-drug conjugate (SC16LD6.5) exhibited some tumor growth inhibition without tumor regression effect in this tumor model. On the other hand, both 2 antibody-drug conjugates (H2-C8-A-3-Conjugate, H10-O18-A-3-Conjugate) decreased tumor volume, and all the tumors were smaller at 28 days after administration than their initial volume in these groups. H2-C8-A-3-Conjugate and H10-O18-A-3-Conjugate further decreased tumor volume than SC16LD6.5. Thus, two antibody-drug conjugates (H2-C8-A-3-Conjugate, H10-O18-A-3-Conjugate) of the present invention are superior as antibody-drug conjugates acting as antitumor agents as compared with the SC16LD6.5 antibody drug conjugate (FIG. 26). In addition, mice treated with each antibody-drug conjugate showed inconsiderable weight loss or no weight loss, suggesting that these antibody-drug conjugates (H2-C8-A-3-Conjugate, H10-O18-A-3-Conjugate) are low toxic and safe. The Vehicle and anti-LPS antibody-Conjugate group were euthanized when the estimated tumor volume of at least one of the mice of the groups exceeded 3000 mm³ or the long diameter of the tumor of at least one of the mice of the groups exceeded 20 mm.

22)-2 Antitumor Effect—(2)

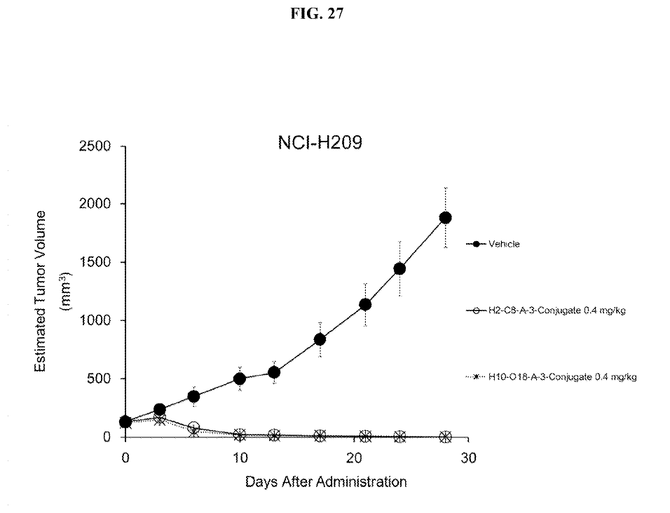

The DLL3-positive human small cell lung cancer cell line NCI-H209 (ATCC) was suspended in Matrigel (Corning Inc.), and the cell suspension was subcutaneously inoculated at a dose of 4×10⁶ cells to the right flank region of each female nude mouse. After the tumors were established, the mice were randomly grouped (5 mice per group). On the day of grouping, each of the 2 antibody-drug conjugates (H2-C8-A-3-Conjugate, H10-O18-A-3-Conjugate) was intravenously administered at a dose of 0.4 mg/kg to the tail of each mouse. The results are shown in FIG. 27. The abscissa depicts the days after administration, and the ordinate depicts estimated tumor volume. The error range depicts a SE value.

Both 2 antibody-drug conjugates (H2-C8-A-3-Conjugate, H10-O18-A-3-Conjugate) decreased tumor volume, and all the tumors were smaller at 28 days after administration than their initial volume in these groups. In addition, mice treated with each antibody-drug conjugate showed inconsiderable weight loss or no weight loss, suggesting that both the 2 antibody-drug conjugates (H2-C8-A-3-Conjugate, H10-O18-A-3-Conjugate) are low toxic and safe.

22)-3 Antitumor Effect—(3)

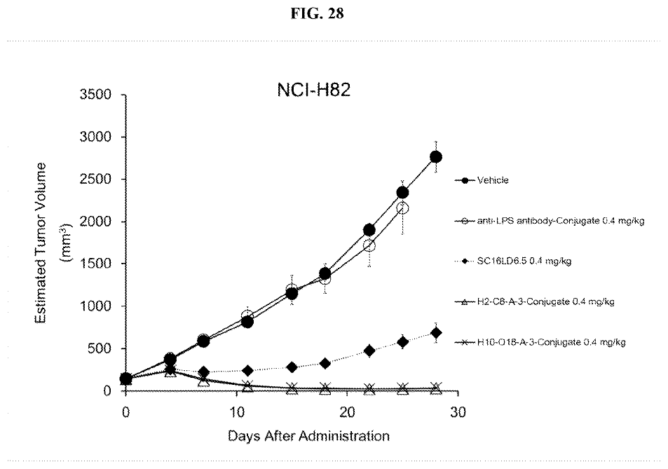

The DLL3-positive human small cell lung cancer cell line NCI-H82 (ATCC) was suspended in Matrigel (Corning Inc.), and the cell suspension was subcutaneously inoculated at a dose of 1×10⁶ cells to the right flank region of each female nude mouse. After the tumors were established, the mice were randomly grouped (6 mice per group). On the day of grouping, each of the 2 antibody-drug conjugates (H2-C8-A-3-Conjugate, H10-O18-A-3-Conjugate), the reference anti-DLL3 antibody-Conjugate (SC16LD6.5) or anti-LPS antibody-Conjugate was intravenously administered at a dose of 0.4 mg/kg to the tail of each mouse. The results are shown in FIG. 28. The abscissa depicts the days after administration, and the ordinate depicts estimated tumor volume. The error range depicts a SE value.

Anti-LPS antibody-Conjugate exhibited no meaningful antitumor effect in this tumor model. The reference anti-DLL3-drug conjugate (SC16LD6.5) exhibited some tumor growth inhibition without tumor regression effect in this tumor model. On the other hand, both 2 antibody-drug conjugates (H2-C8-A-3-Conjugate, H10-O18-A-3-Conjugate) decreased tumor volume, and all the tumors were smaller at 28 days after administration than their initial volume in these groups. H2-C8-A-3-Conjugate and H10-O18-A-3-Conjugate further decreased tumor volume than SC16LD6.5. Thus, two antibody-drug conjugates (H2-C8-A-3-Conjugate, H10-O18-A-3-Conjugate) of the present invention are superior as antibody-drug conjugates acting as antitumor agents as compared with the SC16LD6.5 antibody drug conjugate. In addition, mice treated with each antibody-drug conjugate showed inconsiderable weight loss or no weight loss, suggesting that both the 2 antibody-drug conjugates (H2-C8-A-3-Conjugate, H10-O18-A-3-Conjugate) are low toxic and safe. Anti-LPS antibody-Conjugate group was euthanized when the estimated tumor volume of at least one of the mice of the groups exceeded 3000 mm³.

### Example 23—Toxic or Tolerable Doses of Antibody-Drug Conjugate in ICR Mice

23)-1 Tolerable Doses in ICR Mice—(1)

Crl:CD1(ICR) male mice (Charles River Laboratories Japan, Inc.) at 5-week of age were randomly grouped (5 mice per group). On the day of grouping, each of the 2 antibody-drug conjugates (H2-C8-A-3-Conjugate, or H10-O18-A-3-Conjugate) was intravenously administered at doses of 3, 6, or 10 mg/kg to the tail of each mouse. Mice were observed several times a week for mortality, and body weight was measured for 41 days after the administration.

Mice treated with H2-C8-A-3-Conjugate were all alive during this experiment. Mice treated with H2-C8-A-3-Conjugate showed inconsiderable weight loss or no weight loss at any doses tested (3, 6, 10 mg/kg) during this experiment. Mice treated with H10-O18-A-3-Conjugate were all alive during this experiment. Mice treated with H10-O18-A-3-Conjugate showed inconsiderable weight loss or no weight loss at any doses tested (3, 6, 10 mg/kg) during this experiment.

23)-2 Tolerable Doses in ICR Mice—(2)

Crl:CD1(ICR) female mice (Charles River Laboratories Japan, Inc.) at 5-week of age were randomly grouped (5 mice per group). On the day of grouping, each of the 2 antibody-drug conjugates (H2-C8-A-3-Conjugate, or H10-O18-A-3-Conjugate) was intravenously administered at doses of 3, 6, or 10 mg/kg to the tail of each mouse. Mice were observed several times a week for mortality, and body weight was measured for 41 days after the administration.

Mice treated with H2-C8-A-3-Conjugate were all alive during this experiment. Mice treated with H2-C8-A-3-Conjugate showed no weight loss or inconsiderable weight loss at any doses tested (3, 6, 10 mg/kg) during this experiment. Mice treated with H10-O18-A-3-Conjugate were all alive during this experiment. Mice treated with H10-O18-A-3-Conjugate showed inconsiderable weight loss or no weight loss at any doses tested (3, 6, 10 mg/kg) during this experiment.

### INDUSTRIAL APPLICABILITY

The present invention provides an anti-DLL3 antibody having internalization activity and an antibody-drug conjugate comprising the antibody. The antibody-drug conjugate can be used as a therapeutic drug for cancer, and the like. Indeed, the foregoing examples and disclosure demonstrate that the ADC compositions of the present technology are useful in methods for treating a subject suffering from a DLL3-associated cancer (e.g., small cell lung cancer (SCLC), large cell neuroendocrine carcinoma (LCNEC), neuroendocrine tumors of various tissues including kidney, genitourinary tract (bladder, prostate, ovary, cervix, and endometrium), gastrointestinal tract (stomach, colon), thyroid (medullary thyroid cancer), pancreas and lung, gliomas or pseudo neuroendocrine tumors (pNETs)).

1. An antibody-drug conjugate represented by the following formula: 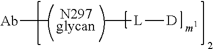
wherein
m¹ represents an integer of 1 to 2;
D represents a drug represented by one of the following formulas: 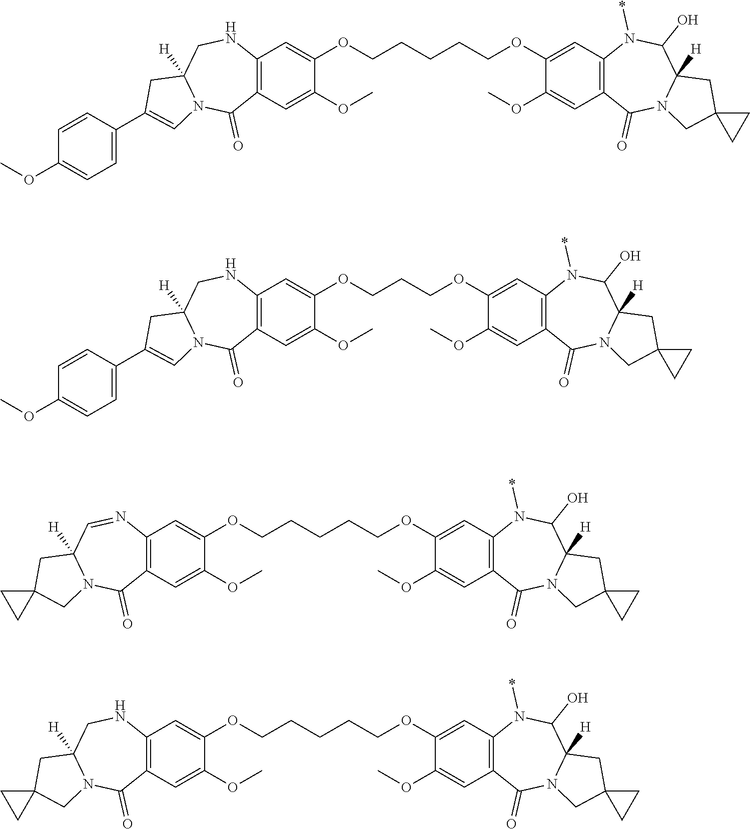 [SMILES: *N1c2cc(OCCCCCOc3cc4c(cc3OC)C(=O)N3C=C(c5ccc(OC)cc5)C[C@H]3CN4)c(OC)cc2C(=O)N2CC3(CC3)C[C@H]2C1O ; *N1c2cc(OCCCOc3cc4c(cc3OC)C(=O)N3C=C(c5ccc(OC)cc5)C[C@H]3CN4)c(OC)cc2C(=O)N2CC3(CC3)C[C@H]2C1O ; *N1c2cc(OCCCCCOc3cc4c(cc3OC)C(=O)N3CC5(CC5)C[C@H]3C=N4)c(OC)cc2C(=O)N2CC3(CC3)C[C@H]2C1O ; *N1c2cc(OCCCCCOc3cc4c(cc3OC)C(=O)N3CC5(CC5)C[C@H]3CN4)c(OC)cc2C(=O)N2CC3(CC3)C[C@H]2C1O]
wherein the asterisk represents bonding to L;
L is a linker linking the N297 glycan of Ab and D;
N297 glycan optionally is remodeled;
Ab represents an anti-DLL3 antibody that specifically binds to DLL-3 or a functional fragment of the antibody.

2. The antibody drug conjugate according to claim 1, wherein the anti-DLL3 antibody comprises a heavy chain immunoglobulin variable domain (V_{H}) and a light chain immunoglobulin variable domain (V_{L}), wherein (a) the V_{H} comprises a V_{H}-CDR1 sequence, a V_{H}-CDR2 sequence, and a V_{H}-CDR3 sequence selected from the group consisting of
(i) SEQ ID NO: 3, SEQ ID NO: 4, and SEQ ID NO: 5, respectively;
(ii) SEQ ID NO: 13, SEQ ID NO: 14, and SEQ ID NO: 15, respectively;
(iii) SEQ ID NO: 23, SEQ ID NO: 24, and SEQ ID NO: 25, respectively; and
(iv) SEQ ID NO: 33, SEQ ID NO: 34, and SEQ ID NO: 35, respectively; and/or
(b) the V_{L} comprises a V_{L}-CDR1 sequence, a V_{L}-CDR2 sequence, and a V_{L}-CDR3 sequence selected from the group consisting of:
(i) SEQ ID NO: 8, SEQ ID NO: 9, and SEQ ID NO: 10, respectively;
(ii) SEQ ID NO: 18, SEQ ID NO: 19, and SEQ ID NO: 20, respectively;
(iii) SEQ ID NO: 28, SEQ ID NO: 29, and SEQ ID NO: 30, respectively; and
(iv) SEQ ID NO: 38, SEQ ID NO: 39, and SEQ ID NO: 40, respectively.

3. The antibody drug conjugate according to claim 1 or 2, wherein the anti-DLL3 antibody comprises a heavy chain immunoglobulin variable domain (V_{H}) and a light chain immunoglobulin variable domain (V_{L}), wherein the combination of (a) the V_{H} comprising a V_{H}-CDR1 sequence, a V_{H}—CDR2 sequence, and a V_{H}-CDR3 sequence and (b) the V_{L} comprising a V_{L}-CDR1 sequence, a V_{L}—CDR2 sequence, and a V_{L}-CDR3 sequence is selected from the group consisting of:
(i) (a) SEQ ID NO: 3, SEQ ID NO: 4, and SEQ ID NO: 5 and (b) SEQ ID NO: 8, SEQ ID NO: 9, and SEQ ID NO: 10, respectively;
(ii) (a) SEQ ID NO: 13, SEQ ID NO: 14, and SEQ ID NO: 15 and (b) SEQ ID NO: 18, SEQ ID NO: 19, and SEQ ID NO: 20, respectively;
(iii) (a) SEQ ID NO: 23, SEQ ID NO: 24, and SEQ ID NO: 25 and (b) SEQ ID NO: 28, SEQ ID NO: 29, and SEQ ID NO: 30, respectively; and
(iv) (a) SEQ ID NO: 33, SEQ ID NO: 34, and SEQ ID NO: 35 and (b) SEQ ID NO: 38, SEQ ID NO: 39, and SEQ ID NO: 40, respectively.

4. The antibody drug conjugate according to any one of claims 1-3, wherein, the anti-DLL3 antibody comprises one or more of the following characteristics:
(a) a light chain immunoglobulin variable domain sequence that is at least 80%, at least 85%, at least 90%, at least 95%, or at least 99% identical to the light chain immunoglobulin variable domain sequence present in any one of SEQ ID NOs: 7, 17, 27, or 37, ; and/or
(b) a heavy chain immunoglobulin variable domain sequence that is at least 80%, at least 85%, at least 90%, at least 95%, or at least 99% identical to the heavy chain immunoglobulin variable domain sequence present in any one of SEQ ID NOs: 2, 12, 22, or 32

5. The antibody drug conjugate according to any one of claims 1-4, wherein, the anti-DLL3 antibody comprises one or more of the following characteristics:
(a) a light chain immunoglobulin variable domain sequence that is at least 80%, at least 85%, at least 90%, at least 95%, or at least 99% identical to the light chain immunoglobulin variable domain sequence present in any one of SEQ ID NOs: 17, 27 or 37; and
(b) a heavy chain immunoglobulin variable domain sequence that is at least 80%, at least 85%, at least 90%, at least 95%, or at least 99% identical to the heavy chain immunoglobulin variable domain sequence present in any one of SEQ ID NOs: 12, 22 or 32.

6. The antibody drug conjugate according to any one of 1-5, wherein the anti-DLL3 antibody comprises a heavy chain immunoglobulin variable domain (V_{H}) and a light chain immunoglobulin variable domain (V_{L}), wherein: (a) the V_{H} comprises an amino acid sequence selected from the group consisting of SEQ ID NO: 2, SEQ ID NO: 12, SEQ ID NO: 22, and SEQ ID NO: 32; and/or (b) the V_{L} comprises an amino acid sequence selected from the group consisting of SEQ ID NO: 7, SEQ ID NO: 17, SEQ ID NO: 27, and SEQ ID NO: 37.

7. The antibody drug conjugate according to any one of claims 1-6, wherein the anti-DLL3 antibody comprises a heavy chain immunoglobulin variable domain (V_{H}) and a light chain immunoglobulin variable domain (V_{L}), wherein: (a) the V_{H} comprises an amino acid sequence selected from SEQ ID NO: 12, SEQ ID NO: 22 or SEQ ID NO: 32, and (b) the V_{L} comprises an amino acid sequence selected from SEQ ID NO: 17, SEQ ID NO: 27 or SEQ ID NO: 37.

8. The antibody drug conjugate according to claim 6, wherein, the V_{H} amino acid sequence and the V_{L} amino acid sequence is selected from the group consisting of: SEQ ID NO: 2 and SEQ ID NO: 7 (7-I1-B), respectively; SEQ ID NO: 12 and SEQ ID NO: 17 (2-C8-A), respectively; SEQ ID NO: 22 and SEQ ID NO: 27 (10-O18-A), respectively; and SEQ ID NO: 32 and SEQ ID NO: 37 (6-G23-F), respectively.

9. The antibody-drug conjugate according to any one of claims 1-8, wherein the antibody-drug conjugate undergoes intracellular internalization when the anti-DLL3 antibody binds to a DLL3 polypeptide expressed on a cell surface (e.g., a tumor cell surface).

10. The antibody-drug conjugate according to any one of claims 1-9, wherein the anti-DLL3 comprises a Fc domain of an isotype selected from the group consisting of IgG1 or the variant thereof, IgG2, IgG3, IgG4, IgA1, IgA2, IgM, IgD, and IgE.

11. The antibody-drug conjugate according to any one of claims 1-10, wherein the anti-DLL3 comprises a heavy chain constant region of SEQ ID NO: 42, 57 or 58.

12. The antibody-drug conjugate according to any one of claims 1-7, wherein the anti-DLL3 antibody is a monoclonal antibody, a chimeric antibody, a humanized antibody, a human antibody or a bispecific antibody.

13. The antibody-drug conjugate according to any one of claims 1-12, wherein the antibody comprises:
(a) a heavy chain comprises amino acid sequence of any one of SEQ ID NOs: 59 to 61 and a light chain comprises amino acid sequence of SEQ ID NO: 62;
(b) a heavy chain comprises amino acid sequence of any one of SEQ ID NOs: 63 to 65 and a light chain comprises amino acid sequence of SEQ ID NO: 66; or
(c) a heavy chain comprises amino acid sequence of any one of SEQ ID NOs: 67 to 69 and a light chain comprises amino acid sequence of SEQ ID NO: 70.

14. The antibody-drug conjugate according to any one of claims 1-13, wherein the antibody comprises:
(a) a heavy chain comprises amino acid sequence of SEQ ID NO: 61 and a light chain comprises amino acid sequence of SEQ ID NO: 62;
(b) a heavy chain comprises amino acid sequence of SEQ ID NO: 65 and a light chain comprises amino acid sequence of SEQ ID NO: 66; or
(c) a heavy chain comprises amino acid sequence of SEQ ID NO: 69 and a light chain comprises amino acid sequence of SEQ ID NO: 70.

15. The antibody drug conjugate according to claim 1, wherein the anti-DLL-3 antibody competes with the antibody according to claim 9 or claim V for binding to DLL-3.

16. The antibody-drug conjugate according to any one of claims 1-15, wherein the anti-DLL3 antibody binds to an epitope on DLL3 that is a conformational epitope or a non-conformational epitope.

17. The antibody-drug conjugate according to any one of claims 1-16, wherein the anti-DLL3 antibody binds to a mammalian DLL3 polypeptide that has an amino acid sequence comprising amino acid residues 27-492 of SEQ ID NO: 50 or SEQ ID NO: 51.

18. The antibody-drug conjugate according to any one of claims 1-17, wherein the heavy chain or the light chain of the antibody has one or two or more modifications or sets of amino acid residues selected from the group consisting of N-linked glycosylation, O-linked glycosylation, N-terminal processing, C-terminal processing, deamidation, isomerization of aspartic acid, oxidation of methionine, addition of a methionine residue to the N-terminus, amidation of a proline residue, the substitutions of two leucine (L) residues to alanine (A) at position 234 and 235 (EU Numbering) of the heavy chain (LALA), a set of amino acid residues Glu (E) at positions 356 and Met (M) at position 358 (EU Numbering) of the heavy chain, a set of Asp (D) at positions 356 and Leucine (L) at position 358 (EU Numbering) of the heavy chain or any combination thereof, conversion of N-terminal glutamine or N-terminal glutamic acid to pyroglutamic acid, and a deletion of one or two amino acids from the carboxyl terminus.

19. The antibody-drug conjugate according to claim 18, wherein one or two amino acids are deleted from the carboxyl terminus of a heavy chain thereof.

20. The antibody-drug conjugate according to claim 19, wherein one amino acid is deleted from each of the carboxyl termini of both of the heavy chains thereof.

21. The antibody-drug conjugate according to any one of claims 18-20, wherein a proline residue at the carboxyl terminus of a heavy chain thereof is further amidated.

22. The antibody-drug conjugate according to any one of claims 1-21, wherein the anti-DLL3 antibody comprises a sugar chain modification that is regulated in order to enhance antibody-dependent cellular cytotoxic activity.

23. The antibody-drug conjugate according to any one of claims 1-22, wherein D is:

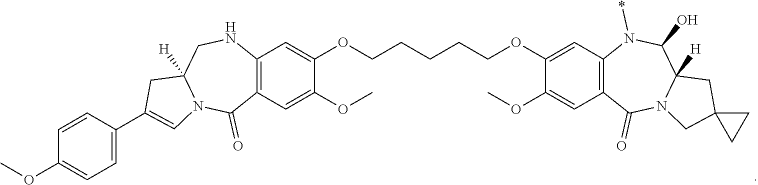 [SMILES: *N1c2cc(OCCCCCOc3cc4c(cc3OC)C(=O)N3C=C(c5ccc(OC)cc5)C[C@H]3CN4)c(OC)cc2C(=O)N2CC3(CC3)C[C@H]2[C@@H]1O]

24. The antibody-drug conjugate according to any one of claims 1-22, wherein D is:

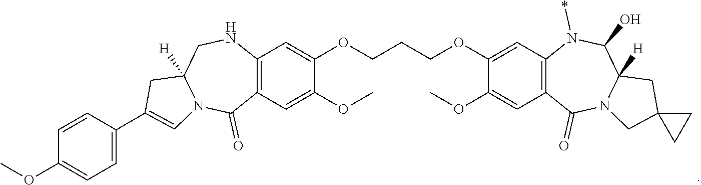 [SMILES: *N1c2cc(OCCCOc3cc4c(cc3OC)C(=O)N3C=C(c5ccc(OC)cc5)C[C@H]3CN4)c(OC)cc2C(=O)N2CC3(CC3)C[C@H]2[C@@H]1O]

25. The antibody-drug conjugate according to any one of claims 1-22, wherein D is:

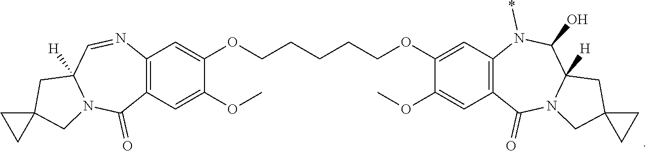 [SMILES: *N1c2cc(OCCCCCOc3cc4c(cc3OC)C(=O)N3CC5(CC5)C[C@H]3C=N4)c(OC)cc2C(=O)N2CC3(CC3)C[C@H]2[C@@H]1O]

26. The antibody-drug conjugate according to any one of claims 1-22, wherein D is:

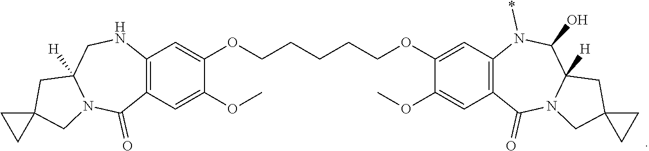 [SMILES: *N1c2cc(OCCCCCOc3cc4c(cc3OC)C(=O)N3CC5(CC5)C[C@H]3CN4)c(OC)cc2C(=O)N2CC3(CC3)C[C@H]2[C@@H]1O]

27. The antibody-drug conjugate according to any one of claims 23-26, wherein the —OH is at the position 11′.

28. The antibody-drug conjugate according to any one of claims 1-27, wherein
L is represented by -Lb-La-Lp-NH—B—CH₂—O(C═O)—*, the asterisk representing bonding to D;
B represents a phenyl group or a heteroaryl group;
Lp represents a linker consisting of an amino acid sequence cleavable in a target cell;
La represents any one selected from the group:
—C(═O)—(CH₂CH₂)n²-C(═O)—,
—C(═O)—(CH₂CH₂)n²-C(═O)—NH—(CH₂CH₂)n³-C(═O)—,
—C(═O)—(CH₂CH₂)n²-C(═O)—NH—(CH₂CH₂O)n³-CH₂—C(═O)—,
—C(═O)—(CH₂CH₂)n²-NH—C(═O)—(CH₂CH₂O)n³-CH₂CH₂—C(═O)—, and
—(CH₂)n⁴-O—C(═O)—;
n² represents an integer of 1 to 3, n³ represents an integer of 1 to 5, and n⁴ represents an integer of 0 to 2; and
Lb represents a spacer bonding La and a glycan or remodeled glycan of Ab.

29. The antibody-drug conjugate according to claim 28, wherein B is any one selected from a 1,4-phenyl group, a 2,5-pyridyl group, a 3,6-pyridyl group, a 2,5-pyrimidyl group, and a 2,5-thienyl group.

30. The antibody-drug conjugate according to claim 29, wherein B is a 1,4-phenyl group.

31. The antibody-drug conjugate according to any one of claims 28-30, wherein Lp is amino acid residues composed of two to seven amino acids.

32. The antibody-drug conjugate according to any one of claims 28-31, wherein Lp is amino acid residues consisting of amino acids selected from glycine, valine, alanine, phenylalanine, glutamic acid, isoleucine, proline, citrulline, leucine, serine, lysine, and aspartic acid.

33. The antibody-drug conjugate according to any one of claims 28-32, wherein Lp is selected from the following group: -GGVA- (SEQ ID NO: 85), -GG-(D-)VA-, -VA-, -GGFG- (SEQ ID NO: 86), -GGPI- (SEQ ID NO: 87), -GGVCit- (SEQ ID NO: 88), -GGVK- (SEQ ID NO: 89), -GG(D-)PI-, and -GGPL- (SEQ ID NO: 90).

34. The antibody-drug conjugate according to any one of claims 28-33, wherein La is selected from the following group:
—C(═O)—CH₂CH₂—C(═O)—, —C(═O)—(CH₂CH₂)₂—C(═O)—,
—C(═O)—CH₂CH₂—C(═O)—NH—(CH₂CH₂)₂—C(═O)—,
—C(═O)—CH₂CH₂—C(═O)—NH—(CH₂CH₂O)₂—CH₂—C(═O)—,
—C(═O)—CH₂CH₂—NH—C(═O)—(CH₂CH₂O)₄—CH₂CH₂—C(═O)—,
—CH₂—OC(═O)—, and —OC(═O)—.

35. The antibody-drug conjugate according to any one of claims 28-34, wherein Lb is represented by the following formula: 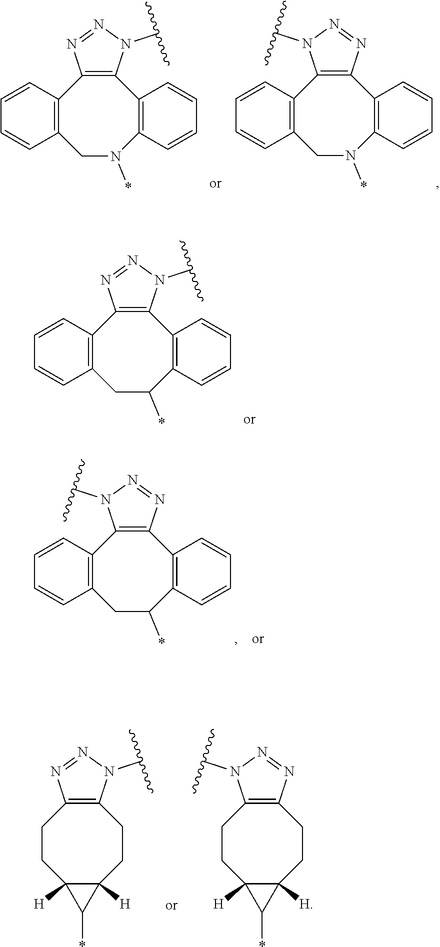
wherein, in each structural formula for Lb shown above,
each asterisk represents bonding to La, and each wavy line represents bonding to a glycan or remodeled glycan of Ab.

36. The antibody-drug conjugate according to any one of claims 28-35, wherein
L is represented by -Lb-La-Lp-NH—B—CH₂—O(C═O)—*, wherein
B is a 1,4-phenyl group;
Lp represents any one selected from the following group: -GGVA- (SEQ ID NO: 85), -GG-(D-)VA-, -VA-, -GGFG- (SEQ ID NO: 86), -GGPI- (SEQ ID NO: 87), -GGVCit- (SEQ ID NO: 88), -GGVK- (SEQ ID NO: 89), and -GGPL- (SEQ ID NO: 90);
La represents any one selected from the following group:
—C(═O)—CH₂CH₂—C(═O)—, —C(═O)—(CH₂CH₂)₂—C(═O)—,
—C(═O)—CH₂CH₂—C(═O)—NH—(CH₂CH₂)₂—C(═O)—,
—C(═O)—CH₂CH₂—C(═O)—NH—(CH₂CH₂O)₂—CH₂—C(═O)—,
—C(═O)—CH₂CH₂—NH—C(═O)—(CH₂CH₂O)₄—CH₂CH₂—C(═O)—,
—CH₂—OC(═O)—, and —OC(═O)—; and
Lb is represented by the following formula: 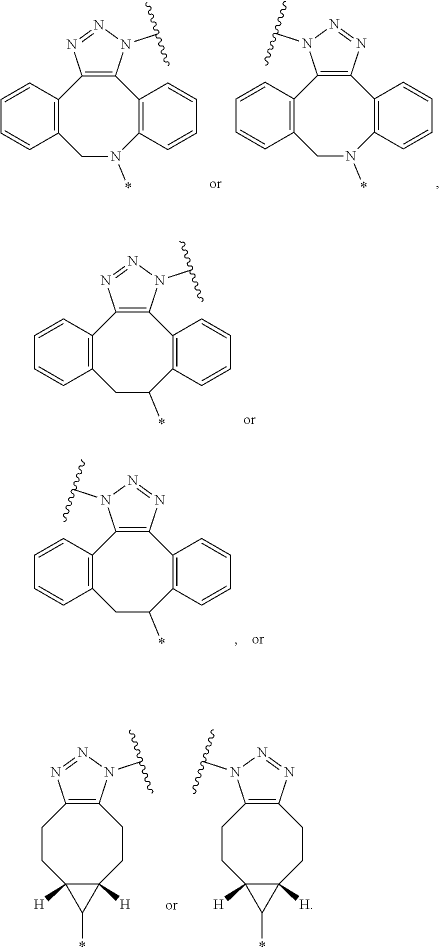
wherein, in each structural formula for Lb shown above,
each asterisk represents bonding to La, and each wavy line represents bonding to a glycan or remodeled glycan of Ab.

37. The antibody-drug conjugate according to any one of claims 28-36, wherein
L is selected from the following group:
—Z¹—C(═O)—CH₂CH₂—C(═O)-GGVA-NH—B—CH₂—OC(═O)— (“GGVA” disclosed as SEQ ID NO: 85),
—Z¹—C(═O)—CH₂CH₂—C(═O)-GG-(D-)VA-NH—B—CH₂—OC(═O)—,
—Z¹—C(═O)—CH₂CH₂—C(═O)—VA-NH—B—CH₂—OC(═O)—,
—Z¹—C(═O)—(CH₂CH₂)₂—C(═O)—VA-NH—B—CH₂—OC(═O)—,
—Z¹—C(═O)—CH₂CH₂—C(═O)-GGPI-NH—B—CH₂—OC(═O)— (“GGPI” disclosed as SEQ ID NO: 87),
—Z¹—C(═O)—CH₂CH₂—C(═O)-GGFG-NH—B—CH₂—OC(═O)— (“GGFG” disclosed as SEQ ID NO: 86),
—Z¹—C(═O)—CH₂CH₂—C(═O)-GGVCit-NH—B—CH₂—OC(═O)— (“GGVCit” disclosed as SEQ ID NO: 88),
—Z¹—C(═O)—CH₂CH₂—C(═O)-GGVK-NH—B—CH₂—OC(═O)— (“GGVK” disclosed as SEQ ID NO: 89),
—Z¹—C(═O)—CH₂CH₂—C(═O)-GGPL-NH—B—CH₂—OC(═O)— (“GGPL” disclosed as SEQ ID NO: 90),
—Z¹—C(═O)—CH₂CH₂—C(═O)—NH—(CH₂CH₂)₂—C(═O)—VA-NH—B—CH₂—OC(═O)—,
—Z¹—C(═O)—CH₂CH₂—C(═O)—NH—(CH₂CH₂O)₂—CH₂—C(═O)—VA-NH—B—CH₂—OC(═O)—,
—Z¹—C(═O)—CH₂CH₂—NH—C(═O)—(CH₂CH₂O)₄—CH₂CH₂—C(═O)—VA-NH—B—CH₂—OC(═O)—,
—Z²—OC(═O)-GGVA-NH—B—CH₂—OC(═O)— (“GGVA” disclosed as SEQ ID NO: 85), and
—Z³—CH₂—OC(═O)-GGVA-NH—B—CH₂—OC(═O)— (“GGVA” disclosed as SEQ ID NO: 85), wherein
Z¹ represents the following structural formula: 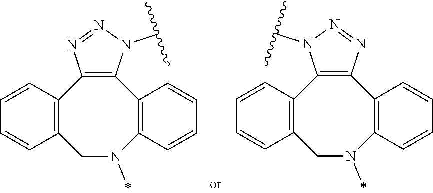
Z² represents the following structural formula: 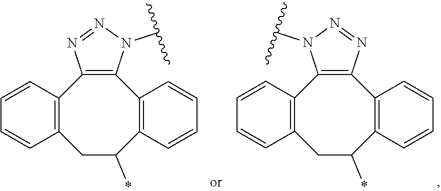
and
Z³ represents the following structural formula: 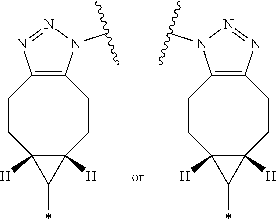
wherein, in each structural formula for Z¹, Z², and Z³,
each asterisk represents bonding to La, each wavy line represents bonding to a glycan or remodeled glycan of Ab; and
B represents a 1,4-phenyl group.

38. The antibody-drug conjugate according to claim 37, wherein
L is selected from the following group:
—Z¹—C(═O)—CH₂CH₂—C(═O)-GGVA-NH—B—CH₂—OC(═O)— (“GGVA” disclosed as SEQ ID NO: 85),
—Z¹—C(═O)—CH₂CH₂—C(═O)—VA-NH—B—CH₂—OC(═O)—,
—Z¹—C(═O)—(CH₂CH₂)₂—C(═O)—VA-NH—B—CH₂—OC(═O)—,
—Z¹—C(═O)—CH₂CH₂—C(═O)-GGVCit-NH—B—CH₂—OC(═O)— (“GGVCit” disclosed as SEQ ID NO: 88), —Z¹—C(═O)—CH₂CH₂—C(═O)—NH—(CH₂CH₂)₂—C(═O)—VA-NH—B—CH₂—OC(═O)—,
—Z¹—C(═O)—CH₂CH₂—C(═O)—NH—(CH₂CH₂O)₂—CH₂—C(═O)—VA-NH—B—CH₂—OC(═O)—, and
—Z¹—C(═O)—CH₂CH₂—NH—C(═O)—(CH₂CH₂O)₄—CH₂CH₂—C(═O)—VA-NH—B—CH₂—OC(═O)—, wherein
B is a 1,4-phenyl group, and Z¹ represents the following structural formula: 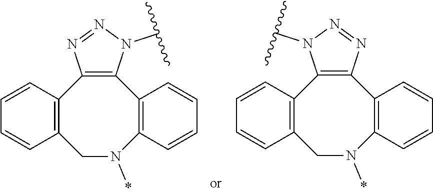
wherein, in the structural formula for Z¹,
each asterisk represents bonding to C(═O) neighboring to Z¹, and each wavy line represents bonding to a glycan or remodeled glycan of Ab.

39. The antibody-drug conjugate according to any one of claims 1-38, wherein the antibody is IgG.

40. The antibody-drug conjugate according to claim 39, wherein the antibody is IgG1 or the variant thereof, IgG2, or IgG4.

41. The antibody-drug conjugate according to any one of claims 1-40, wherein the antibody binds to a tumor cell, and is incorporated and internalizes in the tumor cell.

42. The antibody-drug conjugate according to claim 41, wherein the antibody further has antitumor effect.

43. The antibody-drug conjugate according to any one of claims 1-42, wherein the N297 glycan is a remodeled glycan.

44. The antibody-drug conjugate according to claim 43, wherein the N297 glycan is N297-(Fuc)MSG1, N297-(Fuc)MSG2, or a mixture thereof, or N297-(Fuc)SG, with N297-(Fuc)MSG1, N297-(Fuc)MSG2, and N297-(Fuc)SG having structures represented by the following formulas: 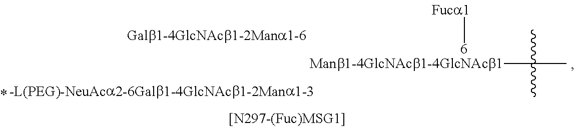
wherein
the wavy line represents bonding to Asn297 of the antibody;
L(PEG) represents —(CH₂CH₂—O)n⁵-CH₂CH₂—NH—, wherein the amino group at the right end is bound via an amide bond to carboxylic acid at the 2-position of a sialic acid at the non-reducing terminal in the 1-3 branched chain of β-Man in the N297 glycan;
the asterisk represents bonding to linker L; and
n⁵ represents an integer of 2 to 10, 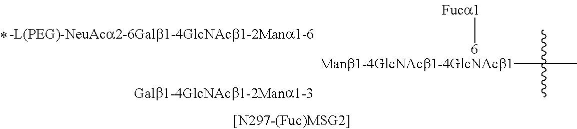
wherein
the wavy line represents bonding to Asn297 of the antibody;
L(PEG) represents —(CH₂CH₂—O)n⁵-CH₂CH₂—NH—, wherein the amino group at the right end is bound via an amide bond to carboxylic acid at the 2-position of a sialic acid at the non-reducing terminal in the 1-6 branched chain of β-Man in the N297 glycan;
the asterisk represents bonding to linker L; and
n⁵ represents an integer of 2 to 10, and 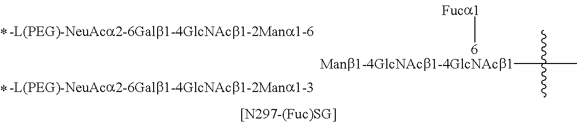
wherein
the wavy line represents bonding to Asn297 of the antibody;
L(PEG) represents —(CH₂CH₂—O)n⁵-CH₂CH₂—NH—, wherein the amino group at the right end is bound via an amide bond to carboxylic acid at the 2-position of a sialic acid at the non-reducing terminal in each of the 1-3 and 1-6 branched chains of β-Man in the N297 glycan;
the asterisk represents bonding to linker L; and
n⁵ represents an integer of 2 to 10.

45. The antibody-drug conjugate according to claim 44, wherein n⁵ is an integer of 2 to 5.

46. The antibody-drug conjugate according to any one of claims 1-45, wherein
m² represents an integer of 1 or 2;
L is selected from the following group:
—Z¹—C(═O)—CH₂CH₂—C(═O)-GGVA-NH—B—CH₂—OC(═O)— (“GGVA” disclosed as SEQ ID NO: 85),
—Z¹—C(═O)—CH₂CH₂—C(═O)—VA-NH—B—CH₂—OC(═O)—,
—Z¹—C(═O)—(CH₂CH₂)₂—C(═O)—VA-NH—B—CH₂—OC(═O)—,
—Z¹—C(═O)—CH₂CH₂—C(═O)-GGVCit-NH—B—CH₂—OC(═O)— (“GGVCit” disclosed as SEQ ID NO: 88),
—Z¹—C(═O)—CH₂CH₂—C(═O)—NH—(CH₂CH₂)₂—C(═O)—VA-NH—B—CH₂—OC(═O)—,
—Z¹—C(═O)—CH₂CH₂—C(═O)—NH—(CH₂CH₂O)₂—CH₂—C(═O)—VA-NH—B—CH₂—OC(═O)—, and —Z¹—C(═O)—CH₂CH₂—NH—C(═O)—(CH₂CH₂O)₄—CH₂CH₂—C(═O)—VA-NH—B—CH₂—OC(═O)—, wherein
B is a 1,4-phenyl group, and Z¹ represents the following structural formula: 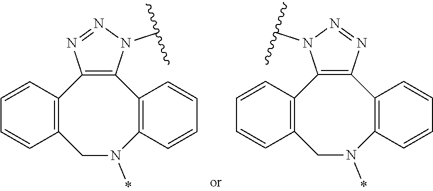
wherein, in the structural formulas for Z¹,
each asterisk represents bonding to C(═O) neighboring to Z¹, and each wavy line represents bonding to the N297 glycan of Ab;
Ab represents an IgG antibody;
the N297 glycan of Ab represents any one of N297-(Fuc)MSG1, N297-(Fuc)MSG2, and a mixture thereof, and N297-(Fuc)SG, with N297-(Fuc)MSG1, N297-(Fuc)MSG2, and N297-(Fuc)SG having structures represented by the following formulas: 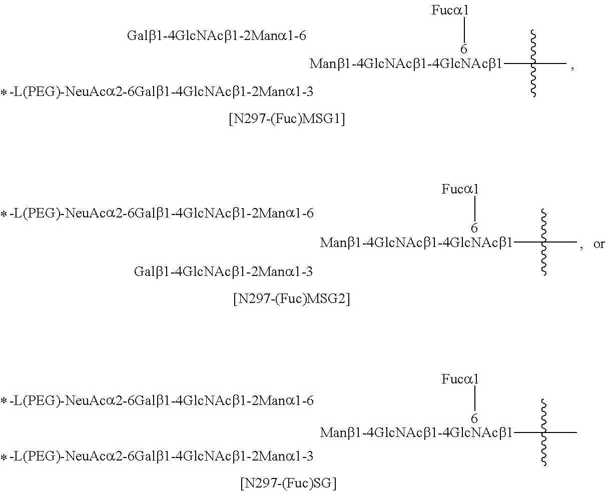
wherein
each wavy line represents bonding to Asn297 of the antibody,
L(PEG) in the N297 glycan represents —NH—CH₂CH₂—(O—CH₂CH₂)n⁵-*, wherein
n⁵ represents an integer of 2 to 5, the amino group at the left end is bound via an amide bond to carboxylic acid at the 2-position of a sialic acid at the non-reducing terminal in each or either one of the 1-3 and 1-6 branched chains of β-Man in the N297 glycan, and each asterisk represents bonding to a nitrogen atom at the 1- or 3-position of the triazole ring of Z¹ in linker L.

47. An antibody-drug conjugate selected from the following group: 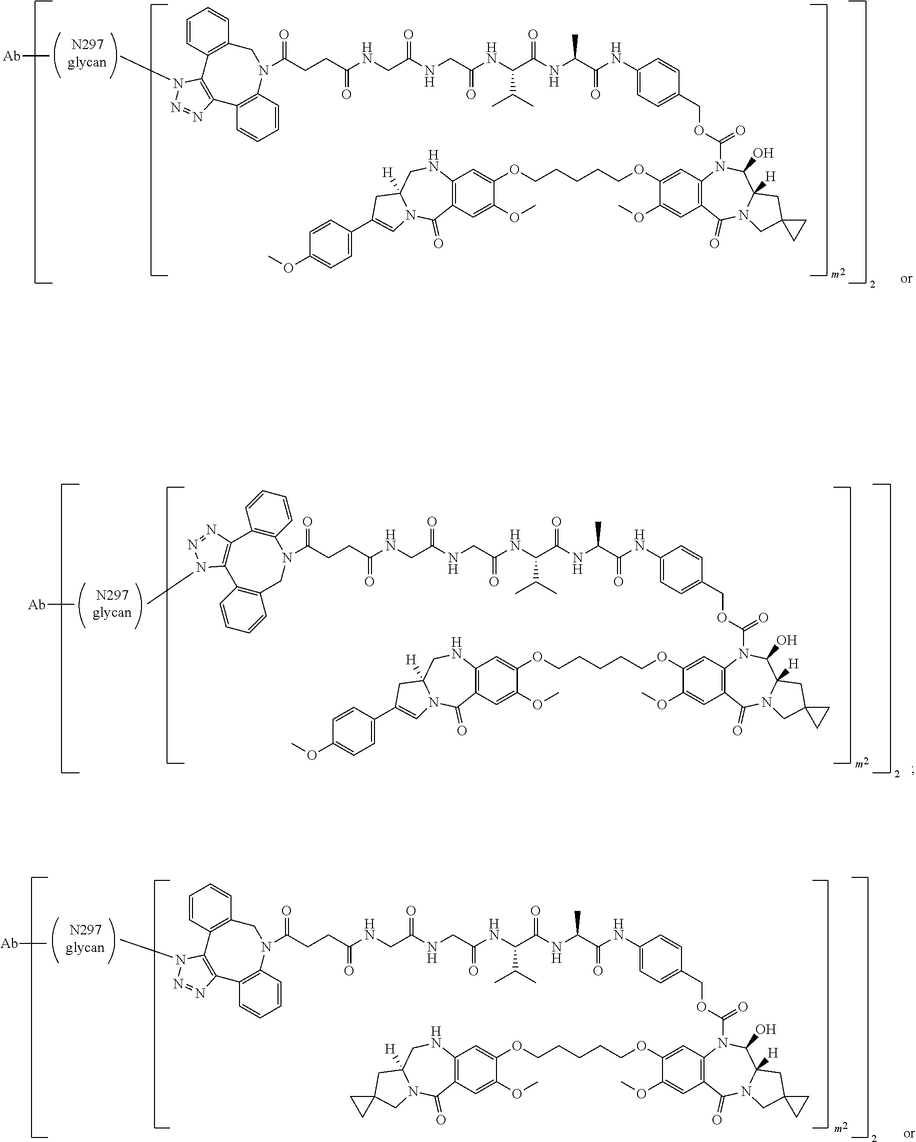 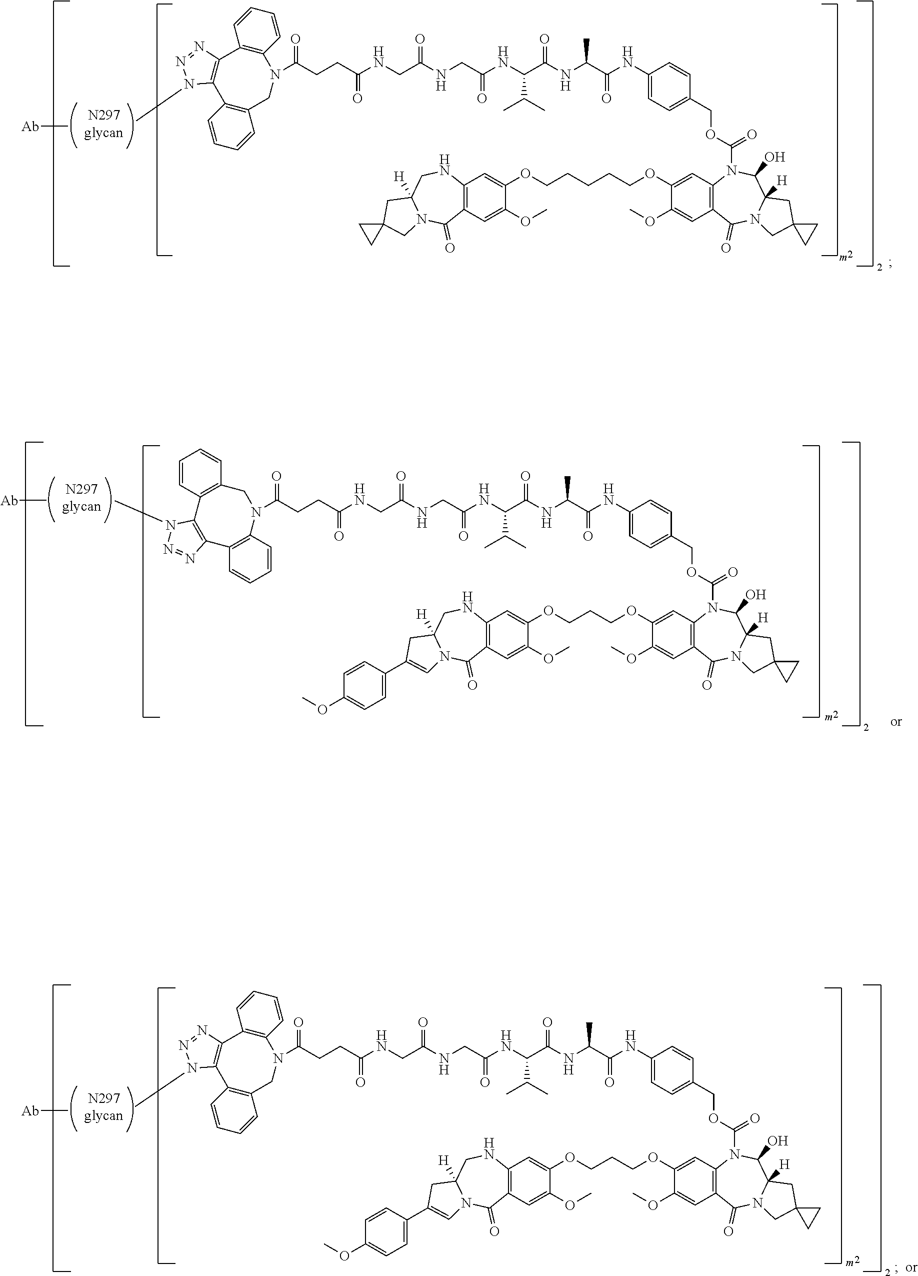 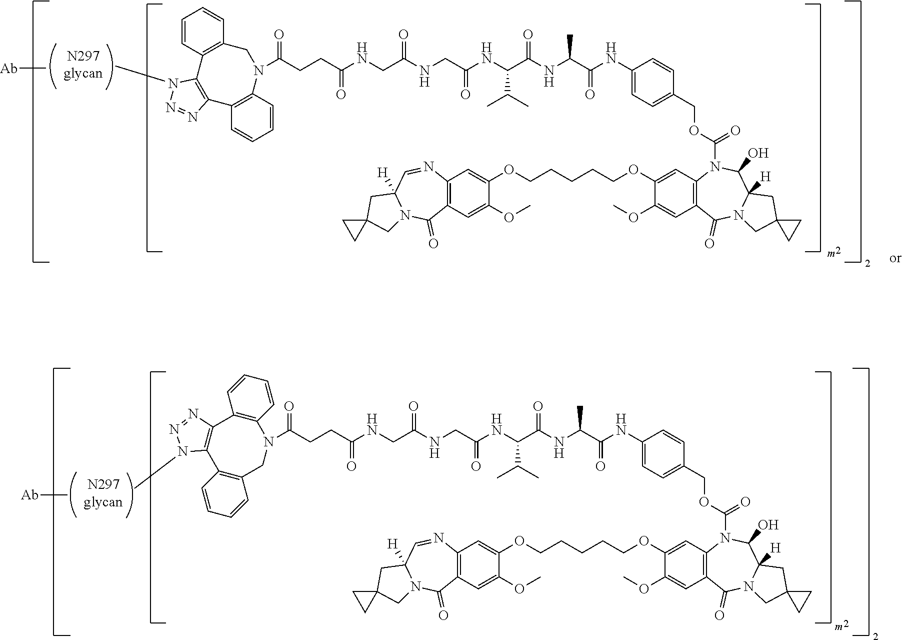
wherein, in each structural formula shown above,
m² represents an integer of 1 or 2;
Ab represents an anti-DLL3 IgG antibody or a functional fragment of the antibody;
the N297 glycan of Ab represents any one of N297-(Fuc)MSG1, N297-(Fuc)MSG2, and a mixture thereof, and N297-(Fuc)SG, with N297-(Fuc)MSG1, N297-(Fuc)MSG2, and N297-(Fuc)SG having structures represented by the following formulas: 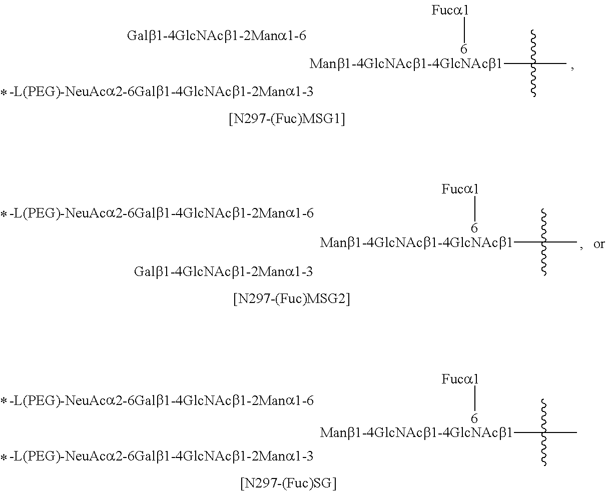
wherein
each wavy line represents bonding to Asn297 of the antibody,
L(PEG) in the N297 glycan represents —NH—CH₂CH₂—(O—CH₂CH₂)₃—*, wherein
the amino group at the left end is bound via an amide bond to carboxylic acid at the 2-position of a sialic acid at the non-reducing terminal in each or either one of the 1-3 and 1-6 branched chains of β-Man in the N297 glycan, and each asterisk represents bonding to a nitrogen atom at the 1- or 3-position of the triazole ring in the corresponding structural formula.

48. The antibody drug conjugate according to claim 47, wherein the antibody comprising a heavy chain constant region of human IgG1 or the variant thereof, human IgG2, or human IgG4.

49. The antibody-drug conjugate according to claim 47 or 48, wherein the antibody comprises a heavy chain constant region of SEQ ID NO: 42, 57 or 58.

50. The antibody drug conjugate according to any one of claims 46-49, wherein the anti-DLL3 antibody comprises a heavy chain immunoglobulin variable domain (V_{H}) and a light chain immunoglobulin variable domain (V_{L}), wherein (a) the V_{H} comprises a V_{H}-CDR1 sequence, a V_{H}-CDR2 sequence, and a V_{H}-CDR3 sequence selected from the group consisting of
(i) SEQ ID NO: 3, SEQ ID NO: 4, and SEQ ID NO: 5, respectively;
(ii) SEQ ID NO: 13, SEQ ID NO: 14, and SEQ ID NO: 15, respectively;
(iii) SEQ ID NO: 23, SEQ ID NO: 24, and SEQ ID NO: 25, respectively; and
(iv) SEQ ID NO: 33, SEQ ID NO: 34, and SEQ ID NO: 35, respectively; and/or
(b) the V_{L} comprises a V_{L}-CDR1 sequence, a V_{L}-CDR2 sequence, and a V_{L}-CDR3 sequence selected from the group consisting of:
(i) SEQ ID NO: 8, SEQ ID NO: 9, and SEQ ID NO: 10, respectively;
(ii) SEQ ID NO: 18, SEQ ID NO: 19, and SEQ ID NO: 20, respectively;
(iii) SEQ ID NO: 28, SEQ ID NO: 29, and SEQ ID NO: 30, respectively; and
(iv) SEQ ID NO: 38, SEQ ID NO: 39, and SEQ ID NO: 40, respectively.

51. The antibody drug conjugate according to claim 50, wherein the anti-DLL3 antibody comprises a heavy chain immunoglobulin variable domain (V_{H}) and a light chain immunoglobulin variable domain (V_{L}), wherein the combination of (a) the V_{H} comprising a V_{H}-CDR1 sequence, a V_{H}—CDR2 sequence, and a V_{H}-CDR3 sequence and (b) the V_{L} comprising a V_{L}-CDR1 sequence, a V_{L}—CDR2 sequence, and a V_{L}-CDR3 sequence is selected from the group consisting of:
(i) (a) SEQ ID NO: 3, SEQ ID NO: 4, and SEQ ID NO: 5 and (b) SEQ ID NO: 8, SEQ ID NO: 9, and SEQ ID NO: 10, respectively;
(ii) (a) SEQ ID NO: 13, SEQ ID NO: 14, and SEQ ID NO: 15 and (b) SEQ ID NO: 18, SEQ ID NO: 19, and SEQ ID NO: 20, respectively;
(iii) (a) SEQ ID NO: 23, SEQ ID NO: 24, and SEQ ID NO: 25 and (b) SEQ ID NO: 28, SEQ ID NO: 29, and SEQ ID NO: 30, respectively; and
(iv) (a) SEQ ID NO: 33, SEQ ID NO: 34, and SEQ ID NO: 35 and (b) SEQ ID NO: 38, SEQ ID NO: 39, and SEQ ID NO: 40, respectively.

52. The antibody-drug conjugate according to any one of claims 47-51, wherein the antibody is an antibody comprising a light chain and a heavy chain in any one combination selected from the group consisting of the following combinations (1) to (4), or a functional fragment of the antibody:
(1) a light chain comprising a variable domain sequence of SEQ ID NO: 7 and a heavy chain comprising a variable domain sequence of SEQ ID NO: 2,
(2) a light chain comprising a variable domain sequence of SEQ ID NO: 17 and a heavy chain comprising a variable domain sequence of SEQ ID NO: 12,
(3) a light chain comprising a variable domain sequence of SEQ ID NO: 27 and a heavy chain comprising a variable domain sequence of SEQ ID NO: 22, and
(4) a light chain comprising a variable domain sequence of SEQ ID NO: 37 and a heavy chain comprising a variable domain sequence of SEQ ID NO: 32.

53. The antibody-drug conjugate according to claim 52, wherein the antibody is an antibody comprising a light chain comprising a variable domain sequence of SEQ ID NO: 17 and a heavy chain comprising a variable domain sequence of SEQ ID NO: 12.

54. The antibody-drug conjugate according to claim 52, wherein the antibody is an antibody comprising a light chain comprising a variable domain sequence of SEQ ID NO: 27 and a heavy chain comprising a variable domain sequence of SEQ ID NO: 22.

55. The antibody-drug conjugate according to claim 52, wherein the antibody is an antibody comprising a light chain comprising a variable domain sequence of SEQ ID NO: 37 and a heavy chain comprising a variable domain sequence of SEQ ID NO: 32.

56. The antibody-drug conjugate according to claim 52, wherein the antibody is an antibody comprising a light chain comprising a variable domain sequence of SEQ ID NO: 7 and a heavy chain comprising a variable domain sequence of SEQ ID NO: 2.

57. The antibody-drug conjugate according to any one of claims 47-56, wherein the antibody comprises:
(a) a heavy chain comprises amino acid sequence of any one of SEQ ID NOs: 59 to 61 and a light chain comprises amino acid sequence of SEQ ID NO: 62;
(b) a heavy chain comprises amino acid sequence of any one of SEQ ID NOs: 63 to 65 and a light chain comprises amino acid sequence of SEQ ID NO: 66; or
(c) a heavy chain comprises amino acid sequence of any one of SEQ ID NOs: 67 to 69 and a light chain comprises amino acid sequence of SEQ ID NO: 70.

58. The antibody-drug conjugate according to any one of claims 47-57, wherein the antibody comprises:
(a) a heavy chain comprises amino acid sequence of SEQ ID NO: 61 and a light chain comprises amino acid sequence of SEQ ID NO: 62;
(b) a heavy chain comprises amino acid sequence of SEQ ID NO: 65 and a light chain comprises amino acid sequence of SEQ ID NO: 66; or
(c) a heavy chain comprises amino acid sequence of SEQ ID NO: 69 and a light chain comprises amino acid sequence of SEQ ID NO: 70.

59. The antibody-drug conjugate according to any one of claims 47-58, wherein the heavy chain or the light chain has one or two or more modifications or sets of amino acid residues selected from the group consisting of N-linked glycosylation, O-linked glycosylation, N-terminal processing, C-terminal processing, deamidation, isomerization of aspartic acid, oxidation of methionine, addition of a methionine residue to the N-terminus, amidation of a proline residue, the substitutions of two leucine (L) residues to alanine (A) at position 234 and 235 (according to EU index) of the heavy chain (LALA), a set of amino acid residues Glu (E) at positions 356 and Met (M) at position 358 (according to EU index) of the heavy chain, a set of Asp (D) at positions 356 and Leucine (L) at position 358 (according to EU index) of the heavy chain or any combination thereof, conversion of N-terminal glutamine or N-terminal glutamic acid to pyroglutamic acid, and a deletion of one or two amino acids from the carboxyl terminus.

60. An antibody-drug conjugate represented by the following formula: 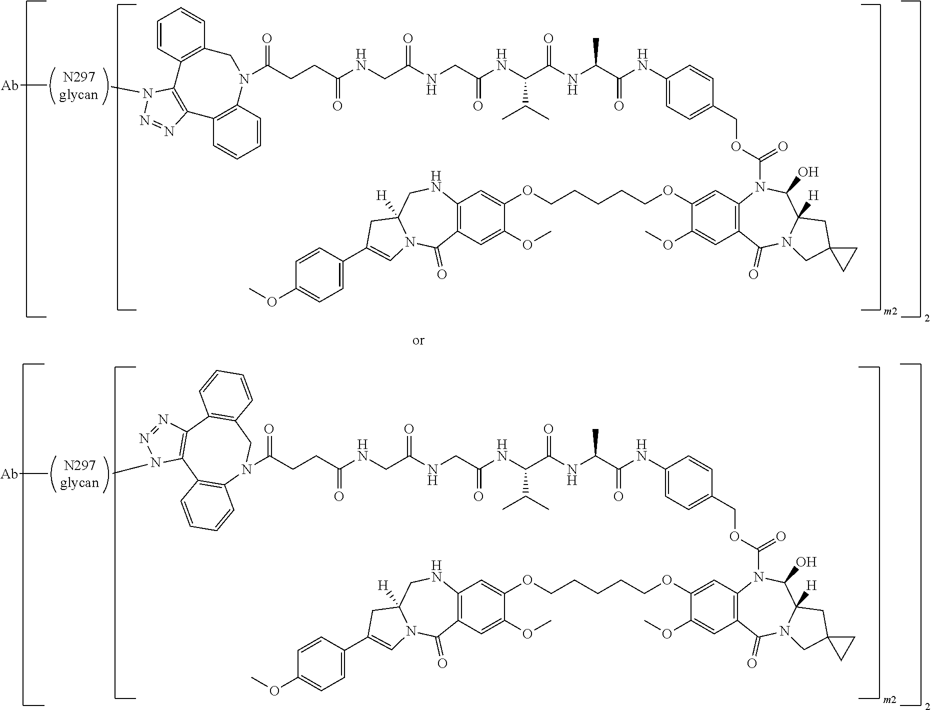
wherein,
m² represents an integer of 1 or 2;
Ab represents an anti-DLL3 IgG antibody which comprises a heavy chain of an amino acid sequence of SEQ ID NO: 61 and a light chain of an amino acid sequence of SEQ ID NO: 62;
the N297 glycan of Ab represents any one of N297-(Fuc)MSG1, N297-(Fuc)MSG2, and a mixture thereof, and N297-(Fuc)SG, with N297-(Fuc)MSG1, N297-(Fuc)MSG2, and N297-(Fuc)SG having structures represented by the following formulas: 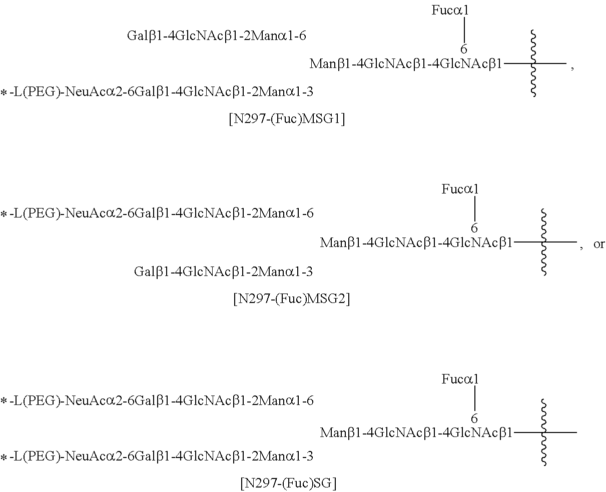
wherein
each wavy line represents bonding to Asn297 of the antibody,
L(PEG) in the N297 glycan represents —NH—CH₂CH₂—(O—CH₂CH₂)₃—*, wherein
the amino group at the left end is bound via an amide bond to carboxylic acid at the 2-position of a sialic acid at the non-reducing terminal in each or either one of the 1-3 and 1-6 branched chains of β-Man in the N297 glycan, and each asterisk represents bonding to a nitrogen atom at the 1- or 3-position of the triazole ring in the corresponding structural formula.

61. An antibody-drug conjugate represented by the following formula: 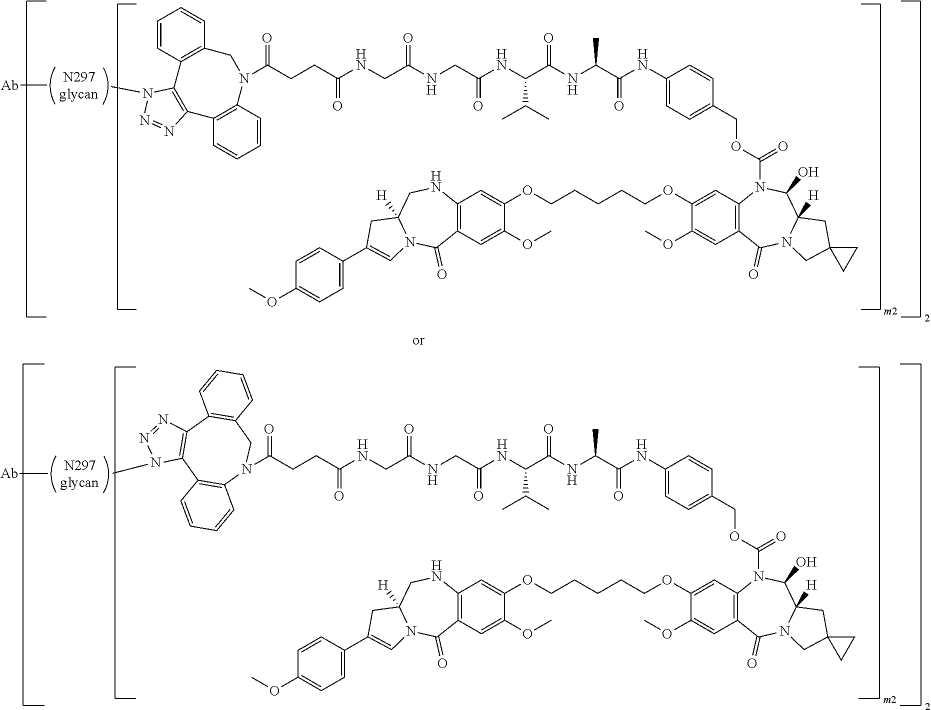
wherein,
m² represents an integer of 1 or 2;
Ab represents an anti-DLL3 IgG antibody which comprises a heavy chain of an amino acid sequence of SEQ ID NO: 65 and a light chain of an amino acid sequence of SEQ ID NO: 66;
the N297 glycan of Ab represents any one of N297-(Fuc)MSG1, N297-(Fuc)MSG2, and a mixture thereof, and N297-(Fuc)SG, with N297-(Fuc)MSG1, N297-(Fuc)MSG2, and N297-(Fuc)SG having structures represented by the following formulas: 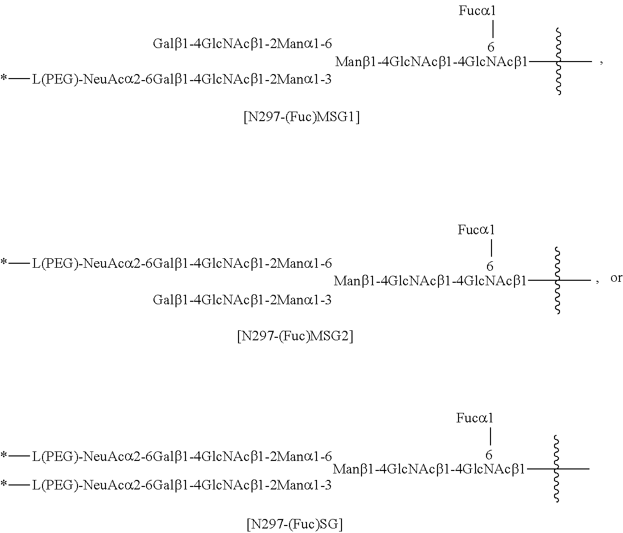
wherein
each wavy line represents bonding to Asn297 of the antibody,
L(PEG) in the N297 glycan represents —NH—CH₂CH₂—(O—CH₂CH₂)₃—*, wherein
the amino group at the left end is bound via an amide bond to carboxylic acid at the 2-position of a sialic acid at the non-reducing terminal in each or either one of the 1-3 and 1-6 branched chains of β-Man in the N297 glycan, and each asterisk represents bonding to a nitrogen atom at the 1- or 3-position of the triazole ring in the corresponding structural formula.

62. An antibody-drug conjugate represented by the following formula: 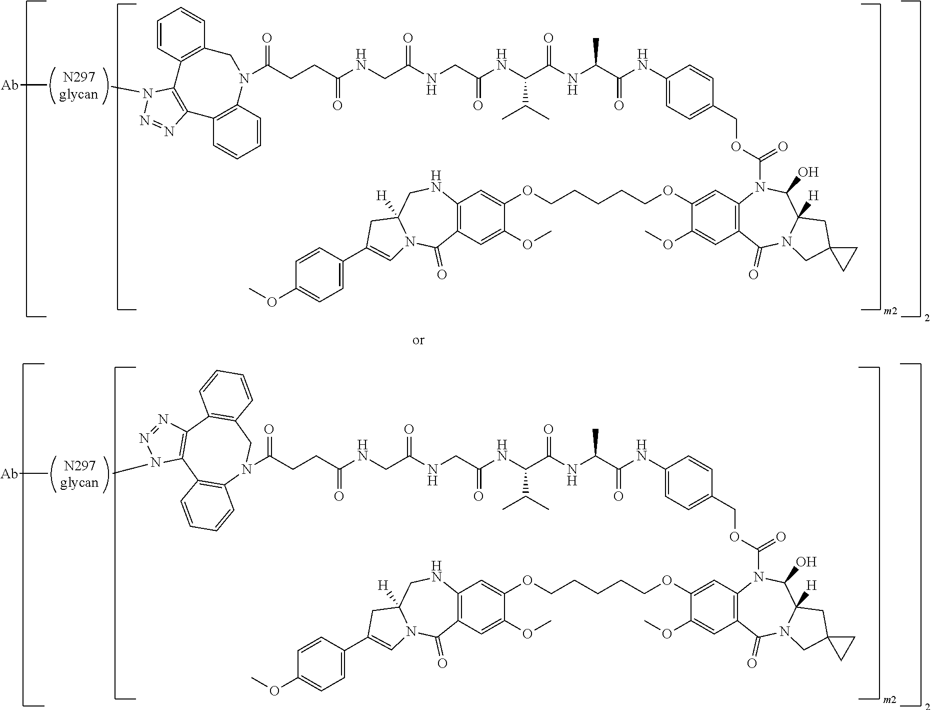
wherein,
m² represents an integer of 1 or 2;
Ab represents an anti-DLL3 IgG antibody which comprises a heavy chain of an amino acid sequence of SEQ ID NO: 69 and a light chain of an amino acid sequence of SEQ ID NO: 70;
the N297 glycan of Ab represents any one of N297-(Fuc)MSG1, N297-(Fuc)MSG2, and a mixture thereof, and N297-(Fuc)SG, with N297-(Fuc)MSG1, N297-(Fuc)MSG2, and N297-(Fuc)SG having structures represented by the following formulas: 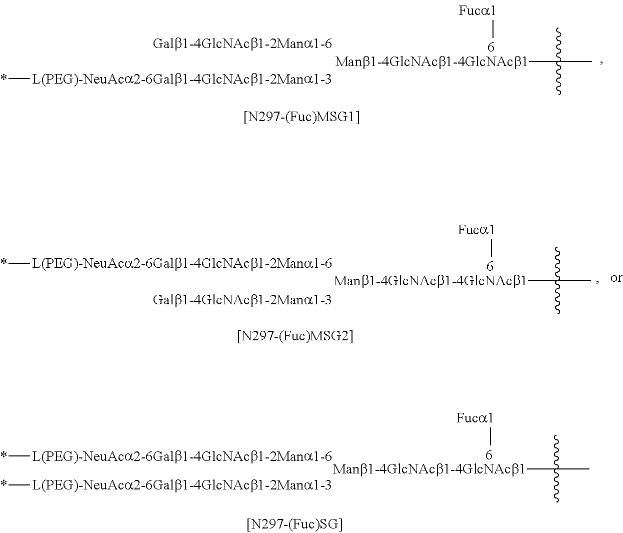
wherein
each wavy line represents bonding to Asn297 of the antibody,
L(PEG) in the N297 glycan represents —NH—CH₂CH₂—(O—CH₂CH₂)₃—*, wherein
the amino group at the left end is bound via an amide bond to carboxylic acid at the 2-position of a sialic acid at the non-reducing terminal in each or either one of the 1-3 and 1-6 branched chains of β-Man in the N297 glycan, and each asterisk represents bonding to a nitrogen atom at the 1- or 3-position of the triazole ring in the corresponding structural formula.

63. A pharmaceutical composition comprising the antibody-drug conjugate according to any one of claims 1-62, a salt thereof, or a hydrate of the conjugate or the salt.

64. The pharmaceutical composition according to claim 63, which is an antitumor drug.

65. The pharmaceutical composition according to claim 63 or 64, for use in treating a tumor, wherein the tumor is a tumor expressing DLL3.

66. The pharmaceutical composition according to claim 64 or 65, wherein the tumor is small cell lung cancer (SCLC), large cell neuroendocrine carcinoma (LCNEC), neuroendocrine tumors of various tissues including kidney, genitourinary tract (bladder, prostate, ovary, cervix, and endometrium), gastrointestinal tract (stomach, colon), thyroid (medullary thyroid cancer), pancreas and lung, gliomas or pseudo neuroendocrine tumors (pNETs).

67. A method for producing a glycan-remodeled antibody, the method comprising the steps of:
i) culturing the host cell comprising a nucleic acid encoding an anti-DLL3 antibody and collecting a targeted antibody from the culture obtained;
ii) treating the antibody obtained in step i) with hydrolase to produce a (Fucα1,6)GlcNAc-antibody; and
iii)-1 reacting a glycan donner molecule and the (Fucα1,6)GlcNAc-antibody in the presence of transglycosidase, the glycan donner molecule obtained by introducing a PEG linker having an azide group to the carbonyl group of carboxylic acid at the 2-position of a sialic acid in MSG (9) or SG (10) and oxazolinating the reducing terminal, or
iii)-2 reacting the (Fucα1,6)GlcNAc-antibody and a glycan donner molecule in the presence of transglycosidase, the glycan donner molecule obtained by introducing a PEG linker having an azide group to the carbonyl group of carboxylic acid at the 2-position of a sialic acid in (MSG-)Asn or (SG-)Asn with an α-amino group optionally protected and to the carbonyl group of carboxylic acid in the Asn, causing action of hydrolase, and then oxazolinating the reducing terminal or
iii)-3 reacting the (Fucα1,6)GlcNAc-antibody and a glycan donner molecule in the presence of two glycosyltransferases, wherein the glycan donner molecule is (SG-)Asn or (MSG-)Asn with an azide group introduced in sialic acid thereof.

68. The method according to claim 67, further comprising the step of purifying the (Fucα1,6)GlcNAc-antibody through purification of a reaction solution in step ii) with a hydroxyapatite column.

69. A method for producing the antibody-drug conjugate form according to any one of claims 1-62, the method comprising the steps of:
i) producing a glycan-remodeled antibody by using the method according to claim 56 or 57; and
ii) reacting a drug-linker having DBCO and an azide group in a glycan of the glycan-remodeled antibody made in step i).

70. A glycan-remodeled antibody obtained by using the method according to claim 67 or 68.

71. An antibody-drug conjugate obtained by using the method according to claim 69.

72. A method for treating a tumor, comprising administering to an individual with a tumor an antibody-drug conjugate according to any one of claims 1 to 62, a salt of the antibody-drug conjugate, or a hydrate of the antibody-drug conjugate or the salt.

73. A method for treating a tumor, which comprises administering a pharmaceutical composition comprising at least one component selected from the antibody-drug conjugate according to any one of claims 1 to 62, a salt thereof, and a hydrate of the conjugate or the salt, and at least one antitumor drug to an individual, simultaneously, separately, or continuously.

74. The method according to claim 72 or 73, wherein the tumor is a tumor expressing DLL3.

75. The method according to any one of claims 72-74, wherein the tumor is small cell lung cancer (SCLC), large cell neuroendocrine carcinoma (LCNEC), neuroendocrine tumors of various tissues including kidney, genitourinary tract (bladder, prostate, ovary, cervix, and endometrium), gastrointestinal tract (stomach, colon), thyroid (medullary thyroid cancer), pancreas and lung, gliomas or pseudo neuroendocrine tumors (pNETs).
